(19) United States
(12) Patent Application Publication
(10) Pub. No.: US 2019/0033329 A1
(43) Pub. Date: Jan. 31, 2019

(54) METHODS AND COMPOSITIONS FOR DETECTING AND TREATING SCHIZOPHRENIA

(71) Applicants: PRESIDENT AND FELLOWS OF HARVARD COLLEGE, CAMBRIDGE, MA (US); CHILDREN'S MEDICAL CENTER CORPORATION, BOSTON, MA (US)

(72) Inventors: STEVEN A. MCCARROLL, CAMBRIDGE, MA (US); ASWIN SEKAR, CAMBRIDGE, MA (US); MICHAEL C. CARROLL, BOSTON, MA (US); BETH STEVENS, BOSTON, MA (US)

(73) Assignees: PRESIDENT AND FELLOWS OF HARVARD COLLEGE, CAMBRIDGE, MA (US); CHILDREN'S MEDICAL CENTER CORPORATION, BOSTON, MA (US)

(21) Appl. No.: 16/072,382

(22) PCT Filed: Jan. 24, 2017
(86) PCT No.: PCT/US2017/014757

§ 371 Date: Jul. 24, 2018

Related U.S. Application Data
(60) Provisional application No. 62/286,867, filed on Jan. 25, 2016.

(51) Int. Cl. G01N 33/68; G01N 33/50; C07K 14/47; C12Q 1/6883

(52) U.S. Cl. CPC G01N 33/6896; G01N 33/5023; C07K 14/472; C12Q 2600/156; G01N 2800/302; G01N 2333/4716; C12Q 2600/158; C12Q 1/6883

(57) ABSTRACT

The invention provides methods of treating schizophrenia in a subject, including for example, administering to the subject an agent that inhibits expression or activity of a C4A polynucleotide or polypeptide. The invention also provides methods of identifying a subject having or at risk of developing schizophrenia involving measuring or detecting an alteration in the level, copy number, and/or sequence of complement component C4A or complement component C4B relative to a reference.

### CROSS-REFERENCE TO RELATED APPLICATIONS

This application claims priority to and the benefit of U.S. Provisional Application No. 62/286,867, filed Jan. 25, 2016, the disclosure of which is incorporated herein by reference in its entirety.

### STATEMENT OF RIGHTS TO INVENTIONS MADE UNDER FEDERALLY SPONSORED RESEARCH

This invention was made with government support under Grant Nos. R01 HG006855, U01 MH105641, and R01 MH077139 awarded by the National Institutes of Health. The government has certain rights in the invention.

### BACKGROUND OF THE INVENTION

Schizophrenia is a heritable psychiatric disorder involving impairments in cognition, perception and motivation that usually manifest late in adolescence or early in adulthood. The pathogenic mechanisms underlying schizophrenia are unknown, but observers have repeatedly noted pathological features involving excessive loss of gray matter and reduced numbers of synaptic structures on neurons. While treatments exist for the psychotic symptoms of schizophrenia, there is no mechanistic understanding of, nor effective therapies to prevent or treat, the cognitive impairments and deficit symptoms of schizophrenia, its earliest and most constant features. New methods of identifying and treating patients having or at risk of developing schizophrenia are urgently needed.

### SUMMARY OF THE INVENTION

As described below, the present invention features compositions and methods for (i) identifying a subject having or at risk of developing schizophrenia, (ii) monitoring treatment for schizophrenia, and (iii) treating or preventing schizophrenia in a subject.

In one aspect, the invention provides a method of treating schizophrenia in a subject. The method contains the step of administering to the subject an agent that inhibits the expression or activity of a complement component 4A (C4A) polypeptide or polynucleotide.

In another aspect, the invention provides a method of treating a subject having a neurodegenerative disease or disorder characterized by increased levels, activity, or expression of a complement component 4A (C4A) polypeptide or polynucleotide (e.g. Alzheimer's Disease, glaucoma, or age-related macular degeneration) by administering to the subject an agent that inhibits the expression or activity of a complement component 4A (C4A) polypeptide or polynucleotide.

In another aspect, the invention provides a method of reducing an interaction between a neuron and microglia and/or reducing synaptic elimination in a subject, the method involving the step of contacting a microglia or neuron (e.g., at a synapse) with an agent that inhibits the expression or activity of a complement component 4A (C4A) polypeptide or polynucleotide. In various embodiments, one or more of the microglia or neuron is contacted with the agent in vitro or in vivo (e.g., in a subject). In certain embodiments, engulfment of synapses by microglia is reduced. In some embodiments, the method involves administering an agent that inhibits the expression or activity of a complement component 4A (C4A) polypeptide or polynucleotide to the subject. In various embodiments, the agent is administered to the subject intrathecally.

In various embodiments, the agent inhibits the expression or activity of a complement component 4A (C4A) polypeptide or polynucleotide. In some embodiments, the agent inhibits the expression or activity of a complement component 4B (C4B) polypeptide or polynucleotide. In some other embodiments, the agent does not inhibit the expression or activity of a complement component 4B (C4B) polypeptide or polynucleotide. In some embodiments, the agent is an antibody or an inhibitory nucleic acid. In certain embodiments, the antibody specifically binds an epitope containing the amino acid sequence PCPVLD. In particular embodiments, the antibody does not bind an epitope containing the amino acid sequence LSPVIH. In various embodiments of any one of the aspects delineated herein, the subject is human.

In another aspect, the invention provides a method of treating schizophrenia in a pre-selected subject, the method containing the step of administering a schizophrenia treatment to the subject, where the subject is pre-selected by detecting an increase in a level of a complement component 4A (C4A) polynucleotide or polypeptide, an increase in a combined level of C4A and complement component 4B (C4B) polynucleotide or polypeptide, an increase in copy number of complement component 4A (C4A), and/or an alteration in a sequence of C4A or C4B polynucleotide relative to a reference in a biological sample obtained from the subject.

In yet another aspect, the invention provides a method of monitoring treatment progress in a subject having schizophrenia and administered with a schizophrenia treatment. The method contains the step of measuring a level of C4A polypeptide or polynucleotide or a combined level of C4A and C4B polypeptide or polynucleotide relative to a reference level in a biological sample obtained from the subject, where a decrease in the level or combined level indicates the subject is responsive to the schizophrenia treatment.

In still another aspect, the invention provides a method of determining efficacy of a schizophrenia treatment in a subject. The method contains the step of measuring a level of C4A polypeptide or polynucleotide or a combined level of C4A and C4B polypeptide or polynucleotide relative to a reference level in a biological sample obtained from the subject, where a decrease in the level or combined level indicates the the schizophrenia treatment is efficacious.

In another aspect, the invention provides method of characterizing a subject having a mental disorder. The method contains the step of measuring a level of a complement component 4A (C4A) polynucleotide or polypeptide, a combined level of C4A and complement component 4B (C4B) polynucleotide or polypeptide, a copy number of C4A polynucleotide, and/or a sequence of C4A and/or C4B polynucleotide relative to a reference in a biological sample obtained from the subject, where an increase in the level of C4A polynucleotide or polypeptide, an increase in the combined level of C4A and C4B polynucleotide or polypeptide, an increase in C4A copy number and/or an alteration in a sequence of C4A or C4B polynucleotide indicates the subject has schizophrenia or is at risk of developing schizophrenia.

In yet another aspect, the invention provides a method of identifying a subject having or at risk of developing schizophrenia, the method containing the step of measuring a level of a complement component 4A (C4A) polynucleotide or polypeptide, a combined level of C4A and complement component 4B (C4B) polynucleotide or polypeptide, a copy number of C4A polynucleotide, and/or a sequence of C4A and/or C4B polynucleotide relative to a reference in a biological sample obtained from the subject, where the subject is identified as having or at risk of developing schizophrenia if the level of C4A polynucleotide or polypeptide is increased, the combined level of C4A and C4B polynucleotide or polypeptide is increased, the copy number of C4A polynucleotide is increased, and/or the sequence of C4A or C4B polynucleotide is altered.

In another aspect, the invention provides a method of characterizing risk of schizophrenia in a subject, the method containing the step of measuring a level of a complement component 4A (C4A) polynucleotide or polypeptide, a combined level of C4A and complement component 4B (C4B) polynucleotide or polypeptide, a copy number of C4A polynucleotide, and/or a sequence of C4A and/or C4B polynucleotide relative to a reference in a biological sample obtained from the subject, where an increase in the level of C4A polynucleotide or polypeptide, an increase in the combined level of C4A and C4B polynucleotide or polypeptide, an increase in C4A copy number and/or an alteration in a sequence of C4A or C4B polynucleotide indicates the subject has schizophrenia or is at risk of developing schizophrenia.

In another aspect, the invention provides a transgenic mouse containing a polynucleotide sequence encoding a human complement component 4A (huC4A) or human complement component 4B (huC4B) polypeptide, where the polynucleotide sequence is operatively linked to a promoter sequence. In various embodiments, the transgenic mouse expresses the human complement component 4A (huC4A) or human complement component 4B (huC4B) polypeptide in the central nervous system. In various embodiments, the mouse complement component 4 (C4) gene is deleted or inactivated in the transgenic mouse.

In various embodiments, the method further contains the step of recommending the subject for schizophrenia treatment or for further evaluation for schizophrenia if the subject is identified as having or at risk of developing schizophrenia. In some other embodiments, the method further contains the step of administering a schizophrenia treatment to the subject if the subject is identified as having or at risk of developing schizophrenia. In some embodiments, the schizophrenia treatment involves inhibiting the expression or activity of a complement component 4A (C4A) polypeptide or polynucleotide, including for example, inhibiting the complement pathway with a complement inhibitor (e.g., anti-C1q, Eculizumab/Soliris and Cetor/Sanquin, etc.)

In some embodiments, the alteration in sequence is insertion of a human endogenous retrovirus (HERV) sequence. In some other embodiments, an increase in copy number of C4A polynucleotide and insertion of a human endogenous retrovirus (HERV) sequence in a C4A and/or C4B polynucleotide is detected. In still other embodiments, an increase in a level of C4A polynucleotide or polypeptide is detected. In some embodiments, an increase in a combined level of C4A and C4B polynucleotide or polypeptide is detected.

In various embodiments of any one of the aspects delineated herein, the biological sample is plasma, serum, or cerebrospinal fluid (CSF). In certains embodiments, schizophrenia or neurodegenerative disease is characterized by detecting changes in activated microglia/exosomes present in CSF. In various embodiments, the schizophrenia treatment is an antipsychotic agent or psychosocial therapy.

In another aspect, the invention provides a kit containing a capture reagent for detecting the sequence of complement component 4A (C4A) polynucleotide or complement component 4B (C4B), and an antipsychotic agent. In some embodiments, the kit further contains a capture reagent for detecting the sequence of a HERV. In some other embodiments embodiments, the capture reagent is a probe or a primer. In various embodiments, the level, copy number, and/or sequence of complement component 4A (C4A) polynucleotide or complement component 4B (C4B) is measured using the kit of any one of the aspects delineated herein.

In yet another aspect, the invention provides a method of identifying an agent that inhibits schizophrenia. The method contains the step of (a) contacting a cell or organism with a candidate agent, and (b) measuring a level of complement component 4A (C4A) polynucleotide or polypeptide in the cell or organism contacted with the candidate agent relative to a reference level, where a decrease in the level indicates the candidate agent inhibits schizophrenia.

In another aspect, the invention provides an expression vector contains an isolated polynucleotide encoding complement component 4A (C4A).

In still another aspect, the invention provides a host cell or host organism contains an expression vector that contains an isolated polynucleotide encoding complement component 4A (C4A). In various embodiments, the host cell or host organism is mammalian.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof. In some embodiments, the agent is a small molecule chemical compound. In particular embodiments, the agent is an antipsychotic agent. Exemplary antipsychotic agents include, but are not limited to, aripiprazole, asenapine, clozapine, iloperidone, lurasidone, olanzapine, paliperidone, quetiapine, risperidone, ziprasidone, chlorpromazine, fluphenazine, haloperidol, and perphenazine.

By “alteration” is meant a change (increase or decrease) in the expression levels, copy number, or sequence of a gene or polypeptide as detected by standard art known methods such as those described herein. In some embodiments, an alteration in expression level includes a 10% change in expression levels, a 25% change, a 40% change, and a 50% or greater change in expression levels. In some other embodiments, an alteration in copy number includes an increase or a decrease by at least 1, at least 2, at least 3, at least 4, or at least 5 copies of the gene in a genome. In some embodiments, the alteration in copy number is an increase by at least 1, at least 2, at least 3, at least 4, or at least 5 copies of the gene.

The term “antibody,” as used herein, refers to an immunoglobulin molecule which specifically binds with an antigen. Methods of preparing antibodies are well known to those of ordinary skill in the science of immunology. Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. Antibodies also include dimers that may be naturally occurring or constructed from single chain antibodies or antibody fragments. The antibodies in the present invention may exist in a variety of forms including, for example, polyclonal antibodies, monoclonal antibodies, Fv, Fab and F(ab′) 2, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antibody specifically binds to C4A polypeptide.

The term “antibody fragment” refers to a portion of an intact antibody and refers to the antigenic determining variable regions of an intact antibody. Examples of antibody fragments include, but are not limited to, Fab, Fab′, F(ab′) 2, and Fv fragments, linear antibodies, scFv antibodies, single-domain antibodies, such as camelid antibodies (Riechmann, 1999, Journal of Immunological Methods 231:25-38), composed of either a VL or a VH domain which exhibit sufficient affinity for the target, and multispecific antibodies formed from antibody fragments. The antibody fragment also includes a human antibody or a humanized antibody or a portion of a human antibody or a humanized antibody.

“Biological sample” as used herein means a biological material isolated from a subject, including any tissue, cell, fluid, or other material obtained or derived from the subject. In some embodiments, the subject is human. The biological sample may contain any biological material suitable for detecting the desired analytes, and may comprise cellular and/or non-cellular material obtained from the subject. In various embodiments, the biological sample may be obtained from the brain. In particular embodiments, the biological sample is blood. In certain embodiments, the biological sample is cerebrospinal fluid (CSF). Biological samples include tissue samples (e.g., cell samples, biopsy samples), such as tissue from the brain. Biological samples also include bodily fluids, including, but not limited to, cerebrospinal fluid, blood, blood serum, plasma, saliva, and urine.

By “capture reagent” is meant a reagent that specifically binds a nucleic acid molecule or polypeptide to select or isolate the nucleic acid molecule or polypeptide.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

A “complement component 4 polypeptide” or “C4 polypeptide” is a complement component 4A (C4A) polypeptide or a complement component 4B (C4B) polypeptide. By “complement component 4A polypeptide” or “C4A polypeptide” is meant a polypeptide or fragment thereof having at least about 85% amino acid identity to GenBank Accession No. AAA51855.1 and having activities that include binding to antigen-antibody complex and binding to other complement components. Human C4 exists as two paralogous genes (isotypes), C4A and C4B; the encoded polypeptides are distinguished at a key site that determines which molecular targets they bind. The sequence of C4A polypeptide provided at GenBank Accession No. AAA51855.1 is shown below:

| | |
|---|---|
| 1 | mrllwgliwa ssfftlslqk prlllfspsv vhlgvplsvg vqlqdvprgq vvkgsvflrn |
| 61 | psrnnvpcsp kvdftlsser dfallslqvp lkdakscglh qllrgpevql vahspwlkds |
| 121 | lsrttniqgi nllfssrrgh lflqtdqpiy npgqrvryrv faldqkmrps tdtitvmven |
| 181 | shglrvrkke vympssifqd dfvipdisep gtwkisarfs dglesnsstq fevkkyvlpn |
| 241 | fevkitpgkp yiltvpghld emqldiqary iygkpvqgva yvrfgllded gkktffrgle |
| 301 | sqtklvngqs hislskaefq daleklnmgi tdlqglrlyv aaaiieypgg emeeaeltsw |
| 361 | yfvsspfsld lsktkrhlvp gapfllqalv remsgspasg ipvkvsatvs spgsvpevqd |
| 421 | iqqntdgsgq vsipiiipqt iselqlsvsa gsphpaiarl tvaappsggp gflsierpds |
| 481 | rpprvgdtln lnlravgsga tfshyyymil srgqivfmnr epkrtltsvs vfvdhhlaps |
| 541 | fyfvafyyhg dhpvanslrv dvqagacegk lelsvdgakq yrngesvklh letdslalva |
| 601 | lgaldtalya agskshkpln mgkvfeamns ydlgcgpggg dsalqvfqaa glafsdgdqw |
| 661 | tlsrkrlscp kekttrkkrn vnfqkainek lgqyasptak rccqdgvtrl pmmrsceqra |
| 721 | arvqqldcre pflsccqfae slrkksrdkg qaglqralei lqeedlided dipvrsffpe |
| 781 | nwlwrvetvd rfqiltlwlp dslttweihg lslsktkglc vatpvqlrvf refhlhlrlp |
| 841 | msvrrfeqle lrpvlynyld knltvsvhvs pveglclagg gglaqqvlvp agsarpvafs |
| 901 | vvptaaaavs lkvvargsfe fpvgdavskv lqiekegaih reelvyelnp ldhrgrtlei |
| 961 | pgnsdpnmip dgdfnsyvrv tasdpldtlg segalspggv asllrlprgc geqtmiylap |
| 1021 | tlaasryldk teqwstlppe tkdhavdliq kgymriqqfr kadgsyaawl srdsstwlta |
| 1081 | fvlkvlslaq eqvggspekl qetsnwllsq qqadgsfqdp cpvldrsmqg glvgndetva |
| 1141 | ltafvtialh hglavfqdeg aeplkqrvea siskansflg ekasagllga haaaitayal |
| 1201 | tltkapvdll gvahnnlmam aqetgdnlyw gsvtgsqsna vsptpaprnp sdpmpqapal |
| 1261 | wiettayall hlllhegkae madqaaawlt rqgsfqggfr stqdtviald alsaywiash |
| 1321 | tteerglnvt lsstgrngfk shalqlnnrq irgleeelqf slgskinvkv ggnskgtlkv |
| 1381 | lrtynvldmk nttcqdlqie vtvkghveyt meanedyedy eydelpakdd pdaplqpvtp |
| 1441 | lqlfegrrnr rrreapkvve eqesrvhytv ciwrngkvgl sgmaiadvtl lsgfhalrad |
| 1501 | lekltslsdr yvshfetegp hvllyfdsvp tsrecvgfea vqevpvglvq pasatlydyy |
| 1561 | nperrcsvfy gapsksrlla tlcsaevcqc aegkcprqrr alerglqded gyrmkfacyy |
| 1621 | prveygfqvk vlredsraaf rlfetkitqv lhftkdvkaa anqmrnflvr ascrlrlepg |
| 1681 | keylimgldg atydleghpq ylldsnswie empserlcrs trqraacaql ndflqeygtq |
| 1741 | gcqv |

By “complement component 4 polynucleotide” or “C4 polynucleotide” is meant a polynucleotide encoding a complement component 4A (C4A) polypeptide or a complement component 4B (C4B) polypeptide. By “complement component 4A polynucleotide” or “C4A polynucleotide” is meant a polynucleotide encoding a C4A polypeptide. An exemplary C4A polynucleotide sequence is provided at NCBI Accession No. NG_011638.1 (genomic sequence) and is reproduced below.

| | |
|---|---|
| 1 | tgtcttttgg ggtttgtttt tattctctct ttgagttttg tttccttatg cgcccagtta |
| 61 | cttttgaaaa tgttctgggc agatttgcct agattaataa atgccctcca tgttccaatt |
| 121 | actttttttt ttttgagaca gtgtcttacc ctgtcaccaa gctggagtgc agtggtatga |
| 181 | tcttggctca ctgcaacctc tgcctcctga gttcaagtga ttctcctgcc tcagcctccc |
| 241 | aagtagctgg cattacaggc acctgacacc acgcccagct aatttttttt tttttttttt |
| 301 | ttttgagacg gagtctcgct ctgtcaccca ggctggagtt cagtggcatg atcttggctt |
| 361 | actgcaagct ctgcctcctg ggttcaccca ttctcccgcc tcagcctccc gagtagctgg |
| 421 | gactacaggt gcccgccact atgcctggct aattgttttt ttttttgtat ttttagtaga |
| 481 | gatggggttt caccgtgtta gccaggatgg tcttgatctc cggacctcgt gatccacccg |
| 541 | tctcagcctg ccaaagtgct gggattacag gcatgagcca ccgcatctgg cctatttttg |
| 601 | tatttttaat ggagaccggg tttcatcatg ttggccaggc tggtcttgaa cttgaacttc |
| 661 | tgacctcaag tgatccaccc ttagcgtccc aaagtgctgg gattacaggc atgagccacc |
| 721 | gtgcccggcc ccagttattt ttatttttat tttttgagtt agagtctcac tctgtcaccc |
| 781 | aggctggagc gcagtggcat gatctcggct cacagcaact ttctgggttc aagcagttct |
| 841 | cctgtgtcag cctcctgagt agctgggact acaggcacac atcaccacgc ccggctaatt |
| 901 | tttgtagttt tagtagagac ggggttttac catattggtc aggctgatat tgaactcctg |
| 961 | acctcaggtg atccacccac gtcagcctcc caaagtgccg ggattacagg cttgagccat |
| 1021 | ctcgcccggc ctacttagat gttatattag tggtaattcc tgttatcctg tgagctcttt |
| 1081 | agtgtctaaa caattttttt taagagatgg ggtctcactg tgttgcccag ttgcaatcat |
| 1141 | atcttactgc agcctcaaac tcctgggtca agtgatcctc ttgccttagt ctcccaagta |
| 1201 | gctaggacca taggtgtctg cccccacgcc tggctgtttt tacatttttt gtagagatgt |
| 1261 | ggcgggtggg ggggtctcac tgtgttgccc agactggtct cgaactcctg tcctcaattg |
| 1321 | atcctgctac ctcagcctcc caaaatgctg aattacaggc atgagccact gtacctggtc |
| 1381 | ttaaacaatt ttaaaataac atttttatcc aggattttag ttaattttca acaggtggat |
| 1441 | tagttcttgc tgtattctcg taaacagaag tcctggttta tttttatttg ttttaaacat |
| 1501 | tgaatcccat actcctcccc accttaccct acccagaatt tagactgtta atgttttgaa |
| 1561 | gccacagcct gcatcttaat cactatttta tcttagtgcc tggtcttaga aattatattg |
| 1621 | actctttgat agaccatata taaggcaggt ggatgagaat gtgggtagct agttggaaaa |
| 1681 | ggctgcttgg tcatttgctt gattattttc tcacacagtt tttcctttac taagagaaaa |
| 1741 | tgcccccata ttggcaaaca aaatctccct gcctgagagc gcccagagta tagcagagca |
| 1801 | tcttaccctg atacgcctct tttcactctc ttctctgtgg agacagaagg agcttcaaga |
| 1861 | gcagggggag atcagaatcg tccagctggg cttcgacttg gatgcccatg gaattatctt |
| 1921 | cactgaggac tacaggacca gagtatgtga ctgtgtgcgt caggggtgct ggggggaggg |
| 1981 | cacaggttgg gggagacagg gaacttggga aacagaaata aaaacaaaag aaagaatttc |
| 2041 | cctgccccca catcccatgg agagggcaca gggccctggt aaatagtaat atgagggaga |
| 2101 | gagacaggag ggaaagaggg aggagtgaga gggtaaagag ggggggagag gagggggagg |
| 2161 | aggaggaagg aaggaggggg aggaggaggg ggggaggaag agggggagga ggatgaagag |
| 2221 | gaggaggaag aagaagggta tgagaggtgg aaggatctga gcaagaggta agacaggaag |
| 2281 | agaaatgctg tcctgggggt ggaggttggt agagagtgag ggtggggatg gaccatgtct |
| 2341 | ctcatctctg cttgtaggtc ctcaaggcct gtgatggccg accgtatgct ggggcagtgc |
| 2401 | agaaatttct agcttcagta cttccagcct gtggggacct tagtttccag caggaccaaa |
| 2461 | tgacacagac ctttggcttc agggactcag aaatcacgtg agacttgtgg aaccaaccaa |
| 2521 | agtcaggcat ctggtgcttc cctgcctccc tccagttcca tccagcctgt cctcctgttt |
| 2581 | ttttggtgaa cctgccagaa aagctgccaa aaagctgact cttcttgtta ataaaatgac |
| 2641 | ccaagtttgt attcctcccc acaagagagg aggcctatct tacctgggcc ttagaaagag |
| 2701 | ccctgaaata gaattcagtt cttggtggct tatcaaaagc acacaggggc ctggcaggaa |
| 2761 | gtgtaaaagc ttgatgttaa tcatactggg actaagagga tagagaatgg taggagctgg |
| 2821 | gataccccta aacattcaca ttaaaacaaa aaaaacccaa agctaaaaaa caactgggca |
| 2881 | ggagctaaat aaaaatctaa ttttgagagg ctgtatctgg ctcaggcctc ctactttgta |
| 2941 | acccatggaa tatgtgaaag catttgaaaa actatagcac tgatctcaca tgggcagaca |
| 3001 | cactctcaga gagatgtggt gggagccatg gcgcagtctg cctaggcagt ggcaggagcg |
| 3061 | cagaagactc tgattcctct cctcggtcct aagaccgaat gtgtgtcagg acatgtggtc |
| 3121 | agggaagaga agctatttaa ctgaaccagt aatagtagca ggaaaagaaa aagtggaggg |
| 3181 | agggcagtcc aggtaggggg cctggaacaa gcaactgcac caacagaggc agttggtgcg |
| 3241 | agcacagaac caccccaggc tgggattttg ttatccagtc tctcttgcat ggttgcccgt |
| 3301 | gtttctggag acttgtgtaa acattaatgg atgaggagga gagatggttc tcagagccca |
| 3361 | gccctcatct ctgctggctt cccactgccc tcaggcatct ggtgaatgct ggagtcctca |
| 3421 | ccgtccgaga tgctgggagc tggtggctag ctgtgcctgg agctgggaga ttcatcaagt |
| 3481 | actttgttaa aggtatccca tctgcagctc aagcctgcag cccctcacct tttggtggct |
| 3541 | cctcaggcct ctaggcctta ttcacctttc ccctttcctg tgccacttct cctctagggc |
| 3601 | gccaggctgt ccttagcatg gtccggaagg caaagtaccg ggaactgctc ctatcagagc |
| 3661 | tcctgggccg gcgggcgcct gtcgtggtgc ggcttggcct cacctaccat gtgcacgacc |
| 3721 | tcattggggc ccagctagtg gactggtgag tctttccctg gcctctggca gattatggag |
| 3781 | caatgaccca aagtgggatt tcctcccagc tcatgcttag tttcctagtg aaggccagtg |
| 3841 | gctctcattc ttctctggaa cccgggagca ccccttccca agttctaagt tctcctcaca |
| 3901 | gcttgagcct aggcgtctgg ctccagcctt gtctttctcc tgcacagcat ctctaccact |
| 3961 | tcaggaaccc tcctccgcct gccagagaca tgaagattct gctcatcatt gctcagctcc |
| 4021 | tcagagtggg ccgggagggg actagaagag ctgcatgatg gtggctgaga cagggtcacc |
| 4081 | ttgggaaggc ttgggagcca ggatgagtgt cgggctctcg tgtgtgcaaa aggtcagatg |
| 4141 | tgactgctgc tgtttgcctg gtttctgacc cagtggtggg gtttgagcaa tgcttctctg |
| 4201 | cccttccatg gaaagtggaa ccagaaatgg tgccaaggct gtggctgttc cctttcgtgt |
| 4261 | aaaatggtgc tgttattact ctgtcttgaa ataggaaggt gggatttctg gggaggctgg |
| 4321 | tgaaggaggg cagggttctt ttctctacgt gtcatgttaa aattgccaaa taaagtacct |
| 4381 | ctgcctgtga tattttctgg atgtccttta tttactgtga cgtgtgtttg ggtgccttgt |
| 4441 | ttaggggtag aggtgaagtc tgagctttgc ctcattcaga gaggaaaggg gtcaggggtt |
| 4501 | cactctgacg ttcaggccat tctccctgtg gagtggtgag ggtgtaccta atctcctaaa |
| 4561 | ccacggaatt tctgttaggg cctaaaaaag caaaagccta gtatagttca atttgtgttg |
| 4621 | gaatgaaagt aagagacaag tgtcttagaa gcctgtcatt gttttgtgag ggcctttaaa |
| 4681 | tatcctgtac tcgtgggcca tgttgggccc ttgtacgccc aggtatacat gagcttgtgt |
| 4741 | gcacctatac cctgatacag atatacctgg tagggggagg tgctcaggca ctggaatgag |
| 4801 | aggagttaac ggggaaggac agggttattt ctgggccaag attcagagtt tcccatggac |
| 4861 | acccaggtgt ccggggtgcc cccacaactc tgggcctgag gccagttgca cttcttggct |
| 4921 | gtcacgtggt ttcccagctt agctgggctg ggggaggagc aaggtccaga gtcaactctg |
| 4981 | ccccgaggcc tagcttggcc agaaggtagc agacagacag acggatctaa cctctcttgg |
| 5041 | atcctccagc catgaggctg ctctgggggc tgatctgggc atccagcttc ttcaccttat |
| 5101 | ctctgcagaa gcccaggtcc tggaggcggg atgctgggtg cttggattgg ggcagggctg |
| 5161 | gcatcgggac ccgattcagg agtgagggag agcaggggtg gaggtgtcag agcgaagtct |
| 5221 | gactgctgat cctgtctgtt ctccccaggt tgctcttgtt ctctccttct gtggttcatc |
| 5281 | tgggggtccc cctatcggtg ggggtgcagc tccaggatgt gccccgagga caggtagtga |
| 5341 | aaggatcagt gttcctgaga aacccatctc gtaataatgt cccctgctcc ccaaaggtgg |
| 5401 | acttcaccct tagctcagaa agagacttcg cactcctcag tctccaggta accagacccc |
| 5461 | atgccctcct gctgcttgtg ggggcctcct gccctgttcc catctgtctt gtaagtgtca |
| 5521 | tcatcttccc actggcctcc tcccctcctg tcttcccacc ctggcattct ccttccacgt |
| 5581 | ttctcccttg gtctctgtcc tttttggtca gctgtctctt gctctgtgac ccgctccctc |
| 5641 | tccctctccc tctcctgaca ggtgcccttg aaagatgcga agagctgtgg cctccatcaa |
| 5701 | ctcctcagag gccctgaggt ccagctggtg gcccattcgc catggctaaa ggactctctg |
| 5761 | tccagaacga caaacatcca gggtatcaac ctgctcttct cctctcgccg ggggcacctc |
| 5821 | tttttgcaga cggaccagcc catttacaac cctggccagc ggggtgagtc tcagccccag |
| 5881 | ggcctcaacc tttaaccccc tccgagccct ctcaggatga gtttggtgcc ccctaagtga |
| 5941 | gataacctga aagaaagtgc cacacagaag gggtgcttag gaaacatttg tcccctgctc |
| 6001 | cctctgtgga gtttgaccca ccctcccctt gcacatggac ccctgctcac ctctctcctc |
| 6061 | ctccactccc agttcggtac cgggtctttg ctctggatca gaagatgcgc ccgagcactg |
| 6121 | acaccatcac agtcatggtg gaggtgagtc cccgacctct ggccttcctg atcctggcca |
| 6181 | ctgatgtgac ctcctgcctg tgagcacttc tccccttgca gaactctcac ggcctccgcg |
| 6241 | tgcggaagaa ggaggtgtac atgccctcgt ccatcttcca ggatgacttt gtgatcccag |
| 6301 | acatctcaga gtgagcgctc ccaatgtggg ggctgccccc aagctacacc accccaattc |
| 6361 | ctgttaggct ctccacctcc cacacagagg cacgtcccca gatgccctga ccctcagcct |
| 6421 | cctgagcctc tggttaaccc ccacagtcct cttcccaggg aagcaggctg ctggctctcc |
| 6481 | gtgccccact gtacagatgg gctgagcccc ttccttgtcc attctcaggc cagggacctg |
| 6541 | gaagatctca gcccgattct cagatggcct ggaatccaac agcagcaccc agtttgaggt |
| 6601 | gaagaaatat ggtgagagct ggaaactgga gggacaggca gctgctttcc tgaaggaaat |
| 6661 | aagggtggaa ggagaggtac tgggagcagc tcagggcagg gagatatggg tgccacagcc |
| 6721 | ctgagcagag gggagtcttt gagctggagt ctgacctgcc tatcccttca ccctgggtca |
| 6781 | gtccttccca actttgaggt gaagatcacc cctggaaagc cctacatcct gacggtgcca |
| 6841 | ggccatcttg atgaaatgca gttagacatc caggccaggt aatacctccc tccccacctc |
| 6901 | tgcccaccag caccgggtcc tgctccctac tcagtatgaa tgggctcctg cttccctgcc |
| 6961 | ctcgggccat tattcccccc agcccttggc ccaccctctt ctctctgcca cgacaggtac |
| 7021 | atctatggga agccagtgca gggggtggca tatgtgcgct ttgggctcct agatgaggat |
| 7081 | ggtaagaaga ctttctttcg ggggctggag agtcagacca aggtaggaag gagaataggg |
| 7141 | gctggggagg ggaaggggca agggaggtga ggtgggagac tcagtctcac cctatgtcct |
| 7201 | gtttctttct atgccccagc tggtgaatgg acagagccac atttccctct caaaggcaga |
| 7261 | gttccaggac gccctggaga agctgaatat gggcattact gacctccagg ggctgcgcct |
| 7321 | ctacgttgct gcagccatca ttgagtctcc aggtgggtga ctttccctta ttgtaacccc |
| 7381 | agacccttgc ctctgacctc tgagctaacc ctctgtcctc cggcaccaac accaccccac |
| 7441 | ttctcacatc tcatctcaga ctcaaaacca ggaaacaccc aggagacctg gtttctctcc |
| 7501 | aactctgtct ctgtgactcg gcccttttcc ctggctgagt ttatttattt ctttgctcgt |
| 7561 | tctgctcatt ccttcactcc tccagtggac atgtgttgtt caatgccccg tgctaggcct |
| 7621 | cagcatgcac agacatgttg gggaccagcc tcaacgccac ccgtagggtt cctgaagtcc |
| 7681 | attggtgaca caggaatgag aagagacagg ttaagagttc ataaagagtg ggggccaggg |
| 7741 | ggccaattgc aaaatggagg ctgcaaaagg ctcagagctc tggtctccac actatttttt |
| 7801 | gagtacagtc actcagatct aagaagcaga tgttcaggga gaaacagtga aagggaggca |
| 7861 | gtgggtcata ggcgtaatct atagcaatag agttttaaat gaatctcctt tgtgctcaaa |
| 7921 | cagcatgtct ttaaattatc ggagagtagc tggtggaagt gggcttagct agaagactgc |
| 7981 | atgtctgtcc aatgcttcaa aggagggtct ttctccttga acagagtgtt tacagataag |
| 8041 | acagggggtc tcactctgag catgggaaca tgatggcaat taggaggctt ttcttctcag |
| 8101 | aggcctcttg tggctttcca caacttattg tctcatattt ttatggacag tttatacagg |
| 8161 | caccccacaa gtccttttcc caacatgccc ccctcccttt tttttttttt aaccgctatt |
| 8221 | gctattatgg cttatttgtg gtgtttggtc tgttttcaga agtgtctttt gcatctgtag |
| 8281 | actaaaagta aacagcataa acagatacac attaaagtaa aatttgtaat agttgatcct |
| 8341 | ttaatggtct taatctgttt aagaggattt atgtttgaaa gtccgtcagt agctccaatg |
| 8401 | agaatgtcag tctcaggcag gagggttaaa tgagcctgag atgctttaaa aacctgtttt |
| 8461 | tttaaaattt ggttatattt aatgttaaat ttttattttt ttcttttaga tgatgtctaa |
| 8521 | ctttttaaaa atgatgttta gtagtattat acgaatgggg agttatgtag aaattggaag |
| 8581 | tatttcaatt acattgtact tctaattgat gttttaagtt tattgtacga tcttccattt |
| 8641 | aaataacagt ctgtctaaga tcatttgttt gatttgtcaa ttgttggtct atttgggtct |
| 8701 | gagaattcca caattttgag gaattttttg ttaactattt atatattttg tagtttgaac |
| 8761 | agaggagtgt aaagcaattc cagcagccgc agcagtagct gtgactgcaa taaggcccat |
| 8821 | aagactgtta taagggtaaa aataaatctc tttgttttgg taaacacttt tttttaaaac |
| 8881 | atttttgtga caatatgaat ggaaggagag gctttctaag gtctattgag ggaaaccagt |
| 8941 | atccaaactc ctttcttagt ttttatcagt aacacagatg tttttacacc gaacgtggaa |
| 9001 | ttaatacagg tgaaaaggtg acagttttga caagtaatag tttgagaatt aggtcgaatg |
| 9061 | tcaatatttt tgaccattaa cataaaagga gggttgacac aactctgaat gggcactgtt |
| 9121 | ttgttggaag aaaactgata cgcaaattga agtttttaac cttttttttt taaagataat |
| 9181 | atattttttt ctaaacttaa atatgagatt gggccattat taactttcat aatttggagt |
| 9241 | gtttagggcc tattattgga ttaattattt tgggatgtgg gccagctgta ctaaaattgg |
| 9301 | tccaaattat gggaaaatga gcacgttttt cagtgtaagt agtgttacct ttttgatagt |
| 9361 | atagtttctg ttttagtttt gtcttgtatt tattattttg atgggtacaa ttaactgtaa |
| 9421 | aggtcccctc aggggaccaa ttaatgacaa tttcatagga attattttgt agtaccatag |
| 9481 | tgtgatcaga gatgtaattt tttttaatta atatttttaa attatttgac cattgttaag |
| 9541 | gttgttggca cctctttttt gggggcttaa actgttaatt gaattgaact ctgtgaatga |
| 9601 | tccgggctcc atccagaaaa taaatgatag gatactggtc tttgattatg acctggaatt |
| 9661 | ttaactagtc aatgttgtcg gtagcctttt aggcaaccga tagttggcct tatgtaaaga |
| 9721 | ggggggaact gataacctat ggacacattt attaactttt ttttttttcc tttgggtgag |
| 9781 | agggcccatg agtatttgta ggcttaggga tccaaacgct attattaaca taaacttcaa |
| 9841 | ctgggggttt taaccatgtg acaggcctaa ttaaaggcag gaatgggaca catgcccaat |
| 9901 | aggtataatt ttgggctgtt gtagccacag gtttgttagg cgaggaggtc actgttttta |
| 9961 | ttttggcttt gtattctagg attagtaaat aacagaagac aaacatgagt ataattagta |
| 10021 | actttttttt ttagtaaaag agtgacctgt agtgttactt ggcatcttag tttactatat |
| 10081 | gttattaatg aggaacccca ctgggggtat gttaatttat tctagctaag cagttatgtt |
| 10141 | attagaagct gagaaggggg tgtttgttaa agtaacaggg cagaagaaag gcggatttaa |
| 10201 | gatacgagct taatacagtg tagcaggtat aggtagtagg caaagtgaga gaattaaaaa |
| 10261 | tgaataaatt atttggctta gacttttgtt tttttagtat aatgtctgag gcctgtgttg |
| 10321 | tttgtggaag tcgcattgtt gaggctgtag ttcctgtagg gtctttttta ggctggttca |
| 10381 | aatgtttttt tattttttaa ttttttatcc tttgatgagg atgtagtctt taggctggta |
| 10441 | ctggaaattt taggagtggc gtctgtgtta agagactttt tacaattttt aaagagcagg |
| 10501 | ttagtgtttt aagaaaaact tgtgttttat tttaatgttt agtttataga aaactggatg |
| 10561 | atatcttttt aactttagta aatacgttta cacacggaat tttttacaat tatcatttta |
| 10621 | aaacttgttt agatctttaa aacaaaatta aacaaccttt tttgtataaa ttttttataa |
| 10681 | ctttttttat gacttttaca gacaattttt aacatgtctt aactttttat gttttataat |
| 10741 | ttttttacta aaggtacatt tttataactt tttaaatttt tttacttttt tgtatttttt |
| 10801 | tgatttttgt cttagtcttt tttttacttt tattttttta aatgtgtaat aattagatga |
| 10861 | gtgttggtaa caatggatgt atgtacatat tttagttttt aaaatttagg gatgtgttta |
| 10921 | acatctgttt gccagaactg actaggttcc aattctttac ggttaacacc tattgaagga |
| 10981 | gggtatgtgc ctgtgagctg gtaatctggg cattgtggga taatttgttt agccagcctc |
| 11041 | tgtgtaagtt gaaattattt agataagttt ctccaatttt ggtggaataa tcgatgtgat |
| 11101 | tgggtggctt ggtcaagcag tgatgtcata acctgaaggt ctgcttgatt attgccgtaa |
| 11161 | gccaatgggc caggcagaga gctgtgggct cgaatgtgtg taataaaagt aggatgtgta |
| 11221 | ccttggtcta gtaattgttg aagttgaaga aaaagaccac acagagtggg ctccagagca |
| 11281 | aacttaaggc tgtaatagtt tttaaataaa tacacagaat aaccttagct ctctgaatgt |
| 11341 | tagtaaattc agatcaagtg attggattat gtggtctcca ccagactgtt gctttttcat |
| 11401 | gtttaccaga cccaccagta aaaacagcta tggctccttc caaaggggca tcacaagtaa |
| 11461 | tttttggaag aacctatgta gttaatttta agaattgaaa agtttttagg ataatgatta |
| 11521 | ttaatacatc caacaaattt tgttaaatta atctgtcatg taactgagtt aataaatgcc |
| 11581 | tgtttaacct gatttttatt tattggaact ataattttta ttgggctcag tgccacaaaa |
| 11641 | tttaataatt catatatgag cctgtccaat tagaattgcc atctgattta agtatactgt |
| 11701 | aagtgctttt atggtattat gtggcaaaaa ggaccattta actaaatcat cattttgaac |
| 11761 | aataaccccc attattgtgt ggttagtgtg aagtagggaa cacaatgaat tataaaggca |
| 11821 | agtctgagtc aatcctactg acctgggctt gctgaatttt gttttcaatt actgataact |
| 11881 | ctttcatggc ctcgggtgtt agttctctgt tactgcgtaa gttggtattt cccctcaata |
| 11941 | ttgagaagag attagacata gcataagtag gaattgctaa attgggccaa atccaattaa |
| 12001 | tatcttctaa caatttttga aaattattta aggttttgaa agaatctctt ctaatttgaa |
| 12061 | ccttttgagg cttaatggct ctatcctgta cttgtatttt caaatactga aaaggagtgg |
| 12121 | ttgtttgaat tttgtcaggt gctataagta attcagcatt tgtaattgtc ttttgcaaag |
| 12181 | attaataata ttgaataagt tggtctctac tttttgctgc acaaatctgg aaactgatct |
| 12241 | ctaacaggct ggatagttct gcctacaaaa gtttgacaaa ctgtgggact atttaacata |
| 12301 | ccctggggca aaactttcca atgatatttg gctgcaggtt ttttgttatt aacggcagga |
| 12361 | atggtaaagg caaatttttt gaaatctgcc tctgctaaag gaattgtaaa aaagcagtct |
| 12421 | tttaaatcta taataacaag cggtcagtct ttagggagca cagtggggga tgggagccca |
| 12481 | ggttgtaagg ctcccatcgg ttgaattaca gcgttgacgc catctaccgg actttttctt |
| 12541 | aattacaaat actggggaat tccaaggaga gaaagtgggt gaaatatatc ctttttttag |
| 12601 | tagtttattt tataaagcac ccccaacttt tccttaggga gcggccactg ttcaacccag |
| 12661 | acggggcgcc gggtcatcca ttttaaggga aattgctcct tcactgtaat aactgtaggg |
| 12721 | tgaacctgaa ttgccccatc tccataatga actgtgggtc gggcaataat gggcacggtg |
| 12781 | agccaagtct cgggctccct ccccctgcac ccactcggct gaggaggagg tggccattct |
| 12841 | ggacatttct ctacaggaac cgtgggctga acaatttttt gagtaggttt agggagactg |
| 12901 | gggagattgg cataaatcat cttcagactc tcctttttgt tagtactcgg tagaggtggt |
| 12961 | tcagagttct gattatcaaa ctcctctctc tcctcctctg actcagcctc attatctgtc |
| 13021 | tgaaaaggct ccagtgctgc atgcaccaat gaccaaagcg accaaacagg caaaggaatt |
| 13081 | tcctttcctt ctctatatgc tcttttaagg tcctttccaa ctccttctta atgttttaat |
| 13141 | ttcaaagttt cctgttttgg gaaccaaggg caaaattgtt ccatagcatg aaacaaatcc |
| 13201 | ataagatttt ccgtatcaac ttttacccca ccatgcatgc ttgaagagct gccgtaggaa |
| 13261 | gctcaaatac gtggtgtact tactttcagt ttttcccatt gtgtccctag ctttctctgg |
| 13321 | gcgccccgct tacctgtaga ggttaaaact tttatgtcct tgggagtcct ttgttcgttg |
| 13381 | gtcctctgtt tcacatgctt gagcgtttcc tcaccagatt cttttgggcc ccacgttggg |
| 13441 | cgccagaatg ttggggacca gcctcaacac cacctgtagg gtacctgaag tctggtggtg |
| 13501 | acaaaggaat gagaagagac aggttaagag ttcataaaga gtggaggcca gggggccaat |
| 13561 | tgcaaaatgg aggctgcaaa aggctcagag ctctggtctc cacactattt attgagtaca |
| 13621 | ataacttaga tctaagaagc agatgttcag ggcaaaacag tgaaagggta gcagtgcgtc |
| 13681 | acaggcataa tctacagcag aagcgcttta aatgaatctc ctttgtgctc aaacagcata |
| 13741 | tctttaactt atcggagagt agctagtggg agtgggctta actaggagcc tgcacgtctg |
| 13801 | tccacattcc aatgcttcaa aggagggtct ttctccttga atacagtgtt tacagataag |
| 13861 | agagagcagg tctcgctctg agcatggcaa ttaggaggct tttctcctca gaggcctctt |
| 13921 | gtggctttcc acaacttatt gtcccatatt tttatggcca gtttatacag gcaccccaca |
| 13981 | agtccttttc ccaacacaga caggaatacg gcagcctgtg ccctgggagc tcactgtctt |
| 14041 | gtgggaggga accactcaag ccactcccca cttgtcctcc tgtccctctc ttcttgggct |
| 14101 | ctgtccccca cctctctctg tcctttgtct tgcaggtggg gagatggagg aggcagagct |
| 14161 | cacatcctgg tattttgtgt catctccctt ctccttggat cttagcaaga ccaagcgaca |
| 14221 | ccttgtgcct ggggccccct tcctgctgca ggtttcttcc agaggggaag gatgagtagg |
| 14281 | gaggatgtgg tagttaggag ggctcagggt ctgaccactc tcttttgcct gccctccttt |
| 14341 | acctgcctag gccttggtcc gtgagatgtc aggctcccca gcttctggca ttcctgtcaa |
| 14401 | agtttctgcc acggtgtctt ctcctgggtc tgttcctgaa gtccaggaca ttcagcaaaa |
| 14461 | cacagacggg agcggccaag tcagcattcc aataattatc cctcagacca tctcagagct |
| 14521 | gcagctctca gtaggactcc tcggacccct gggagatggt gggggaaggg gaggagggtg |
| 14581 | agctggggtc ccaaggatcc atggcctgac ttggggggaa ggtggggtac ttggctctga |
| 14641 | gctactaccc tattcgcacc tgaccccctc tccaggtatc tgcaggctcc ccacatccag |
| 14701 | cgatagccag gctcactgtg gcagccccac cttcaggagg ccccgggttt ctgtctattg |
| 14761 | agcggccgga ttctcgacct cctcgtgttg gggacactct gaacctgaac ttgcgagccg |
| 14821 | tgggcagtgg ggccaccttt tctcattact actacatggt gtgcatgagc tggggagtca |
| 14881 | cggagggctg gggtgcaggg aagagccctc tgggtggggc tgggggggtt caaggctgag |
| 14941 | gctgtcccat gaagaggcaa ccactcttgt ccctcccatt cttggcccag atcctatccc |
| 15001 | gagggcagat cgtgttcatg aatcgagagc ccaagaggac cctgacctcg gtctcggtgt |
| 15061 | ttgtggacca tcacctggca ccctccttct actttgtggc cttctactac catggagacc |
| 15121 | acccagtggc caactccctg cgagtggatg tccaggctgg ggcctgcgag ggcaaggtga |
| 15181 | ccggggtcag gagagatggc acttgtgccg agggggttga ggacagggtg attgccaaca |
| 15241 | gggcatggat ttagcttggg ggcagtgagg ataccgggac tgaaggaagc tctcccactc |
| 15301 | tgaccgcccc cacctgccgc ccctgccagc tggagctcag cgtggacggt gccaagcagt |
| 15361 | accggaacgg ggagtccgtg aagctccact tagaaaccga ctccctagcc ctggtggcgc |
| 15421 | tgggagcctt ggacacagct ctgtatgctg caggcagcaa gtcccacaag cccctcaaca |
| 15481 | tgggcaaggt ttgtccagac cctctccaca gctctctcac ccctccatgg ctcatccccc |
| 15541 | tgcttccctg agccttgggc gcagcccctg gatcccactg aggctcccca cagtctcttc |
| 15601 | cccacttggc cctgtggtct ccatctcctg gctctgtatc ctttcctatc cccccatgtg |
| 15661 | ctgccctctc acctgtgccg agtgctcagt cctgcccctc agccacactt ggctcctagc |
| 15721 | attcctgcct ttcttgcagg tctttgaagc tatgaacagc tatgacctcg gctgtggtcc |
| 15781 | tgggggtggg gacagtgccc ttcaggtgtt ccaggcagcg ggcctggcct tttctgatgg |
| 15841 | agaccagtgg accttatcca gaaagagtga gaacagagaa ggaaggggag tgggtggcgg |
| 15901 | gaagataagg aaggaggaag ggcctgaggg gaccagctgg aagagtccgg gcaggaaggg |
| 15961 | ctgggcaggg gaaggggagg aggggaggag gccgagtgcc tgacggctgg actgcagcct |
| 16021 | ttctctctac caggactaag ctgtcccaag gagaagacaa cccggaaaaa gagaaacgtg |
| 16081 | aacttccaaa aggcgattaa tgagaaatgt gagttgcggg tgcctaggca gtagcttggg |
| 16141 | ctctccacct gggatccggg ttgggggtct gcctctctgc ccctcggctc cttgctgaac |
| 16201 | ccacgtgtgg tatttggggc cagagatccg aattccggga ttacgagtgg aaggtgggca |
| 16261 | gctctctcca gcagcctctc ttatgttgct ggtctcaagg ggtcggggcg ggggctgagg |
| 16321 | tgtatgtcct ttttgtcctc tcatgctcac ccccacctgg ccctgcagtg ggtcagtatg |
| 16381 | cttccccgac agccaagcgc tgctgccagg atggggtgac acgtctgccc atgatgcgtt |
| 16441 | cctgcgagca gcgggcagcc cgcgtgcagc agccggactg ccgggagccc ttcctgtcct |
| 16501 | gctgccaatt tgctgagagt ctgcgcaaga agagcaggga caagggccag gcgggcctcc |
| 16561 | aacgaggtga ggggctgggt ggggctaggg cacaggtggc ggcgcttgga aaggcagaac |
| 16621 | ggtcccctcc tcactcccgt ccaccgtggt cccccagccc tggagatcct gcaggaggag |
| 16681 | gacctgattg atgaggatga cattcccgtg cgcagcttct tcccagagaa ctggctctgg |
| 16741 | agagtggaaa cagtggaccg ctttcaaatg tgagagtgtg tgccggcccg gccttttctc |
| 16801 | tgtgctgtgt ctcggggcca gccggggtag acgggccttc tctgcctttc cctacacaga |
| 16861 | ttgacactgt ggctccccga ctctctgacc acgtgggaga tccatggcct gagcctgtcc |
| 16921 | aaaaccaaag gtgatgtcac cctgtctggg cctcaggtga ccctgcttcc atttccctgt |
| 16981 | accccagctc cctgttccct ttgctcttag tgtaggaaga gggtccagtg atctggggag |
| 17041 | gtctgtgcca gcgtgcagct ggcgtgggcc agagggcaga ggcggactga gacagagctg |
| 17101 | ggtcaccccc acccctccct cctgtggccc tgaagctttg atggcccctc tgatctctgc |
| 17161 | ccctgtgccc acgcttcctt tccctcaggc ctatgtgtgg ccaccccagt ccagctccgg |
| 17221 | gtgttccgcg agttccacct gcacctccgc ctgcccatgt ctgtccgccg ctttgagcag |
| 17281 | ctggagctgc ggcctgtcct ctataactac ctggataaaa acctgactgt gaggccccat |
| 17341 | aggagcctga gcatacagga gttgggggag ccagggccca gtgaggggtg gggaggctaa |
| 17401 | ccgggccagg actctggcca tcctcgtttt cctgccctca ggtgagcgtc cacgtgtccc |
| 17461 | cagtggaggg gctgtgcctg gctgggggcg gagggctggc ccagcaggtg ctggtgcctg |
| 17521 | cgggctctgc ccggcctgtt gccttctctg tggtgcccac ggcagccgcc gctgtgtctc |
| 17581 | tgaaggtggt ggctcgaggg tccttcgaat tccctgtggg agatgcggtg tccaaggttc |
| 17641 | tgcagattga ggtgaatgga gcacccctga atataagtcc ccgggccccc agctttgtcc |
| 17701 | tccaccctca gcactctctc tgctggccag gccaggggcc caacacccaa accaatgcct |
| 17761 | tggtctgttc ccatcttcta caattctgat ccaactctgt ccctggagtt gaaactcaaa |
| 17821 | gttctggggg agtctgcgct agcagggcag gctgtagtcc tgtgtgacct cacaaccatg |
| 17881 | ttttccctga gacagaagga aggggccatc catagagagg agctggtcta tgaactcaac |
| 17941 | cccttgggtg agtgaccctc tacctccagc cattggtttc ctaagtgggt acaggtggtg |
| 18001 | ggggatgtgg acagcaggac aggctgccaa cttcccccat ttccccagac caccgaggcc |
| 18061 | ggaccttgga aatacctggc aactctgatc ccaatatgat ccctgatggg gactttaaca |
| 18121 | gctacgtcag ggttacaggt gggagtgccc tttagtccct tcccagtggc caccttcgga |
| 18181 | ttcatgtggg acttgtggat ccctgcttgg tcccactccc cgtgagcctc tgacacagag |
| 18241 | tcctcagacc tccaccctct ccctcccatg tagcctcaga tccattggac actttaggct |
| 18301 | ctgagggggc cttgtcacca ggaggcgtgg cctccctctt gaggcttcct cgaggctgtg |
| 18361 | gggagcaaac catgatctac ttggctccga cactggctgc ttcccgctac ctggacaaga |
| 18421 | cagagcagtg gagcacactg cctcccgaga ccaaggacca cgccgtggat ctgatccaga |
| 18481 | aaggttctgg gtgcaagggc aagcaggagg ggggccagga aaggacagtt actggaagat |
| 18541 | ggacagccca ggaggctaca gagggaaaga aagggggccc ctgatgagga tggggagcat |
| 18601 | ggccttgggc tcaaacagca gaagggtgag tgtcacctga gcggccacct ctcctctcca |
| 18661 | aggctacatg cggatccagc agtttcggaa ggcggatggt tcctatgcgg cttggttgtc |
| 18721 | acgggacagc agcacctggt gagcttggga gagtggttcc agggttctga gggggtcagg |
| 18781 | gctggggcag gggtgggaca gagctggtat gatgggaggg tggataacca ggcacctggg |
| 18841 | ggcgtgggca taatgagaag caagtcctta tccccaaccc tcctttcctg ccctccaggc |
| 18901 | tcacagcctt tgtgttgaag gtcctgagtt tggcccagga gcaggtagga ggctcgcctg |
| 18961 | agaaactgca ggagacatct aactggcttc tgtcccagca gcaggctgac ggctcgttcc |
| 19021 | aggacccctg tccagtgtta gacaggagca tgcaggtgcg ggcatgctgg ggctggcccg |
| 19081 | agaagcgcct gtcggaggac tctctttgcc ccttccccct cctgtttgac atcttttctc |
| 19141 | cccttactag gggggtttgg tgggcaatga tgagactgtg gcactcacag cctttgtgac |
| 19201 | catcgccctt catcatgggc tggccgtctt ccaggatgag ggtgcagagc cattgaagca |
| 19261 | gagagtggta agttcagtgg cgtttctgcc ctctgctggc ccccagctct ctcccttttt |
| 19321 | cctcaggaac ccaggggtcc aggcccaaga ccctcctccc gttttcttcc aggaagcctc |
| 19381 | catctcaaag gcaaactcat ttttggggga gaaagcaagt gctgggctcc tgggtgccca |
| 19441 | cgcagctgcc atcacggcct atgccctgac actgaccaag gcgcctgtgg acctgctcgg |
| 19501 | tgttgcccac aacaacctca tggcaatggc ccaggagact ggaggtgagg ggtgaggcgc |
| 19561 | tcctggcagt gagcctgagg cccaggggac cttaggatcc ctgagtgtgc ccagagggag |
| 19621 | aggctggatg aagactcaga ggaggaatga agttataagc aggggtgggt tgggggagac |
| 19681 | tcaggagagc ccagcagggg gtggctaagg gccaggggac caggctcttc tccctgcctt |
| 19741 | cctgtttact cgtggtctcc cttcactttc agataacctg tactggggct cagtcactgg |
| 19801 | ttctcagagc aatgccgtgt cgcccacccc ggctcctcgc aacccatccg accccatgcc |
| 19861 | ccaggcccca gccctgtgga ttgaaaccac agcctacgcc ctgctgcacc tcctgcttca |
| 19921 | cgagggcaaa gcagagatgg cagaccaggc ttcggcctgg ctcacccgtc agggcagctt |
| 19981 | ccaaggggga ttccgcagta cccaagtagg ggccgtcccc gggctctggc gggggtgggt |
| 20041 | agtcctcaga ccaagggctt gcttgagtcc tggctcaacc tccctaggac acggtgattg |
| 20101 | ccctggatgc cctgtctgcc tactggattg cctcccacac cactgaggag aggggtctca |
| 20161 | atgtgactct cagctccaca ggccggaatg ggttcaagtc ccacgcgctg cagctgaaca |
| 20221 | accgccagat tcgcggcctg gaggaggagc tgcaggtgaa ccactccctg gtgaaccact |
| 20281 | ccctcgcctg ggtagccagg acacctgggc ctcgtggcca ggccagaagc cgtccccacc |
| 20341 | ctcccacccg tggaatcccc gcagcacttc ttcctggggt cttcggggga agactgactt |
| 20401 | cctggctgtg tgacctggag ctctgagctt cagttttctc acttgtagag taacatacac |
| 20461 | agagttcacc ctacagggtc gttagaaggc tgaagtgaga taattcatgt gctggtataa |
| 20521 | actttgtgga aatgtgaggt ggggagagga ggtggggctg ttttgaggaa ggagataagt |
| 20581 | tattggagcc gcaaaaacag gtttgcttgt gcccttctaa catcgccttc ccttttctgt |
| 20641 | tgctgaagtt ttccttgggc agcaagatca atgtgaaggt gggaggaaac agcaaaggaa |
| 20701 | ccctgaaggt gagggccagg gaaggggtgg ggccaggcac tggtggagga gagggtgtgg |
| 20761 | agtgagaggc ctgtgggcag aggcacatgg tccggggaag gaggcagaca cctcagggtt |
| 20821 | ggtgtcccgt gcttccgtcc tgggtgtttt tccccctgct tgctttcgct tgctctcccc |
| 20881 | atctctgggt acctgttgtt tcctttaccc gcctcagtgc tggtggctcc gaatcccact |
| 20941 | cctcagccca ggcctcttcc ctgaaccatg ggccccactc gtcccactcc cacagcacct |
| 21001 | cagacgaggc atgtcccaaa gcccttcttc attctgtgtc tcttgtctgg ctggtgggag |
| 21061 | cccctcccag ccaggagccc agccactact ctagaggccg tgttagtggc ccctctccca |
| 21121 | agcctgtcct tatgtcccta gtgactcctc ctctgctccc ctgctgcctg tggcccttgg |
| 21181 | tgctgcatcc tagattctgt gctgagacgg ccttctccct acctggaact tctctctacc |
| 21241 | tcctgtctcc cctgtctgat ccactgtcca cacggcagtg acactgacct tccaaaagcc |
| 21301 | ccagccagat cagccttggg gaaaagtcac tccccgctgc ccacggctca gatggctggg |
| 21361 | cctctgccca cccctccggc cagacagctc tccttgtcta cacagatccc cttgcctttc |
| 21421 | ctgtccttcc ctgcttcttg gcccacagga caagctcttt cttctccttc aagccttggc |
| 21481 | cagaagcctt tcctgagctt ttcagtccag cctcttccca gcacagtctg gagtgttggc |
| 21541 | ctctgggggc aggcccctgc ttctttacct ctctgtctcg cctgacgcct gtggcgaatg |
| 21601 | tggtgccact cgtgtgtgtg gactgtgcag tgacggggag gaaaaggggc tgaaggcctc |
| 21661 | aaatcctgta gcccagggag atgcccttag gtatggcacc agagaggtct gtggcctcac |
| 21721 | atgtcccacg tcctctccct gccccttgct gagccaggtc cttcgtacct acaatgtcct |
| 21781 | ggacatgaag aacacgacct gccaggacct acagatagaa gtgacagtca aaggccacgt |
| 21841 | cgagtacacg agtgagtgtg ggggttggga ggccttgggg ccaggcaggg gctggcgcag |
| 21901 | ggagccgggt ggccatccca gccctcctca caatgcttcc ctgtgcagtg gaagcaaacg |
| 21961 | aggactatga ggactatgag tacgatgagc ttccagccaa ggatgaccca gatgcccctc |
| 22021 | tgcagcccgt gacacccctg cagctgtttg agggtcggag gaaccgccgc aggagggagg |
| 22081 | cgcccaaggt ggtggaggag caggagtcca gggtgcacta caccgtgtgc atctggtggg |
| 22141 | cgccgggagc tgccctgggc caggggaggg agggcaggac ccaggctggg gctgggcttc |
| 22201 | tggagcccgc gcaggcagaa cctggacgac agctcacacg tctccacagg cggaacggca |
| 22261 | aggtggggct gtctggcatg gccatcgcgg acgtcaccct cctgagtgga ttccacgccc |
| 22321 | tgcgtgctga cctggagaag gtgtggtcag ccacccaggg caaccccctc tgtcccaggt |
| 22381 | actgagccct gtcatgtgca gggcctgtga ccaactcccc ttttccacag ctgacctccc |
| 22441 | tctctgaccg ttacgtgagt cactttgaga ccgaggggcc ccacgtcctg ctgtattttg |
| 22501 | actcggtgag tggggagaga tgaggcagga agggactcga tggcaccggg tttactgagt |
| 22561 | atgcgttagg aggtttctca ggagacagct gtgtcagcgg ctggtgctct tgagaacttg |
| 22621 | tgatgtcatc agagagaagg acaagaatgt gagcccgtga gacacagcag agtaaggggc |
| 22681 | agacctgcag gcggcaggga ccgatgccag tcagcaggga ccctcagggt ttgagaggga |
| 22741 | gtctttccta atgctggttt tattcagctt gaggggctgc ctttgttttt ttgttgaact |
| 22801 | tcctatcttt tttttaatat taaagcgtat tttcctttac aaagtgatgg tggccataga |
| 22861 | tgatagttgt atttgtcttt tcacgacctt atttggctaa aatagttatc aaccctctta |
| 22921 | cggctctcaa aacattttta tttatttatt tagtaaagac agggtctcgc tctgttgccc |
| 22981 | aggctggtct tgaactcccg gcctcaagcg atcctctggc ctaggccttt caaagtaccg |
| 23041 | gatttacagg ccagagccac catgcccggc cttcaaaaaa agttttggaa catttactgt |
| 23101 | aacctctggg agaaaatgtg agaaaggtgt ggtggctgtc attagccagc tgtttgtagg |
| 23161 | tcagggagac ccctacccag tgtgtgcaga ggggccagcc cccatcagct ggggaagcct |
| 23221 | ggctgacaca tctgggttga acacaataga aaacacagag ccaacaagat tcccggatag |
| 23281 | ggagctgacg gtgcagcagc ctagctcagg agggacactg gcacggcacc gtgtggactg |
| 23341 | ggcccgcgtg ggcacgagga ggggtcaggc ctgggacctg agtcgggggg tcaggcagga |
| 23401 | tgacagaacc tgcagttagg ttgtggcaaa taaaggagga cccagttgta tccatgacaa |
| 23461 | agatgaggcc gcgaggaggg cgagtgggtt tgggggcagg cagagtgcct tggagaactt |
| 23521 | acaggtcctg ccacaatcct aatgcaagga tggagctgca agttcagttt gggaatcatc |
| 23581 | agcctggatt ggtttggtgg aagccaggga gtggttgaga cccccacagg ggagctctga |
| 23641 | ggaaggaagt tccgaaggag ggaacgtaag aaatgaccag gtcagaacca agggtggtcc |
| 23701 | agaagctaac ccttagctta gggacagttt cacagagaac acgtccatga tgcaagactc |
| 23761 | tgctgagggc ctggagcagt gaagactggg gcaaggtcac cctctgggaa gtgaagtcac |
| 23821 | cagagacctt gcggagcagc tttgagagtt ctctgagtag gaaggtaaca gaatgtgaag |
| 23881 | gacactggag agaaggccaa taggaagcaa acaaaaacag gccaaggaaa cccagtacag |
| 23941 | ggggctgcag ggcccaggga gtgggtccct catctctcct ccccacgctt ggccaggtcc |
| 24001 | ccacctcccg ggagtgcgtg ggctttgagg ctgtgcagga agtgccggtg gggctggtgc |
| 24061 | agccggccag cgcaaccctg tacgactact acaaccccgg tgagcactgc aggacaccct |
| 24121 | gaaattcagg agaactttgg cataggtgcc ctcctatggg acaatggaca ccggggtagt |
| 24181 | gagggggcag agagccctgg ggctccctgg gactgaggag gcagaatgga ggggcctgtg |
| 24241 | ccctaactcc tctctgttct ccagagcgca gatgttctgt gttttacggg gcaccaagta |
| 24301 | agagcagact cttggccacc ttgtgttctg ctgaagtctg ccagtgtgct gagggtgaga |
| 24361 | ctgagggcct ggggcggggc agtggaggcg ggatggccgg ggcccccccc acactgtctg |
| 24421 | atgggttccc caacttcagg gaagtgccct cgccagcgtc gcgccctgga gcggggtctg |
| 24481 | caggacgagg atggctacag gatgaagttt gcctgctact acccccgtgt ggagtacggt |
| 24541 | cagtcttccc accgaggccc tggcctgacc ctccctcggg gaccggccgt tttggtctct |
| 24601 | ctgggtgtag cctgctcctc ttacaggtca tgcacgcagc ctgtttgctc tgacaccaac |
| 24661 | ttcctaccct ctcagcctca aagtaactca cctttccccc ttctcctcac cccctcttag |
| 24721 | gcttccaggt taaggttctc cgagaagaca gcagagctgc tttccgcctc tttgagacca |
| 24781 | agatcaccca agtcctgcac ttcagtatga agcaaaccgg agaggcgggc agggctgggg |
| 24841 | ggagacaggg aggctgaggt gtggccgagg acctgaccat ctggaagtgt gaaaatcccc |
| 24901 | ttgggctgtc agaagccttg ggcttggcca taaataggga ggcagtggca cctctccatg |
| 24961 | ggggtggcga aggtggaatg agaggatcta cacagagtcc ccagcctggg ctcaccctgc |
| 25021 | accttctctt cccctctgac cacttttgcg cacgtcatcc ccgcagccaa ggatgtcaag |
| 25081 | gccgctgcta atcagatgcg caacttcctg gttcgagcct cctgccgcct tcgcttggaa |
| 25141 | cctgggaaag aatatttgat catgggtctg gatggggcca cctatgacct cgagggacag |
| 25201 | tgagtcatct ggtcccctca gtctcttgtc ctccccatgc ctcgccacct aggccttgcc |
| 25261 | cctcagaagc cagatgcctg tgctctccgt ttccacctgc catcctcccg agccctgctg |
| 25321 | actgcccctt tgccccctgc agcccccagt acctgctgga ctcgaatagc tggatcgagg |
| 25381 | agatgccctc tgaacgcctg tgccggagca cccgccagcg ggcagcctgt gcccagctca |
| 25441 | acgacttcct ccaggagtat ggcactcagg ggtgccaggt gtgagggctg ccctcccacc |
| 25501 | tccgctggga ggaacctgaa cctgggaacc atgaagctgg aagcactgct gtgtccgctt |
| 25561 | tcatgaacac agcctgggac cagggcatat taaaggcttt tggcagcaaa gtgtcagtgt |
| 25621 | tggcagtgaa gtgtcagtgt gtgttgctag ggctgagagc agtgcccctg cccgatgcag |
| 25681 | ttctgggcag gccaggttga cataacctta gactctctga gccctgatga cccttgggct |
| 25741 | gttcagctct gctagaacct cccagatgac ccgctaggag tctagtgctt cacaggacca |
| 25801 | ccccgagcag aactgggacc caagagcctg caccccaagg accagagtcc atgccaagac |
| 25861 | cacccttcag cttccaaggc cctccactgc ccggctgtcg ccagtcacca cggcctcaga |
| 25921 | cagggcttgt gctcagctga cacctgtgac acagctcttc tgcctcatga gctgttgtcc |
| 25981 | agctacacct ccccgactct gtcctcgtgc tgctggcggt tctgaggtct gcagatttta |
| 26041 | gctgagttcc gggctgttga aagcctgctg acgcttggtt ctgttatcag tggaatgagg |
| 26101 | tgactttccc ggagttgtgc aatcctcagg tccggcagtg tcttcttcca gttactggtt |
| 26161 | tcaaacaagc caaaagtctg actttggtgt gtttgtgaat cctctgagga agccgctgtt |
| 26221 | ctcctggggt ctccccttcc caccggacct gcctaacttt cccccattta gtggcacacc |
| 26281 | tggggtcttc agagatgact ccgcgtctgt ccaaagaagt ttggtgagat cagtttccgt |
| 26341 | agaggtcatg acagttcagc agcctgccat ccagtcattc gacagaaatt cgggaatctt |
| 26401 | tcacttcatg ccatgccctg tgccaggtgc cagagataca gctgctcact ccagggctca |
| 26461 | tcgctgggga gacagataag aggacgggca gtccccaccc tctgtgaaag atgtgatgtc |
| 26521 | agggagcagt gtggtcctgt ggggcatcta accaagtcag gggcattgcc aggcagggac |
| 26581 | agggaaggct tcctggagca ggtggcctcc aagtggggct ctgaagactg agaaggagcc |
| 26641 | aggaaaagag caggggtaga tgagggcatc tggggcagaa ggagaatata caaaggccca |
| 26701 | gaggccgggg gcaggacagg gtacctttgg ggacattgca tgtaattgac cacattcgga |
| 26761 | gtttggattt ggaagtggtg gaagagatgg agatggtgag acaagtagta agcacgtcag |
| 26821 | ccttccaggt gcgctccttt ccgatgagca ctgtcttatc ccacgtaact ttgagaagtt |
| 26881 | tgggcctttc ccactgtggc agaggtttcc tgaggctctt gcatacatgg ccctatggtt |
| 26941 | gctcatcaga tctttctccc agtagctgct cagcatggtg gtggcataag cccattttcc |
| 27001 | ggagccaggg attcagttgc agcaagacct ggcccggtct gggaggtcaa ccatgaagaa |
| 27061 | ggcagtagct gtcattgccc aaccccagaa atcccaatcc tgttttctcc ctctcagtcc |
| 27121 | tgatcatgga ttcagcagca gcgaactcgc caatgtagtg ggtggcacag ccagggtctt |
| 27181 | gactctggct ctgcagtagc acagtctgga aaagctctga ggggagagag acccccactg |
| 27241 | gtccgagggt ctggcacaga gccagaaatg ggggggaagg tatggggctg ggtcgcctct |
| 27301 | gacctctcag gtaccatcca ggaggccctg gcctctcact gaacccggcc actcctcttt |
| 27361 | ggcatggcct cttcccaaat ccccaaactg cctccttact cacaaaagtg gtctctgagt |
| 27421 | gtcagtccag tgggaccccc accccttatg gcttcagttc cccaaatagg gctggaccct |
| 27481 | tgatcctgat ccagctgtgg ctatccagcc ccttcctggg gactttggac tttgaggggg |
| 27541 | ggcatgccca gttgtgctgg gaatccatac tttccctggc tggagtagaa cctgtggact |
| 27601 | gtagtcctga gggcagtcat gttc |

By “complement component 4B polypeptide” or “C4B polypeptide” is meant a polypeptide or fragment thereof having at least about 85% amino acid identity to NCBI Accession No. NP_001002029.3 and having activities that include binding to antigen-antibody complex and binding to other complement components. The sequence at NCBI Accession No. NP_001002029.3 is shown below:

| | |
|---|---|
| 1 | mrllwgliwa ssfftlslqk prlllfspsv vhlgvplsvg vqlqdvprgq vvkgsvflrn |
| 61 | psrnnvpcsp kvdftlsser dfallslqvp lkdakscglh qllrgpevql vahspwlkds |
| 121 | lsrttniqgi nllfssrrgh lflqtdqpiy npgqrvryrv faldqkmrps tdtitvmven |
| 181 | shglrvrkke vympssifqd dfvipdisep gtwkisarfs dglesnsstq fevkkyvlpn |
| 241 | fevkitpgkp yiltvpghld emqldiqary iygkpvqgva yvrfgllded gkktffrgle |
| 301 | sqtklvngqs hislskaefq daleklnmgi tdlqglrlyv aaaiiespgg emeeaeltsw |
| 361 | yfvsspfsld lsktkrhlvp gapfllqalv remsgspasg ipvkvsatvs spgsvpevqd |
| 421 | iqqntdgsgq vsipiiipqt iselqlsvsa gsphpaiarl tvaappsggp gflsierpds |
| 481 | rpprvgdtln lnlravgsga tfshyyymil srgqivfmnr epkrtltsvs vfvdhhlaps |
| 541 | fyfvafyyhg dhpvanslrv dvqagacegk lelsvdgakq yrngesvklh letdslalva |
| 601 | lgaldtalya agskshkpln mgkvfeamns ydlgcgpggg dsalqvfqaa glafsdgdqw |
| 661 | tlsrkrlscp kekttrkkrn vnfqkainek lgqyasptak rccqdgvtrl pmmrsceqra |
| 721 | arvqqpdcre pflsccqfae slrkksrdkg qaglqralei lqeedlided dipvrsffpe |
| 781 | nwlwrvetvd rfqiltlwlp dslttweihg lslsktkglc vatpvqlrvf refhlhlrlp |
| 841 | msvrrfeqle lrpvlynyld knltvsvhvs pveglclagg gglaqqvlvp agsarpvafs |
| 901 | vvptaatavs lkvvargsfe fpvgdavskv lqiekegaih reelvyelnp ldhrgrtlei |
| 961 | pgnsdpnmip dgdfnsyvrv tasdpldtlg segalspggv asllrlprgc geqtmiylap |
| 1021 | tlaasryldk teqwstlppe tkdhavdliq kgymriqqfr kadgsyaawl srgsstwlta |
| 1081 | fvlkvlslaq eqvggspekl qetsnwllsq qqadgsfqdl spvihrsmqg glvgndetva |
| 1141 | ltafvtialh hglavfqdeg aeplkqrvea siskassflg ekasagllga haaaitayal |
| 1201 | tltkapadlr gvahnnlmam aqetgdnlyw gsvtgsqsna vsptpaprnp sdpmpqapal |
| 1261 | wiettayall hlllhegkae madqaaawlt rqgsfqggfr stqdtviald alsaywiash |
| 1321 | tteerglnvt lsstgrngfk shalqlnnrq irgleeelqf slgskinvkv ggnskgtlkv |
| 1381 | lrtynvldmk nttcqdlqie vtvkghveyt meanedyedy eydelpakdd pdaplqpvtp |
| 1441 | lqlfegrrnr rrreapkvve eqesrvhytv ciwrngkvgl sgmaiadvtl lsgfhalrad |
| 1501 | lekltslsdr yvshfetegp hvllyfdsvp tsrecvgfea vqevpvglvq pasatlydyy |
| 1561 | nperrcsvfy gapsksrlla tlcsaevcqc aegkcprqrr alerglqded gyrmkfacyy |
| 1621 | prveygfqvk vlredsraaf rlfetkitqv lhftkdvkaa anqmrnflvr ascrlrlepg |
| 1681 | keylimgldg atydleghpq ylldsnswie empserlcrs trqraacaql ndflqeygtq |
| 1741 | gcqv |

By “complement component 4B polynucleotide” or “C4B polynucleotide” is meant a polynucleotide encoding a C4B polypeptide. An exemplary C4B polynucleotide sequence is provided at NCBI Accession No. NG_011639.1 (genomic sequence) and is reproduced below.

| | |
|---|---|
| 1 | atggtgctgg tcctggaggc accggctccg ttctgcatct cctccccgca gtccctgggg |
| 61 | aaggggatcc gcagcccacc tgggagagga gagcaggggc cagtcctttt ccaagcctta |
| 121 | ggccctggct gcccacccag cccccggccc cgggcccgtg cgtccaggta cccgtggtga |
| 181 | aagaggtgga cacgggcggc aggaggctct ggccccacat ggcctggagc cgtgcattgt |
| 241 | aggaggtgga gggaaagagg ccaaggagct ggtgagatgt gatccctcct gggagcagga |
| 301 | tctcctgtgg gacagacaag ggggggtcag gggagaggga ggtggagacc ctccgggagg |
| 361 | gccagaggca gcacctcctg gaatcaccca gggaggggag ttgggtcagt ggggccgggg |
| 421 | cacctggttc tgtccaccag gggtgtggaa gctgagcagg tagcctgcgg gccggactgg |
| 481 | gggctcagtc caagtgagca gggcggtgcg gggggtcact tccttggcct ccaagtcccg |
| 541 | aggggcctct agccctagga gggaaagcag gaagaggaga tggggatgag gcccaacctg |
| 601 | gctccctcta cctcctctcc ctgtcccaca caccccacag accctacctg tggtgaaggt |
| 661 | gatgctggct ggggaagtga ggttggggcc ccgcaggcca cgcactgtgg cggtgtagtt |
| 721 | ggtgtggagg acaaggtcat gcagggggta gtccaccgcg ctgcctgggg tctccgcctg |
| 781 | cagaggcggg gctgggagtg tagagagggg catcaaggcc tgccccctcc atcctcggcc |
| 841 | agagtccagc ctcccccctg caatccccac cctgaacaag tcccctccag aggcctcagg |
| 901 | cctgctcacc cccaggggct gtgacctgga cgtcataggt gtccacagga ttctgggggg |
| 961 | gcttccagtg cagcacggcg aatccctcgg tcaagttcag tgcacgcaac tgtgtgggac |
| 1021 | cgtcaggaac tgggggaagg ggaggggctc agaagggtcc ccgcggctct ctctactccg |
| 1081 | tgcctcccca gactccactg gcctcccgtc cgcaatcgga gcctccacca cctccctttc |
| 1141 | accctcctcg ttctctctca actcccaccc atgccgtttt cttgactccc acctggagtt |
| 1201 | tctgggtccg ggcccggccg tccacctgca cactctgagg ctcccctgaa aacgttgggg |
| 1261 | atcgagggtt acccagggaa ccccagggcg gctggagggt gggcagagtg caggggggag |
| 1321 | aggaaatgcg aggcgatgag cacatggcaa aggcaccacc tccgtccgcc agctggtagg |
| 1381 | agactttgaa gctgtccgcc cgggatggtg ggggcatcca gttgaccttg gctgaggtct |
| 1441 | ccctgatttc actgaattgg aggtcacggg ggctctccag aactgcagag gggtcaagga |
| 1501 | acaatgacgc aggcaggggc agggaggctc ctccctgcga gtccccccct cgcctctgct |
| 1561 | ccagcacagg ctcaccaccc cttttcctct agtccccagg aatggaagtc gctctgcaga |
| 1621 | ttcctccagg cccaccacca actcgcccac ccccaccgct ggctgaggca ctaggtcccc |
| 1681 | cccgtgaagt acaaagaccc ccactttggg gcagagtgtg tgtgggtcct tacctgggct |
| 1741 | gagggtgcgg gcggttccct ggatgctgtc ggccttgtgg ggtcctcgca gcccatacag |
| 1801 | tgtcaggctg tacagagtcc cggaacgcag gtcccggagc acggccgagt gccgcgtccc |
| 1861 | cggcaccatc agctcgcgct gcagcagtgg acgcggatgc ggctccagag tgcttggtga |
| 1921 | tggaacccca aagcggagca ggaaggagtc gaaggccccc ggtggggcct cccagttgag |
| 1981 | cctcagtgaa ctggtggtca cgtcagtcac agacagctgg gacaggcggg gccttgactc |
| 2041 | ctctgaggtc tgaccagcag gagccagccc tgcacggagt gggtggggga gaagggattg |
| 2101 | gagacagaag cacaccagct tggtgaccca gagcacgtcc cttccacccc cctccctgcc |
| 2161 | cccgtttctc tatctgtaac cagggacttg cagccacagg ggggtcctgt ggggcagagc |
| 2221 | taaaggccac tcgcatccag cccatccatc ctctctccct ggtacccgcc tcacgctctt |
| 2281 | tccctgcgac caccccttct gagcccccgt ttctcccttc tgagtcctag gctagaggcc |
| 2341 | ggagacgcct ggtggtacct gtggtgccct cagctgagag gggccccagg cgcttccctt |
| 2401 | catggaggcc atagaggagg aacctgtagc gggtgctggg ctccaggcct gagatgagga |
| 2461 | tcttgctctg gtcgccgtcc acgagcaagg cctggggctg cccattcgtg tcctcatact |
| 2521 | ggaccacgaa ggaatcaaag gggccctggg ccacgctcca cgagaggcgc atggagtctg |
| 2581 | gggttgtgtc ggtcacggtc agcactccta ggcggggctc ttcaggaggc tcaggggcct |
| 2641 | ctggggctaa ctctggggct ggtgtgtcct cttctggggc tgcgtgggag aagcccaggg |
| 2701 | gagaatctga gtgaggggcg ccatggggtg ctccattttt atcttccagg cttggcccaa |
| 2761 | ggctgaggtg ggaagtttat aggtccaggc ccagtcagac aatgaagtcg ctgtggcctc |
| 2821 | gtgactcctg cgagctcccg cgctgtctga gtcaggtgct cgcttccccc ttccacaccc |
| 2881 | cggtgtcctg ccgagcccac ctcgagatat cacaggctct ggccccaccc atgccgggat |
| 2941 | acattcactg agcttgagga gtgtggtgct cccttctgag agaagctgag ggtggaactg |
| 3001 | gctggttgag gtgactggca aatcccacca gccgtgccgt ggtcaggcct gtctgaggtg |
| 3061 | ggcatcagcg agctctggaa gaggagcctg taccacaaat gcagccactg ctgttggttt |
| 3121 | ctgtgtcccc gctcattttg ttttccagtg atgttcctct taagaaaatg ctcctgactc |
| 3181 | atccacggca gggaggtttg ccactatctg gacaaggcca cccttcgggg aggcgacagc |
| 3241 | agccccagcg agtaatgagg agcagcggca gtgacggggc agagtcgggg ctgggagatt |
| 3301 | agagagcccc tcccagggcc tttccctccc gcctggcctg gctcctgctc tggactcctt |
| 3361 | gatggatgtt gaagcccaca gggctgcaga ctcctcctcc ttcctgggca caggccaggt |
| 3421 | caccccactc cggcctgccc actcctgcag tcatctttgt cttcagacca aatgcacaag |
| 3481 | tactttgtta aaggtatccc atctgcagct caagcctgca gcccctcacc ttttggtggc |
| 3541 | tcctcaggcc tctaggcctt attcaccttt cccctttcct gtgccacttc tcctctaggg |
| 3601 | cgccaggctg tccttggcat ggtccggaag gcaaagtacc gggagctgct cctatcagag |
| 3661 | ctcctgggcc ggcgggtgcc tgtcgtggtg cggcttggcc tcacctacca tgtgcacgac |
| 3721 | ctcattgggg cccagctagt ggactggtga gtctttccct ggcctctggc agattatgga |
| 3781 | gcaatgaccc aaagtgggat ttcctcccag ctcatgctta gtttcctagt gaaggccagt |
| 3841 | ggctctcatt cttctctgga acccgggagc accccttccc aagttctaag ttctcctcac |
| 3901 | agcttgagcc taggcgtctg gctccagcct tgtctttctc ctgcacagca tctctaccac |
| 3961 | ttcaggaacc ctcctccgcc tgccagagac atgaagattc tgctcatcat tgctcagctc |
| 4021 | ctcagagtgg gccgggaggg gactagaaga gctgcatgat ggtggctgag acagggtcac |
| 4081 | cttgggaagg cttgggagcc aggatgagtg tcgggctctc gtgtgtgcaa aaggtcagat |
| 4141 | gtgactgctg ctgtttgcct ggtttctgac ccagtggtgg ggtttgagca atgcttctct |
| 4201 | gcccttccat ggaaagtgga accagaaatg gtgccaaggc tgtggctgtt ccctttcgtg |
| 4261 | taaaatggtg ctgttattac tctgtcttga aataggaagg tgggatttct ggggaggctg |
| 4321 | gtgaaggagg gcagggttct tttctctacg tgtcatgtta aaattgccaa ataaagtacc |
| 4381 | tctgcctgtg atattttctg gatgtccttt atttactgtg acgtgtgttt gggtgccttg |
| 4441 | tttaggggta gaggtgaagt ctgagctttg cctcattcag agaggaaagg ggtcaggggt |
| 4501 | tcactctgac gttcaggcca ttctccctgt ggagtggtga gggtgtacct aatctcctaa |
| 4561 | accacggaat ttctgttagg gcctaaaaaa gcaaaagcct agtatagttc aatttgtgtt |
| 4621 | ggaatgaaag taagagacaa gtgtcttaga agcctgtcat tgttttgtga gggcctttaa |
| 4681 | atatcctgta ctcgtgggcc atgttgggcc cttgtacgcc caggtataca tgagcttgtg |
| 4741 | tgcacctata ccctgataca gatatacctg gtagggggag gtgctcaggc actggaatga |
| 4801 | gaggagttaa cggggaagga cagggttatt tctgggccaa gattcagagt ttcccatgga |
| 4861 | cacccaggtg tccggggtgc ccccacaact ctgggcctga ggccagttgc acttcttggc |
| 4921 | tgtcacgtgg tttcccagct tagctgggct gggggaggag caaggtccag agtcaactct |
| 4981 | gccccgaggc ctagcttggc cagaaggtag cagacagaca gacggatcta acctctcttg |
| 5041 | gatcctccag ccatgaggct gctctggggg ctgatctggg catccagctt cttcacctta |
| 5101 | tctctgcaga agcccaggtc ctggaggcgg gatgctgggt gcttggattg gggcagggct |
| 5161 | ggcatcggga cccgattcag gagtgaggga gagcaggggt ggaggtgtca gagcgaagtc |
| 5221 | tgactgctga tcctgtctgt tctccccagg ttgctcttgt tctctccttc tgtggttcat |
| 5281 | ctgggggtcc ccctatcggt gggggtgcag ctccaggatg tgccccgagg acaggtagtg |
| 5341 | aaaggatcag tgttcctgag aaacccatct cgtaataatg tcccctgctc cccaaaggtg |
| 5401 | gacttcaccc ttagctcaga aagagacttc gcactcctca gtctccaggt aaccagaccc |
| 5461 | catgccctcc tgctgcttgt gggggcctcc tgccctgttc ccatctgtct tgtaagtgtc |
| 5521 | atcatcttcc cactggcctc ctcccctcct gtcttcccac cctggcattc tccttccacg |
| 5581 | tttctccctt ggtctctgtc ctttttggtc agctgtctct tgctctgtga cccgctccct |
| 5641 | ctccctctcc ctctcctgac aggtgccctt gaaagatgcg aagagctgtg gcctccatca |
| 5701 | actcctcaga ggccctgagg tccagctggt ggcccattcg ccatggctaa aggactctct |
| 5761 | gtccagaacg acaaacatcc agggtatcaa cctgctcttc tcctctcgcc gggggcacct |
| 5821 | ctttttgcag acggaccagc ccatttacaa ccctggccag cggggtgagt ctcagcccca |
| 5881 | gggcctcaac ctttaacccc ctccgagccc tctcaggatg agtttggtgc cccctaagtg |
| 5941 | agataacctg aaagaaagtg ccacacagaa ggggtgctta ggaaacattt gtcccctgct |
| 6001 | ccctctgtgg agtttgaccc accctcccct tgcacatgga cccctgctca cctctctcct |
| 6061 | cctccactcc cagttcggta ccgggtcttt gctctggatc agaagatgcg cccgagcact |
| 6121 | gacaccatca cagtcatggt ggaggtgagt ccccgacctc tggccttcct gatcctggcc |
| 6181 | actgatgtga cctcctgcct gtgagcactt ctccccttgc agaactctca cggcctccgc |
| 6241 | gtgcggaaga aggaggtgta catgccctcg tccatcttcc aggatgactt tgtgatccca |
| 6301 | gacatctcag agtgagcgct cccaatgtgg gggctgcccc caagctacac caccccaatt |
| 6361 | cctgttaggc tctccacctc ccacacagag gcacgtcccc agatgccctg accctcagcc |
| 6421 | tcctgagcct ctggttaacc cccacagtcc tcttcccagg gaagcaggct gctggctctc |
| 6481 | cgtgccccac tgtacagatg ggctgagccc cttccttgtc cattctcagg ccagggacct |
| 6541 | ggaagatctc agcccgattc tcagatggcc tggaatccaa cagcagcacc cagtttgagg |
| 6601 | tgaagaaata tggtgagagc tggaaactgg agggacaggc agctgctttc ctgaaggaaa |
| 6661 | taagggtgga aggagaggta ctgggagcag ctcagggcag ggagatatgg gtgccacagc |
| 6721 | cctgagcaga ggggagtctt tgagctggag tctgacctgc ctatcccttc accctgggtc |
| 6781 | agtccttccc aactttgagg tgaagatcac ccctggaaag ccctacatcc tgacggtgcc |
| 6841 | aggccatctt gatgaaatgc agttagacat ccaggccagg taatacctcc ctccccacct |
| 6901 | ctgcccacca gcaccgggtc ctgctcccta ctcagtatga atgggctcct gcttccctgc |
| 6961 | cctcgggcca ttattccccc cagcccttgg cccaccctct tctctctgcc acgacaggta |
| 7021 | catctatggg aagccagtgc agggggtggc atatgtgcgc tttgggctcc tagatgagga |
| 7081 | tggtaagaag actttctttc gggggctgga gagtcagacc aaggtaggaa ggagaatagg |
| 7141 | ggctggggag gggaaggggc aagggaggtg aggtgggaga ctcagtctca ccctatgtcc |
| 7201 | tgtttctttc tatgccccag ctggtgaatg gacagagcca catttccctc tcaaaggcag |
| 7261 | agttccagga cgccctggag aagctgaata tgggcattac tgacctccag gggctgcgcc |
| 7321 | tctacgttgc tgcagccatc attgagtctc caggtgggtg actttccctt attgtaaccc |
| 7381 | cagacccttg cctctgacct ctgagctaac cctctgtcct ccggcaccaa caccacccca |
| 7441 | cttctcacat ctcatctcag actcaaaacc aggaaacacc caggagacct ggtttctctc |
| 7501 | caactctgtc tctgtgactc ggcccttttc cctggctgag tttatttatt tctttgctcg |
| 7561 | ttctgctcat tccttcactc ctccagtgga catgtgttgt tcaatgcccc gtgctaggcc |
| 7621 | tcagcatgca cagacatgtt ggggaccagc ctcaacgcca cccgtagggt tcctgaagtc |
| 7681 | cattggtgac acaggaatga gaagagacag gttaagagtt cataaagagt gggggccagg |
| 7741 | gggccaattg caaaatggag gctgcaaaag gctcagagct ctggtctcca cactattttt |
| 7801 | tgagtacagt cactcagatc taagaagcag atgttcaggg agaaacagtg aaagggaggc |
| 7861 | agtgggtcat aggcgtaatc tatagcaata gagttttaaa tgaatctcct ttgtgctcaa |
| 7921 | acagcatgtc tttaaattat cggagagtag ctggtggaag tgggcttagc tagaagactg |
| 7981 | catgtctgtc caatgcttca aaggagggtc tttctccttg aacagagtgt ttacagataa |
| 8041 | gacagggggt ctcactctga gcatgggaac atgatggcaa ttaggaggct tttcttctca |
| 8101 | gaggcctctt gtggctttcc acaacttatt gtctcatatt tttatggaca gtttatacag |
| 8161 | gcaccccaca agtccttttc ccaacatgcc cccctccctt tttttttttt taaccgctat |
| 8221 | tgctattatg gcttatttgt ggtgtttggt ctgttttcag aagtgtcttt tgcatctgta |
| 8281 | gactaaaagt aaacagcata aacagataca cattaaagta aaatttgtaa tagttgatcc |
| 8341 | tttaatggtc ttaatctgtt taagaggatt tatgtttgaa agtccgtcag tagctccaat |
| 8401 | gagaatgtca gtctcaggca ggagggttaa atgagcctga gatgctttaa aaacctgttt |
| 8461 | ttttaaaatt tggttatatt taatgttaaa tttttatttt tttcttttag atgatgtcta |
| 8521 | actttttaaa aatgatgttt agtagtatta tacgaatggg gagttatgta gaaattggaa |
| 8581 | gtatttcaat tacattgtac ttctaattga tgttttaagt ttattgtacg atcttccatt |
| 8641 | taaataacag tctgtctaag atcatttgtt tgatttgtca attgttggtc tatttgggtc |
| 8701 | tgagaattcc acaattttga ggaatttttt gttaactatt tatatatttt gtagtttgaa |
| 8761 | cagaggagtg taaagcaatt ccagcagccg cagcagtagc tgtgactgca ataaggccca |
| 8821 | taagactgtt ataagggtaa aaataaatct ctttgttttg gtaaacactt ttttttaaaa |
| 8881 | catttttgtg acaatatgaa tggaaggaga ggctttctaa ggtctattga gggaaaccag |
| 8941 | tatccaaact cctttcttag tttttatcag taacacagat gtttttacac cgaacgtgga |
| 9001 | attaatacag gtgaaaaggt gacagttttg acaagtaata gtttgagaat taggtcgaat |
| 9061 | gtcaatattt ttgaccatta acataaaagg agggttgaca caactctgaa tgggcactgt |
| 9121 | tttgttggaa gaaaactgat acgcaaattg aagtttttaa cctttttttt ttaaagataa |
| 9181 | tatatttttt tctaaactta aatatgagat tgggccatta ttaactttca taatttggag |
| 9241 | tgtttagggc ctattattgg attaattatt ttgggatgtg ggccagctgt actaaaattg |
| 9301 | gtccaaatta tgggaaaatg agcacgtttt tcagtgtaag tagtgttacc tttttgatag |
| 9361 | tatagtttct gttttagttt tgtcttgtat ttattatttt gatgggtaca attaactgta |
| 9421 | aaggtcccct caggggacca attaatgaca atttcatagg aattattttg tagtaccata |
| 9481 | gtgtgatcag agatgtaatt ttttttaatt aatattttta aattatttga ccattgttaa |
| 9541 | ggttgttggc acctcttttt tgggggctta aactgttaat tgaattgaac tctgtgaatg |
| 9601 | atccgggctc catccagaaa ataaatgata ggatactggt ctttgattat gacctggaat |
| 9661 | tttaactagt caatgttgtc ggtagccttt taggcaaccg atagttggcc ttatgtaaag |
| 9721 | aggggggaac tgataaccta tggacacatt tattaacttt tttttttttc ctttgggtga |
| 9781 | gagggcccat gagtatttgt aggcttaggg atccaaacgc tattattaac ataaacttca |
| 9841 | actgggggtt ttaaccatgt gacaggccta attaaaggca ggaatgggac acatgcccaa |
| 9901 | taggtataat tttgggctgt tgtagccaca ggtttgttag gcgaggaggt cactgttttt |
| 9961 | attttggctt tgtattctag gattagtaaa taacagaaga caaacatgag tataattagt |
| 10021 | aacttttttt tttagtaaaa gagtgacctg tagtgttact tggcatctta gtttactata |
| 10081 | tgttattaat gaggaacccc actgggggta tgttaattta ttctagctaa gcagttatgt |
| 10141 | tattagaagc tgagaagggg gtgtttgtta aagtaacagg gcagaagaaa ggcggattta |
| 10201 | agatacgagc ttaatacagt gtagcaggta taggtagtag gcaaagtgag agaattaaaa |
| 10261 | atgaataaat tatttggctt agacttttgt ttttttagta taatgtctga ggcctgtgtt |
| 10321 | gtttgtggaa gtcgcattgt tgaggctgta gttcctgtag ggtctttttt aggctggttc |
| 10381 | aaatgttttt ttatttttta attttttatc ctttgatgag gatgtagtct ttaggctggt |
| 10441 | actggaaatt ttaggagtgg cgtctgtgtt aagagacttt ttacaatttt taaagagcag |
| 10501 | gttagtgttt taagaaaaac ttgtgtttta ttttaatgtt tagtttatag aaaactggat |
| 10561 | gatatctttt taactttagt aaatacgttt acacacggaa ttttttacaa ttatcatttt |
| 10621 | aaaacttgtt tagatcttta aaacaaaatt aaacaacctt ttttgtataa attttttata |
| 10681 | acttttttta tgacttttac agacaatttt taacatgtct taacttttta tgttttataa |
| 10741 | tttttttact aaaggtacat ttttataact ttttaaattt ttttactttt ttgtattttt |
| 10801 | ttgatttttg tcttagtctt ttttttactt ttattttttt aaatgtgtaa taattagatg |
| 10861 | agtgttggta acaatggatg tatgtacata ttttagtttt taaaatttag ggatgtgttt |
| 10921 | aacatctgtt tgccagaact gactaggttc caattcttta cggttaacac ctattgaagg |
| 10981 | agggtatgtg cctgtgagct ggtaatctgg gcattgtggg ataatttgtt tagccagcct |
| 11041 | ctgtgtaagt tgaaattatt tagataagtt tctccaattt tggtggaata atcgatgtga |
| 11101 | ttgggtggct tggtcaagca gtgatgtcat aacctgaagg tctgcttgat tattgccgta |
| 11161 | agccaatggg ccaggcagag agctgtgggc tcgaatgtgt gtaataaaag taggatgtgt |
| 11221 | accttggtct agtaattgtt gaagttgaag aaaaagacca cacagagtgg gctccagagc |
| 11281 | aaacttaagg ctgtaatagt ttttaaataa atacacagaa taaccttagc tctctgaatg |
| 11341 | ttagtaaatt cagatcaagt gattggatta tgtggtctcc accagactgt tgctttttca |
| 11401 | tgtttaccag acccaccagt aaaaacagct atggctcctt ccaaaggggc atcacaagta |
| 11461 | atttttggaa gaacctatgt agttaatttt aagaattgaa aagtttttag gataatgatt |
| 11521 | attaatacat ccaacaaatt ttgttaaatt aatctgtcat gtaactgagt taataaatgc |
| 11581 | ctgtttaacc tgatttttat ttattggaac tataattttt attgggctca gtgccacaaa |
| 11641 | atttaataat tcatatatga gcctgtccaa ttagaattgc catctgattt aagtatactg |
| 11701 | taagtgcttt tatggtatta tgtggcaaaa aggaccattt aactaaatca tcattttgaa |
| 11761 | caataacccc cattattgtg tggttagtgt gaagtaggga acacaatgaa ttataaaggc |
| 11821 | aagtctgagt caatcctact gacctgggct tgctgaattt tgttttcaat tactgataac |
| 11881 | tctttcatgg cctcgggtgt tagttctctg ttactgcgta agttggtatt tcccctcaat |
| 11941 | attgagaaga gattagacat agcataagta ggaattgcta aattgggcca aatccaatta |
| 12001 | atatcttcta acaatttttg aaaattattt aaggttttga aagaatctct tctaatttga |
| 12061 | accttttgag gcttaatggc tctatcctgt acttgtattt tcaaatactg aaaaggagtg |
| 12121 | gttgtttgaa ttttgtcagg tgctataagt aattcagcat ttgtaattgt cttttgcaaa |
| 12181 | gattaataat attgaataag ttggtctcta ctttttgctg cacaaatctg gaaactgatc |
| 12241 | tctaacaggc tggatagttc tgcctacaaa agtttgacaa actgtgggac tatttaacat |
| 12301 | accctggggc aaaactttcc aatgatattt ggctgcaggt tttttgttat taacggcagg |
| 12361 | aatggtaaag gcaaattttt tgaaatctgc ctctgctaaa ggaattgtaa aaaagcagtc |
| 12421 | ttttaaatct ataataacaa gcggtcagtc tttagggagc acagtggggg atgggagccc |
| 12481 | aggttgtaag gctcccatcg gttgaattac agcgttgacg ccatctaccg gactttttct |
| 12541 | taattacaaa tactggggaa ttccaaggag agaaagtggg tgaaatatat ccttttttta |
| 12601 | gtagtttatt ttataaagca cccccaactt ttccttaggg agcggccact gttcaaccca |
| 12661 | gacggggcgc cgggtcatcc attttaaggg aaattgctcc ttcactgtaa taactgtagg |
| 12721 | gtgaacctga attgccccat ctccataatg aactgtgggt cgggcaataa tgggcacggt |
| 12781 | gagccaagtc tcgggctccc tccccctgca cccactcggc tgaggaggag gtggccattc |
| 12841 | tggacatttc tctacaggaa ccgtgggctg aacaattttt tgagtaggtt tagggagact |
| 12901 | ggggagattg gcataaatca tcttcagact ctcctttttg ttagtactcg gtagaggtgg |
| 12961 | ttcagagttc tgattatcaa actcctctct ctcctcctct gactcagcct cattatctgt |
| 13021 | ctgaaaaggc tccagtgctg catgcaccaa tgaccaaagc gaccaaacag gcaaaggaat |
| 13081 | ttcctttcct tctctatatg ctcttttaag gtcctttcca actccttctt aatgttttaa |
| 13141 | tttcaaagtt tcctgttttg ggaaccaagg gcaaaattgt tccatagcat gaaacaaatc |
| 13201 | cataagattt tccgtatcaa cttttacccc accatgcatg cttgaagagc tgccgtagga |
| 13261 | agctcaaata cgtggtgtac ttactttcag tttttcccat tgtgtcccta gctttctctg |
| 13321 | ggcgccccgc ttacctgtag aggttaaaac ttttatgtcc ttgggagtcc tttgttcgtt |
| 13381 | ggtcctctgt ttcacatgct tgagcgtttc ctcaccagat tcttttgggc cccacgttgg |
| 13441 | gcgccagaat gttggggacc agcctcaaca ccacctgtag ggtacctgaa gtctggtggt |
| 13501 | gacaaaggaa tgagaagaga caggttaaga gttcataaag agtggaggcc agggggccaa |
| 13561 | ttgcaaaatg gaggctgcaa aaggctcaga gctctggtct ccacactatt tattgagtac |
| 13621 | aataacttag atctaagaag cagatgttca gggcaaaaca gtgaaagggt agcagtgcgt |
| 13681 | cacaggcata atctacagca gaagcgcttt aaatgaatct cctttgtgct caaacagcat |
| 13741 | atctttaact tatcggagag tagctagtgg gagtgggctt aactaggagc ctgcacgtct |
| 13801 | gtccacattc caatgcttca aaggagggtc tttctccttg aatacagtgt ttacagataa |
| 13861 | gagagagcag gtctcgctct gagcatggca attaggaggc ttttctcctc agaggcctct |
| 13921 | tgtggctttc cacaacttat tgtcccatat ttttatggcc agtttataca ggcaccccac |
| 13981 | aagtcctttt cccaacacag acaggaatac ggcagcctgt gccctgggag ctcactgtct |
| 14041 | tgtgggaggg aaccactcaa gccactcccc acttgtcctc ctgtccctct cttcttgggc |
| 14101 | tctgtccccc acctctctct gtcctttgtc ttgcaggtgg ggagatggag gaggcagagc |
| 14161 | tcacatcctg gtattttgtg tcatctccct tctccttgga tcttagcaag accaagcgac |
| 14221 | accttgtgcc tggggccccc ttcctgctgc aggtttcttc cagaggggaa ggatgagtag |
| 14281 | ggaggatgtg gtagttagga gggctcaggg tctgaccact ctcttttgcc tgccctcctt |
| 14341 | tacctgccta ggccttggtc cgtgagatgt caggctcccc agcttctggc attcctgtca |
| 14401 | aagtttctgc cacggtgtct tctcctgggt ctgttcctga agtccaggac attcagcaaa |
| 14461 | acacagacgg gagcggccaa gtcagcattc caataattat ccctcagacc atctcagagc |
| 14521 | tgcagctctc agtaggactc ctcggacccc tgggagatgg tgggggaagg ggaggagggt |
| 14581 | gagctggggt cccaaggatc catggcctga cttgggggga aggtggggta cttggctctg |
| 14641 | agctactacc ctattcgcac ctgaccccct ctccaggtat ctgcaggctc cccacatcca |
| 14701 | gcgatagcca ggctcactgt ggcagcccca ccttcaggag gccccgggtt tctgtctatt |
| 14761 | gagcggccgg attctcgacc tcctcgtgtt ggggacactc tgaacctgaa cttgcgagcc |
| 14821 | gtgggcagtg gggccacctt ttctcattac tactacatgg tgtgcatgag ctggggagtc |
| 14881 | acggagggct ggggtgcagg gaagagccct ctgggtgggg ctgggggggt tcaaggctga |
| 14941 | ggctgtccca tgaagaggca accactcttg tccctcccat tcttggccca gatcctatcc |
| 15001 | cgagggcaga tcgtgttcat gaatcgagag cccaagagga ccctgacctc ggtctcggtg |
| 15061 | tttgtggacc atcacctggc accctccttc tactttgtgg ccttctacta ccatggagac |
| 15121 | cacccagtgg ccaactccct gcgagtggat gtccaggctg gggcctgcga gggcaaggtg |
| 15181 | accggggtca ggagagatgg cacttgtgcc gagggggttg aggacagggt gattgccaac |
| 15241 | agggcatgga tttagcttgg gggcagtgag gataccggga ctgaaggaag ctctcccact |
| 15301 | ctgaccgccc ccacctgccg cccctgccag ctggagctca gcgtggacgg tgccaagcag |
| 15361 | taccggaacg gggagtccgt gaagctccac ttagaaaccg actccctagc cctggtggcg |
| 15421 | ctgggagcct tggacacagc tctgtatgct gcaggcagca agtcccacaa gcccctcaac |
| 15481 | atgggcaagg tttgtccaga ccctctccac agctctctca cccctccatg gctcatcccc |
| 15541 | ctgcttccct gagccttggg cgcagcccct ggatcccact gaggctcccc acagtctctt |
| 15601 | ccccacttgg ccctgtggtc tccatctcct ggctctgtat cctttcctat ccccccatgt |
| 15661 | gctgccctct cacctgtgcc gagtgctcag tcctgcccct cagccacact tggctcctag |
| 15721 | cattcctgcc tttcttgcag gtctttgaag ctatgaacag ctatgacctc ggctgtggtc |
| 15781 | ctgggggtgg ggacagtgcc cttcaggtgt tccaggcagc gggcctggcc ttttctgatg |
| 15841 | gagaccagtg gaccttatcc agaaagagtg agaacagaga aggaagggga gtgggtggcg |
| 15901 | ggaagataag gaaggaggaa gggcctgagg ggaccagctg gaagagtccg ggcaggaagg |
| 15961 | gctgggcagg ggaaggggag gaggggagga ggccgagtgc ctgacggctg gactgcagcc |
| 16021 | tttctctcta ccaggactaa gctgtcccaa ggagaagaca acccggaaaa agagaaacgt |
| 16081 | gaacttccaa aaggcgatta atgagaaatg tgagttgcgg gtgcctaggc agtagcttgg |
| 16141 | gctctccacc tgggatccgg gttgggggtc tgcctctctg cccctcggct ccttgctgaa |
| 16201 | cccacgtgtg gtatttgggg ccagagatcc gaattccggg attacgagtg gaaggtgggc |
| 16261 | agctctctcc agcagcctct cttatgttgc tggtctcaag gggtcggggc gggggctgag |
| 16321 | gtgtatgtcc tttttgtcct ctcatgctca cccccacctg gccctgcagt gggtcagtat |
| 16381 | gcttccccga cagccaagcg ctgctgccag gatggggtga cacgtctgcc catgatgcgt |
| 16441 | tcctgcgagc agcgggcagc ccgcgtgcag cagccggact gccgggagcc cttcctgtcc |
| 16501 | tgctgccaat ttgctgagag tctgcgcaag aagagcaggg acaagggcca ggcgggcctc |
| 16561 | caacgaggtg aggggctggg tggggctagg gcacaggtgg cggcgcttgg aaaggcagaa |
| 16621 | cggtcccctc ctcactcccg tccaccgtgg tcccccagcc ctggagatcc tgcaggagga |
| 16681 | ggacctgatt gatgaggatg acattcccgt gcgcagcttc ttcccagaga actggctctg |
| 16741 | gagagtggaa acagtggacc gctttcaaat gtgagagtgt gtgccggccc ggccttttct |
| 16801 | ctgtgctgtg tctcggggcc agccggggta gacgggcctt ctctgccttt ccctacacag |
| 16861 | attgacactg tggctccccg actctctgac cacgtgggag atccatggcc tgagcctgtc |
| 16921 | caaaaccaaa ggtgatgtca ccctgtctgg gcctcaggtg accctgcttc catttccctg |
| 16981 | taccccagct ccctgttccc tttgctctta gtgtaggaag agggtccagt gatctgggga |
| 17041 | ggtctgtgcc agcgtgcagc tggcgtgggc cagagggcag aggcggactg agacagagct |
| 17101 | gggtcacccc cacccctccc tcctgtggcc ctgaagcttt gatggcccct ctgatctctg |
| 17161 | cccctgtgcc cacgcttcct ttccctcagg cctatgtgtg gccaccccag tccagctccg |
| 17221 | ggtgttccgc gagttccacc tgcacctccg cctgcccatg tctgtccgcc gctttgagca |
| 17281 | gctggagctg cggcctgtcc tctataacta cctggataaa aacctgactg tgaggcccca |
| 17341 | tgggagcctg agcatacagg agttggggga gccagggccc agtgaggggt ggggaggcta |
| 17401 | accgggccag gactctggcc atcctcgttt tcctgccctc aggtgagcgt ccacgtgtcc |
| 17461 | ccagtggagg ggctgtgcct ggctgggggc ggagggctgg cccagcaggt gctggtgcct |
| 17521 | gcgggctctg cccggcctgt tgccttctct gtggtgccca cggcagccac cgctgtgtct |
| 17581 | ctgaaggtgg tggctcgagg gtccttcgaa ttccctgtgg gagatgcggt gtccaaggtt |
| 17641 | ctgcagattg aggtgaatgg agcacccctg aatataagtc cccgggcccc cagctttgtc |
| 17701 | ctccaccctc agcactctct ctgctggcca ggccaggggc ccaacaccca aaccaatgcc |
| 17761 | ttggtctgtt cccatcttct acaattctga tccaactctg tccctggagt tgaaactcaa |
| 17821 | agttctgggg gagtctgcgc tagcagggca ggctgtagtc ctgtgtgacc tcacaaccat |
| 17881 | gttttccctg agacagaagg aaggggccat ccatagagag gagctggtct atgaactcaa |
| 17941 | ccccttgggt gagtgaccct ctacctccag ccattggttt cctaagtggg tacaggtggt |
| 18001 | gggggatgtg gacagcagga caggctgcca acttccccca tttccccaga ccaccgaggc |
| 18061 | cggaccttgg aaatacctgg caactctgat cccaatatga tccctgatgg ggactttaac |
| 18121 | agctacgtca gggttacagg tgggagtgcc ctttagtccc ttcccagtgg ccaccttcgg |
| 18181 | attcatgtgg gacttgtgga tccctgcttg gtcccactcc ccgtgagcct ctgacacaga |
| 18241 | gtcctcagac ctccaccctc tccctcccat gtagcctcag atccattgga cactttaggc |
| 18301 | tctgaggggg ccttgtcacc aggaggcgtg gcctccctct tgaggcttcc tcgaggctgt |
| 18361 | ggggagcaaa ccatgatcta cttggctccg acactggctg cttcccgcta cctggacaag |
| 18421 | acagagcagt ggagcacact gcctcccgag accaaggacc acgccgtgga tctgatccag |
| 18481 | aaaggttctg ggtgcaaggg caagcaggag gggggccagg aaaggacagt tactggaaga |
| 18541 | tggacagccc aggaggctac agagggaaag aaagggggcc cctgatgagg atggggagca |
| 18601 | tggccttggg ctcaaacagc agaagggtga gtgtcacctg agcggccacc tctcctctcc |
| 18661 | aaggctacat gcggatccag cagtttcgga aggcggatgg ttcctatgcg gcttggttgt |
| 18721 | cacggggcag cagcacctgg tgagcttggg agagtggttc cagggttctg agggggtcag |
| 18781 | ggctggggca ggggtgggac agagctggta tgatgggagg gtggataacc aggcacctgg |
| 18841 | gggcgtgggc ataatgagaa gcaagtcctt atccccaacc ctcctttcct gccctccagg |
| 18901 | ctcacagcct ttgtgttgaa ggtcctgagt ttggcccagg agcaggtagg aggctcgcct |
| 18961 | gagaaactgc aggagacatc taactggctt ctgtcccagc agcaggctga cggctcgttc |
| 19021 | caggacctct ctccagtgat acataggagc atgcaggtgc gggcatgctg gggctggccc |
| 19081 | gagaagcgcc tgtcggagga ctctctttgc cccttccccc tcctgtttga catcttttct |
| 19141 | ccccttacta ggggggtttg gtgggcaatg atgagactgt ggcactcaca gcctttgtga |
| 19201 | ccatcgccct tcatcatggg ctggccgtct tccaggatga gggtgcagag ccattgaagc |
| 19261 | agagagtggt aagttcagtg gcgtttctgc cctctgctgg cccccagctc tctccctttt |
| 19321 | tcctcaggaa cccaggggtc caggcccaag accctcctcc cgttttcttc caggaagcct |
| 19381 | ccatctcaaa ggcaagctca tttttggggg agaaagcaag tgctgggctc ctgggtgccc |
| 19441 | acgcagctgc catcacggcc tatgccctga cactgaccaa ggcccctgcg gacctgcggg |
| 19501 | gtgttgccca caacaacctc atggcaatgg cccaggagac tggaggtgag gggtgagggg |
| 19561 | ctctggcagt gagcctgagg cccaggggac cttaggatcc ctgagtgtgc ccagagggag |
| 19621 | aggctggatg aagactcaga ggaggaatga agttataagc aggggtgggt tgggggagac |
| 19681 | tcaggagagc ccagcagggg gtggctaagg gccaggggac caggctcttc tccctgcctt |
| 19741 | cctgtttact cgtggtctcc cttcactttc agataacctg tactggggct cagtcactgg |
| 19801 | ttctcagagc aatgccgtgt cgcccacccc ggctcctcgc aacccatccg accccatgcc |
| 19861 | ccaggcccca gccctgtgga ttgaaaccac agcctacgcc ctgctgcacc tcctgcttca |
| 19921 | cgagggcaaa gcagagatgg cagaccaggc tgcggcctgg ctcacccgtc agggcagctt |
| 19981 | ccaaggggga ttccgcagta cccaagtagg ggccgtcccc gggctctggc gggggtgggt |
| 20041 | agtcctcaga ccaagggctt gcttgagtcc tggctcaacc tccctaggac acggtgattg |
| 20101 | ccctggatgc cctgtctgcc tactggattg cctcccacac cactgaggag aggggtctca |
| 20161 | atgtgactct cagctccaca ggccggaatg ggttcaagtc ccacgcgctg cagctgaaca |
| 20221 | accgccagat tcgcggcctg gaggaggagc tgcaggtgaa ccactccctg gtgaaccact |
| 20281 | ccctcgcctg ggtagccagg acacctgggc ctcgtggcca ggccagaagc cgtccccacc |
| 20341 | ctcccacccg tggaatcccc gcagcacttc ttcctggggt cttcggggga agactgactt |
| 20401 | cctggctgcg tgacctggag ctctgagctt cagttttctc acttgtagag taacatacac |
| 20461 | agagttcacc ctacagggtc gttagaaggc tgaagtgaga taattcatgt gctggtataa |
| 20521 | actttgtgga aatgtgaggt ggggagaggg ggtggggctg ttttgaggaa ggagataagt |
| 20581 | tattggagcc gcaaaaacag gtttgcttgt gcccttctaa catcgccttc ccttttctgt |
| 20641 | tgctgaagtt ttccttgggc agcaagatca atgtgaaggt gggaggaaac agcaaaggaa |
| 20701 | ccctgaaggt gagggccagg gaaggggtgg ggccaggcac tggtggagga gagggtgtgg |
| 20761 | agtgagaggc ctgtgggcag aggcacatgg tccggggaag gaggcagaca cctcagggtt |
| 20821 | ggtgtcccgt gcttccgtcc tgggtgtttt tccccctgct tgctttcgct tgctctcccc |
| 20881 | atctctgggt acctgttgtt tcctttaccc gcctcagtgc tggtggctcc gaatcccact |
| 20941 | cctcagccca ggcctcttcc ctgaaccatg ggccccactc gtcccactcc cacagcacct |
| 21001 | cagacgaggc atgtcccaaa gcccttcttc attctgtgtc tcttgtctgg ctggtgggag |
| 21061 | cccctcccag ccaggagccc agccactact ctagaggccg tgttagtggc ccctctccca |
| 21121 | agcctgtcct tatgtcccta gtgactcctc ctctgctccc ctgctgcctg tggcccttgg |
| 21181 | tgctgcatcc tagattctgt gctgagacgg ccttctccct acctggaact tctctctacc |
| 21241 | tcctgtctcc cctgtctgat ccactgtcca cacggcagtg acactgacct tccaaaagcc |
| 21301 | ccagccagat cagccttggg gaaaagtcac tccccgctgc ccacggctca gatggctggg |
| 21361 | cctctgccca cccctccggc cagacagctc tccttgtcta cacagatccc cttgcctttc |
| 21421 | ctgtccttcc ctgcttcttg gcccacagga caagctcttt cttctccttc aagccttggc |
| 21481 | cagaagcctt tcctgagctt ttcagtccag cctcttccca gcacagtctg gagtgttggc |
| 21541 | ctctgggggc aggcccctgc ttctttacct ctctgtctcg cctgacgcct gtggcgaatg |
| 21601 | tggtgccact cgtgtgtgtg gactgtgcag tgacggggag gaaaaggggc tgaaggcctc |
| 21661 | aaatcctgta gcccagggag atgcccttag gtatggcacc agagaggtct gtggcctcac |
| 21721 | atgtcccacg tcctctccct gccccttgct gagccaggtc cttcgtacct acaatgtcct |
| 21781 | ggacatgaag aacacgacct gccaggacct acagatagaa gtgacagtca aaggccacgt |
| 21841 | cgagtacacg agtgagtgtg ggggttggga ggccttgggg ccaggcaggg gctggcgcag |
| 21901 | ggagccgggt ggccatccca gccctcctca caatgcttcc ctgtgcagtg gaagcaaacg |
| 21961 | aggactatga ggactatgag tacgatgagc ttccagccaa ggatgaccca gatgcccctc |
| 22021 | tgcagcccgt gacacccctg cagctgtttg agggtcggag gaaccgccgc aggagggagg |
| 22081 | cgcccaaggt ggtggaggag caggagtcca gggtgcacta caccgtgtgc atctggtggg |
| 22141 | cgccgggagc tgccctgggc caggggaggg agggcaggac ccaggctggg gctgggcttc |
| 22201 | tggagcccgc gcaggcagaa cctggacgac agctcacacg tctccacagg cggaacggca |
| 22261 | aggtggggct gtctggcatg gccatcgcgg acgtcaccct cctgagtgga ttccacgccc |
| 22321 | tgcgtgctga cctggagaag gtgtggtcag ccacccaggg caaccccctc tgtcccaggt |
| 22381 | actgagccct gtcatgtgca gggcctgtga ccaactcccc ttttccacag ctgacctccc |
| 22441 | tctctgaccg ttacgtgagt cactttgaga ccgaggggcc ccacgtcctg ctgtattttg |
| 22501 | actcggtgag tggggagaga tgaggcagga agggactcga tggcaccggg tttactgagt |
| 22561 | atgcgttagg aggtttctca ggagacagct gtgtcagcgg ctggtgctct tgagaacttg |
| 22621 | tgatgtcatc agagagaagg acaagaatgt gagcccgtga gacacagcag agtaaggggc |
| 22681 | agacctgcag gcggcaggga ccgatgccag tcagcaggga ccctcagggt ttgagaggga |
| 22741 | gtctttccta atgctggttt tattcagctt gaggggctgc ctttgttttt ttgttgaact |
| 22801 | tcctatcttt tttttaatat taaagcgtat tttcctttac aaagtgatgg tggccataga |
| 22861 | tgatagttgt atttgtcttt tcacgacctt atttggctaa aatagttatc aaccctctta |
| 22921 | cggctctcaa aacattttta tttatttatt tagtaaagac agggtctcgc tctgttgccc |
| 22981 | aggctggtct tgaactcccg gcctcaagcg atcctctggc ctaggccttt caaagtaccg |
| 23041 | gatttacagg ccagagccac catgcccggc cttcaaaaaa agttttggaa catttactgt |
| 23101 | aacctctggg agaaaatgtg agaaaggtgt ggtggctgtc attagccagc tgtttgtagg |
| 23161 | tcagggagac ccctacccag tgtgtgcaga ggggccagcc cccatcagct ggggaagcct |
| 23221 | ggctgacaca tctgggttga acacaataga aaacacagag ccaacaagat tcccggatag |
| 23281 | ggagctgacg gtgcagcagc ctagctcagg agggacactg gcacggcacc gtgtggactg |
| 23341 | ggcccgcgtg ggcacgagga ggggtcaggc ctgggacctg agtcgggggg tcaggcagga |
| 23401 | tgacagaacc tgcagttagg ttgtggcaaa taaaggagga cccagttgta tccatgacaa |
| 23461 | agatgaggcc gcgaggaggg cgagtgggtt tgggggcagg cagagtgcct tggagaactt |
| 23521 | acaggtcctg ccacaatcct aatgcaagga tggagctgca agttcagttt gggaatcatc |
| 23581 | agcctggatt ggtttggtgg aagccaggga gtggttgaga cccccacagg ggagctctga |
| 23641 | ggaaggaagt tccgaaggag ggaacgtaag aaatgaccag gtcagaacca agggtggtcc |
| 23701 | agaagctaac ccttagctta gggacagttt cacagagaac acgtccatga tgcaagactc |
| 23761 | tgctgagggc ctggagcagt gaagactggg gcaaggtcac cctctgggaa gtgaagtcac |
| 23821 | cagagacctt gcggagcagc tttgagagtt ctctgagtag gaaggtaaca gaatgtgaag |
| 23881 | gacactggag agaaggccaa taggaagcaa acaaaaacag gccaaggaaa cccagtacag |
| 23941 | ggggctgcag ggcccaggga gtgggtccct catctctcct ccccacgctt ggccaggtcc |
| 24001 | ccacctcccg ggagtgcgtg ggctttgagg ctgtgcagga agtgccggtg gggctggtgc |
| 24061 | agccggccag cgcaaccctg tacgactact acaaccccgg tgagcactgc aggacaccct |
| 24121 | gaaattcagg agaactttgg cataggtgcc ctcctatggg acaatggaca ccggggtagt |
| 24181 | gagggggcag agagccctgg ggctccctgg gactgaggag gcagaatgga ggggcctgtg |
| 24241 | ccctaactcc tctctgttct ccagagcgca gatgttctgt gttttacggg gcaccaagta |
| 24301 | agagcagact cttggccacc ttgtgttctg ctgaagtctg ccagtgtgct gagggtgaga |
| 24361 | ctgagggcct ggggcggggc agtggaggcg ggatggccgg ggcccccccc acactgtctg |
| 24421 | atgggttccc caacttcagg gaagtgccct cgccagcgtc gcgccctgga gcggggtctg |
| 24481 | caggacgagg atggctacag gatgaagttt gcctgctact acccccgtgt ggagtacggt |
| 24541 | cagtcttccc accgaggccc tggcctgacc ctccctcggg gaccggccgt tttggtctct |
| 24601 | ctgggtgtag cctgctcctc ttacaggtca tgcacgcagc ctgtttgctc tgacaccaac |
| 24661 | ttcctaccct ctcagcctca aagtaactca cctttccccc ttctcctcac cccctcttag |
| 24721 | gcttccaggt taaggttctc cgagaagaca gcagagctgc tttccgcctc tttgagacca |
| 24781 | agatcaccca agtcctgcac ttcagtatga agcaaaccgg agaggcgggc agggctgggg |
| 24841 | ggagacaggg aggctgaggt gtggccgagg acctgaccat ctggaagtgt gaaaatcccc |
| 24901 | ttgggctgtc agaagccttg ggcttggcca taaataggga ggcagtggca cctctccatg |
| 24961 | ggggtggcga aggtggaatg agaggatcta cacagagtcc ccagcctggg ctcaccctgc |
| 25021 | accttctctt cccctctgac cacttttgcg cacgtcatcc ccgcagccaa ggatgtcaag |
| 25081 | gccgctgcta atcagatgcg caacttcctg gttcgagcct cctgccgcct tcgcttggaa |
| 25141 | cctgggaaag aatatttgat catgggtctg gatggggcca cctatgacct cgagggacag |
| 25201 | tgagtcatct ggtcccctca gtctcttgtc ctccccatgc ctcgccacct aggccttgcc |
| 25261 | cctcagaagc cagatgcctg tgctctccgt ttccacctgc catcctcccg agccctgctg |
| 25321 | actgcccctt tgccccctgc agcccccagt acctgctgga ctcgaatagc tggatcgagg |
| 25381 | agatgccctc tgaacgcctg tgccggagca cccgccagcg ggcagcctgt gcccagctca |
| 25441 | acgacttcct ccaggagtat ggcactcagg ggtgccaggt gtgagggctg ccctcccacc |
| 25501 | tccgctggga ggaacctgaa cctgggaacc atgaagctgg aagcactgct gtgtccgctt |
| 25561 | tcatgaacac agcctgggac cagggcatat taaaggcttt tggcagcaaa gtgtcagtgt |
| 25621 | tggcagtgaa gtgtcagtgt gtgttgctag ggctgagagc agtgcccctg cccgatgcag |
| 25681 | ttctgggcag gccaggttga cataacctta gactctctga gccctgatga cccttgggct |
| 25741 | gttcagctct gctagaacct cccagatgac ccgctaggag tctagtgctt cacaggacca |
| 25801 | ccccgagcag aactgggacc caagagcctg caccccaagg accagagtcc atgccaagac |
| 25861 | cacccttcag cttccaaggc cctccactgc ccggctgtcg ccagtcacca cggcctcaga |
| 25921 | cagggcttgt gctcagctga cacctgtgac acagctcttc tgcctcatga gctgttgtcc |
| 25981 | agctacacct ccccgactct gtcctcgtgc tgctggcggt tctgaggtct gcagatttta |
| 26041 | gctgagttcc gggctgttga aagcctgctg acgcttggtt ctgttatcag tggaatgagg |
| 26101 | tgactttccc ggagttgtgc aatcctcagg tccggcagtg tcttcttcca gttactggtt |
| 26161 | tcaaacaagc caaaagtctg actttggtgt gtttgtgaat cctctgagga agccgctgtt |
| 26221 | ctcctggggt ctccccttcc caccggacct gcctaacttt cccccattta gtggcacacc |
| 26281 | tggggtcttc agagatgact ccgcgtctgt ccaaagaagt ttggtgagat cagtttccgt |
| 26341 | agaggtcatg acagttcagc agcctgccat ccagtcattc gacagaaatt cgggaatctt |
| 26401 | tcacttcatg ccatgccctg tgccaggtgc cagagataca gctgctcact ccagggctca |
| 26461 | tcgctgggga gacagataag aggacgggca gtccccaccc tctgtgaaag atgtgatgtc |
| 26521 | agggagcagt gtggtcctgt ggggcatcta accaagtcag gggcattgcc aggcagggac |
| 26581 | agggaaggct tcctggagca ggtggcctcc aagtggggct ctgaagactg agaaggagcc |
| 26641 | aggaaaagag caggggtaga tgagggcatc tggggcagaa ggagaatata caaaggccca |
| 26701 | gaggccgggg gcaggacagg gtacctttgg ggacattgca tgtaattgac cacattcgga |
| 26761 | gtttggattt ggaagtggtg gaagagatgg agatggtgag acaagtagta agcacgtcag |
| 26821 | ccttccaggt gcgctccttt ccgatgagca ctgtcttatc ccacgtaact ttgagaagtt |
| 26881 | tgggcctttc ccactgtggc agaggtttcc tgaggctctt gcatacatgg ccctatggtt |
| 26941 | gctcatcaga tctttctccc agtagctgct cagcatggtg gtggcataag cccattttcc |
| 27001 | ggagccaggg attcagttgc agcaagacat ggcccggtct gggaggtcaa ccatgaagaa |
| 27061 | ggcagtagct gtcattgccc aaccccagaa atcccaatcc tgttttctcc ctctcagtcc |
| 27121 | tgatcatgga ttcagcagca gcgaactcgc caatgtagtg ggtggcacag ccagggtctt |
| 27181 | gactctggct ctgcagtagc acagtctgga aaagctctga ggggagagag acccccactg |
| 27241 | gtccgagggt ctggcacaga gccagaaatg ggggggaagg tatggggctg ggtcgcctct |
| 27301 | gacctctcag gtaccatcca ggaggccctg gcctctcact gaacccggcc actcctcttt |
| 27361 | ggcatggcct cttcccaaat ccccaaactg cctccttacc cacaaaagtg gtctctgagt |
| 27421 | gtcagtccag tgggaccccc accccttatg gcttcagttc cccaaatagg gctggaccct |
| 27481 | tgatcctgat ccagctgtgg ctatccagcc ccttcctggg gactttggac tttgaggggg |
| 27541 | gcatgcccag ttgtgctggg aatccatact ttccctggct ggagtagaac ctgtggactg |
| 27601 | tagtcctgag ggcagtcatg ttct |

“Detect” refers to identifying the presence, absence or amount of the analyte to be detected. In some embodiments, a copy number of complement component 4A (C4A) or complement component 4B (C4B) is detected. In other embodiments, presence of a human endogenous retrovirus (HERV) sequence is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. In some embodiments, the detectable label is a fluorescent polypeptide.

By “disease” is meant any condition or disorder that damages or interferes with the normal function of a cell, tissue, or organ. Examples of diseases include schizophrenia, Alzheimer's Disease, glaucoma, and age-related macular degeneration. Such diseases are characterized by undesirably increased levels of complement component 4A (C4A) and/or synaptic pruning.

By “effective amount” is meant the amount of a required to ameliorate the symptoms of a disease relative to an untreated patient. In particular embodiments, the disease is schizophrenia. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “expression” as used herein is defined as the transcription and/or translation of a particular nucleotide sequence driven by its promoter.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, a “human endogenous retrovirus” or “HERV” polynucleotide sequence is a polynucleotide sequence that occurs in the human genome that is substantially identical to a sequence in a retrovirus or that was derived from a retrovirus. In some embodiments, the HERV sequence is a human endogenous retrovirus type K (HERV-K) sequence. In some other embodiments, the HERV sequence is a C4-HERV sequence. In certain embodiments, a retroviral (C4-HERV) sequence in intron 9 is inserted within a C4A polynucleotide sequence or a C4B polynucleotide sequence. An exemplary HERV sequence is provided at GenBank Accession No. AF164613.1, and is reproduced below.

| | |
|---|---|
| 1 | tgtggggaaa agcaagagag atcaaattgt tactgtgtct gtgtagaaag aagtagacat |
| 61 | aggagactcc attttgttat gtgctaagaa aaattcttct gccttgagat tctgttaatc |
| 121 | tatgacctta cccccaaccc cgtgctctct gaaacgtgtg ctgtgtcaac tcagggttga |
| 181 | atggattaag ggcggtgcag gatgtgcttt gttaaacaga tgcttgaagg cagcatgctc |
| 241 | cttaagagtc atcaccactc cctaatctca agtacccagg gacacaaaaa ctgcggaagg |
| 301 | ccgcagggac ctctgcctag gaaagccagg tattgtccaa ggtttctccc catgtgatag |
| 361 | tctgaaatat ggcctcgtgg gaagggaaag acctgaccgt cccccagccc gacacctgta |
| 421 | aagggtctgt gctgaggagg attagtaaaa gaggaaggaa tgcctcttgc agttgagaca |
| 481 | agaggaaggc atctgtctcc tgcctgtccc tgggcaatgg aatgtctcgg tataaaaccc |
| 541 | gattgtatgc tccatctact gagataggga aaaaccgcct tagggctgga ggtgggacct |
| 601 | gcgggcagca atactgcttt gtaaagcatt gagatgttta tgtgtatgca tatccaaaag |
| 661 | cacagcactt aatcctttac attgtctatg atgccaagac ctttgttcac gtgtttgtct |
| 721 | gctgaccctc tccccacaat tgtcttgtga ccctgacaca tccccctctt tgagaaacac |
| 781 | ccacagatga tcaataaata ctaagggaac tcagaggctg gcgggatcct ccatatgctg |
| 841 | aacgctggtt ccccgggtcc ccttatttct ttctctatac tttgtctctg tgtctttttc |
| 901 | ttttccaaat ctctcgtccc accttacgag aaacacccac aggtgtgtag gggcaaccca |
| 961 | cccctacatc tggtgcccaa cgtggaggct tttctctagg gtgaaggtac gctcgagcgt |
| 1021 | ggtcattgag gacaagtcga cgagagatcc cgagtacatc tacagtcagc cttacggtaa |
| 1081 | gcttgcgcgc tcggaagaag ctagggtgat aatggggcaa actaaaagta aaattaaaag |
| 1141 | taaatatgcc tcttatctca gctttattaa aattctttta aaaagagggg gagttaaagt |
| 1201 | atctacaaaa aatctaatca agctatttca aataatagaa caattttgcc catggtttcc |
| 1261 | agaacaagga acttcagatc taaaagattg gaaaagaatt ggtaaggaac taaaacaagc |
| 1321 | aggtaggaag ggtaatatca ttccacttac agtatggaat gattgggcca ttattaaagc |
| 1381 | agctttagaa ccatttcaaa cagaagaaga tagcatttca gtttctgatg cccctggaag |
| 1441 | ctgtttaata gattgtaatg aaaacacaag gaaaaaatcc cagaaagaaa ccgaaagttt |
| 1501 | acattgcgaa tatgtagcag agccggtaat ggctcagtca acgcaaaatg ttgactataa |
| 1561 | tcaattacag gaggtgatat atcctgaaac gttaaaatta gaaggaaaag gtccagaatt |
| 1621 | aatggggcca tcagagtcta aaccacgagg cacaagtcct cttccagcag gtcaggtgct |
| 1681 | cgtaagatta caacctcaaa agcaggttaa agaaaataag acccaaccgc aagtagccta |
| 1741 | tcaatactgg ccgctggctg aacttcagta tcggccaccc ccagaaagtc agtatggata |
| 1801 | tccaggaatg cccccagcac cacagggcag ggcgccatac catcagccgc ccactaggag |
| 1861 | acttaatcct atggcaccac ctagtagaca gggtagtgaa ttacatgaaa ttattgataa |
| 1921 | atcaagaaag gaaggagata ctgaggcatg gcaattccca gtaacgttag aaccgatgcc |
| 1981 | acctggagaa ggagcccaag agggagagcc tcccacagtt gaggccagat acaagtcttt |
| 2041 | ttcgataaaa atgctaaaag atatgaaaga gggagtaaaa cagtatggac ccaactcccc |
| 2101 | ttatatgagg acattattag attccattgc ttatggacat agactcattc cttatgattg |
| 2161 | ggagattctg gcaaaatcgt ctctctcacc ctctcaattt ttacaattta agacttggtg |
| 2221 | gattgatggg gtacaagaac aggtccgaag aaatagggct gccaatcctc cagttaacat |
| 2281 | agatgcagat caactattag gaataggtca aaattggagt actattagtc aacaagcatt |
| 2341 | aatgcaaaat gaggccattg agcaagttag agctatctgc cttagagcct gggaaaaaat |
| 2401 | ccaagaccca ggaagtacct gcccctcatt taatacagta agacaaggtt caaaagagcc |
| 2461 | ctaccctgat tttgtggcaa ggctccaaga tgttgctcaa aagtcaattg ccgatgaaaa |
| 2521 | agccggtaag gtcatagtgg agttgatggc atatgaaaac gccaatcctg agtgtcaatc |
| 2581 | agccattaag ccattaaaag gaaaggttcc tgcaggatca gatgtaatct cagaatatgt |
| 2641 | aaaagcctgt gatggaatcg gaggagctat gcataaagct atgcttatgg ctcaagcaat |
| 2701 | aacaggagtt gttttaggag gacaagttag aacatttgga ggaaaatgtt ataattgtgg |
| 2761 | tcaaattggt cacttaaaaa agaattgccc agtcttaaac aaacagaata taactattca |
| 2821 | agcaactaca acaggtagag agccacctga cttatgtcca agatgtaaaa aaggaaaaca |
| 2881 | ttgggctagt caatgtcgtt ctaaatttga taaaaatggg caaccattgt cgggaaacga |
| 2941 | gcaaaggggc cagcctcagg ccccacaaca aactggggca ttcccaattc agccatttgt |
| 3001 | tcctcagggt tttcagggac aacaaccccc actgtcccaa gtgtttcagg gaataagcca |
| 3061 | gttaccacaa tacaacaatt gtccctcacc acaagcggca gtgcagcagt agatttatgt |
| 3121 | actatacaag cagtctctct gcttccaggg gagcccccac aaaaaatccc tacaggggta |
| 3181 | tatggcccac tgcctgaggg gactgtagga ctaatcttgg gaagatcaag tctaaatcta |
| 3241 | aaaggagttc aaattcatac tagtgtggtt gattcagact ataaaggcga aattcaattg |
| 3301 | gttattagct cttcaattcc ttggagtgcc agtccaagag acaggattgc tcaattatta |
| 3361 | ctcctgccat atattaaggg tggaaatagt gaaataaaaa gaataggagg gcttgtaagc |
| 3421 | actgatccaa caggaaaggc tgcatattgg gcaagtcagg tctcagagaa cagacctgtg |
| 3481 | tgtaaggcca ttattcaagg aaaacagttt gaagggttgg tagacactgg agcagatgtc |
| 3541 | tctattattg ctttaaatca gtggccaaaa aactggccta aacaaaaggc tgttacagga |
| 3601 | cttgtcggca taggcacagc ctcagaagtg tatcaaagta tggagatttt acattgctta |
| 3661 | gggccagata atcaagaaag tactgttcag ccaatgatta cttcaattcc tcttaatctg |
| 3721 | tggggtcgag atttattaca acaatggggt gcggaaatca ccatgcccgc tccattatat |
| 3781 | agccccacga gtcaaaaaat catgaccaag atgggatata taccaggaaa gggactaggg |
| 3841 | aaaaatgaag atggcattaa agttccagtt gaggctaaaa taaatcaaga aagagaagga |
| 3901 | atagggtatc ctttttaggg gcggtcactg tagagcctcc taaacccata ccactaactt |
| 3961 | ggaaaacaga aaaaccggtg tgggtaaatc agtggccgct accaaaacaa aaactggagg |
| 4021 | ctttacattt attagcaaat gaacagttag aaaagggtca cattgagcct tcgttctcac |
| 4081 | cttggaattc tcctgtgttt gtaattcaga agaaatcagg caaatggcat acgttaactg |
| 4141 | acttaagggc tgtaaacgcc gtaattcaac ccatggggcc tctccaaccc gggttgccct |
| 4201 | ctccggccat gatcccaaaa gattggcctt taattataat tgatctaaag gattgctttt |
| 4261 | ttaccatccc tctggcagag caggattgtg aaaaatttgc ctttactata ccagccataa |
| 4321 | ataataaaga accagccacc aggtttcagt ggaaagtgtt acctcaggga atgcttaata |
| 4381 | gtccaactat ttgtcagact tttgtaggtc gagctcttca accagtgaga gaaaagtttt |
| 4441 | cagactgtta tattattcat tatattgatg atattttatg tgctgcagaa acgaaagata |
| 4501 | aattaattga ctgttataca tttctgcaag cagaggttgc caatgctgga ctggcaatag |
| 4561 | catctgataa gatccaaacc tctactcctt ttcattattt agggatgcag atagaaaata |
| 4621 | gaaaaattaa gccacaaaaa atagaaataa gaaaagacac attaaaaaca ctaaatgatt |
| 4681 | ttcaaaaatt actaggagat attaattgga ttcggccaac tctaggcatt cctacttatg |
| 4741 | ccatgtcaaa tttgttctct atcttaagag gagactcaga cttaaatagt caaagaatat |
| 4801 | taaccccaga ggcaacaaaa gaaattaaat tagtggaaga aaaaattcag tcagcgcaaa |
| 4861 | taaatagaat agatccctta gccccactcc aacttttgat ttttgccact gcacattctc |
| 4921 | caacaggcat cattattcaa aatactgatc ttgtggagtg gtcattcctt cctcacagta |
| 4981 | cagttaagac ttttacattg tacttggatc aaatagctac attaatcggt cagacaagat |
| 5041 | tacgaataac aaaattatgt ggaaatgacc cagacaaaat agttgtccct ttaaccaagg |
| 5101 | aacaagttag acaagccttt atcaattctg gtgcatggca gattggtctt gctaattttg |
| 5161 | tgggacttat tgataatcat tacccaaaaa caaagatctt ccagttctta aaattgacta |
| 5221 | cttggattct acctaaaatt accagacgtg aacctttaga aaatgctcta acagtattta |
| 5281 | ctgatggttc cagcaatgga aaagcagctt acacagggcc gaaagaacga gtaatcaaaa |
| 5341 | ctccatatca atcggctcaa agagcagagt tggttgcagt cattacagtg ttacaagatt |
| 5401 | ttgaccaacc tatcaatatt atatcagatt ctgcatatgt agtacaggct acaagggatg |
| 5461 | ttgagacagc tctaattaaa tatagcatgg atgatcagtt aaaccagcta ttcaatttat |
| 5521 | tacaacaaac tgtaagaaaa agaaatttcc cattttatat tactcatatt cgagcacaca |
| 5581 | ctaatttacc agggcctttg actaaagcaa atgaacaagc tgacttactg gtatcatctg |
| 5641 | cactcataaa agcacaagaa cttcatgctt tgactcatgt aaatgcagca ggattaaaaa |
| 5701 | acaaatttga tgtcacatgg aaacaggcaa aagatattgt acaacattgc acccagtgtc |
| 5761 | aagtcttaca cctgcccact caagaggcag gagttaatcc cagaggtctg tgtcctaatg |
| 5821 | cattatggca aatggatgtc acgcatgtac cttcatttgg aagattatca tatgttcatg |
| 5881 | taacagttga tacttattct tattcacatt tcatatgggc aacttgccaa acaggagaaa |
| 5941 | gtacttccca tgttaaaaaa catttattgt cttgttttgc tgtaatggga gttccagaaa |
| 6001 | aaatcaaaac tgacaatgga ccaggatatt gtagtaaagc tttccaaaaa ttcttaagtc |
| 6061 | agtggaaaat ttcacataca acaggaattc cttataattc ccaaggacag gccatagttg |
| 6121 | aaagaactaa tagaacactc aaaactcaat tagttaaaca aaaagaaggg ggagacagta |
| 6181 | aggagtgtac cactcctcag atgcaactta atctagcact ctatacttta aattttttaa |
| 6241 | acatttatag aaatcagact actacttctg cagaacaaca tcttactggt aaaaagaaca |
| 6301 | gcccacatga aggaaaacta atttggtgga aagataataa aaataagaca tgggaaatag |
| 6361 | ggaaggtgat aacgtgaggg agaggttttg cttgtgtttc accaggagaa aatcagcttc |
| 6421 | ctgtttggtt acccactaga catttgaagt tctacaatga acccatcgga gatgcaaaga |
| 6481 | aaagggcctc cacggagagg gtaacaccag tcacatggat ggataatcct atagaagtat |
| 6541 | atgttaatga tagtgtatgg gtacctggcc ccatagatga tcgctgccct gccaaacctg |
| 6601 | aggaagaagg gatgatgata aatatttcca ttgggtatcg ttatcctcct atttgcctag |
| 6661 | ggagagcacc aggatgttta atgcctgcag tccaaaattg gttggtagaa gtacctactg |
| 6721 | tcagtcccat cagtagattc acttatcaca tggtaagcgg gatgtcactc aggccacggg |
| 6781 | taaattattt acaagacttt tcttatcaaa gatcattaaa atttagacct aaagggaaac |
| 6841 | cttgccccaa ggaaattccc aaagaatcaa aaaatacaga agttttagtt tgggaagaat |
| 6901 | gtgtggccaa tagtgcggtg atattataaa acaatgaatt tggaactatt atagattggg |
| 6961 | cacctcgagg tcaattctac cacaattgct caggacaaac tcagtcgtgt ccaagtgcac |
| 7021 | aagtgagtcc agctgttgat agcgacttaa cagaaagttt agacaaacat aagcataaaa |
| 7081 | aattgcagtc tttctaccct tgggaatggg gagaaaaagg aatctctacc ccaagaccaa |
| 7141 | aaatagtaag tcctgtttct ggtcctgaac atccagaatt atggaggctt actgtggcct |
| 7201 | cacaccacat tagaatttgg tctggaaatc aaactttaga aacaagagat tgtaagccat |
| 7261 | tttatactgt cgacctaaat tccagtctaa cagttccttt acaaagttgc gtaaagcccc |
| 7321 | cttatatgct agttgtagga aatatagtta ttaaaccaga ctcccagact ataacctgtg |
| 7381 | aaaattgtag attgcttact tgcattgatt caacttttaa ttggcaacac cgtattctgc |
| 7441 | tggtgagagc aagagagggc gtgtggatcc ctgtgtccat ggaccgaccg tgggaggcct |
| 7501 | caccatccgt ccatattttg actgaagtat taaaaggtgt tttaaataga tccaaaagat |
| 7561 | tcatttttac tttaattgca gtgattatgg gattaattgc agtcacagct acggctgctg |
| 7621 | tagcaggagt tacattgcac tcttctgttc agtcagta |

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. The preparation can be at least 75%, at least 90%, and at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level, copy number, sequence, or activity that is associated with a disease or disorder or risk of disease or disorder. In some embodiments, an alteration in the copy number and/or sequence of C4A polynucleotide and/or C4B polynucleotide is associated with risk of schizophrenia.

By “microglia” is meant an immune cell of myeloid lineage resident in the central nervous system.

As used herein, “obtaining” as in “obtaining an agent” includes synthesizing, purchasing, or otherwise acquiring the agent.

As used herein a “probe” or “nucleic acid or oligonucleotide probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition. In some embodiments, a “reference copy number” is a copy number of 0 or 1. In some other embodiments, a “reference level” is a level of C4A or C4B polynucleotide, such as C4A or C4B RNA, in a healthy, normal subject or in a subject that does not have schizophrenia.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, or at least about 25 amino acids. The length of the reference polypeptide sequence can be about 35 amino acids, about 50 amino acids, or about 100 amino acids. For nucleic acids, the length of the reference nucleic acid sequence will generally be at least about 50 nucleotides, at least about 60 nucleotides, or at least about 75 nucleotides. The length of the reference nucleic acid sequence can be about 100 nucleotides, about 300 nucleotides or any integer thereabout or therebetween.

In some embodiments, the reference sequence is a sequence of a “short form” of complement component 4A (C4A) genomic polynucleotide. In some other embodiments, the reference sequence is the sequence of a short form of complement component 4B (C4B) genomic polynucleotide. As used herein, a “short form” of a C4A or C4B polynucleotide is a C4A or C4B polynucleotide that does not contain an insertion of a human endogenous retrovirus (HERV) sequence. As used herein, a “long form” of a C4A or C4B polynucleotide is a C4A or C4B polynucleotide that contains an insertion of a human endogenous retrovirus (HERV) sequence.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant an agent that recognizes and binds a polypeptide or polynucleotide of the invention, but which does not substantially recognize and bind other molecules in a sample, for example, a biological sample, which naturally includes a polynucleotide of the invention. In some embodiments, the agent is a nucleic acid molecule. In some embodiments, the agent is an antibody that specifically binds C4A polypeptide.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., at least about 37° C., and at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 .mu.g/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., and at least about 68° C. In one embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In yet another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60%, at least 80%, at least 85%, at least 90%, at least 95% or even at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. As used herein, “schizophrenia treatment” or “treatment for schizophrenia” includes, without limitation, antipsychotic agents and psychosocial therapy. Psychosocial therapy for schizophrenia includes individual therapy and family therapy.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1C are schematics showing structural variation of the complement component 4 (C4) gene. FIG. 1A shows the location of the C4 genes within the Major Histocompatibility Complex (MHC) locus on human chromosome 6. FIG. 1B shows human C4 exists as two paralogous genes (isotypes), C4A and C4B; the encoded proteins are distinguished at a key site that determines which molecular targets they bind^{19,20}. Both C4A and C4B also exist in both long (L) and short (S) forms distinguished by an endogenous retroviral (C4-HERV) sequence in intron 9. FIG. 1C shows structural forms of the C4 locus and their frequencies among a European-ancestry population sample (222 chromosomes from 111 genetically unrelated individuals, HapMap CEU), inferred as described in FIGS. 9A-9E. Asterisks indicate allele frequencies too low to be well estimated.

FIG. 2 is a set of plots and schematics showing haplotypes formed by C4 structures and SNPs. SNP haplotype(s) on which common C4 structures were present. Each thin horizontal line represents the series of SNP alleles (haplotype) along a 250-kilobase chromosomal segment. Each column represents a SNP; gray and black indicate which allele is present on each haplotype. The SNP haplotypes are grouped into 13 sets of haplotypes associating with each of the four most common C4 structures. Three C4 structures (AL-BS, AL-BL, and AL-AL) each segregated on multiple SNP haplotypes (numbered at right).

FIGS. 3A-3C are plots showing brain RNA expression of C4A and C4B in relation to copy numbers of C4A, C4B, and the C4-HERV. FIG. 3A shows mRNA expression of C4A. FIG. 3B shows mRNA expression of C4B. mRNA expression shown in FIGS. 3A-3B was measured (by ddPCR) in brain tissue from 244 individuals. Copy number of C4A, C4B, and the C4-HERV were measured (by ddPCR analysis of genomic DNA) in the brain donors. The results were consistent across 8 panels of brain tissue representing 5 brain regions and 3 distinct sets of donors (one set shown here, with data from 101 individuals; all panels in FIGS. 11A-11H; a few outlier points are beyond the range of these plots but are shown in FIGS. 11A-11H). P-values were obtained by a Spearman rank correlation test. FIG. 3C shows expression of C4A (per genomic copy) is normalized to expression of C4B (per genomic copy) to control for trans-acting influences shared by C4A and C4B.

FIGS. 4A-4F are plots showing association of schizophrenia to C4 and the extended MHC locus. Association of schizophrenia to 7,751 SNPs across the MHC locus and to genetically predicted expression levels of C4A and C4B in the brain (represented in the genomic location of the C4 gene). The data shown are based on analysis of 28,799 schizophrenia cases and 35,986 controls of European ancestry from the Psychiatric Genomics Consortium. The height of each point represents the statistical strength (−log₁₀ (p)) of association with schizophrenia. FIG. 4A shows association of schizophrenia to SNPs in the MHC locus and to genetically predicted expression of C4A and C4B. FIG. 4B shows association of schizophrenia to SNPs in the MHC locus and to genetically predicted expression of C4A and C4B, with genetic variants are colored by their levels of correlation to rs13194504 (upper panel) or by their levels of correlation to genetically predicted brain C4A expression levels (lower panel). FIG. 4C, FIG. 4D, FIG. 4E, and FIG. 4F each shows conditional association analysis. The red dashed line indicates the statistical threshold for genome-wide significance (p=5×10⁻⁸). See also FIG. 12, FIGS. 13A-13E, and FIG. 14 for detailed association analyses involving C4 locus structures and HLA alleles.

FIGS. 5A-5D are plots showing C4 structures, C4A expression, and schizophrenia risk. FIG. 5A shows schizophrenia risk associated with four common structural forms of C4 in analysis of 28,799 schizophrenia cases and 35,986 controls. FIG. 5B shows brain C4A RNA expression levels associated with four common structural forms of C4. β was calculated from fitting C4A RNA expression (in brain tissue) to the number of chromosomes (0, 1, or 2) carrying each C4 structure (across 120 individuals sampled). FIG. 5C shows schizophrenia risk associated with 13 combinations of C4 structural allele and MHC SNP haplotype. The numbers on the y-axis adjacent to the C4 structures indicate the “haplogroup”, the MHC SNP haplotype background on which the C4 structure segregates, and correspond to FIG. 2. Statistical tests of heterogeneity yielded p=0.55 for AL-AL alleles; p=0.93 for AL-BL alleles; p=0.06 for AL-BS alleles; and p=5.7×10⁻⁵ across the overall allelic series. FIG. 5D shows expression levels of C4A RNA were directly measured (by RT-ddPCR) in post mortem brain samples from 35 schizophrenia patients and 70 individuals not affected with schizophrenia. Measurements for all five brain regions analyzed exhibited the same relationship (FIG. 15). Horizontal lines show the median value for each group. P-values were derived by a (nonparametric) one-sided Mann-Whitney test. Error bars shown in FIGS. 5A-5C represent 95% confidence intervals around the effect size estimate.

FIGS. 6A-6D are micrograph images showing C4 protein at neuronal cell bodies, processes and synapses. FIG. 6A shows C4 protein localization in human brain tissue. Two representative confocal images (drawn from immunohistochemistry performed on samples from five individuals with schizophrenia and two unaffected individuals) within the hippocampal formation demonstrate localization of C4 in a subset of NeuN⁺ neurons (representative staining for C4 (bottom, left panel); NeuN (bottom, center panels); and Hoechst (bottom, right panels) are shown. FIG. 6B shows high-resolution structured illumination microscopy (SIM) imaging of tissue in the hippocampal formation reveals colocalization of C4 with the presynaptic terminal marker Vglut1/2 and the postsynaptic parker PSD95 (representative staining for C4 (top, left small panel); PSD95 (bottom, left, small panel); Vglut1/2 (top, right, small panel) and Hoechst (bottom, right, small panel) are shown). FIG. 6C shows confocal images of primary human cortical neurons show colocalization of C4, MAP2, and neurofilament along neuronal processes (representative staining for C4 (left panel) and small panels on the right, from top to bottom: C4, MAP2, Neurofilament, and Hoechst). FIG. 6D shows confocal image of primary cortical neurons stained for C4, presynaptic marker synaptotagmin, and postsynaptic marker PSD95. (representative staining for small panels on the right, from top to bottom: C4, Synaptotagmin, PSD95, and Hoechst). Scale bar for FIG. 6A, FIG. 6C, and FIG. 6D=25 μm; FIG. 6B=5 μm; FIG. 6B inset=1 μm. FIGS. 16A-16C contains additional data on antibody specificity.

FIGS. 7A-7D are micrograph images and plots showing C4 in retinogeniculate synaptic refinement. FIG. 7A depicts representative confocal images of immunohistochemistry for C3 in the P5 dLGN showed reduced C3 deposition in the dLGN of C4−/− mice compared to WT littermates (representative staining for small inset panels, from left to right: C3, VGLUT2, and DAPI). FIG. 7B shows quantification confirmed reduced C3 immunoreactivity in the dLGN (N=3 mice/group, p<0.05, f-test; y-axis: mean fluorescence intensity, normalized to WT). FIG. 7C shows co-localization analysis revealed a reduction in the fraction of VGLUT2+ puncta that were C3+ in C4-deficient mice relative to their WT littermates (N=3 mice/group, p=0.0011, two sided f-test). FIG. 7D shows synaptic refinement in mice with 0, 1, or 2 copies of C4. These images represent the segregation of ipsilateral and contralateral RGC projections to the dLGN; two analysis methods were used. The top of FIG. 7D shows projections from the ipsilateral (dark gray) and contralateral (medium gray) eyes show minimal overlap (light gray) in WT mice. The overlapping area is significantly increased in C4−/− mice (N=6 mice/group, p<0.01, ANOVA with Bonferroni post tests). At the bottom of FIG. 7D, threshold independent analysis using the R-value⁵⁰ (R=log₁₀ [Fᵢₚₛᵢ/F_{contra}]) is shown. Pixels are pseudocolored with an R-value heat map (red indicates areas having only contralateral inputs; purple, only ipsilateral inputs). Compared to their WT littermates, C4-deficient mice exhibited lower R-value variance, indicating defects in synaptic refinement (N=6 mice/group, p<0.001, ANOVA with Bonferroni post tests). Control experiments analyzing total dLGN size, dLGN area receiving ipsilateral input, and number of RGCs are shown in FIGS. 17F-17H, respectively. Error bars in FIG. 7B, FIG. 7C, and FIG. 7D represent S.E.M.

FIGS. 8A-8G are plots and schematics showing association of schizophrenia to common variants in the MHC locus in individual case-control cohorts, and the repeat module containing C4. Each of FIGS. 8A-8F shows that data for several schizophrenia case-control cohorts that were genome-scanned before this work was begun (FIGS. 8A-8D) exhibits peaks of association near chr6:32 Mb (blue vertical line) on the human genome reference sequence (GRCh37/hg19). Association patterns vary from cohort to cohort, reflecting statistical sampling fluctuations and potentially fluctuations in allele frequencies of the (unknown) causal variants in different cohorts. Cohorts such as in FIG. 8B, FIG. 8E and FIG. 8F suggest the existence of effects at multiple loci within the MHC region. Even in the cohorts with simpler peaks (FIG. 8A, FIG. 8C, and FIG. 8D), the pattern of association across the individual SNPs at chr6:32 Mb does not correspond to the linkage disequilibrium (LD) around any known variant. This motivated the focus in the current work on cryptic genetic influences in this region that could cause unconventional association signals that do not resemble the LD patterns of individual variants. FIG. 8G shows a complex form of genome structural variation resides near chr6:32 Mb. Shown here are three of the known alternative structural forms of this genomic region. The most prominent feature of this structural variation is the tandem duplication of a genomic segment that contains a C4 gene, 3′ fragments of the STK19 and TNXB genes, and a pseudogenized copy of the CYP21A2 gene. This cassette is present in 1-3 copies on the three alleles depicted above; the boundaries below each haplotype demarcate the sequence that is duplicated. Haplotypes with multiple copies of this module (middle and bottom) contain multiple functional copies of C4, whereas the additional gene fragments or copies denoted STK19P, CYP21A2P, and TNXA are typically pseudogenized. Rare haplotypes with a gain or loss of intact CYP21A2 have also been observed¹⁸. Note that although C4A and C4B contain multiple sequence variants, they are defined based on the differences encoded by exon 26, which determine the relative affinities of C4A and C4B for distinct molecular targets^{19,20} (FIGS. 1A-1C). Many additional forms of this locus appear to have arisen by non-allelic homologous recombination and gene conversion (ref¹⁸ and FIGS. 1A-1C).

FIGS. 9A-9E are schematics showing a strategy for identifying the segregating structural forms of the C4 locus. FIG. 9A shows molecular assays for measuring copy number of the key, variable C4 structural features—the length polymorphism (HERV insertion) that distinguishes the long (L) from the short (S) genomic form of C4, and the C4A/C4B isotypic difference. Each primer-probe-primer assay is represented with the combination of arrows (primers) and asterisk (probe) in its approximate genomic location (though not to scale). FIG. 9B shows measurement of copy number of C4 gene types in the genomes of 162 individuals (from HapMap CEU sample). The absolute, integer copy number of each C4 gene type in each genome is precisely inferred from the resulting data. To ensure high accuracy, the data are further evaluated for a checksum relationship (A+B=L+S) and for concordance with earlier data from Southern blotting of 89 of the same HapMap individuals⁵¹. Shown in FIG. 9C is a molecular assay to measure the copy number of compound structural forms of C4. To measure the copy number of compound structural forms of C4 (involving combinations of L/S and A/B), long-range PCR followed by quantitative measurement of the A/B isotype-distinguishing sequences in droplets was performed. FIG. 9D shows analysis of transmissions in father-mother-offspring trios enables inference of the C4 gene contents of individual copies (alleles) of chromosome 6. Three example trios are shown in this schematic. FIG. 9E shows examples of the inferred structural forms of the C4 locus (more shown in FIG. 1C). For the common C4 structures (AL-BL, AL-BS, AL-AL, and BS), genomic order of the C4 gene copies is known from earlier assemblies of sequence contigs in individuals homozygous for MHC haplotypes due to consanguinity″ and other molecular analyses of the C4 locus¹⁸. For the rarer C4 structures, genomic order of C4 gene copies is hypothesized or provisional.

FIGS. 10A-10B are plots showing linkage disequilibrium relationships (r²) of MHC SNPs to forms of C4 structural variation. FIG. 10A shows correlations of SNPs in the MHC locus with (a) copy number of C4 gene types. FIG. 10B shows correlations of SNPs in the MHC locus with larger-scale structural forms (haplotypes) of the C4 locus. Dashed, vertical lines indicate the genomic location of the C4 locus. Note that C4 structural forms show only partial correlation (r²) to the allelic states of nearby SNPs, reflecting the relationship shown in FIG. 2, in which a structural form of the C4 locus often segregates on multiple different SNP haplotypes.

FIGS. 11A-11H are plots showing RNA expression of C4A and C4B in relation to copy number of C4A, C4B, and the C4-HERV (long form of C4), in eight panels of post mortem brain tissue. Copy number of C4 structural features was measured by ddPCR; RNA expression levels were measured by RT-ddPCR. FIGS. 11A-11E show data for tissues from the Stanley Medical Research Institute (SMRI) Array Consortium. FIG. 11A shows data for anterior cingulate cortex; FIG. 11B shows data for cerebellum; FIG. 11C shows data for corpus callosum; FIG. 11D shows data for orbital frontal cortex; and FIG. 11E shows data for parietal cortex. FIG. 11F shows data for the frontal cortex samples from the NHGRI Genes and Tissues Expression (GTEx) Project. FIGS. 11G-11H show data for tissues from the SMRI Neuropathology Consortium. FIG. 11G shows data for anterior cingulate cortex; FIG. 11H shows data for cerebellum. These data were then used to inform (by linear regression) the derivation of a linear model for predicting each individual's RNA expression of C4A and C4B as a function of the numbers of copies of AL, BL, AS, and BS. The derivation of this model, and the regression coefficients induced, are described elsewhere herein. In the rightmost plot of each of FIGS. 11A-11H, expression of C4A (per genomic copy) is normalized to expression of C4B (per genomic copy) to more specifically visualize the effect of the C4-HERV by controlling for genomic copy number and for any trans-acting influences shared by C4A and C4B; the inferred regression coefficients indicate that the observed effect is mostly due to increased expression of C4A.

FIG. 12 is a table showing a detailed analysis of the association of schizophrenia to genetic variation at and around C4, in data from 28,799 schizophrenia cases and 35,986 controls (Psychiatric Genomics Consortium, ref⁶). SCZ, schizophrenia; β, estimated effect size per copy of the genomic feature or allele indicated; SE, standard error. Detailed association analyses of HLA alleles are in FIGS. 13A-13E and FIG. 14. (*) C4B-null status was specifically tested because a 1985 study⁵² reported an analysis of 165 schizophrenia patients and 330 controls in which rare C4B-null status associated with elevated risk of schizophrenia, though two subsequent studies^{53,54} found no association of schizophrenia to C4B-null genotype. This was evaluated using the large data set in this study, and no association to C4B-null status was found. (**) Total copy number of C4 is also strongly correlated to copy number of the CYP21A2P pseudogene, which is present on duplicated copies of the sequence shown in FIG. 8G.

FIGS. 13A-13E are plots showing evaluation of the association of schizophrenia with HLA alleles and coding-sequence polymorphisms. Each of FIGS. 13A-13E shows associations to HLA alleles and coding-sequence polymorphisms. The associations to HLA alleles and coding-sequence polymorphisms are shown in black; to provide the context of levels of association to nearby SNPs, associations to other SNPs are shown in gray. The series of conditional analyses shown in each of FIG. 13B-13E parallels the analyses in each of FIGS. 4C-4F, respectively. Further detail on the most strongly associating HLA alleles (including conditional association analysis) is provided in FIG. 14.

FIG. 14 is a table showing detailed association analysis for the most strongly associating classical HLA alleles. The most strongly associating HLA loci were HLA-B (in primary analyses, FIG. 4A, FIG. 13A) and HLA-DRB1 and -DQB1 (in analyses controlling for the signal defined by rs13194504, FIG. 4C, FIG. 13B). At these loci, the most strongly associating classical HLA alleles were HLA-B*0801, HLA-DRB1*0301, and HLA-DQB*02, respectively. These HLA alleles are all in strong but partial LD with C4 BS, the most protective of the C4 alleles; they are also in partial LD with the low-risk allele at rs13194505, representing the distinct signal several megabases to the left (FIGS. 4A-4F). In joint analyses with each of these HLA alleles, genetically predicted C4A expression and rs13194505 continued to associate strongly with schizophrenia, while the HLA alleles did not. In further joint analyses with rs13194504 and genetically predicted C4A expression, 0 of 2,514 tested HLA SNP, amino-acid and classical-allele polymorphisms (from ref⁵⁵, including all variants with MAF >0.005) associated to schizophrenia as strongly as rs13194504 or predicted C4A expression did.

FIG. 15 is a set of plots showing Expression of C4A RNA in brain tissue (five brain regions) from 35 schizophrenia cases and 70 non-schizophrenia controls, from the Stanley Medical Research Institute Array Consortium. C4A RNA expression levels were measured by ddPCR.

FIGS. 16A-16C are images showing secretion of C4, and specificity of the monoclonal anti-C4 antibody for C4 protein in human brain tissue and cultured primary cortical neurons. FIG. 16A shows brain tissue (from an individual affected with schizophrenia) was stained with a fluorescent secondary antibody, C4 antibody, or C4 antibody that was pre-adsorbed with purified C4 protein. Confocal images demonstrate the loss of immunoreactivity in the secondary-only and preadsorbed conditions. FIG. 16B shows primary human neurons were stained with a fluorescent secondary antibody, C4 antibody, or C4 antibody that was pre-adsorbed with purified C4 protein. Confocal images demonstrate the loss of immunoreactivity in the secondary-only and pre-adsorbed conditions. Scale bar for all images=25 μm. FIG. 16C shows secretion of C4 protein by cultured primary neurons. Western blot for C4 protein analysis. (+) Purified human C4 protein. (−) Unconditioned medium, a negative control. (HNconditioned) shows the same medium after conditioning by cultured human neurons at days 7 (d7) and 30 (d30). Details of Western blot protocol, antibody catalog numbers and concentrations used are described elsewhere herein. C4 molecular weight ˜210 kDa.

FIGS. 17A-17H are plots and images showing Mouse C4 genes and additional analyses of the dLGN eye segregation phenotype in C4 mutant mice and wild-type and heterozygous littermate controls. FIG. 17A shows that the functional specialization of C4 into C4A and C4B in humans does not have an analogy in mice. Although the mouse genome contains both a C4 gene and a C4-like gene (classically called Slp), and these genes are also present as a tandem duplication within the mouse MHC locus, analysis of the encoded protein sequences indicates a distinct specialization, as illustrated by the protein phylogenetic tree. Above, mouse Slp is indicated in gray to reflect its potential pseudogenization: Slp is already known to have mutations at a Cls cleavage site, which are thought to abrogate activation of the protein through the classical complement pathway⁵⁶; and the M. musculus reference genome sequence (mm10) at Slp shows a 1-bp deletion (relative to C4) within the coding region at chr17:34815158, which would be predicted to cause a premature termination of the encoded protein. In some genome data resources, mouse Slp and C4 have been annotated respectively as “C4a” (e.g. NM 011413.2) and “C4b” (e.g. NM_009780.2) based on synteny with the human C4A and C4B genes, but the above sequence analysis indicates that they are not paralogous to C4A and C4B. FIG. 17B shows that sequence differences between C4A and C4B—which are otherwise 99.5% identical at an amino acid level—are concentrated at the “isotypic site” where they shape each isotype's relative affinity for different molecular targets^{19,20}. At the isotypic site, mouse C4 contains a combination of the residues present in human C4A and C4B. FIG. 17C shows expression of mouse C4 mRNA in whole retina and lateral geniculate nucleus (LGN) from P5 animals and in purified retinal ganglion cells (RGCs) from P5 and P15 animals. These time points were chosen as P5 is a time of more robust synaptic refinement in the retinogeniculate system compared to P15. The same assays detected no C4 RNA in control RNA isolated from C4−/− mice. N=3 samples for p5 retina, LGN, and P15 RGCs, N=4 samples for P5 RGCs; *p<0.05 by ANOVA with post hoc Tukey-Kramer multiple-comparisons test. FIG. 17D depicts representative images of dLGN innervation by contralateral projections (medium gray in bottom image), ipsilateral projections (dark gray in bottom image), and their overlap (light gray in bottom image). Scale bar=100 μm. FIG. 17E shows quantification of the percentage of total dLGN area receiving both contralateral and ipsilateral projections shows a significant increase in C4−/− compared to WT littermates (ANOVA, N=5 mice/group, p<0.01). These data are consistent with results using R-value analysis as shown in FIGS. 7A-7D. FIG. 17F shows quantification of total dLGN area showed no significant difference between WT and C4−/− mice (ANOVA, N=5 per group, p>0.05). FIG. 17G shows quantification of dLGN area receiving ipsilateral innervation showed a significant increase in ipsilateral territory in the C4−/− mice compared to WT littermates (AVOVA, N=5 mice/group, p>0.01). This result is consistent with defects in eye specific segregation. Scale bar=100 μm. FIG. 17H shows the number of RGCs in the retina was estimated by counting the number of Brn3a+ cells in WT and C4−/− mice. No differences were observed between WT and C4−/− (t-test, N=4 mice/group, p>0.05). Scale bar=100 μm.

FIGS. 18A-18D are plots and images showing microglia engulfed more synaptic particles in the presence of C4A in the frontal cortex of young adult mice. FIG. 18A are images of FACS sorted microglia analyzed by confocal imaging showing the co-localization of SV2a proteins (bottom panel) within lysosomes (CD68) (middle panel). Arrows indicate co-localization. CD45 staining is shown in the top panel. FIG. 18B are representative dot plots showing the frequency of SV2 positive cells within the microglia population in C4+/+; C4−/−; and hC4A mice. FIG. 18C is a bar graph representing the frequency of SV2a positive microglia at P40. (C4+/+n=10; C4−/− n=9; hC4A/−n=6; hC4B/−n=2; littermates C4+/+ and C4−/−; C4−/− and hC4A/−; C4−/− and hC4B/−). Each symbol represents an individual mouse. Bars indicate the mean (SD). *P<0.05, ***P<0.001 (unpaired t test). Data are a pool of 3 independent experiments (C). FIG. 18D is a bar graph representing the frequency of SV2a positive microglia at P60. (C4−/− n=3; hC4A/−n=5 littermates). Each symbol represents an individual mouse. Horizontal lines indicate the mean (SD). *P<0.05, ***P<0.001 (unpaired t test). Data show 1 experiment.

FIGS. 19A-19D are plots and images showing Complement C4 regulated synapse number in frontal cortex of P60 mice. FIG. 19A are representative images showing staining for SV2 (light gray) and homer (medium gray). Synapses are defined as co-localized SV2 and Homer puncta (circle). Scale bar=5 um. FIG. 18B is a plot showing Synapse number for each mouse expressed as a fold change normalized to WT mice. FIG. 18C is a plot showing synapse number in females. FIG. 18D is a plot showing synapse number in males. Analyzed with Image J software. Each symbol in FIGS. 19B, 19C, and 19D represents an individual mouse. Horizontal lines indicate the mean (SD). ns, not significant (P>0.05); *P<0.05, **P<0.01 (unpaired t test).

FIGS. 20A and 20B are plots showing C4A preferential binding to synaptic membranes in an in vitro C4 binding assay. FIG. 20A is a representative histogram plot showing C4 staining on synaptosomes (curves, from left to right: C4−/−, hC4B, and hC4A).

FIG. 20B is a plot showing C4 binding fold change after correction for copy number (normalized with hC4B). Analyzed with FlowJo software. Bars indicate mean (SD). Pooled data from 2 independent experiments. **P<0.01 (unpaired t test).

FIGS. 21A-21C are plots and images showing changes in synapse number occurred during development in layer 2/3 of frontal cortex. FIG. 21A are confocal images taken in layer 2/3 of homer-GFP mice, co-stained with anti-GFP and anti-Vglut 1 and 2 antibodies at P25, P63, and P85. FIG. 21B is a plot showing quantification of synapse density (co-localized Homer and Vglut1/2) at each age. FIG. 21C depicts a 3D reconstruction of microglia (MAL dark gray) showing engulfed Vglut1/2+ synaptic material (light gray) at P63. 60× magnification, n=2.

FIG. 22A shows that human C4A and C4B differ by 4 amino acids (C4A: PCPVLD; C4B LSPVIH at amino acids 1120-1125 of the C4 preproprotein (amino acids 1101-1106 of the C4 proprotein) corresponding to Exon 26). Mouse C4 has a chimeric sequence at the corresponding position: PCPVIH (i.e. part huC4A and part huC4B). FIG. 22B shows the construction of human C4 BAC mice. Strains were back-crossed onto C4−/− B6 background.

FIG. 23 is a plot showing levels of C4 protein measured by ELISA in CSF from individuals affected or unaffected with schizophrenia

### DETAILED DESCRIPTION OF THE INVENTION

The invention features compositions and methods that are useful for determining risk of schizophrenia and treating schizophrenia in a subject. The invention is based, at least in part, on the discovery of a relationship between schizophrenia risk and structurally diverse alleles of the complement component 4 (C4) genes.

Schizophrenia is a heritable brain illness with unknown pathogenic mechanisms. Schizophrenia's strongest genetic association at a population level involves variation in the Major Histocompatibility Complex (MHC) locus, but the genes and molecular mechanisms accounting for this have been challenging to recognize. Studies described herein show that schizophrenia's association with the MHC locus arises in substantial part from many structurally diverse alleles of the complement component 4 (C4) genes. It was found that these alleles promoted widely varying levels of C4A and C4B expression and associated with schizophrenia in proportion to their tendency to promote greater expression of C4A in the brain. Human C4 protein localized at neuronal synapses, dendrites, axons, and cell bodies. In mice, C4 mediated synapse elimination during postnatal development. These results implicate excessive complement activity in the development of schizophrenia and may help explain the reduced numbers of synapses in the brains of individuals affected with schizophrenia.

Association of Loci with Schizophrenia Risk

Schizophrenia is a heritable psychiatric disorder involving impairments in cognition, perception and motivation that usually manifest late in adolescence or early in adulthood. The pathogenic mechanisms underlying schizophrenia are unknown, but observers have repeatedly noted pathological features involving excessive loss of gray matter^{1,2} and reduced numbers of synaptic structures on neurons³⁻⁵. While treatments exist for the psychotic symptoms of schizophrenia, there is no mechanistic understanding of, nor effective therapies to prevent or treat, the cognitive impairments and deficit symptoms of schizophrenia, its earliest and most constant features. An important goal in human genetics is to find the biological processes that underlie such disorders.

More than 100 loci in the human genome contain SNP haplotypes that associate with risk of schizophrenia⁶; the functional alleles and mechanisms at these loci remain to be discovered. By far the strongest such genetic relationship is schizophrenia's unexplained association with genetic markers across the Major Histocompatibility Complex (MHC) locus, which spans several megabases of chromosome 66-10. The MHC locus is best known for its role in immunity, containing 18 highly polymorphic human leukocyte antigen (HLA) genes that encode a vast suite of antigen-presenting molecules. In some autoimmune diseases, genetic associations at the MHC locus arise from alleles of HLA genes^{11,12}; however, schizophrenia's association to the MHC is not yet explained.

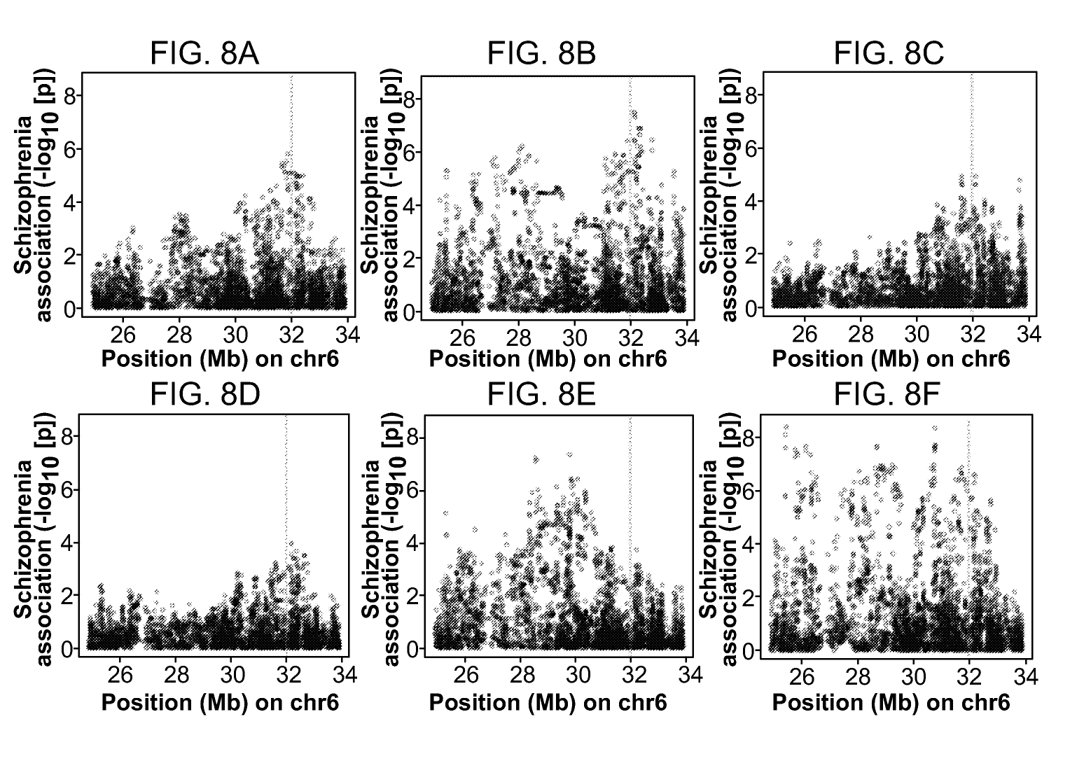

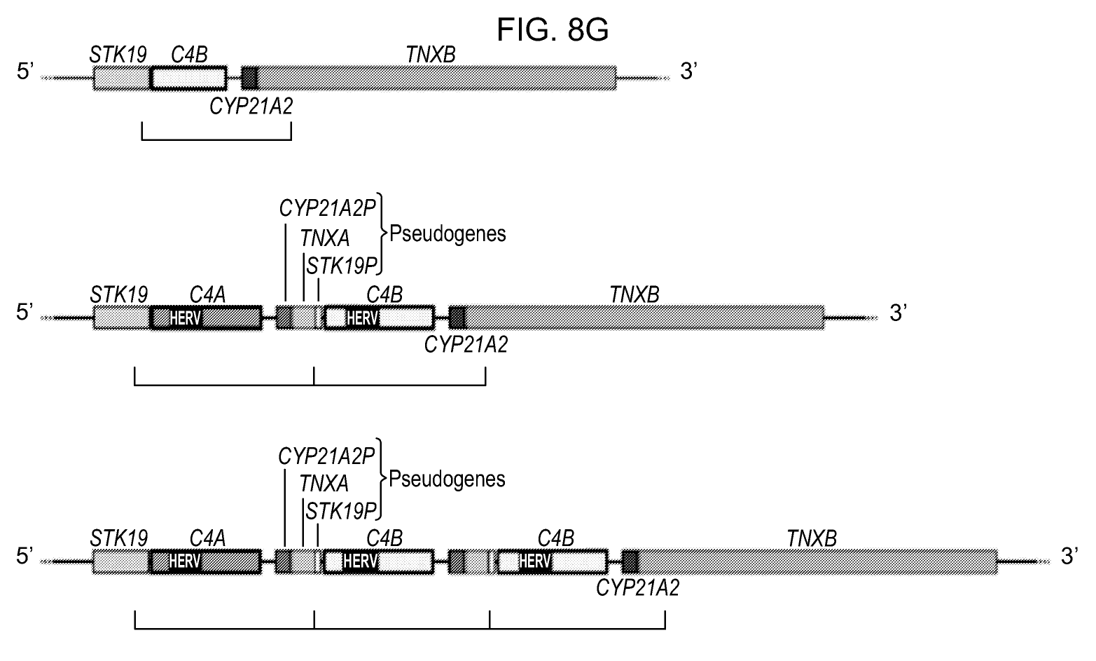

Though the functional alleles that give rise to genetic associations have in general been challenging to find, the schizophrenia-MHC association has been particularly challenging, as schizophrenia's complex pattern of association to markers in the MHC locus spans hundreds of genes and does not correspond to the linkage disequilibrium (LD) around any known variant^{6,10}. The most strongly associated markers in several large case/control cohorts were near a complex, multi-allelic, and only partially characterized form of genome variation that affects the C4 gene encoding complement component 4 (FIGS. 8A-8G). The studies described herein considered cryptic genetic influences that might generate unconventional genetic signals.

### Complement Component 4 (C4) and Schizophrenia Pathogenesis

In humans, adolescence and early adulthood bring extensive elimination of synapses in distributed association regions of cerebral cortex, such as the prefrontal cortex, that have greatly expanded in recent human evolution³⁷⁻⁴⁰. Synapse elimination in human association cortex appears to continue from adolescence into the third decade of life³⁹. This late phase of cortical maturation, which may distinguish humans even from some other primates³⁷, corresponds to the period during which schizophrenia most often becomes clinically apparent and patients' cognitive function declines, a temporal correspondence that others have also noted⁴¹. Principal pathological findings in schizophrenia brains involve loss of cortical gray matter without cell death: affected individuals exhibit abnormal cortical thinning¹² and abnormally reduced numbers of synaptic structures on cortical pyramidal neurons³⁻⁵. The possibility that neuron-microglia interactions via the complement cascade contribute to schizophrenia pathogenesis—for example, that schizophrenia arises or intensifies from excessive or inappropriate synaptic pruning during adolescence and early adulthood—would offer a potential mechanism for these longstanding observations about age of onset and synapse loss. Many other genetic findings in schizophrenia involve genes that encode synaptic proteins^{6,42-44}. Diverse synaptic abnormalities might interact with the complement system and other pathways^{45,46} to cause excessive stimulation of microglia and/or elimination of synapses.

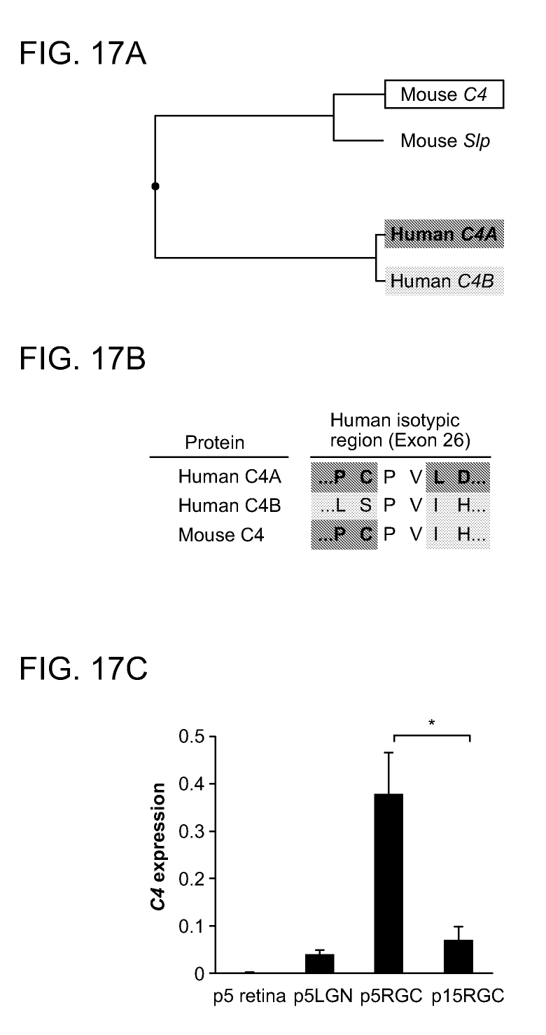

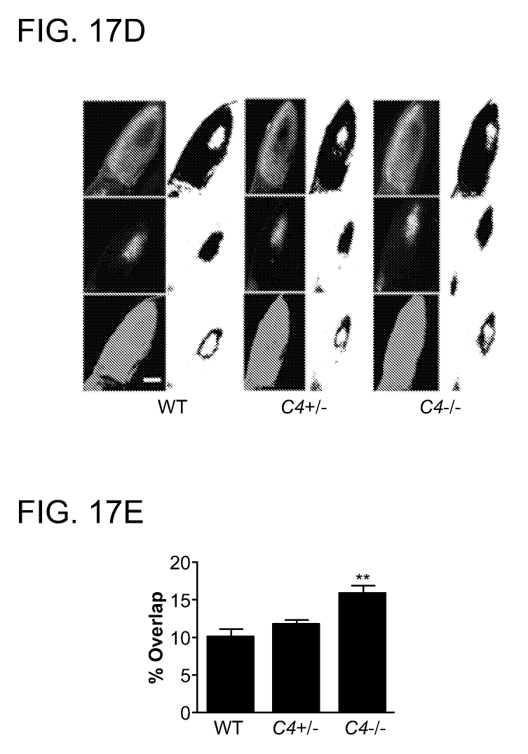

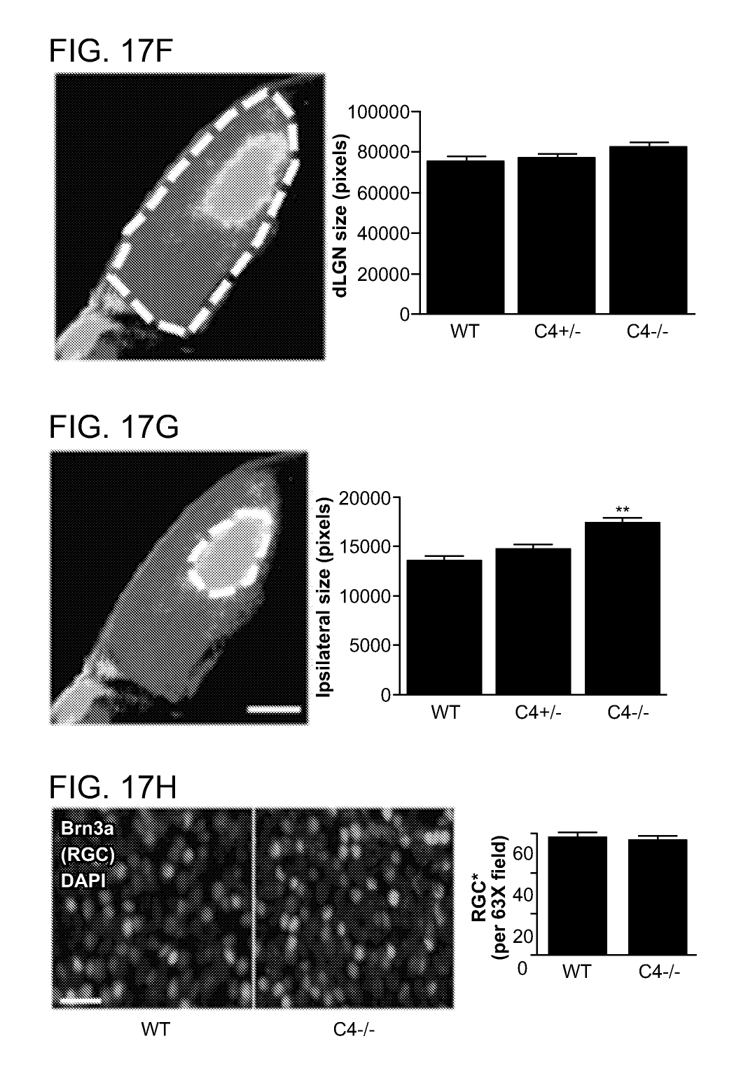

The two human C4 genes (C4A and C4B) exhibited distinct relationships with schizophrenia risk, with increased risk associating most strongly with variation that increases expression of C4A. Human C4A and C4B proteins, whose functional specialization appears to be evolutionarily recent (FIG. 17A), show striking biochemical differences: C4A more readily forms amide bonds with proteins, while C4B favors binding to carbohydrate surfaces^{19,20,} differences with an established basis in C4 protein sequence and structure^{47,48}. An intriguing possibility is that C4A and C4B differ in affinity for an unknown binding site at synapses.

To date, few associations from genomewide association studies (GWAS) have been explained by specific functional alleles. An unexpected finding at C4 involves the large number of common, functionally distinct forms of the same locus that appear to contribute to schizophrenia risk. The human genome contains hundreds of other genes with complex, multi-allelic forms of structural variation⁴⁹. It will be important to learn the extent to which such variation contributes to brain diseases and indeed to all human phenotypes.

Association of Risk of Schizophrenia with Structure of Complement 4 (C4) Alleles

In the studies described herein, allelic structure of complement 4 (C4) genes was found to be associated with risk of schizophrenia. In particular, increased expression of C4A mRNA in the brain was found to correlate with increased risk of schizophrenia. Increased C4A mRNA or C4B expression correlated with increased copy number of C4A or C4B genes. In addition, the presence of a human endogenous retrovirus (HERV) in C4A or C4B was found to increase expression of C4A relative to C4B.

Thus, information on allelic structure of C4 genes (e.g., copy number of C4A and/or C4B; presence or absence of HERV in C4A or C4B) may predict risk of schizophrenia in a subject. Accordingly, in one aspect, the invention provides a method of identifying a subject having or at risk of developing schizophrenia. The method contains the step of measuring copy number and/or sequence of C4A or C4B polynucleotide, where an alteration in copy number and/or sequence of C4A or C4B polynucleotide relative to a reference indicates the subject has or is at risk or developing schizophrenia. In some embodiments, the alteration in copy number is an increase in copy number. In some other embodiments, the alteration in sequence is insertion of a HERV sequence. In particular embodiments, the alteration is an increase in copy number of C4A polynucleotide. In some embodiments, the alteration is an increase in copy number of C4A polynucleotide containing a HERV sequence (i.e., long form of C4A polynucleotide). In certain embodiments, the alteration is any one of more of the following: an increase in copy number of C4A, increase in copy number of C4B, presence of HERV in one or more copies of C4A, and presence of HERV in one or more copies of C4B.

Early identification of risk of schizophrenia in a subject can be important in minimizing or preventing potentially irreversible deconstruction of a life that schizophrenia can bring to an individual and the individual's family and/or peers. If an individual is identified as having or at risk of developing schizophrenia at an early stage, proper treatment or therapy can be administered, which can help reduce symptoms of schizophrenia and/or help the individual (and family members and friends of the individual) cope with the individual's schizophrenia. Thus, in some embodiments, the methods contain the step of recommending an individual for further evaluation or for treatment of schizophrenia, if the individual is identified as having or at risk of developing schizophrenia. In some other embodiments, the methods contain the step of administering a schizophrenia treatment (e.g., antipsychotic agents and/or psychosocial therapy) to the individual if the individual is identified as having or at risk of developing schizophrenia.

In some aspects, the invention provides a method of treating schizophrenia in a pre-selected subject, where the subject is pre-selected for treatment by detecting an alteration in copy number and/or sequence of C4A or C4B polynucleotide relative to a reference. In some embodiments, the alteration in copy number is an increase in copy number. In some other embodiments, the alteration in sequence is insertion of a HERV sequence. In particular embodiments, the alteration is an increase in copy number of C4A polynucleotide. In some embodiments, the alteration is an increase in copy number of C4A polynucleotide containing a HERV sequence (i.e., long form of C4A polynucleotide). In certain embodiments, the alteration is any one of more of the following: an increase in copy number of C4A, increase in copy number of C4B, presence of HERV in one or more copies of C4A, and presence of HERV in one or more copies of C4B. For example, the subject can be diagnosed with schizophrenia and/or administered with schizophrenia treatment based on the results of the methods herein.

Further, studies herein have also found that increased level of C4A RNA, particularly in the brain, was associated with increased incidence of schizophrenia. Without being bound by theory, levels of C4 RNA associated with schizophrenia above and beyond what could be explained by effect of DNA variation at C4, indicate that dynamic biomarkers (that measure expression levels) might provide diagnostic information above and beyond that provided by DNA sequence and structure. Thus, in some aspects, the invention provides methods of identifying a subject having or at risk of developing schizophrenia, methods of treating schizophrenia in a subject, and methods of monitoring treatment progress in a subject, where the method contains the step of detecting an increased level of C4, or more specifically C4A RNA or C4A polypeptide, relative to a reference level.

In other aspects, the invention provides a method of treating schizophrenia in a pre-selected subject, where the subject is pre-selected by detecting an increased level of C4 or C4A protein or RNA relative to a reference level. Since C4 is a secreted protein, it can be detected in cerebrospinal fluid (CSF). Measuring levels of C4 in CSF could offer a way to dynamically measure C4 expression in a subject.

Analysis of C4A and C4B status can be performed in a variety of ways. In various embodiments of any of the aspects delineated herein, alterations in a polynucleotide or polypeptide of C4A and/or C4B (e.g, sequence, copy number, level) are analysed. In some embodiments, the method includes the step of measuring or detecting a level, copy number, or sequence of C4A and/or C4B polynucleotide in a biological sample obtained from the subject relative to a reference level, copy number, or sequence. In particular embodiments, DNA sequencing and copy number analysis are performed on C4A and/or C4B polynucleotide.

As described herein, an increase in copy number of C4A (particularly, the long form of C4A) and increased C4A expression were each associated with increased risk of schizophrenia. Thus, in some embodiments, an increase in copy number C4A is indicative of increased schizophrenia risk. Also, presence of a HERV sequence was found to increase C4A expression (particularly relative to C4B expression). Thus, increased copy number of a HERV sequence can be indicative of increased risk of schizophrenia, with risk increasing with increased numbers of copies. In certain embodiments, increased risk of schizophrenia can be indicated be any one of more of the following: an increase in copy number of C4A, presence of HERV in one or more copies of C4A, and presence of HERV in one or more copies of C4B.

In some embodiments, any one of the following combinations of C4A and C4B can be detected: one copy of C4B (short form), one copy of C4B (short form) and one copy of C4A (long form), one copy of C4B (long form) and one copy of C4A (long form), and two copies each of C4A (long form). In certain embodiments, the risk of schizophrenia associated with the combination of C4A and C4B is increased in the order in which the combination is listed as follows (from lowest to highest risk, respectively): one copy of C4B (short form), one copy of C4B (short form) and one copy of C4A (long form), one copy of C4B (long form) and one copy of C4A (long form), and two copies each of C4A (long form). As described elsewhere herein, the short form of either C4A or C4B does not contain a HERV sequence insertion in intron 9; the long form of either C4A or C4B contains a HERV sequence insertion in intron 9.

Alterations in polynucleotides or polypeptides of C4A and/or C4B (e.g, sequence, copy number, level) are detected in a biological sample obtained from an subject (e.g., a human). Biological samples include tissue samples (e.g., cell samples, biopsy samples), such as brain tissue. Biological samples that are used to evaluate the herein disclosed markers include without limitation brain tissue, blood, serum, plasma, and cerebrospinal fluid (CSF). In one embodiment, the biological sample is blood or serum. In another embodiment, the biological sample is brain tissue. In a particular embodiment, the biological sample is cerebrospinal fluid.

The sequence, level, or copy number of a polypeptide or polynucleotide of C4A and/or C4B detected in the method can be compared to a reference sequence, level, or copy number. The reference level of a C4A or C4B polynucleotide (e.g., a C4A or C4B RNA) can be level of C4A or C4B RNA in healthy normal controls. The reference copy number of C4A or C4B can be 0, 1, 2, or 3 copies. In some embodiments, the reference copy number is 0. The reference sequence of C4A or C4B can be C4A (short form) or C4B (short form) (i.e., C4A or C4B polynucleotide without an insertion of a HERV sequence in intron 9).

While the examples provided below describe specific methods of detecting levels of polynucleotides or polypeptides of the markers C4A and C4B, the skilled artisan appreciates that the invention is not limited to such methods. The biomarkers of this invention can be detected or quantified by any suitable method. For example, methods include, but are not limited to real-time PCR, Southern blot, PCR, mass spectroscopy, ELISA, and/or antibody binding. Methods for detecting a copy number and/or sequence of C4A or C4B or other polynucleotides of the invention include immunoassay, direct sequencing, and probe hybridization to a polynucleotide. In particular embodiments, a sequence and/or copy number of the markers is detected by DNA sequencing and/or copy number analysis.

### Methods of Treatment of Schizophrenia

The present invention provides methods of treating schizophrenia and/or disorders or symptoms thereof which comprise administering a therapeutically effective amount of a pharmaceutical composition comprising an anti-schizophrenia agent (e.g., an antipsychotic agent) herein to a pre-selected subject (e.g., a mammal such as a human). In some embodiments, the subject is pre-selected by detecting an alteration in copy number and/or sequence of C4A and/or C4B polynucleotide relative to a reference. In other embodiments, the subject is pre-identified as having or at risk for schizophrenia, Thus, one embodiment is a method of treating a subject suffering from or susceptible to schizophrenia or disorder or symptom thereof. The method includes the step of administering to the mammal a therapeutic amount of an amount of an agent (e.g., antipsychotic agent) herein sufficient to treat the disease or disorder or symptom thereof, under conditions such that the disease or disorder is treated.

The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g. opinion) or objective (e.g. measurable by a test or diagnostic method, such as the methods described herein).

The therapeutic methods of the invention (which include prophylactic treatment) in general comprise administration of a therapeutically effective amount of the agents herein (such as an antipsychotic agent) to a subject (e.g., animal, human) in need thereof, including a mammal, particularly a human. Such treatment will be suitably administered to subjects, particularly humans, suffering from, having, susceptible to, or at risk for a schizophrenia, disorder, or symptom thereof. In some embodiments, determination of those subjects “at risk” is made by an objective determination using the methods described herein.

In one embodiment, the invention provides a method of monitoring treatment progress. The method includes the step of determining a level of diagnostic marker (e.g., level of a polynucleotide or polypeptide of C4A and/or C4B) or diagnostic measurement (e.g., screen, assay) in a subject suffering from or susceptible to a schizophrenia, or disorder or symptoms thereof, in which the subject has been administered a therapeutic or effective amount of a therapeutic agent described herein sufficient to treat the schizophrenia or symptoms thereof. The level of a polynucleotide or polypeptide of C4A and/or C4B determined in the method can be compared to known levels of a polynucleotide or polypeptide of C4A and/or C4B in either healthy normal controls or in other afflicted patients to establish the subject's disease status. In some embodiments, a level of a polynucleotide or polypeptide of C4A and/or C4B in a cerebrospinal fluid (CSF) sample obtained from the subject is determined. In some embodiments, a second level of a polynucleotide or polypeptide of C4A and/or C4B in the subject is determined at a time point later than the determination of the first level, and the two levels are compared to monitor the course of disease or the efficacy of the therapy. In certain embodiments, a pre-treatment level, sequence, or copy number of a polynucleotide or polypeptide of C4A and/or C4B in the subject is determined prior to beginning treatment according to this invention; this pre-treatment level of a polynucleotide or polypeptide of C4A and/or C4B can then be compared to the level of a polynucleotide or polypeptide of C4A and/or C4B in the subject after the treatment commences, to determine the efficacy of the treatment.

In particular embodiments, the agent is an antipsychotic agent. Exemplary antipsychotic agents approved by the U.S. Food and Drug Administration for treatment of schizophrenia or symptoms thereof include, but are not limited to, aripiprazole, asenapine, clozapine, iloperidone, lurasidone, olanzapine, paliperidone, quetiapine, risperidone, ziprasidone, chlorpromazine, fluphenazine, haloperidol, and perphenazine. Commonly used first-line anti-psychotics for (first-episode) schizophrenia include quetiapine, risperidone, ziprasidone.

In some embodiments, the agent is a complement inhibitor. FDA-approved complement inhibitors that are currently in use for other indications include Eculizumab/Soliris and Cetor/Sanquin. In some embodiments, the complement inhibitor is an anti-C1q antibody or fragment thereof (see, e.g., U.S. Patent Publication No. 2016/0159890). In particular embodiments, the complement inhibitor inhibits synaptic pruning.

In some embodiments, the methods include administering psychosocial therapy or treatment to pre-selected subject. Psychosocial treatments for schizophrenia can include, for example, individual therapy, family therapy, social skills training, and vocational rehabilitation. Individual therapy is aimed at training an individual learn to cope with stress and identify early warning signs of relapse, which can help an individual with schizophrenia manage the illness. Family therapy provides support and education to families dealing with schizophrenia. Social skills training focuses on improving communication and social interactions of the individual with schizophrenia. Vocational rehabilitation focuses on helping individuals with schizophrenia prepare for, find and keep jobs. Most individuals with schizophrenia require some form of daily living support. Many communities have programs to help individuals with schizophrenia with jobs, housing, self-help groups and crisis situations. In some embodiments, a schizophrenia treatment can integrate antipsychotic agents, psychosocial therapies, case management, family involvement, and supported education and employment services, all aimed at reducing symptoms and improving quality of life of the individual with schizophrenia.

### Therapeutic Agents Targeting C4A

In other aspects, the invention provides a method of treating schizophrenia by selectively interfering with the function of C4A polypeptide. In some embodiments, the interference with C4A polypeptide function is achieved using an antibody binding to C4A polypeptide. In some embodiments, the antibody specifically binds to C4A polypeptide, and does not bind C4B polypeptide. In certain embodiments, the antibody binds to both C4A and C4B polypeptide.

In certain embodiments, the antibody disrupts or reduces interaction between a neuron and microglia. Without being bound by theory, it is believed that reduced interaction between a neuron and microglia decreases synaptic pruning. Accordingly, in some embodiments, the antibody reduces synaptic pruning.

Antibodies can be made by any of the methods known in the art utilizing a polypeptide of the invention (e.g., C4A and C4B polypeptide), or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a polypeptide of the invention or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding the polypeptide, or immunogenic fragments thereof, can be expressed in cells in vitro, followed by isolation of the polypeptide and administration of the polypeptide to a suitable host in which antibodies are raised.

Alternatively, antibodies against the polypeptide may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

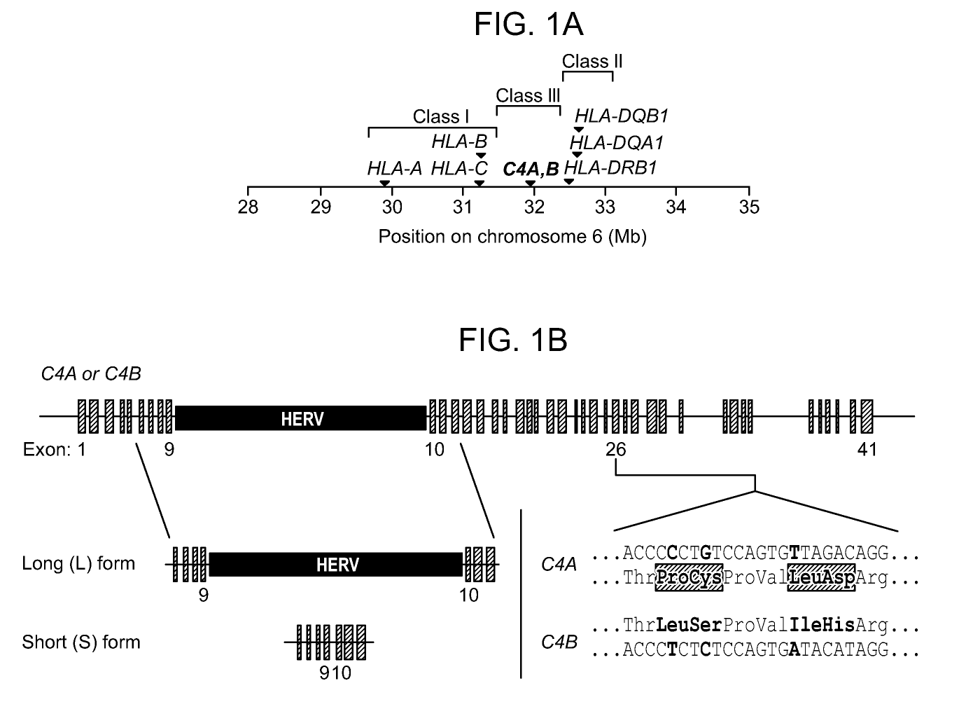

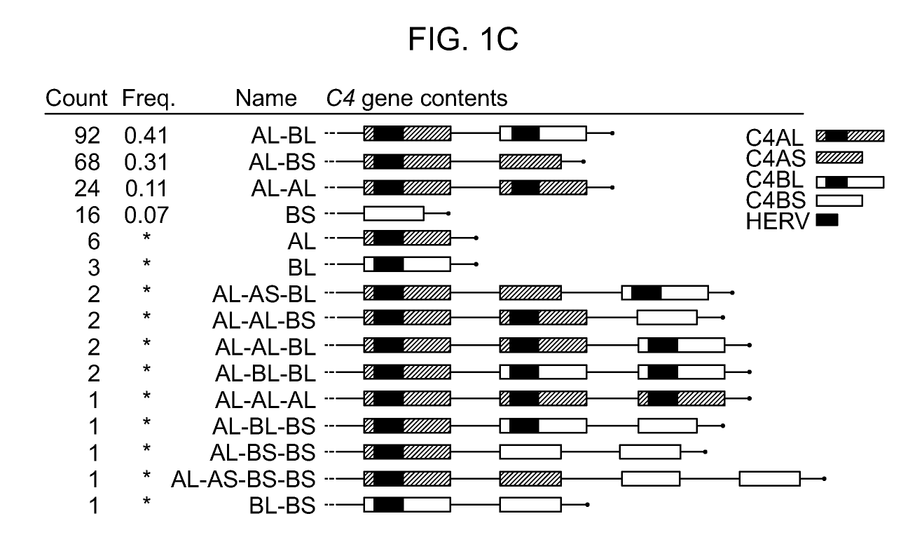

Without intending to be bound by theory, results herein indicate that therapeutically it might be advantageous to selectively interfere with C4A while leaving C4B function intact. This could be important because ideally one would not want to entirely block complement function in the body, since complement is important for protection from immune assault and from auto-immunity. Thus, in some embodiments, therapeutic antibodies that selectively bind to C4A polypeptide and not to C4B polypeptide are generated by exploiting the amino-acid sequence differences between C4A and C4B to identify epitopes for isotope-specific antibodies. In some embodiments, the amino acid sequence difference between C4A and C4B is that shown in FIG. 1B. Thus, in certain embodiments, the antibody specifically binds an epitope containing the sequence PCPVLD. In particular embodiments, the antibody does not bind an epitope containing the sequence LSPVIH.

### Pharmaceutical Compositions

The present invention features compositions useful for treating schizophrenia in a pre-selected subject. The administration of a composition comprising a therapeutic agent herein (e.g., an antipsychotic agent, an inhibitory nucleic acid inhibiting expression for C4A polypeptide, or an antibody specifically binding to C4A polypeptide) for the treatment of schizophrenia may be by any suitable means that results in a concentration of the therapeutic that, combined with other components, is effective in ameliorating, reducing, or stabilizing schizophrenia in a subject. The composition may be administered systemically, for example, formulated in a pharmaceutically-acceptable buffer such as physiological saline. Routes of administration include, for example, intrathecal, subcutaneous, intravenous, interperitoneally, intramuscular, or intradermal injections that provide continuous, sustained levels of the agent in the patient. In particular embodiments, the composition comprising a therapeutic agent herein is administered intrathecally to a subject. In some embodiments, the composition is injected into the spinal canal (in particular, subarachnoid space) of the subject such that the composition reaches the cerebrospinal fluid.

When the binding target is located in the brain, certain embodiments of the invention provide for the antibody or antigen-binding fragment thereof to traverse the blood-brain barrier. Certain neurodegenerative diseases are associated with an increase in permeability of the blood-brain barrier, such that the antibody or antigen-binding fragment can be readily introduced to the brain. When the blood-brain barrier remains intact, several art-known approaches exist for transporting molecules across it, including, but not limited to, physical methods, lipid-based methods, and receptor and channel-based methods.

In certain embodiments, a chimeric molecule is generated comprising a fusion of an antibody or other therapeutic polypeptide with a protein transduction domain which targets the antibody or therapeutic polypeptide for delivery to various tissues and more particularly across the brain blood barrier, using, for example, the protein transduction domain of human immunodeficiency virus TAT protein (Schwarze et al., 1999, Science 285: 1569-72) or BBB peptide (Brainpeps® database; http://brainpeps.ugent.be/; Van Dorpe et al., Brain Structure and Function, 2012, 217(3), 687-718). Other polypeptides facilitating transport across the blood-brain-barrier, include without limitation, transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, Syn-B, penetratin, a poly-arginine peptide, an angiopep peptide, and ANG1005.

In certain embodiments, compositions disclosed herein can be formulated to ensure proper distribution in vivo. For example, the blood-brain barrier (BBB) excludes many highly hydrophilic compounds. To ensure that therapeutic compounds in compositions of the invention cross the BBB, they can be formulated, for example, in liposomes. Lipid-based methods of transporting an antibody or antigen-binding fragment across the blood-brain barrier include, but are not limited to, encapsulating the antibody or antigen-binding fragment in liposomes that are coupled to antibody binding fragments that bind to receptors on the vascular endothelium of the blood-brain barrier (see, e.g., U.S. Patent Application Publication No. 20020025313), and coating the antibody or antigen-binding fragment in low-density lipoprotein particles (see, e.g., U.S. Patent Application Publication No. 20040204354) or apolipoprotein E (see, e.g., U.S. Patent Application Publication No. 20040131692). For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the invention, as well as components of the invented molecules (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Physical methods of transporting the antibody or antigen-binding fragment across the blood-brain barrier include, but are not limited to, circumventing the blood-brain barrier entirely, or by creating openings in the blood-brain barrier. Circumvention methods include, but are not limited to, direct injection into the brain (see, e.g., Papanastassiou et al., Gene Therapy 9: 398-406 (2002); interstitial infusion/convection-enhanced delivery (see, e.g., Bobo et al., Proc. Natl. Acad. Sci. USA 91: 2076-2080 (1994)), and implanting a delivery device in the brain (see, e.g., Gill et al., Nature Med. 9: 589-595 (2003); and Gliadel Wafers™, Guildford Pharmaceutical). Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, vols. 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416), and transfection of neurons that straddle the blood-brain barrier with vectors containing genes encoding the antibody or antigen-binding fragment (see, e.g., U.S. Patent Publication No. 2003/0083299).

Receptor and channel-based methods of transporting the antibody or antigen-binding fragment across the blood-brain barrier include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473); inhibiting ABC drug transporters (see, e.g., U.S. Patent Application Publication No. 2003/0073713); coating antibodies with a transferrin and modulating activity of the one or more transferrin receptors (see, e.g., U.S. Patent Application Publication No. 2003/0129186), and cationizing the antibodies (see, e.g., U.S. Pat. No. 5,004,697).

The amount of the therapeutic agent to be administered varies depending upon the manner of administration, the age and body weight of the patient, and with the clinical symptoms of schizophrenia. Generally, amounts will be in the range of those used for other agents used in the treatment of schizophrenia, although in certain instances lower amounts will be needed because of the increased specificity of the agent. A composition is administered at a dosage that decreases effects or symptoms of schizophrenia as determined by a method known to one skilled in the art.

The therapeutic agent (e.g., an antipsychotic agent herein) may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the liver; (v) formulations that allow for convenient dosing, such that doses are administered, for example, once every one or two weeks; and (vi) formulations that target schizophrenia using carriers or chemical derivatives to deliver the therapeutic agent to a particular cell type (e.g., cells in the brain). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered intrathecally or parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates schizophrenia, the composition may include suitable parenterally acceptable carriers and/or excipients. The active therapeutic agent(s) (e.g., antipsychotic agent) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic (e.g., antipsychotic agent) is formulated for intravenous delivery. As indicated above, the pharmaceutical compositions according to the invention may be in the form suitable for sterile injection. To prepare such a composition, the suitable therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Inhibitory Nucleic Acid Therapy

Another therapeutic approach for treating or slowing progression of schizophrenia is polynucleotide therapy using an inhibitory nucleic acid that inhibits expression of a C4A and/or C4B polynucleotide (in particular, a C4A polynucleotide). Thus, provided herein are inhibitory nucleic acid molecules, such as siRNA, that target C4A and/or C4B polynucleotide. Such nucleic acid molecules can be delivered to cells of a subject having schizophrenia. The nucleic acid molecules are delivered to the cells of a subject in a form in which they can be taken up so that therapeutically effective levels of the inhibitory nucleic acid molecules are introduced.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, an inhibitory nucleic acid as described can be cloned into a retroviral vector and expression can be driven from its endogenous promoter, from the retroviral long terminal repeat, or from a promoter specific for a target cell type of interest. In some embodiments, the target cell type of interest is a neuron. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In some embodiments, a viral vector is used to administer a polynucleotide encoding inhibitory nucleic acid molecules that inhibit C4A and/or C4B expression.

Non-viral approaches can also be employed for the introduction of the therapeutic to a cell of a patient requiring treatment of schizophrenia. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of polynucleotide encoding inhibitory nucleic acid molecules into the affected tissues of a patient can also be accomplished by transferring a polynucleotide encoding the inhibitory nucleic acid into a cultivatable cell type ex vivo (e.g., an autologous or heterologous primary cell or progeny thereof), after which the cell (or its descendants) are injected into a targeted tissue. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In some embodiments, the inhibitory nucleic acid molecule is selectively expressed in a neuron. In some other embodiments, the inhibitory nucleic acid molecule is expressed in a neuron using a lentiviral vector. In still other embodiments, the inhibitory nucleic acid molecule is administered intrathecally. Selective targeting or expression of inhibitory nucleic acid molecules to a neuon is described in, for example, Nielsen et al., J Gene Med. 2009 July; 11(7):559-69. doi: 10.1002/jgm.1333.

### Screening Assays

The present invention further features methods of identifying modulators of a disease, particularly schizophrenia, comprising identifying candidate agents that interact with and/or alter the level or activity of a polynucleotide or polypeptide of C4A or C4B. As described elsewhere herein, increased expression of C4A was associated with increased risk of schizophrenia and increased synaptic elimination. Without being bound by theory, it is believed that interfering with C4A function or activity can decrease synaptic pruning and/or inhibit development or progression of schizophrenia in a subject.

Thus, in some aspects, the invention provides a method of identifying a modulator of schizophrenia, comprising (a) contacting a cell or organism with a candidate agent, and (b) measuring a level of polynucleotide or polypeptide of C4A or C4B in the cell relative to a control level. An alteration in the level of C4A or C4B polypeptide or polynucleotide indicates the candidate agent is a modulator of schizophrenia. In particular, a decrease in the level of C4A polynucleotide or polypeptide indicates the candidate agent is an inhibitor of schizophrenia. In some embodiments, the cell or organism is a recombinant cell or recombinant organism that overexpresses C4A polynucleotide or polypeptide.

Methods of measuring or detecting activity and/or levels of the polypeptide or polynucleotide are known to one skilled in the art. Polynucleotide levels may be measured by standard methods, such as quantitative PCR, Northern Blot, microarray, mass spectrometry, and in situ hybridization. Standard methods may be used to measure polypeptide levels, the methods including without limitation, immunoassay, ELISA, western blotting using an antibody that binds the polypeptide, and radioimmunoassay.

In some embodiments, the C4A polypeptide is fused to a detectable label (e.g., a fluorescent reporter polypeptide). Level(s) of C4A polypeptide in a cell contacted with a candidate agent can then be easily monitored by measuring fluorescence of the reporter polypeptide.

### Recombinant Cells or Organisms

A recombinant cell or organism comprising an isolated C4A or C4B polynucleotide (in particular, a recombinant cell overexpressing C4A polynucleotide or polypeptide) can be useful in screening assays for identifying modulators (e.g., inhibitors) of schizophrenia. Accordingly, the invention provides a recombinant cell or organism heterologously expressing C4A polypeptide. In some embodiments, the cell is a mammalian cell. In some embodiments, the organism is a mouse.

Recombinant cells or organisms of the invention are produced using virtually any method known to the skilled artisan. Typically, recombinant cells are produced by transformation of a suitable host cell with all or part of a polypeptide-encoding nucleic acid molecule or fragment thereof in a suitable expression vehicle. Those skilled in the field of molecular biology will understand that any of a wide variety of expression systems may be used to express (particularly, overexpress) C4A or C4B polypeptide in a host cell or organism. The precise host cell or organism used is not critical to the invention.

In some embodiments, the C4A or C4B polynucleotide or polypeptide is expressed in mammalian cells. Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the expression of the polypeptides (e.g., C4A or C4B) of the invention in a host cell or organism. “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or organism. Expression vectors include all those known in the art, such as plasmids or viral vectors that incorporate the recombinant polynucleotide.

In some embodiments, the expression vector comprises an inducible or constitutive promoter operably linked to a C4A or C4B polynucleotide. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

### Kits

The invention provides kits for treating schizophrenia in a subject and/or identifying a subject having or at risk of developing schizophrenia. A kit of the invention provides a capture reagent (e.g., a primer or hybridization probe specifically binding to a C4A or C4B polynucleotide) for measuring relative expression level, copy number, and/or a sequence of a marker (e.g., C4A or C4B). In other embodiments, the kit further includes reagents suitable for DNA sequencing or copy number analysis of C4A and/or C4B.

In one embodiment, the kit includes a diagnostic composition comprising a capture reagent detecting at least one marker selected from the group consisting of a C4A polynucleotide and a C4B polynucleotide. In one embodiment, the capture reagent detecting a polynucleotide of C4A or C4B is a primer or hybridization probe that specifically binds to a C4A or C4B polynucleotide. The kits may further comprise a therapeutic composition comprising one or more antipsychotic agents. In some embodiments, the antipsychotic agent is aripiprazole, asenapine, clozapine, iloperidone, lurasidone, olanzapine, paliperidone, quetiapine, risperidone, ziprasidone, chlorpromazine, fluphenazine, haloperidol, and perphenazine.

In some embodiments, the kit comprises a sterile container which contains a therapeutic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, the kit further comprises instructions for using the diagnostic agents and/or administering the therapeutic agents of the invention. In particular embodiments, the instructions include at least one of the following: description of the therapeutic agent; dosage schedule and administration for reducing schizophrenia symptoms; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: C4 Structures and MHC SNP Haplotypes

Human C4 exists as two functionally distinct genes (isotypes), C4A and C4B; both vary in structure and copy number. One to three C4 genes (C4A and/or C4B) are commonly present as a tandem array within the MHC class III region (FIG. 1A, FIG. 8G)^{14-18.} The protein products of C4A and C4B bind different molecular targets^{19,20}. C4A and C4B segregate in both long and short genomic forms (C4AL, AS, BL and BS), distinguished by the presence or absence (in intron 9) of a human endogenous retroviral (HERV) insertion that lengthens C4 from 14 to 21 kb without changing the C4 protein sequence¹⁶ (FIG. 1B). The most strongly associated markers in several large case/control cohorts were near a complex, multi-allelic, and only partially characterized form of genome variation that affects the C4 gene encoding complement component 4 (FIGS. 8A-8G).

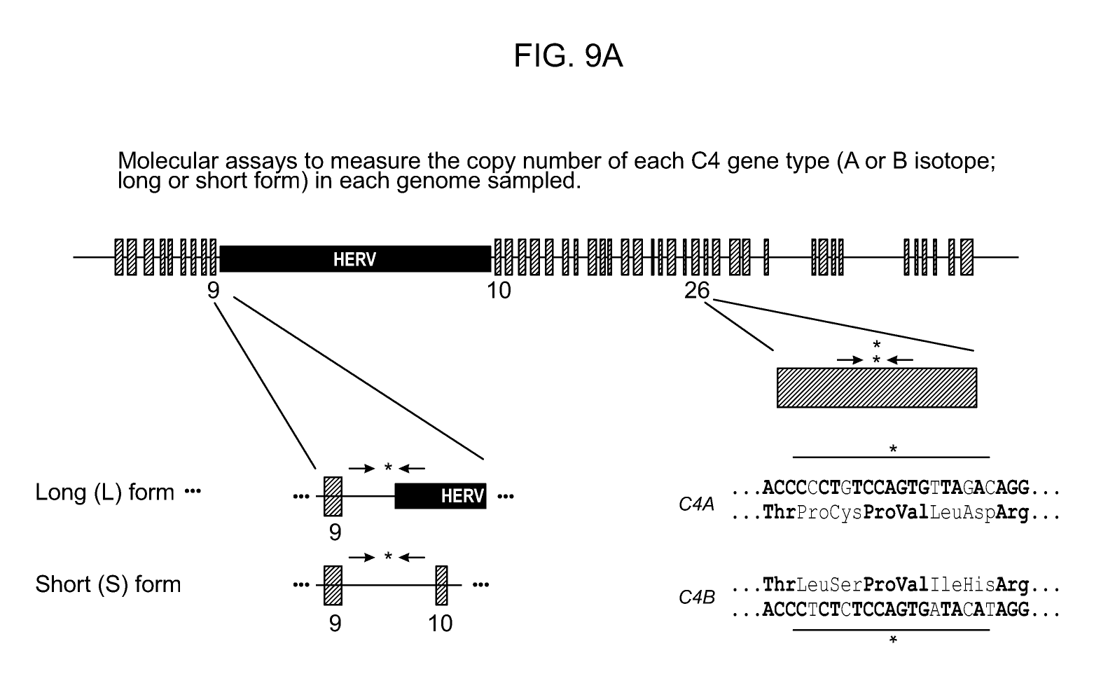

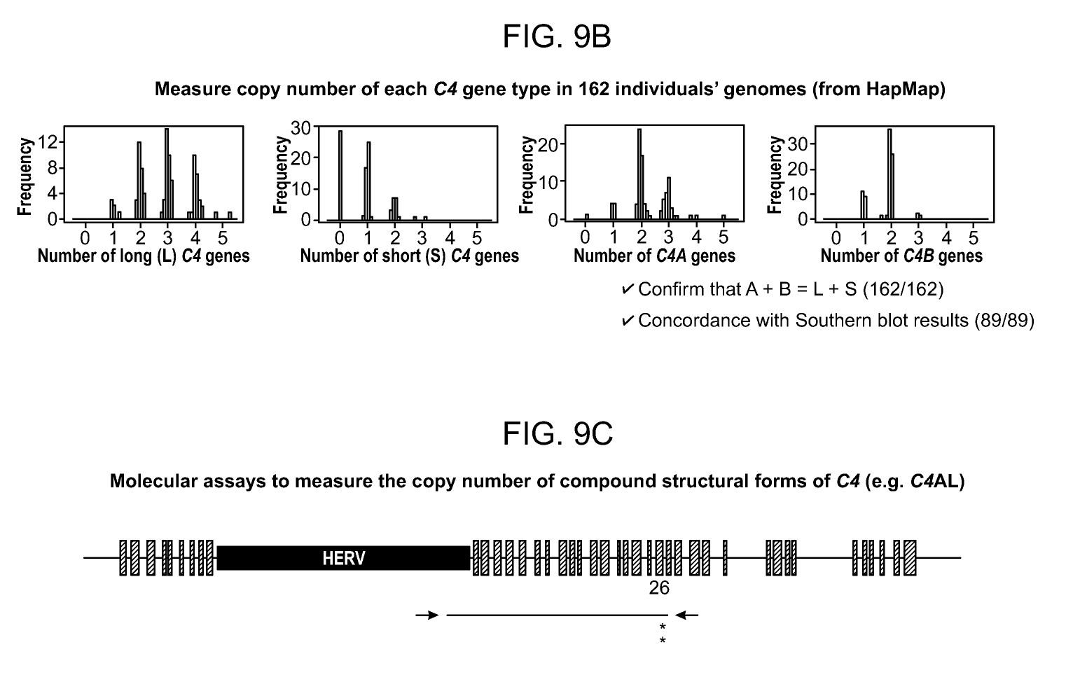

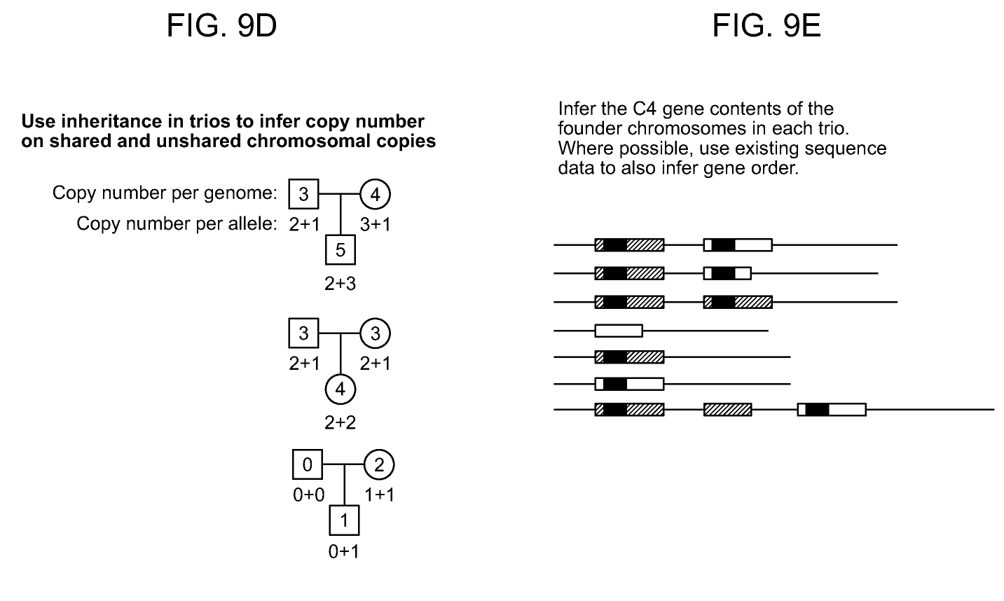

A method (FIGS. 9A-9E) to identify the “structural haplotypes” of C4—the copy number of C4A and C4B and the long/short (HERV) status of each C4A and C4B copy—present on 222 copies of human chromosome 6 was developed. Using droplet digital PCR (ddPCR), it was found that genomes contained 0-5 C4A genes, 0-3 C4B genes, 1-5 long (L) C4 genes, and 0-3 short (S) C4 genes (FIGS. 9A-9B). Assays were developed to determine the long/short status of each C4A and C4B gene copy (FIG. 9C), thus revealing copy number of C4AL, C4BL, C4AS, and C4BS in each genome.

Inheritance in father-mother-offspring trios were analyzed (FIG. 9D) to identify the C4A and C4B contents of individual alleles (FIG. 9E). It was found that 4 common C4 structural haplotypes (AL-BL, AL-BS, AL-AL, and BS) were collectively present on 90% of the 222 independent chromosomes sampled; 11 uncommon C4 haplotypes comprised the other 10% (FIG. 1C).

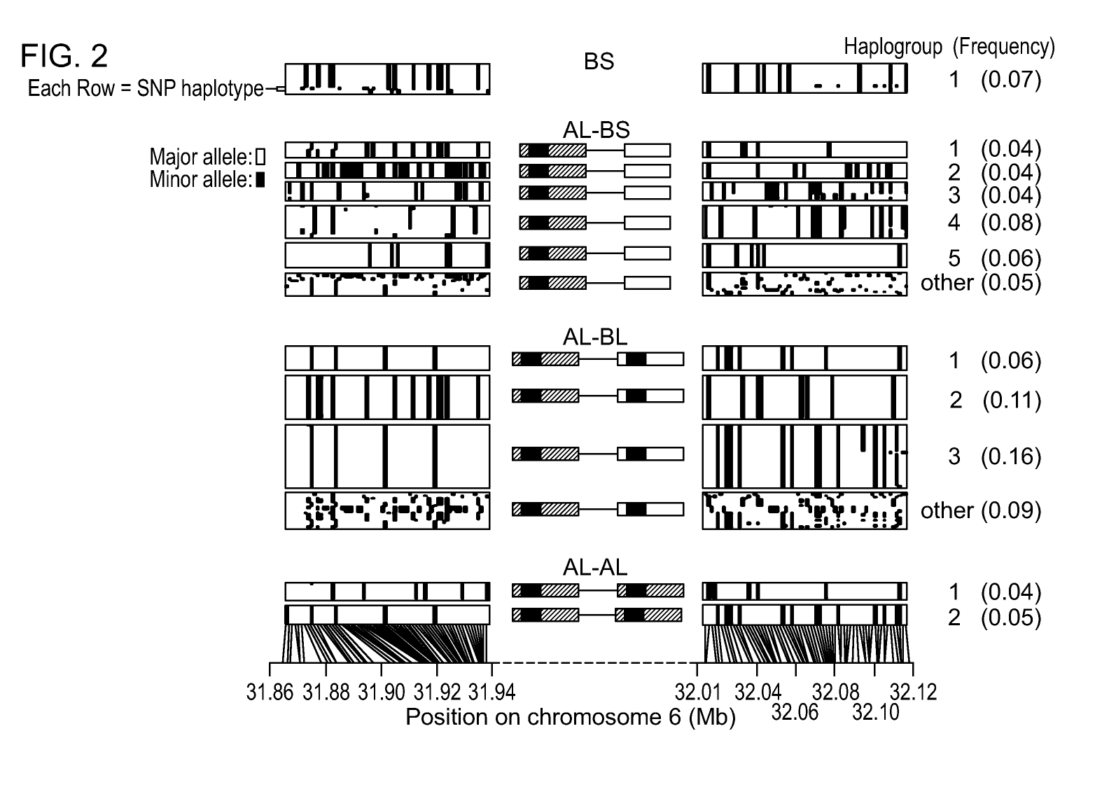

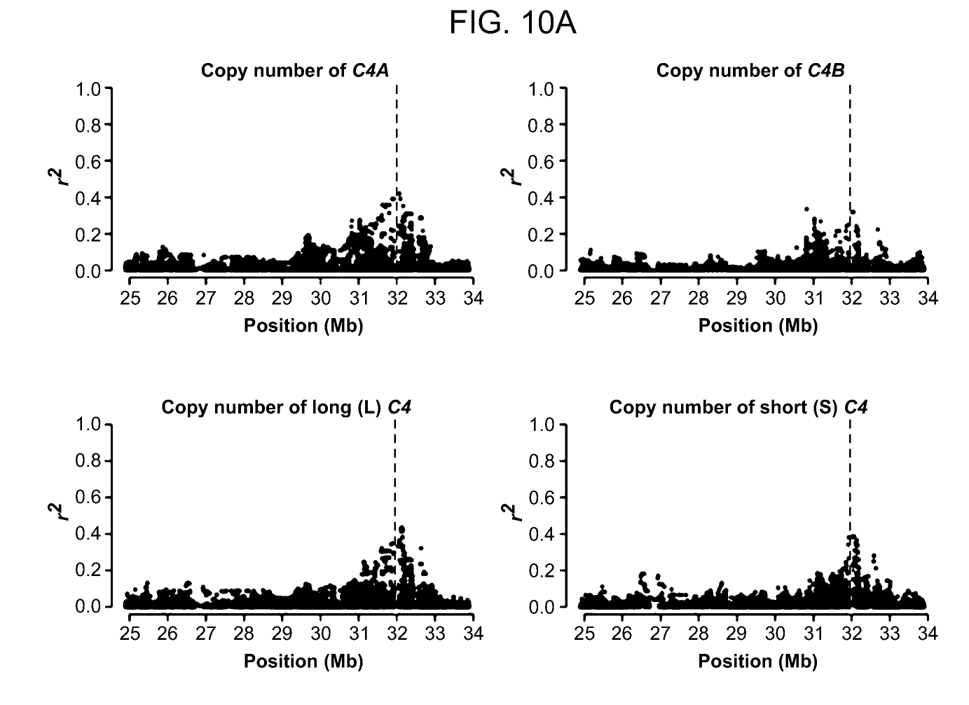

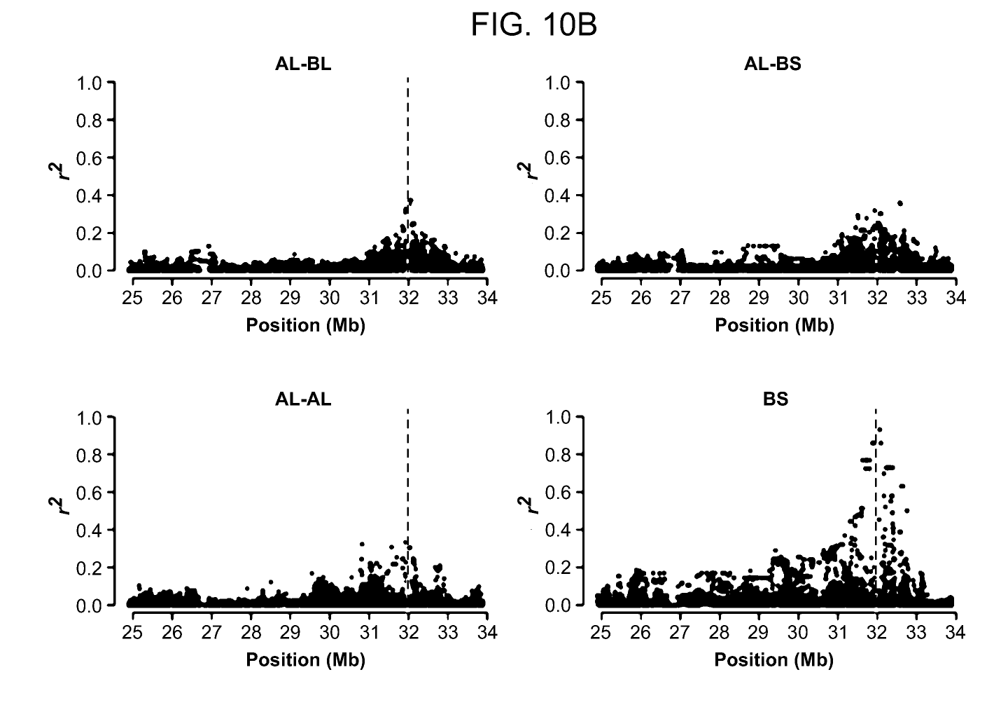

The series of many SNP alleles along a genomic segment (the SNP haplotype) can be used to identify chromosomal segments that come from shared common ancestors. The SNP haplotype(s) on which each C4 locus structure was present were identified (FIG. 2). The three most common C4 locus structures were each present on multiple MHC SNP haplotypes (FIG. 2). For example, the C4 AL-BS structure (frequency 31%) was present on five common haplotypes (frequencies 4%, 4%, 4%, 8%, and 6%) and many rare haplotypes (collective frequency 5%, FIG. 2). Reflecting this haplotype diversity, each of these C4 structures exhibited real but only partial correlation to individual SNPs (FIGS. 10A-10B). The relationship between C4 structures and SNP haplotypes was generally one-to-many: a C4 structure might be present on many haplotypes, but a given SNP haplotype tended to have one characteristic C4 structure (FIG. 2).

### Example 2: C4 Expression Variation in the Brain

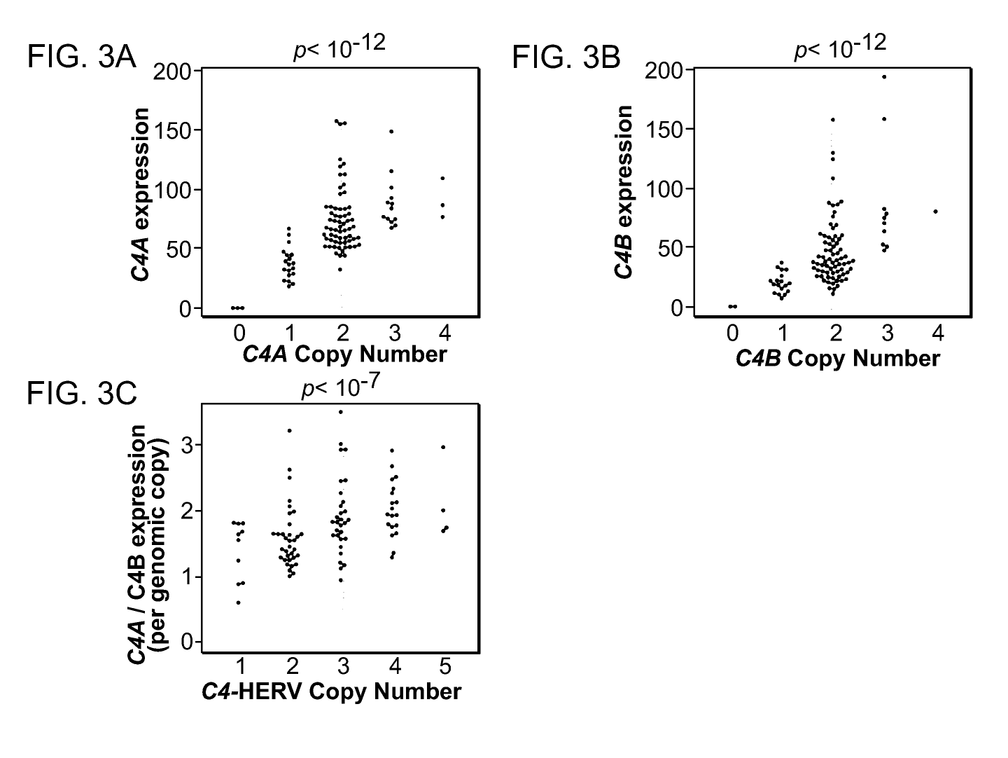

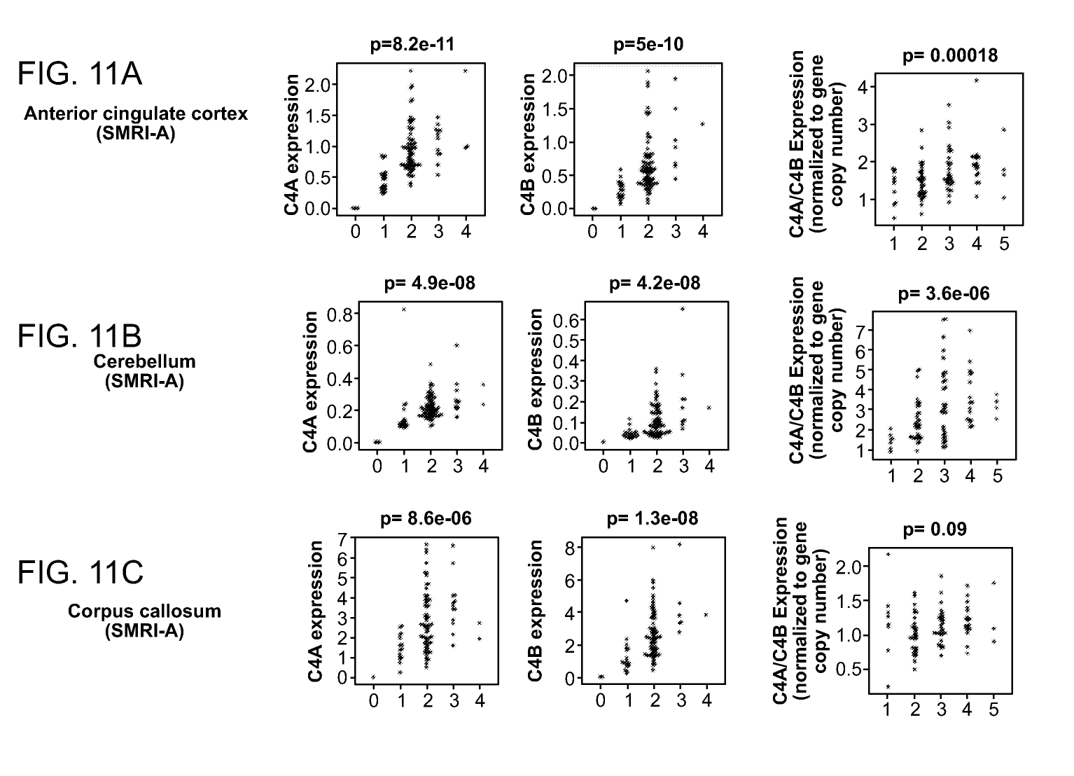

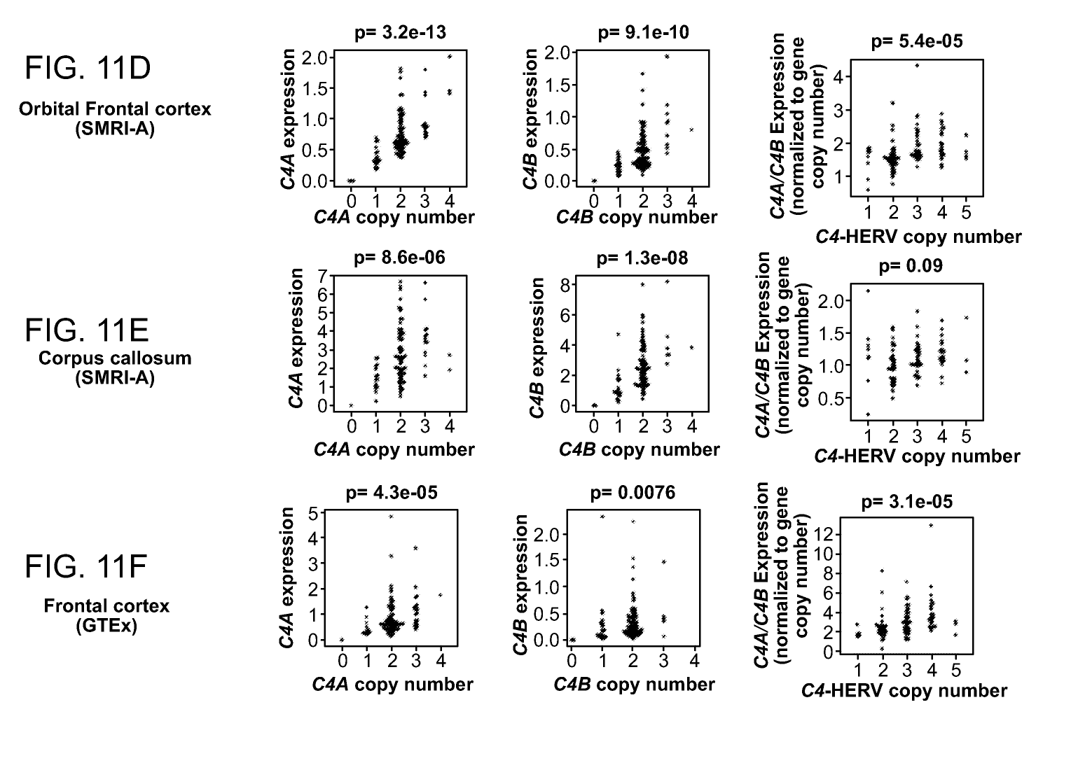

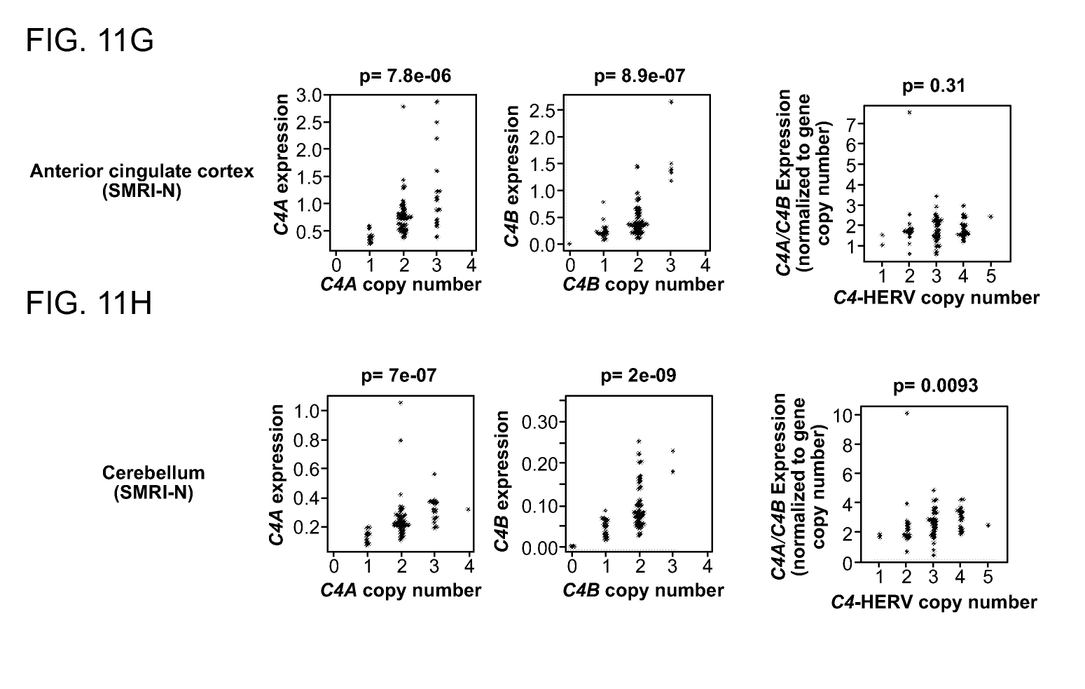

Since C4A and C4B vary in both copy number and C4-HERV status (FIGS. 1A-1C), and because other HERVs can function as enhancers²¹⁻²³, C4 variation might affect C4 genes' expression. It was then assessed how C4 structural variation related to RNA expression of C4A and C4B in eight panels of post mortem human adult brain samples (674 samples from 245 distinct donors in 3 cohorts. The results of this expression analysis were consistent across all five brain regions analyzed. First, RNA expression of C4A and C4B increased proportionally with copy number of C4A and C4B respectively (FIGS. 3A-3B; FIGS. 11A-11H). These observations mirrored earlier observations in human serum²⁴. Second, expression levels of C4A were 2-3 times greater than expression levels of C4B, even after controlling for relative copy number in each genome (FIG. 3C). Third, copy number of the C4-HERV sequence increased the ratio of C4A to C4B expression (p<10⁻⁷, p<10⁻², p<10⁻³) (FIG. 3C, FIGS. 11A-11H). The foregoing data was used to create genetic predictors of C4A and C4B expression levels in the brain. If C4A or C4B expression levels influence a phenotype, then the aggregate genetic predictor would associate to schizophrenia more strongly than individual variants do.

### Example 3: C4 Structural Variation in Schizophrenia

Schizophrenia cases and controls from 22 countries have been analyzed genome-wide for SNPs, implicating the MHC locus as the strongest of more than 100 genome-wide-significant associations⁶. The analysis showed that long haplotypes defined by many SNPs carry characteristic C4 alleles (FIG. 2), potentially making it possible to infer C4 alleles by statistical imputation²⁵ from combinations of many SNPs. The 222 integrated haplotypes of MHC SNPs and C4 alleles (FIG. 2) were used as reference chromosomes for imputation. It was found that the four most common structural forms of the C4A/C4B locus (BS, AL-BS, AL-BL, and AL-AL) could be inferred with reasonably high accuracy (generally 0.70<r²<1.00).

SNP data from 28,799 schizophrenia cases and 35,986 controls, from 40 cohorts in 22 countries contributing to the Psychiatric Genomics Consortium (PGC)⁶ were analyzed. Association to 7,751 SNPs across the extended MHC locus (chr6: 25-34 Mb), to C4 structural alleles (FIG. 1C), and to HLA sequence polymorphisms imputed from the SNP data were evaluated. Levels of C4A and C4B expression from the imputed C4 structural alleles were also predicted.

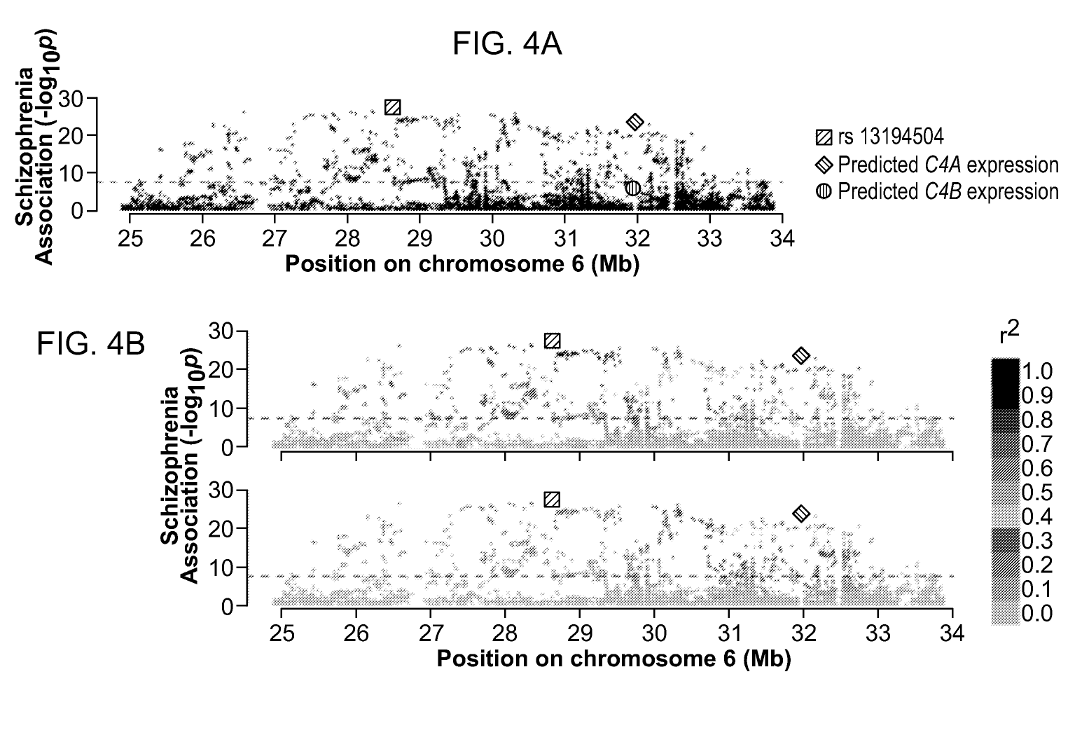

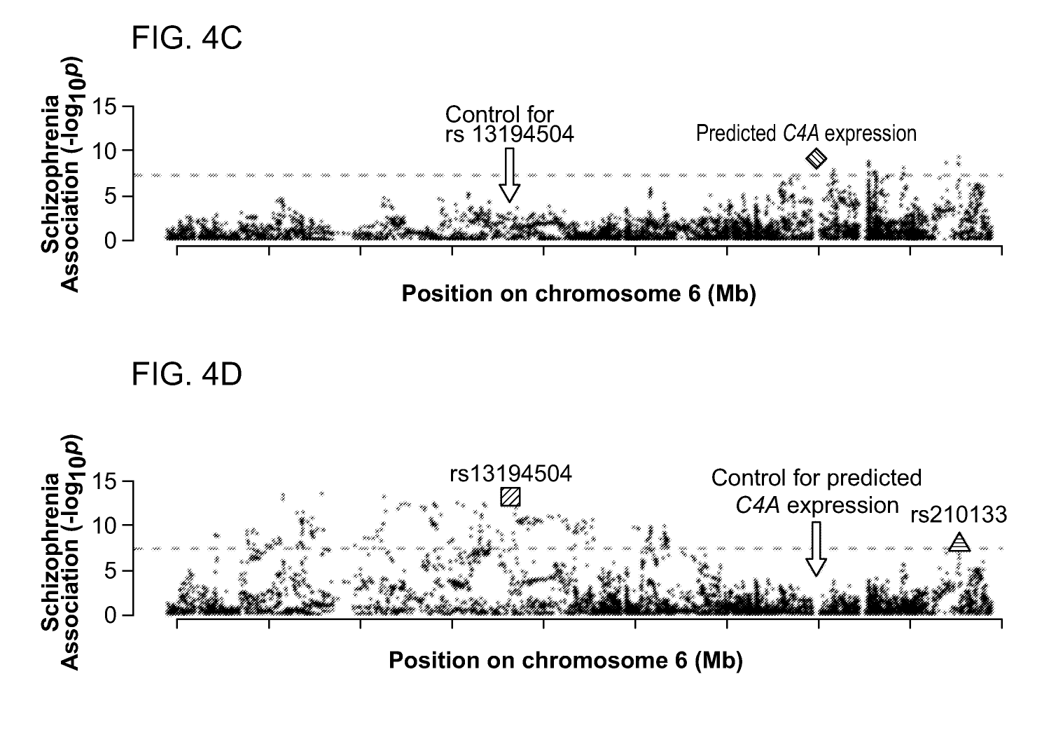

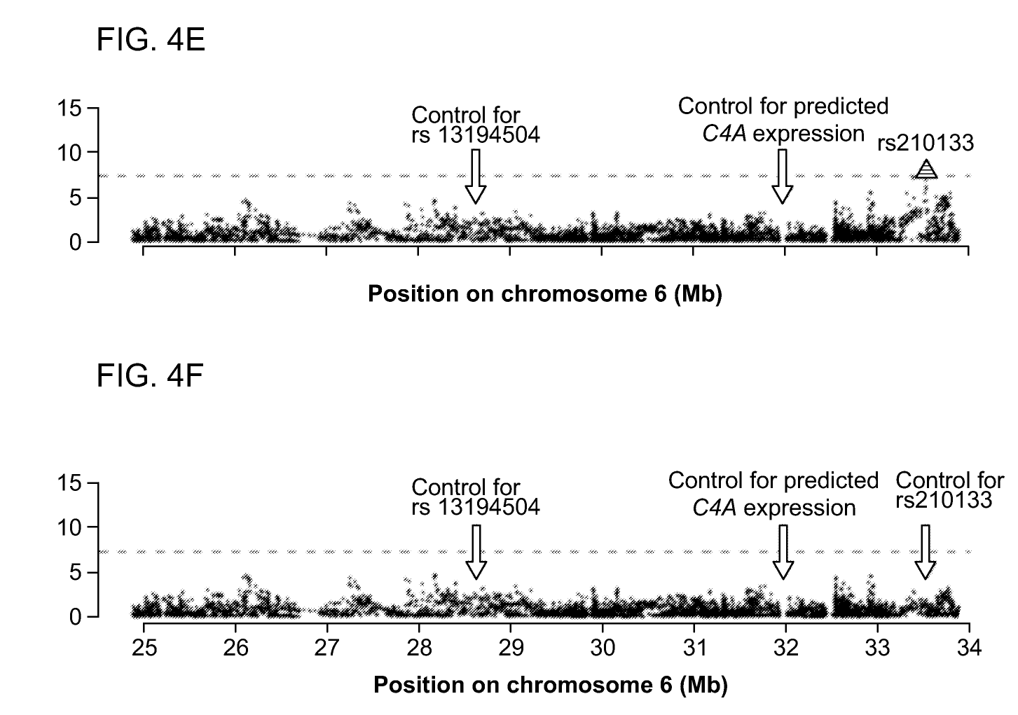

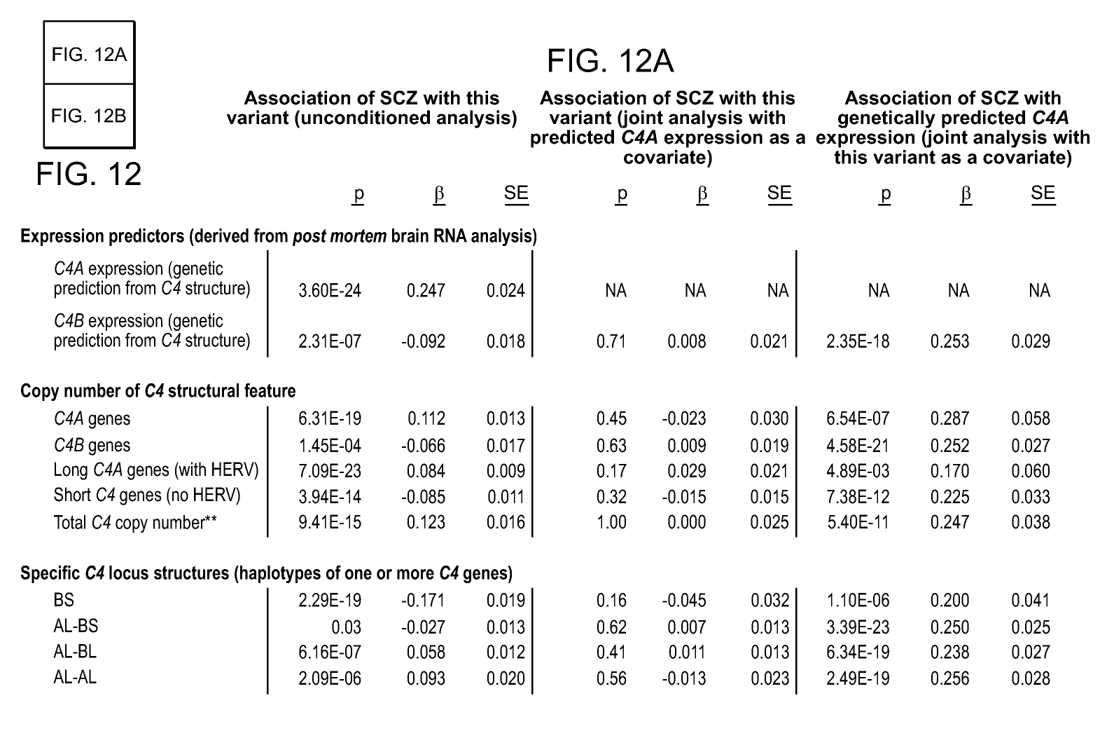

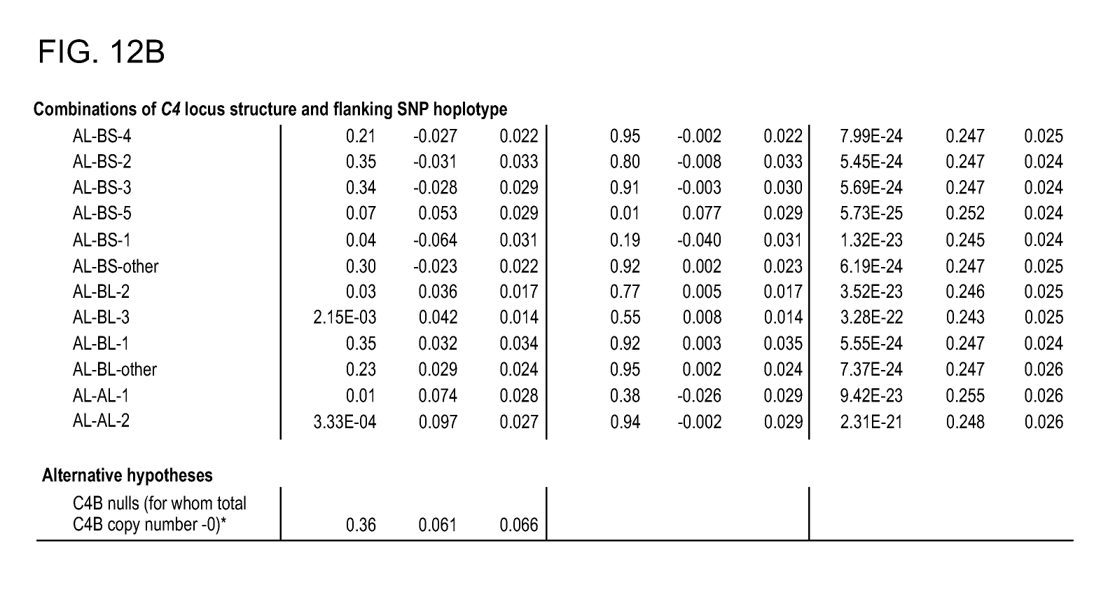

The association of schizophrenia to these genetic variants exhibited two prominent features (FIGS. 4A-4B). One feature involved a large set of similarly-associating SNPs spanning 2 Mb across the distal end of the extended MHC region. In at least some analyses herein, this set's most strongly associating SNP, rs13194504, was used as its genetic proxy. The other peak of association centered at C4, where schizophrenia associated most strongly with the genetic predictor of C4A expression levels (p=3.6×10⁻²⁴) (FIG. 4A, FIG. 12). In the region near C4 (chromosome 6, 31-33 Mb), the more strongly a SNP correlated with predicted C4A expression, the more strongly it associated with schizophrenia (FIG. 4B, bottom).

Although the variation at C4 and in the distal extended MHC region associated to schizophrenia with similar strengths (p=3.6×10⁻²⁴ and 5.5×10⁻²⁸, respectively), their correlation with each other was low (r²=0.18, FIG. 4B), suggesting that they reflect distinct genetic influences. Conditional analysis confirmed this: in analyses controlling for either rs13194504 or genetically predicted C4A expression, the other genetic variable still defined a genome-wide significant association peak (p=7.8×10⁻¹⁰ and 8.0×10¹⁴, FIGS. 4C-4D). Controlling for both genetic variables revealed a third association signal just proximal to the MHC locus (FIG. 4E) involving SNPs around BAK1 and SYNGAP1, the latter of which encodes a major component of the postsynaptic density; de novo loss-of-function mutations in SYNGAP1 associate with autism²⁶. In joint analysis, all three genetic signals remained significant (p=8.0×10⁻¹⁴, 2.8×10⁻⁸, and 1.7×10⁻⁸, respectively) and no additional genome-wide significant signals remained in the MEW locus (FIG. 4F).

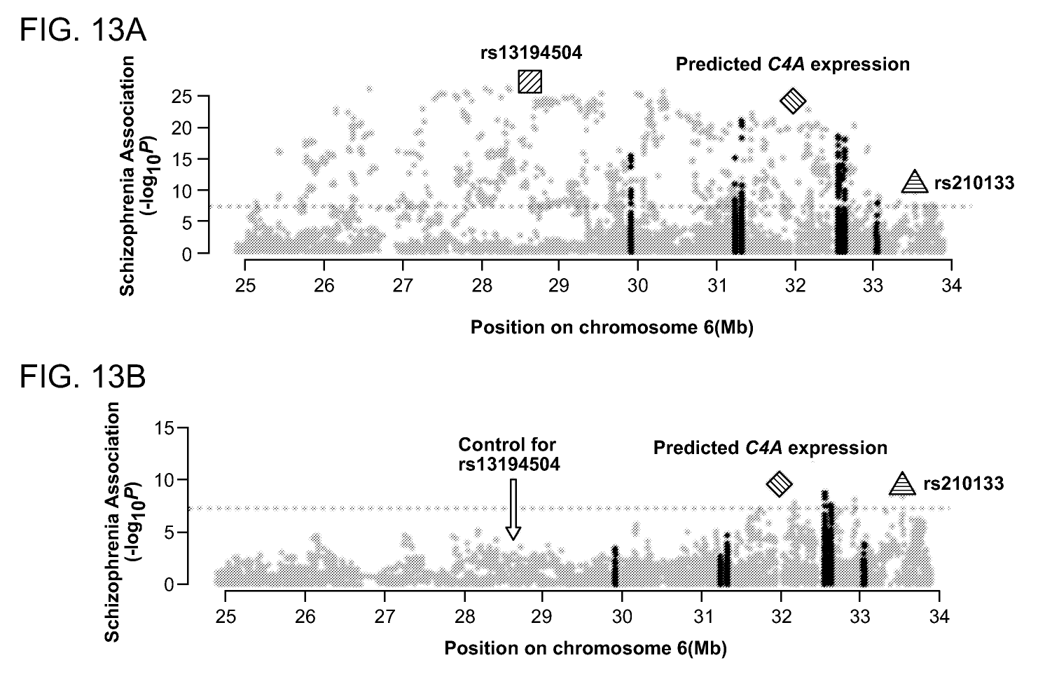

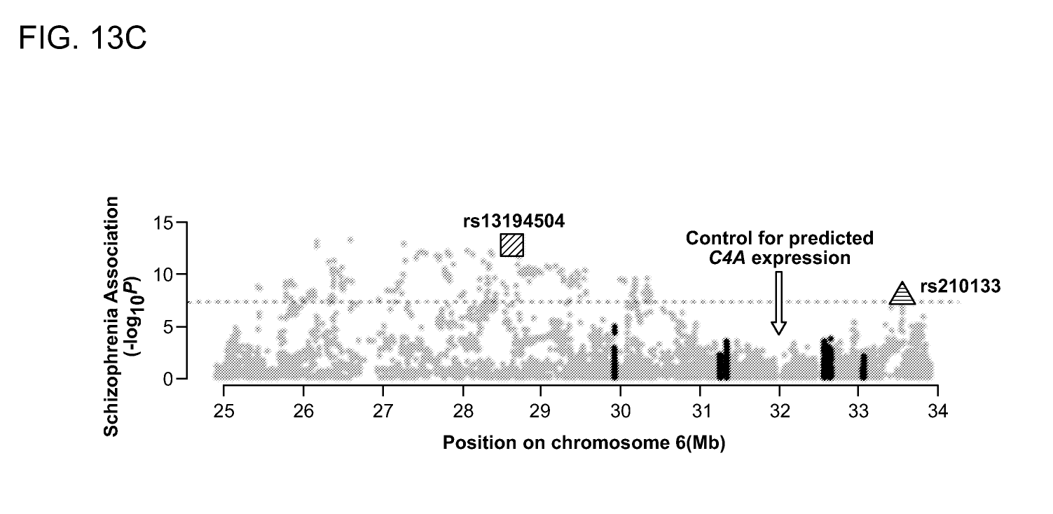

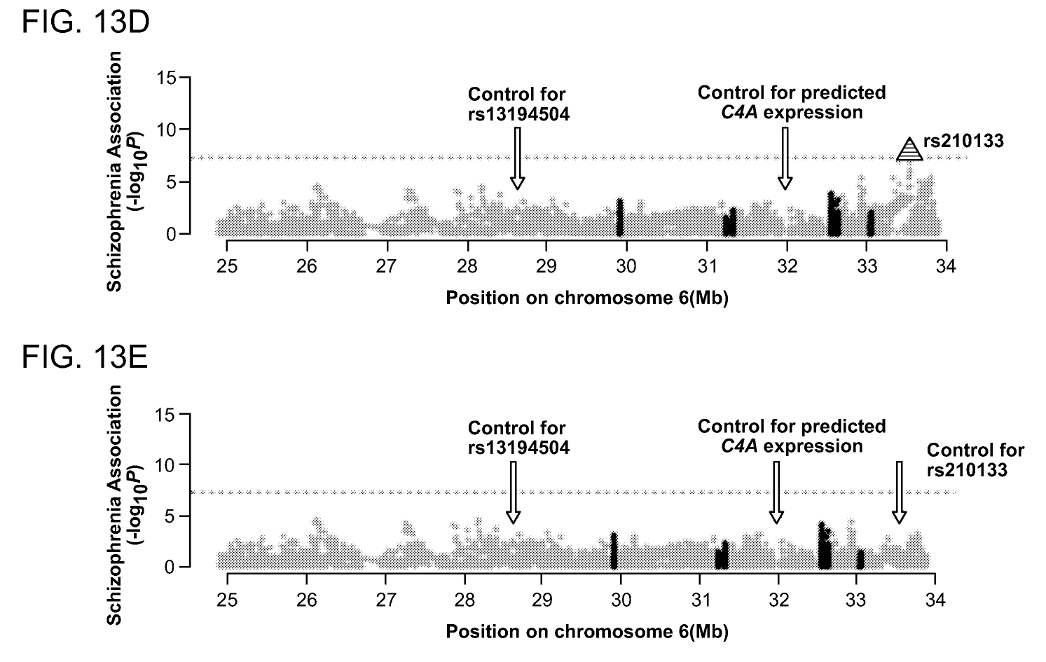

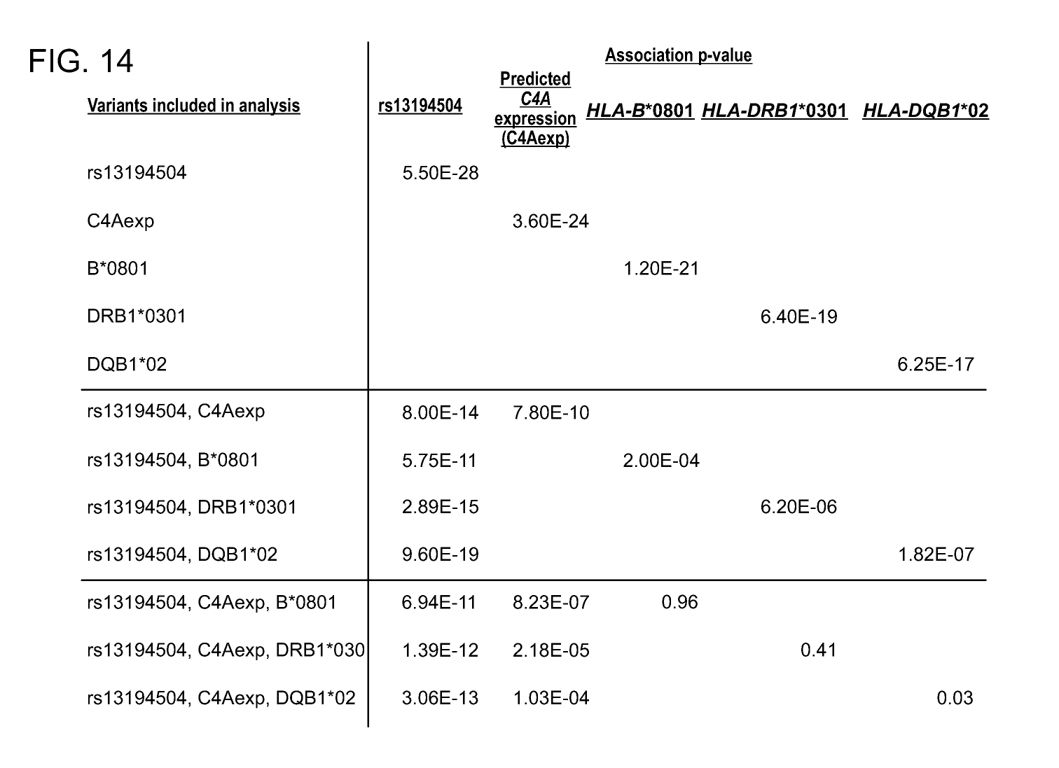

In some autoimmune diseases with genetic associations in the MEW locus, alleles of HLA genes associate more strongly than do other variants in the MEW locus, appearing to explain the associations^{11,12}. In contrast, in schizophrenia, classical HLA alleles associated to schizophrenia less strongly than other genetic variants in the MHC region did (FIGS. 13A-13E). The strongest schizophrenia associations to classical HLA alleles at distinct loci (involving HLA-B*0801, HLA-DRB1*0301, and HLA-DQB1*02) were further considered; conditional analysis indicated that each could be explained by LD to the stronger signals at C4 and rs13194504 (FIG. 14).

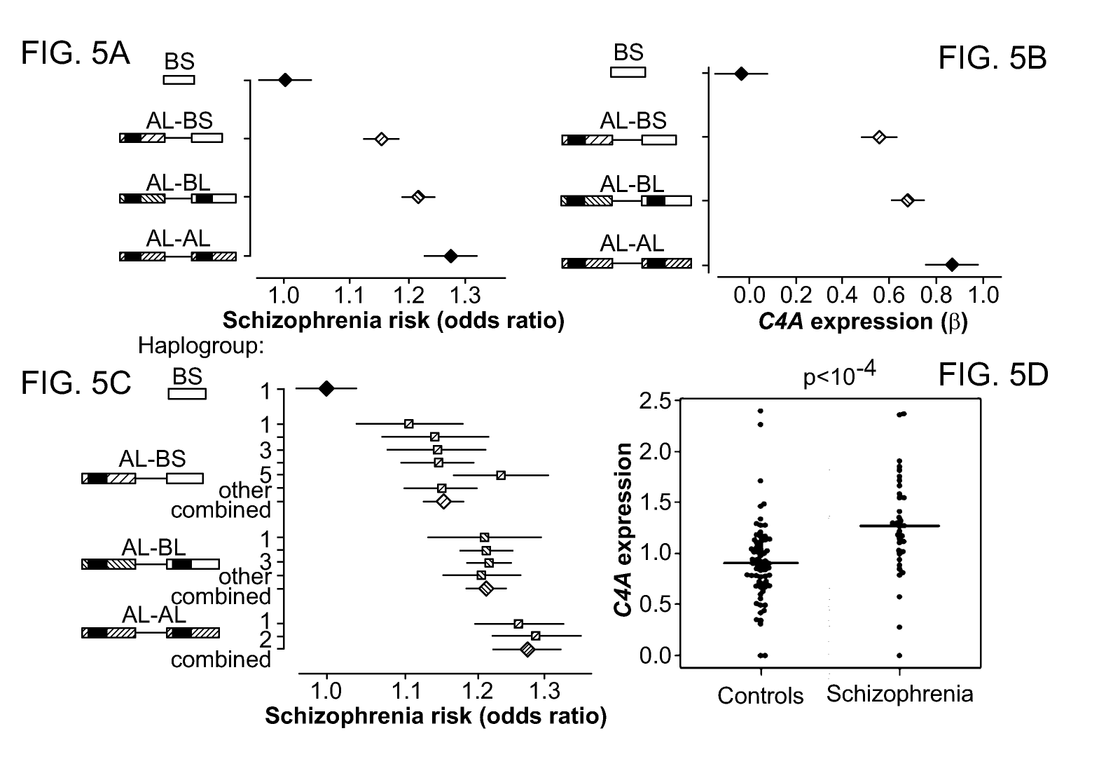

If each C4 allele affects schizophrenia risk via its effect on C4A expression, then this relationship should be visible across specific C4 alleles. Schizophrenia risk levels for the common C4 structural alleles (BS, AL-BS, AL-BL, and AL-AL) were measured; these alleles showed relative risks ranging from 1.00 to 1.27 (FIG. 5A). From the post mortem brain samples, the C4A expression levels generated by these four alleles were also estimated (FIG. 5B). Schizophrenia risk and C4A expression levels yielded the same ordering of the C4 allelic series (FIGS. 5A-5B). An even more stringent test was sought. If this allelic series of relationships to schizophrenia risk (FIG. 5A) arises from C4 locus structure—rather than from other genetic variation in the MEW locus—then a given C4 structure should exhibit the same schizophrenia risk regardless of the MHC haplotype on which it appears. The schizophrenia association of all 13 common combinations of C4 structure and MEW SNP haplotype was measured (FIG. 5C). Across this allelic series, each C4 allele exhibited a characteristic level of schizophrenia risk, regardless of the haplotype on which it appeared (FIG. 5C).

### Example 4: C4A RNA and Polypeptide Expression in Schizophrenia

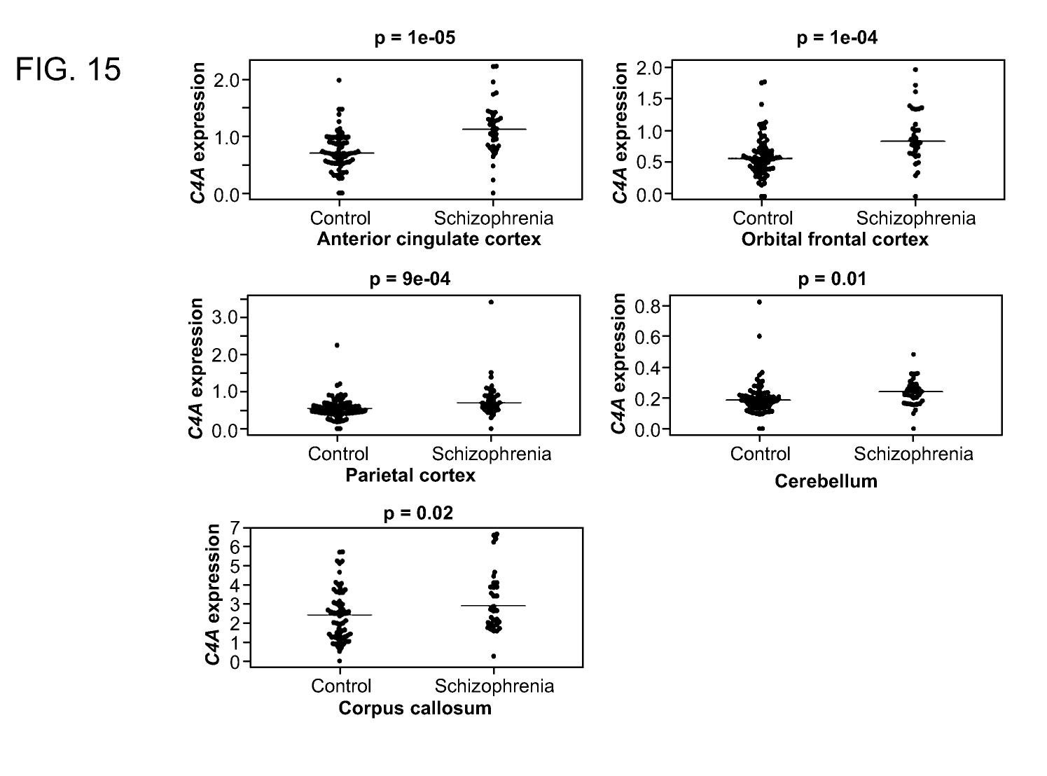

These genetic findings (FIG. 5A, FIG. 5C) predict that C4A expression might be elevated in brain tissue from schizophrenia patients. C4A RNA expression levels were measured in brain tissue from 35 schizophrenia patients and 70 individuals without schizophrenia. The median expression of C4A in brain tissues from schizophrenia patients was 1.4-fold greater (p=2×10⁻⁵ by Mann-Whitney test; FIG. 5D) and was elevated in each of the five brain regions assayed (FIG. 15). This relationship did not meaningfully change in analyses adjusted for age or post mortem interval. The relationship remained significant after correcting for the higher average C4A copy number among the brain donors affected with schizophrenia (1.3-fold greater, p=0.002). Some earlier studies have also reported elevated levels of complement proteins in serum of schizophrenia patients^{27,28}.

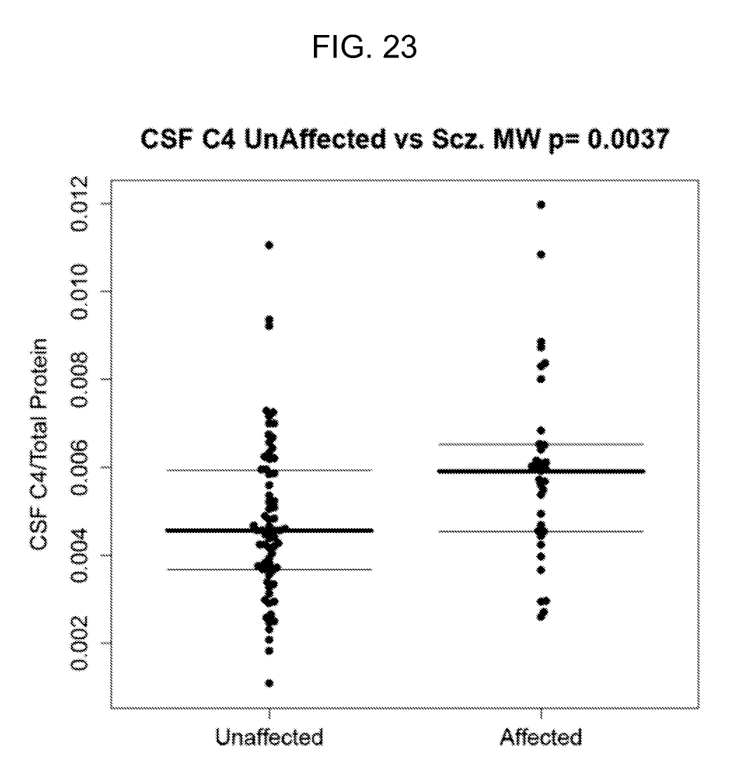

To evaluate the extent to which levels of C4 protein in cerebrospinal fluid (CSF) are informative about disease status, levels of C4 protein were measured (by ELISA assay) in CSF samples derived from a group of 120 individuals who were either affected or unaffected with schizophrenia. CSF from affected individuals exhibited elevated levels of C4 protein (p<0.01; FIG. 23). Thus, high levels of C4 protein in a CSF sample from a subject can be used to identify a subject as having schizophrenia.

### Example 5: C4 in the Central Nervous System

C4 is a critical component of the classical complement cascade, an innate-immune-system pathway that rapidly recognizes and eliminates pathogens and cellular debris. In the brain, other genes in the classical complement cascade have been implicated in the elimination or “pruning” of synapses²⁹⁻³¹.

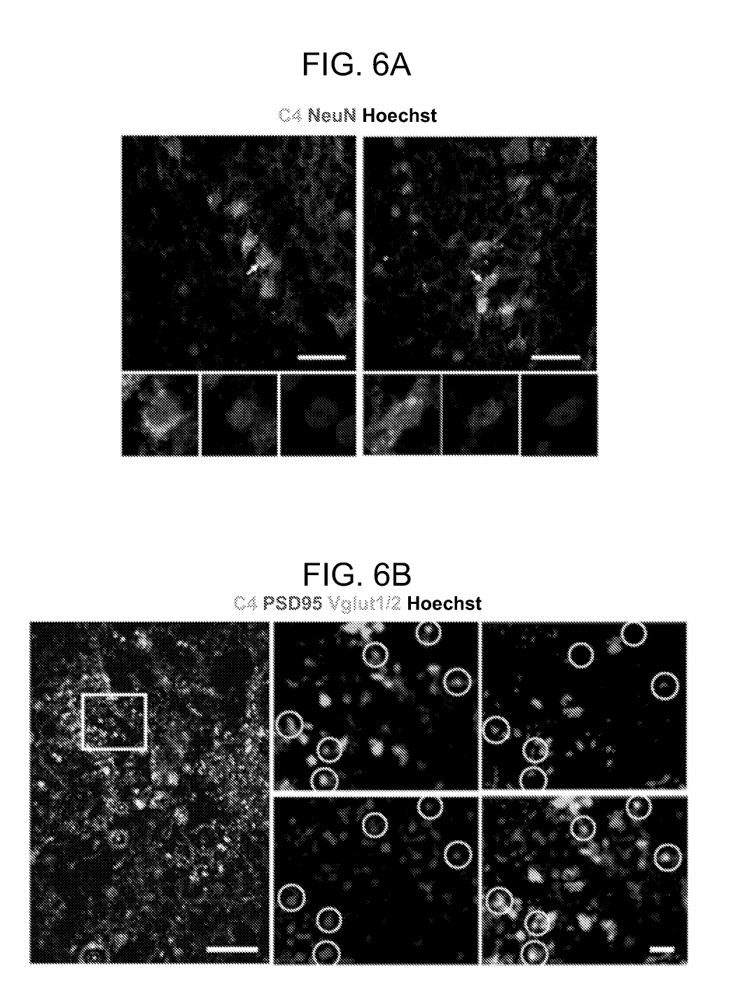

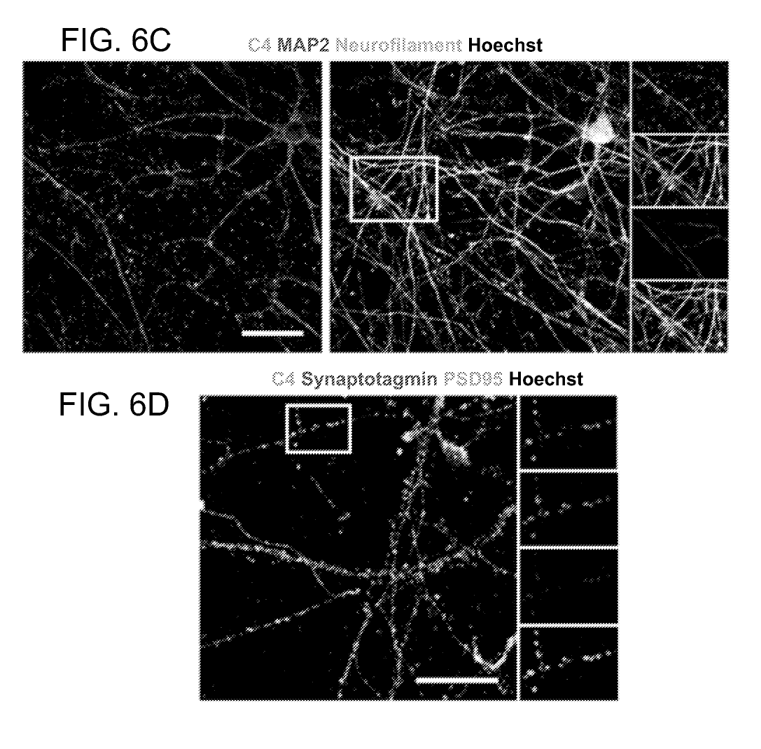

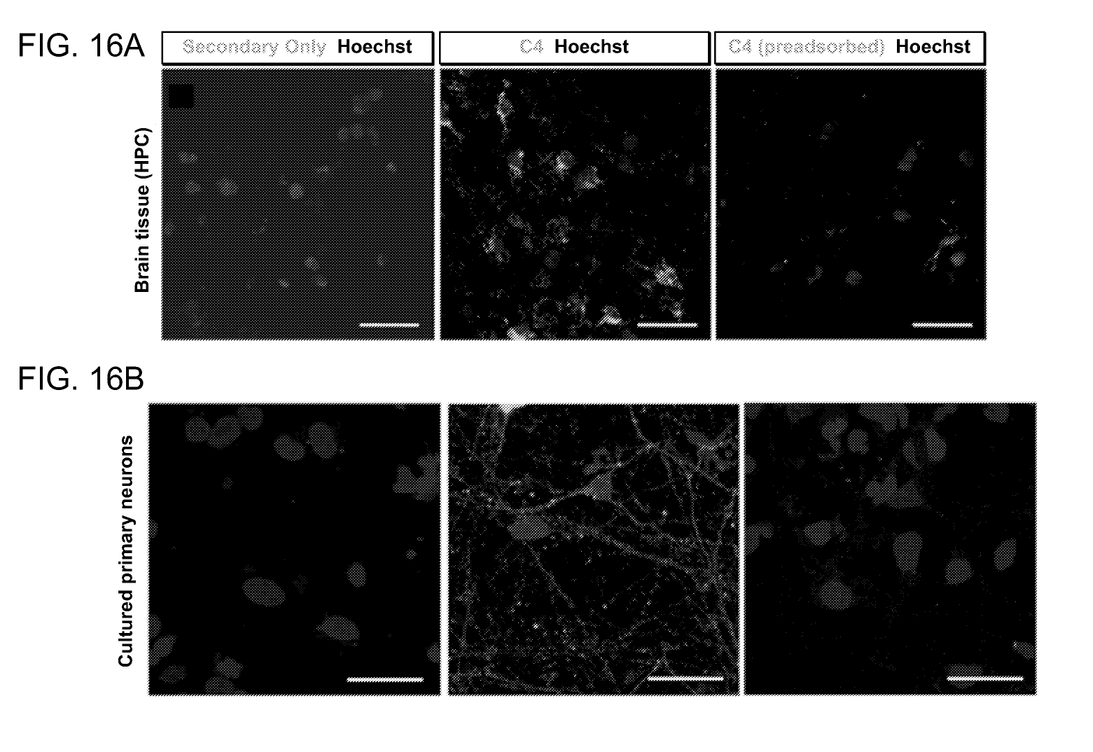

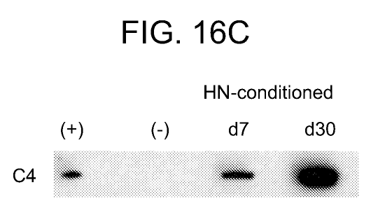

To evaluate the distribution of C4 in human brain, immunohistochemistry on sections of the prefrontal cortex and hippocampus was performed. C4+ cells in the gray and white matter were observed, with the greatest number of C4+ cells detected in the hippocampus. Co-staining with cell-type-specific markers revealed C4 in subsets of NeuN⁺ neurons (FIG. 6A; antibody specificity further evaluated in FIG. 16A) and a subset of astrocytes. Much of the C4 immunoreactivity was punctate (FIG. 6B), colocalizing with synaptic puncta identified by co-immunostaining for the pre- and postsynaptic markers VGLUT1/2 and PSD95 (FIG. 6B). These results suggest that C4 is produced by, or deposited on, neurons and synapses.

To further characterize neuronal C4, human primary cortical neurons were cultured and evaluated C4 expression, localization and secretion. Neurons expressed C4 mRNA and secreted C4 protein (FIG. 16C). Neurons exhibited C4-immunoreactive puncta along their processes and cell bodies (FIG. 6C-6D; antibody specificity further evaluated in FIG. 16B). About 75% of C4 immunoreactivity localized to neuronal processes (FIG. 6C); of the C4 in neuronal processes, approximately 65% was observed in dendrites (MAP2+, NF+ processes) and 35% in axons (MAP2−, NF+ processes). Punctate C4 immunoreactivity was observed at 48% of structural synapses as defined by co-localized synaptotagmin and PSD-95 (FIG. 6D).

The association of increased C4 with schizophrenia (FIGS. 4A-4F, FIGS. 5A-5D), the presence of C4 at synapses (FIG. 6B, FIG. 6D), the involvement of other complement proteins in synapse elimination²⁹⁻³¹, and earlier reports of decreased synapse numbers in schizophrenia patients³⁻⁵, together suggested that C4 might work with other components of the classical complement cascade to promote synaptic pruning. To test this hypothesis, a mouse model was studied. C4A and C4B appear to have functionally specialized outside the rodent lineage, but the mouse genome contains a C4 gene that shares features with both C4A and C4B (FIGS. 17A-17B). Impairments in schizophrenia tend to affect higher cognitive functions and recently-expanded brain regions for which analogies in mice are uncertain³². However, waves of postnatal synapse elimination occur in many brain regions, and strong experimental models have been established in several mammalian visual systems in which synaptic projections from retinal ganglion cells (RGCs) onto thalamic relay neurons within the dorsal lateral geniculate nucleus (dLGN) of the visual thalamus undergo activity-dependent synaptic refinement^{29-31,33-35}. It was found that C4 RNA was expressed in the LGN and in RGCs purified from retina (FIG. 17C).

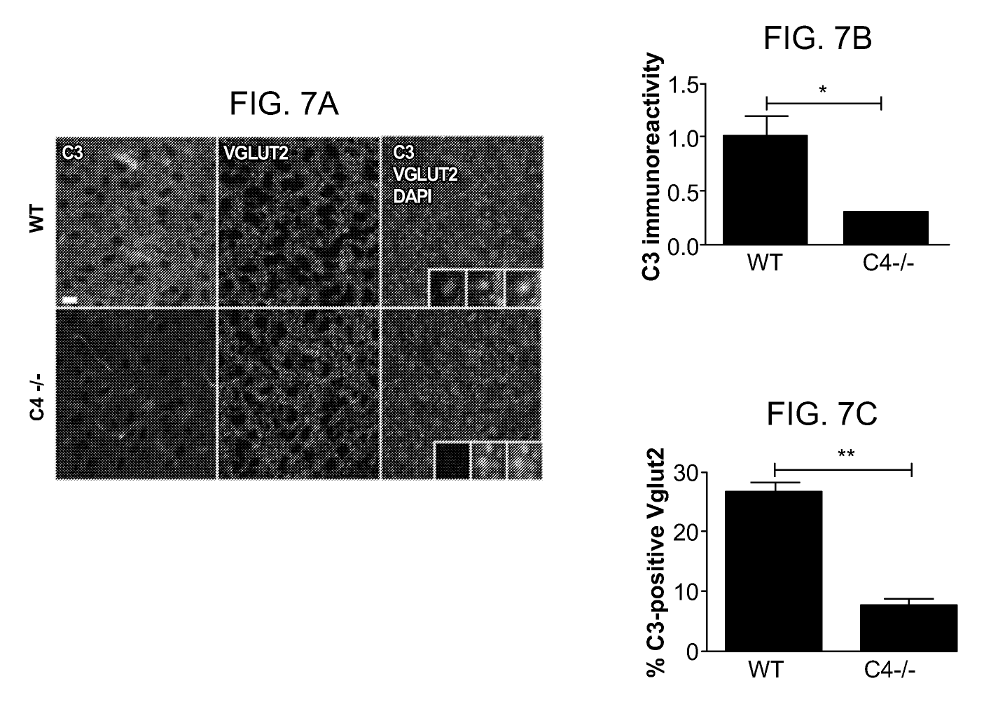

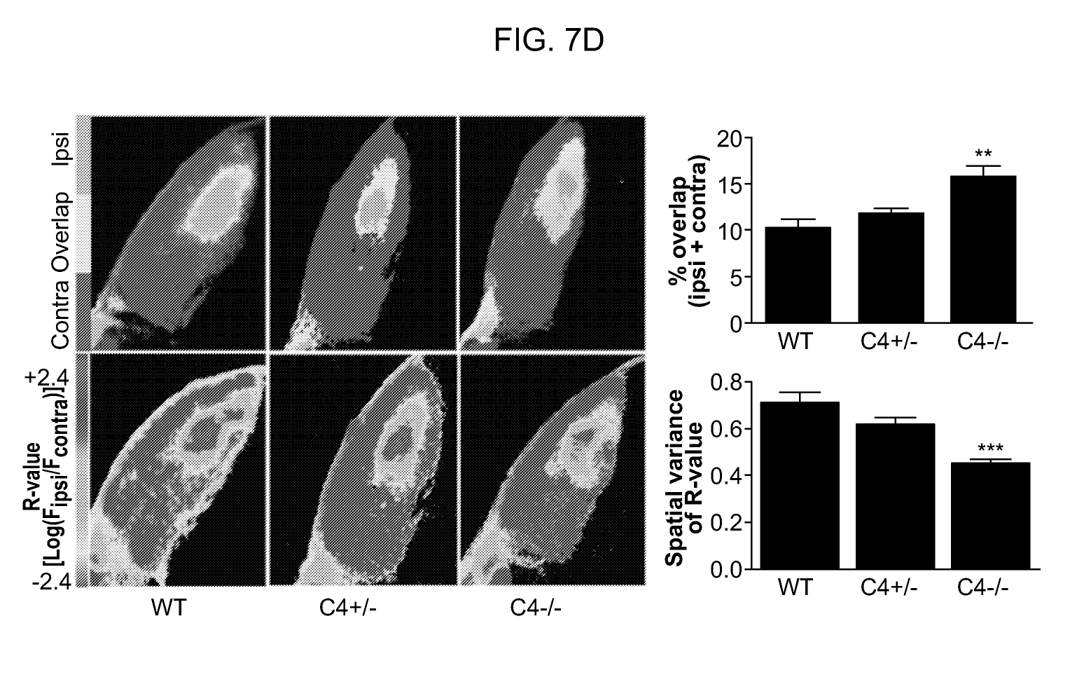

In the immune system, C4 promotes C3 activation, allowing C3 to covalently attach onto its targets and promote their engulfment by phagocytic cells. In the developing mouse brain, C3 targets subsets of synapses and is required for synapse elimination by microglia, the principal CNS cells expressing receptors for complement^{29,30}. It was found that in mice deficient in C4³⁶, C3 immunostaining in the dLGN was greatly reduced compared to WT littermates (FIGS. 7A-7B), with fewer synaptic inputs being C3-positive in the absence of C4 (FIG. 7C). These data demonstrate a role for C4 in complement deposition on synaptic inputs.

Whether mice deficient in C4 had defects in synaptic remodeling was then evaluated, as has been described for C3-deficient mice²⁹. Mice lacking functional C4 exhibited greater overlap between RGC inputs from the two eyes (p<0.001) than wild-type littermate controls, suggesting reduced synaptic pruning (FIG. 7D; FIGS. 17D-17E). The degree of deficit in C4^{-/-} mice was similar to that previously reported for C1q^{-/-} and C3^{-/-} mice^{29,31}. Heterozygous C4^{+/-} mice, with one wild-type copy of C4, had an intermediate phenotype (FIG. 7D). These data provide direct evidence that C4 mediated synaptic refinement in the developing brain.

In summary, described herein are methods to analyze a complex form of genome structural variation that were developed (FIGS. 1A-C; FIG. 2). By use of these methods, it was discovered that schizophrenia's association with variation in the MHC locus involved many common, structurally distinct C4 alleles that affect expression of C4A and C4B in the brain; each allele associated with schizophrenia risk in proportion to its effect on C4A expression (FIGS. 3A-3C; FIGS. 4A-4F; FIGS. 5A-5D). It was found that C4 was expressed by neurons, localized to dendrites, axons, and synapses, and secreted (FIGS. 6A-6D); and that C4 promoted synapse elimination during the developmentally timed maturation of a neuronal circuit (FIGS. 7A-7D; FIGS. 17A-17H).

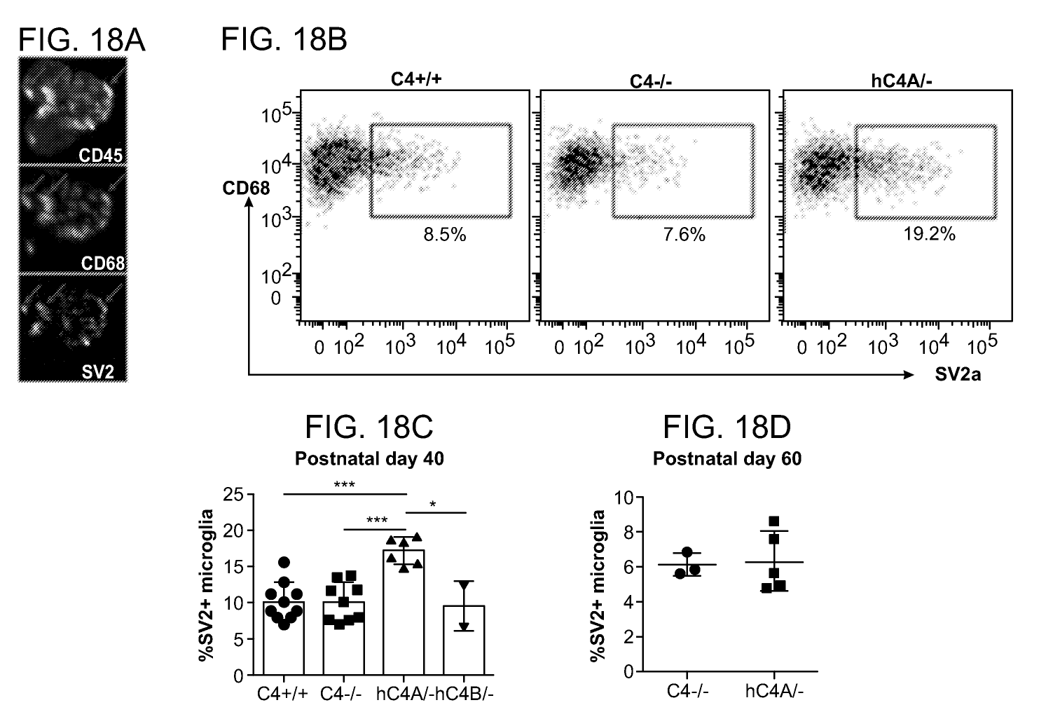

Microglia engulfed more synaptic particles in the presence of C4A in the frontal cortex of young adult mice (FIGS. 18A-18C). Microglia were isolated from frontal cortex at postnatal day 40 (P40) C4+/+, C4−/−, hC4A/− and hC4B/− mice using CD45 microbeads. Cells were stained for surface marker CD45 and CD11b, and for intracellular detection of SV2a and CD68 and analyzed by FACS. Microglia were identified as CD45low and CD11bhigh. FACS sorted microglia analyzed by confocal imaging showed the co-localization of SV2a proteins (white) within lysosomes (CD68) (green) (FIG. 18A). FACS analysis showed the frequency of SV2 positive cells within the microglia population was increased in hC4A/− mice (FIG. 18B). The frequency of SV2a positive microglia at P40 was increased in individual hC4A/− mice. (C4+/+n=10; C4−/− n=9; hC4A/−n=6; hC4B/−n=2; littermates C4+/+ and C4−/−; C4−/− and hC4A/−; C4−/− and hC4B/−) (FIG. 18C). At postnatal day 60 (P60), the frequency of SV2a positive microglia was about the same. (C4−/− n=3; hC4A/−n=5 littermates) (FIG. 18D).

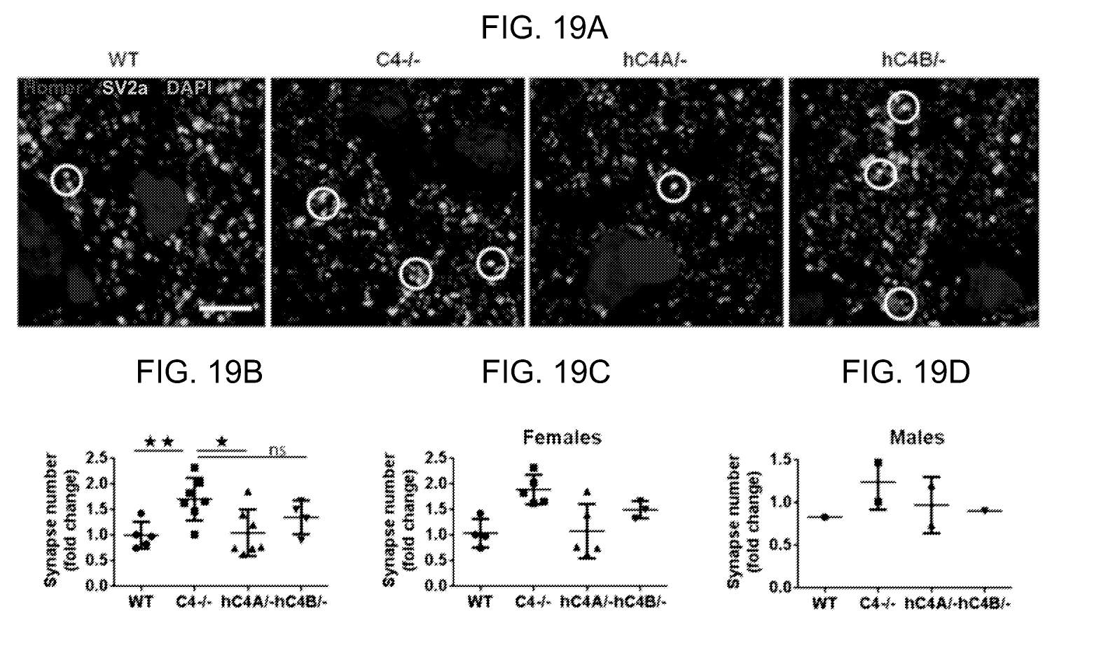

Synapses in frontal cortex of P60 mice were quantified. Postnatal day 60 WT, C4−/−, hC4A/− and hC4B/− mice were perfused with 4% PFA and harvested brains were incubated in 4% PFA prior to cryopreservation in sucrose. Brain sections (12 μm) were stained with anti-SV2 (presynaptic marker) and anti-homer (post-synaptic marker) antibodies and layer of the frontal cortex was imaged using a confocal microscope (4 section/animal; 2 field of view/section). Staining for SV2 and homer identified synapses, defined as co-localized SV2 and Homer puncta (FIG. 19A). Synapse number for each was mouse expressed as a fold change normalized to wild-type (WT) mice. Human C4A/− mice had fewer synapses at P60 compared to C4−/− mice (FIG. 19B). This was seen in female and male animals (FIGS. 19B and 19C). In particular, the difference was significant for the female mice. Without being bound by theory, Complement C4 regulates synapse number in frontal cortex, as observed in mice at P60.

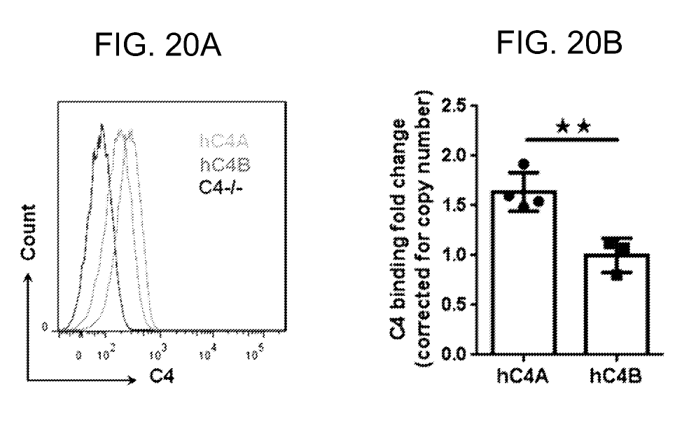

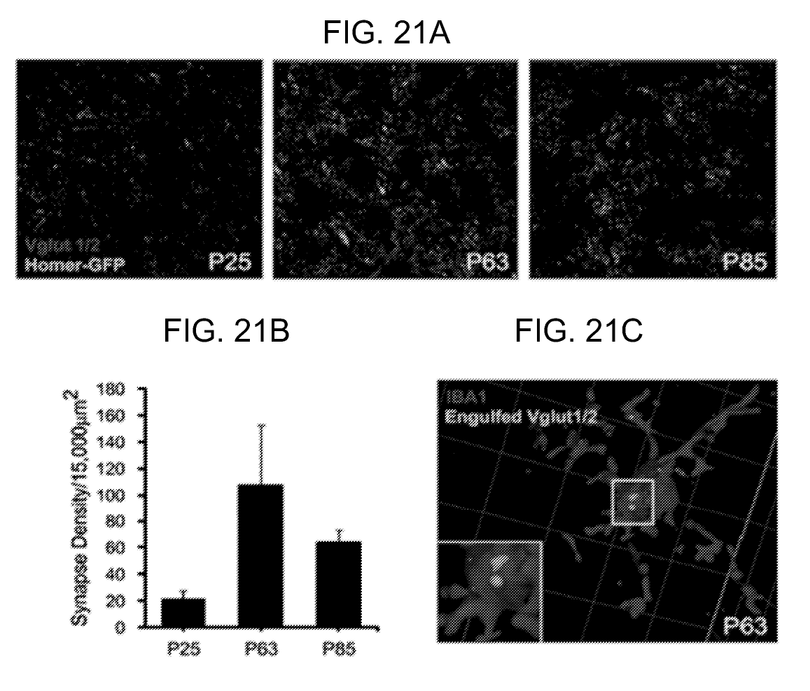

In vitro C4 binding assay showed C4A preferential bound to synaptic membranes compared to C4B (FIGS. 20A and 20B). Cortical synaptosome fraction was isolated from P40 C4−/− mice by sucrose gradient centrifugation. Synaptosomes were incubated with 10% serum from hC4A, hC4B or C4−/− mice at 37° c. for 1 hour, then stained with anti-human C4 FITC Ab. Flow cytometry analysis of synaptic particles revealed that C4A bound more efficiently than C4B (FIG. 21A). C4 binding fold change was obtained after correction for copy number (normalized with hC4B) (FIG. 20B).

Changes in synapse number occurred during development in layer 2/3 of frontal cortex (FIGS. 21A-21C). Confocal images were taken in layer 2/3 of homer-GFP mice, co-stained with anti-GFP and anti-Vglut 1 and 2 antibodies at P25, P63, and P85 (FIG. 21A). Synapse density (co-localized Homer and Vglut1/2) was quantified at each age (FIG. 21B). 3D reconstruction of microglia (IBA1, red) showed engulfed Vglut1/2+ synaptic material (green) at P63 (FIG. 21C).

Results described herein were obtained using the following materials and methods.

### Materials and Methods

### Sources of DNA Samples

Genomic DNA samples for the HapMap CEU population sample were obtained from Coriel Repositories (HapMap CEU plates 1 and 2). DNA samples for two groups of brain tissue donors were obtained from the Stanley Brain Resource of the Stanley Medical Research Institute (SMRI) and corresponded to the SMRI Array (SMRI-A) and SMRI Neuropathology (SMRI-N) collections. DNA samples for a third group of brain tissue donors, comprising 90 tissue donors for the NHGRI Gene and Tissue Expression Project (GTEx), were obtained from GTEx under an approved analysis proposal.

### Molecular Analysis of C4 Structural Elements (A, B, L, S)

Copy number of each individual C4 structural element was first measured (C4A, C4B, C4L, and C4S) using droplet digital PCR (ddPCR)⁵⁷. The following protocol for each genomic DNA sample in the study (including the HapMap CEU samples and the brain tissue donors) was used. First, genomic DNA was digested with AluI so that multiple tandem copies of C4 would then be on separate pieces of genomic DNA. (AluI cuts between structural features of C4 but not within any of the amplicons used for detection of them below.) For each genomic DNA sample, 50 ng of genomic DNA was digested in AluI (1 unit of enzyme in 10 ml of 1× reaction buffer, New England Biolabs) at 37° C. for 1 hour. The digested DNA was then diluted two-fold with water for subsequent analyses.

To measure the precise copy number of each structural element in each genomic DNA sample, digital PCR using nanoliter droplets (ddPCR) was performed, in which individual DNA molecules are dispersed into separate droplets, amplified with fluorescence detection probes (that detect with separate fluorescence colors the sequence of interest and a control, two-copy locus), and fluorescence-positive and -negative droplets of each color are then digitally counted⁵⁷. 6.25 μl of the digested, diluted DNA from the above reaction was mixed with 1 ml of a 20× primer-probe mix (containing 18 μM of forward and reverse primers each and 5 μM of fluorescent probe) for C4 and a reference locus (RPP30) each, and 2×ddPCR Supermix for Probes (Bio-Rad Laboratories). The oligonucleotide sequences for the primers and probes used for assaying copy number of C4A, C4B, C4L, and C4S were from Wu et al⁵⁸ and are listed in Table 1. For each sample, this reaction mixture was then emulsified into approximately 20,000 droplets in an oil/aqueous emulsion, using a microfluidic droplet generator (Bio-Rad). The droplets containing this reaction mixture were subjected to PCR using the following cycling conditions: 95° C. for 10 minutes, 40 cycles of 94° C. for 30 seconds and 60° C. (for C4A and C4L) or 59° C. (for C4B and C4S) for 1 minute, followed by 98° C. for 10 minutes. After PCR, the fluorescence (both colors) in each droplet was read using a QX100 droplet reader (Bio-Rad). Data were analyzed using the QuantaSoft software (Bio-Rad), which estimates absolute concentration of DNA templates by Poisson-correcting the fraction of droplets that are positive for each amplicon (C4 or RPP30). Since there are two copies of RPP30 (the control locus) in each diploid genome, the ratio of the concentration of the C4 amplicon to that of the reference (RPP30) amplicon is multiplied by two to yield the measurement of copy number of the C4 sequence per diploid genome (FIG. 9B). A key feature of these data is that the resulting measurements show a multi-modal distribution in which individual measurements are very close to integers rather than mid-integer (FIG. 9B), allowing a precise integer measurement (rather than a rough estimate) of the copy number of each structural element in each genome.

The accuracy of copy number measurements from the above approach was evaluated in two ways. First, in every genome analyzed, the following relationship between the copy number of C4 structural elements is expected to hold because any given C4 gene is defined by its length (long or short) and its paralogous form (A or B):

C4A+C4B=C4L+C4S

Any deviation from this equality (for any sample) could flag a genotyping error for C4A, C4B, C4L, or C4S. Copy number measurements for all HapMap DNA samples and all brain donor DNA samples in this study satisfied this test in every case. In addition, copy number measurements for C4A and C4B from ddPCR were compared to those for 89 HapMap samples previously evaluated by Fernando et al.⁵⁹ using Southern blot analysis of the same samples; measurements herein agreed with those of Fernando et al. for 89/89 samples.

### Determining Copy Number of the Compound C4 Structural Forms (AL, AS, BL, BS)

The above analysis determines copy number of individual structural elements (A, B, L, S) but not of compound structural forms (AL, AS, BL, BS). Given that (for example) the numbers of copies of C4S are known, determining the ratio of the number of copies of C4AS and C4BS allows the copy number of these compound structural features to be readily calculated.

To determine how the known number of C4S copies (measured above) was composed of C4AS and C4BS copies, PCR was first performed to amplify 5.2-kilobase DNA molecules derived from C4S and spanning to the C4 A/B-defining molecular features (FIG. 9C); this PCR involved a forward primer specific to C4S and reverse primer designed to the right of the C4 A/B defining molecular features in exon 26. The reaction was performed in 50 μl and consisted of 20 ng of input genomic DNA, 10 μl of 5X Long Range Buffer (Mg2+ free) (Kapa Biosystems), 1.75 mM MgCl₂, 0.3 mM of each dNTP, 0.5 μM each of forward and reverse primers, and 1.25 units of Kapa LongRange DNA Polymerase. Cycling conditions were as follows: 94° C. for 2 minutes; 35 cycles of 94° C. for 25 seconds, 61.2° C. for 15 seconds, and 68° C. for 5 minutes and 12 seconds; and 72° C. for 5 minutes and 12 seconds. The PCR product from the long-range PCR was used as input into a ddPCR assay with which the ratio of C4AS to C4BS gene copies could be precisely measured. PCR products were diluted and 1 μl of this diluted DNA was added to a ddPCR mixture containing 1 μl of a 20× primer-probe mixture of the C4A assay (FAM), 1 μl of a 20× primer-probe mixture of the C4B assay (HEX), and 10 μl of 2×ddPCR Supermix for Probes (Bio-Rad). The generation of droplets and the PCR cycling conditions were as described above for the ddPCR assays of C4 copy number, with an annealing temperature of 60° C. After droplets were read, the ratio of C4AS to C4BS was calculated from the relative estimated concentrations of C4A-defining and C4B-defining sequences among the C4S amplicons. The combination of this ratio with the earlier determination of C4S copy number (above) allowed determination of integer copy number of C4AS and C4BS.

Once C4A, C4B, C4L, C4S, C4AS, and C4BS copy numbers are calculated by the above methods, copy number of the remaining compound structural features (C4BL and C4AL) is easily calculated by the following formulas:

$$\begin{matrix}Copy number ( CN ) of C 4 BL = ( CN of C 4 B ) - ( CN of C 4 BS ) \\ Copy number ( CN ) of C 4 AL = ( CN of C 4 A ) - ( CN of C 4 AS ) \\ = ( CN of C 4 L ) - ( CN of C 4 BL )\end{matrix}$$

with the redundant calculation of C4AL copy number (by these two formulas) providing an additional checksum on the accuracy of measurements of copy number state.

### Inference of Allelic Contribution to Copy Number in Diploid Genomes

For a multi-allelic CNV, multiple combinations of alleles can give rise to the same diploid copy number. For example, if a sample has 4 copies of the C4AL gene in a diploid genome, this could be a result of any of the following potential allelic combinations: 0+4, 1+3, or 2+2. To distinguish among these possibilities, we exploited allele frequency information that is implicit in the relative frequencies of the different diploid copy-number genotypes, together with additional constraints placed by inheritance in trios, as described below. An expectation-maximization (EM) algorithm that incorporated this information was applied to each C4 structural form (AL, AS, BL, and BS) separately. In this approach, each allelic configuration that could potentially give rise to each diploid copy number was enumerated. In certain trios only one configuration was possible under Mendelian inheritance (e.g., a trio in which father, mother, and offspring had a copy number of 0, 2, and 1, respectively). In the rest of the trios, allelic contributions were inferred using an EM algorithm with the following steps. First, probabilistic inferences of haploid copy number were made in each sample (with an “initial condition” that all possible combinations were equally likely). These inferences were then used to estimate frequencies of each copy-number allele in the population. The likelihood of each allelic combination in each trio was then re-calculated given these allele-frequency estimates. This allowed new estimates of allele frequency, which were then used to refine likelihoods of observing each allelic combination in each trio. This EM loop was repeated until the allele frequency estimates converged. In practice, these estimates converged very quickly to estimates that had low uncertainty in 45-55 of the 55 trios in the analysis (51 for AL, 55 for AS, 45 for BL, 49 for BS). In the remaining trios, the following further approach was used. First, a reference set of haplotypes was created from the trios in which inference of copy-number alleles had been unambiguous. This core set of haplotypes was then used as a reference to phase the remaining copy number alleles onto SNP haplotypes using Beagle genetic analysis software⁶⁰.

### Imputation of C4 Alleles; Leave-One-Out Trials to Estimate Imputation Accuracy

C4 alleles were imputed from SNP genotypes using Beagle genetic analysis software⁶¹. To estimate the accuracy of inferences using our imputation approach, we performed leave-one-out trials. A different individual was removed from the reference panel in each trial, and the rest of the reference haplotypes were used to impute, using genetic analysis software⁶¹, the C4 structural form and haplogroup, with different subsets of SNPs in the extended MHC locus (chr6: 25-34 Mb): Illumina OmniExpress, Affymetrix 6.0, and Illumina Immunochip. The correlation (r²) between the probabilistic dosage from imputation and the experimentally-determined genotypes was calculated as a metric of imputation accuracy (Table 2). Note that these estimates of imputation efficacy will in many cases be lower bounds: (i) they will be exceeded by what it should be possible to do in the future (with larger reference panels derived from whole genome sequencing of many hundreds of families); and (ii) even in the current analysis, it was frequently observed that SNP haplotypes that were rare or unique in the reference panel (for example, the haplotypes grouped into the “-other” categories) were more common in the PGC cohorts and were presumably imputed with greater accuracy than a leave-one-out analysis would predict.

### Post Mortem Human Brain Tissue RNA Samples

Expression of C4A and C4B was measured in eight panels of post mortem human brain RNA samples derived from three sets of donors. The first set (five brain-region-specific panels from one set of donors) was the Stanley Medical Research Institute Array Collection. This collection consists of 525 samples from 105 individuals. Five brain regions were sampled from each donor: anterior cingulate cortex, orbital frontal cortex, parietal cortex, cerebellum, and corpus callosum. The median age of the donors was 44 (range 19-64). Of the 105 individuals, 102 were of European ancestry and used in the analysis. The median post mortem interval (PMI) was 30 hours (range 9-84). 69 donors were male and 38 were female. Age, sex and PMI were evaluated as potential covariates in all analyses but were found to have insignificant regression coefficients in all analyses described. The second set (two tissue-specific panels) was obtained from the Stanley Medical Research Institute Neuropathology Consortium and contained 120 samples from 60 individuals. Two regions were sampled from each donor: anterior cingulate cortex and cerebellum. 36 donors were male and 24 were female. The median age was 47 (range: 30-68). The median PMI was 27 hours (range: 11-62). Age, sex and PMI were evaluated as potential covariates in all analyses but were found to have insignificant regression coefficients in all analyses described. The third set consisted of 93 samples (frontal cortex) from 93 individuals sampled by the Genotype-Tissue Expression (GTEx) Consortium. 67 donors were male and 26 were female. The median age was 53 (range: 22-59). Age, sex and BMI were evaluated as potential covariates in all analyses but were found to have insignificant regression coefficients in all analyses described. Copy number of C4 structural elements was measured using ddPCR in blood-derived genomic DNA samples from all individuals as described elsewhere herein.

### Molecular Analysis of C4A and C4B Expression Levels

Expression measurements were made using reverse-transcription ddPCR, in which total RNA is dispersed into thousands of nanodroplets; reverse transcription, PCR amplification, and fluorescence detection are then performed in droplets. Gene-expression measurements were normalized to the expression of a control gene (ZNF394) to account for variation in the amount of input RNA across samples; this gene was selected as a normalization control because in earlier brain transcriptomics data it showed uniform (low-variance) expression level across brain tissues sampled from many different individuals. In each reaction, the number of C4A-positive (or C4B-positive) and -negative droplets was counted, as well as the number of ZNF394-positive and -negative droplets. These numbers were then Poisson-corrected to yield an estimate of the underlying expression level, using the QuantaSoft software (Bio-Rad). ZNF394 was used as a normalization control and therefore calculate the ratio of C4A (or C4B) to ZNF394 expression.

For each brain donor in the two SMRI Brain Collection cohorts (each of which sampled multiple brain regions from each donor), a composite measure of expression across multiple brain regions was calculated in the following way. The calculation started with an i×j matrix (i individuals and j brain regions) of gene-expression measurements. A median normalization of the data was then performed for each region (more formally, the expression for i^{th} individual in region j was re-calculated as a percentage of the median expression value across all the individuals for region j). To then obtain an overall summary value (across multiple brain regions) for an individual, the median (across regions) of these median-normalized values (more formally, a median value across the j columns was calculated for each row) was then calculated. Donors for whom measurements were available for at least 3 (of the 5) brain regions were carried into downstream analysis. Association between C4A (or C4B) expression and C4A (or C4B) copy number (FIGS. 3A-3B) was tested using a (non-parametric) Spearman correlation test. In order to evaluate the relationship of C4-HERV (C4L) copy number to C4 expression (FIG. 3C), the effects of gene copy number, linkage disequilibrium, and trans-acting influences was sought to be neutralized by calculating the ratio of C4A expression per copy (C4A expression divided by C4A copy number) to C4B expression per copy (C4B expression divided by C4B copy number). Normalizing for genomic copy number of C4A and C4B allowed for investigation of effects separate from the effect (or in LD with the effect) of increased gene copy number. Normalizing expression of C4A to expression of C4B allowed cleaner analysis of cis-acting effects by controlling for trans-acting effects. (This is analogous to what is done in studies that utilize allele-specific expression, only here with two paralogous genes rather than two alleles of the same gene.). This normalization leaves open the question of whether the observed positive relationship to C4-HERV copy number (FIG. 3C) is due to increased expression of C4A or reduced expression of C4B; regression of C4A and C4B expression against copy number of these structural features (see section below) indicated that it was mostly if not entirely due to increased expression of C4A.

In the SMRI samples, the availability of genome-wide SNP data (together with our measurements of C4A, L, B, S copy number) allowed inference (by imputation) of the complex C4 structures present on each chromosome. To calculate the effect of each of the four common C4 structures on expression of C4A (FIG. 5B), C4A expression was fit to the dosage of that structure across the SMRI post mortem brain samples:

(C4A expression)ᵢ=Σⱼβⱼ×(dose)ᵢⱼ+θ

where (dose)ᵢⱼ is the number of chromosomes in each diploid genome i that carry the structure j and θ is a constant (intercept).

To determine the C4 structural genotype for each individual in the SMRI array collection, copy number data for each C4 structural element (C4A, C4B, C4L, and C4S) from ddPCR were integrated together with SNP genotypes for these samples (from the Illumina Omni 2.5 SNP microarray). For each individual, the list of structural genotypes consistent with the set of copy numbers of C4 structural elements were enumerated, based on the 15 C4 structures that were identified in the HapMap CEU population sample (FIG. 1C). For example, if the copy number of C4A, C4B, C4L, and C4S were 2, 1, 2, and 1, respectively, then two structural genotypes were possible: AL/AL-BS and AL-AL/BS. Given the large number of structural genotypes theoretically possible (120 possible genotypes based on 15 structural haplotypes), more than 5 structural genotypes were consistent with a set of copy number data for C4 structural elements for many individuals. In order to identify the most likely structural genotype, the backbone SNP genotype data were used to estimate the likelihood of observing each structural genotype given a set of copy number as well as SNP genotype data. A vector of genotype likelihoods (of length 120) was provided as input for phasing in Beagle (version 4). Each structural genotype that was consistent with the copy number data was encoded as equally likely, and those that were inconsistent were assigned a log₁₀ likelihood of −1000 (i.e., to indicate that they are extremely unlikely). These likelihoods were then phased together with SNP genotypes to obtain posterior genotype probabilities for each possible structural genotype, for every individual. These probability estimates readily identified the most likely genotype for each individual (with a mean probability of 0.99).

To test association between gene expression and clinical diagnosis, the Mann-Whitney (nonparametric) test was used. The alternative hypothesis was specified based on the direction of effect of C4 structural variation on gene expression and on the risk of schizophrenia—given that C4 structural variants associating to increased risk of schizophrenia also associated to higher expression, it was hypothesized that the expression of C4 would be higher in patients with schizophrenia compared to unaffected controls. A Mann-Whitney test was performed to assess for differences in median normalized C4A expression values between patients with schizophrenia and unaffected controls. In order to test whether the expression of C4A associated with clinical diagnosis independently of structural variation in C4, the C4A expression-per-copy values were used and a Mann-Whitney test was again performed.

Expression of C4A and C4B was also tested for association to potential confounders, including age, sex, post mortem interval, preservation technique, and smoking. Parametric (Pearson) as well as non-parametric (Spearman) tests of correlation were used to evaluate correlation to continuous variables (age and post mortem interval), and association of expression to categorical variables (sex, preservation technique, and smoking) was tested using the Mann-Whitney test.

### Model for Genetically Predicting C4A and C4B Expression

To derive a model for genetically predicting C4A and C4B expression to be used in association analysis of schizophrenia (in which it was expected that numerous genomes will have lower-frequency C4 structural haplotypes that are sparsely represented among the samples with measured expression values), C4A and C4B expression levels were sought to be predicted as a function of the dosage of each structural element (C4 AL, C4BL, C4AS, C4BS). All median-normalized expression data from samples across the SMRI array, SMRI Neuropathology, and GTEx cohorts was used to fit

(C4A or C4B expression)ᵢ=Σⱼβⱼ×(dose)ᵢⱼ+θ

where (dose)ᵢⱼ is the number of structural elements j in sample i. From this model, samples with lower-frequency C4 haplotypes can have expected expression values computed by summing their structural element dosages multiplied by the corresponding coefficients. Regression coefficients that were significantly different from zero were included in the prediction models. The following prediction models were generated:

C4A expression=(0.47*AL)+(0.47*AS)+(0.20*BL)

C4B expression=(1.03*BL)+(0.88*BS)

Note that these are parameterized in internally normalized “expression units” that are not comparable between C4A and C4B, but are comparable across individuals for the same gene. These models explained 71% and 42% of inter-individual variation in measured C4A and C4B expression levels (respectively)—far more than explained by most known cis-eQTLs, but still consistent with a role for additional factors (beyond cis-acting variation at C4) in shaping C4 expression levels.

Case-Control Genotype Data from the Psychiatric Genomics Consortium (PGC)

Data from all 40 of the European-ancestry case-control cohorts for which individual level data could be made available by the PGC for such analyses was used (individual-level data from some cohorts could not be made available due to restricted level of patient consent). As described in the PGC manuscript⁶², all subjects provided written informed consent (or legal guardian consent and subject assent) with the exception of the CLOZUK sample, which obtained anonymous samples via a drug monitoring service under ethical approval and in accordance with the UK Human Tissue Act. The cohorts and array platforms used are listed in Table 3. These samples are further described in ref⁶² and in the individual studies referenced in Table 3.

Relatedness among samples and population structure was previously analyzed by the PGC Statistical Analysis Working Group, using a set of 19,551 autosomal SNPs across all cohorts, removing one member of each pair with π>0.2. The first ten principal components were included as covariates in all of the association analyses (as described below). All analyses were pursued in concordance with an analysis proposal approved by the PGC Schizophrenia Working Group. All analyses of individual-level genotype data were conducted on the PGC's computer server in the Netherlands.

### Quality Control for SNP Data

The SNPs and individuals retained for association analysis were subject to the following quality control (QC) parameters previously applied by the PGC Statistical Analysis Group and including: (i) SNP missingness <0.05 (before sample removal); (ii) subject missingness <0. 02; (iii) autosomal heterozygosity deviation (|Fhet|<0.2); (iv) SNP missingness <0.02 (after sample removal); difference in SNP missingness between cases and controls <0.02; and SNP Hardy-Weinberg equilibrium (p>10⁻⁶ in controls or p>10⁻¹⁰ in cases).

In addition to the above parameters that were analyzed on a genome-wide scale, additional QC filters were applied to the SNP genotype data from the extended MHC locus in each of the 40 cohorts analyzed. SNPs that met the following criteria were removed: (i) those that were within the duplicated C4 locus (chromosome 6:31939608-32014384, hg 19); (ii) SNPs whose allele frequency differed by more than 0.15 from their frequency in our HapMap CEU reference panel for imputation; and (iii) transversion SNPs (A/T and G/C) whose minor allele frequency was greater than 0.35 (as it can be problematic to determine whether they have the same strand assignment as SNPs in the reference panel for imputation).

### Imputation of C4 Structural Variation, Genetically Predicted C4A Expression, and HLA Classical Alleles

Imputation of C4 structural variation into the PGC data set was done with Beagle genetic analysis software⁵, using the HapMap CEU reference panel that we had supplemented with C4 structural alleles. C4 structural variation was imputed into each of the 40 cohorts in the PGC data set separately. Imputation was performed using two approaches, with highly similar results: (i) a “best guess” approach in which each genome is assigned the most likely pair of C4 structural alleles given the SNP data; and (ii) a “dosages” approach in which imputation uncertainty is advanced into subsequent stages of analysis by performing association analysis on the probabilistic “dosages” of each allele in each genome.

The reference panel used consisted of 222 haplotypes from 111 unrelated individuals, with C4 structural variants on haplotypes with HapMap phase III SNPs (see FIG. 2) in the extended MHC locus (chromosome 6: 25-34 Mb). The encoding of C4 structural variation in this reference panel was based on both the C4 structure as well as its MHC haplotype background (FIG. 2). C4 structures that segregated on multiple MHC SNP haplotypes were encoded as separate alleles in the reference panel—AL-AL structures were divided into two alleles, AL-AL-1 and AL-AL-2, based on which of the two MHC SNP haplotypes they segregated on; AL-BL structures into three alleles that were based on the three well-defined haplotype backgrounds and a fourth allele to represent the remaining (“other”) set of rarer haplotypes; and AL-BS structures into six alleles (five of which had common haplotype backgrounds, and the sixth of which collected the other, rarer haplotypes together).

This strategy enabled independent testing of association of each common combination of C4 structure and MHC SNP haplotype background. This strategy also allowed (i) inference of copy number of C4 structural elements (C4A, C4B, C4L, and C4S) based on the C4 alleles imputed in each individual (e.g., an individual with C4 alleles AL-AL-1 and AL-BL-2 has a diploid copy number of 3 for C4A, 1 for C4B, 4 for C4L and 0 for C4S); and (ii) inference of expected expression of C4A and C4B in the brain based on calculated copy number of C4 structural elements in each individual, using the linear model (described above) that was fit to the expression data from post mortem brain samples. A reference panel consisting of 9,956 haplotypes based on data collected by the Type 1 Diabetes Genetics Consortium (T1DGC)⁶³ was used for imputation of HLA classical alleles from both class I and class II genes: HLA-A, B, C, DRB1, DQA1, DQB1, DPB1, DPA1. This reference panel enabled imputation of HLA classical alleles at four-digit resolution, HLA amino acids, intragenic SNPs in the MEW locus, and insertions/deletions.

### Testing Association of C4, SNPs, and HLA Classical Alleles to Schizophrenia

A mega-analysis was performed that utilized individual-level genotype data from all 40 cohorts that were analyzed from the PGC data set. Association analysis was performed in a logistic regression framework that included study indicator variables to account for cohort-specific effects and principal components to control for population stratification:

log(oddsᵢ)=βⱼ×(dose_{i,j})+Σ_{c=1}³⁹β_{c}×(chort_{i,c})+Σₚ₌₁¹⁰βₚ×(PC_{i,p})+θ

where dose_{i,j} is the number of chromosomes in each individual, i, that carried a C4 structural allele, j, and βⱼ is the additive effect per copy of the C4 allele. 39 study indicator variables (the number of cohorts minus 1) were included, with cohort_{i,c} equal to 1 if the ith individual belonged to the c^{th} cohort and equal to 0 otherwise. In addition, ten principal components that associated to phenotype were included as covariates, with PC₄ being the p^{th} principal component for the i^{th} individual. The same framework was used for testing association to (i) individual SNPs and HLA classical alleles, where dose_{i,j} was the dosage of the minor allele, j, of the SNP or HLA classical allele in individual i; (ii) copy number of C4 structural features, where dose_{i,j} was the diploid copy number of the C4 feature in individual i; (iii) genetically predicted expression of C4A and C4B, where dose_{i,j} was calculated from the imputed C4 structures according to the above formulas (see the section, “Model for genetically predicting C4A and C4B expression”). To test association to C4 conditional on rs13194504 and rs210133 (representing the other two genome-wide significant associations within the extended MHC locus), the dosages of the minor alleles of those SNPs were used as additional covariates in the model.

The association of C4 alleles to schizophrenia was tested in multiple ways. The first test used aggregate genetic predictors (of C4A and C4B expression levels) as a composite genetic variable that combined information across many different alleles into an omnibus test; we started with this omnibus test (FIGS. 4A-4F) in order to avoid over-fitting the genetic data to ad hoc combinations of C4 alleles. The schizophrenia association of specific C4 structures (structural forms of the C4 locus) was further measured (FIG. 5A). An estimate of effect size for a C4 structure (e.g., AL-AL) was obtained across all alleles that contained that given structure (e.g., AL-AL-1 and AL-AL-2), by performing an inverse variance meta-analysis based on the effect size and standard error associated with each C4 allele that contained the given C4 structure. These effect size estimates were then normalized to a reference value of 1.0 for the C4 BS allele.

### Immunohistochemistry (Human Tissue)

Fresh frozen hippocampus and frontal cortex sections were obtained from the Stanley Medical Research Institute. Stained tissues were from schizophrenia patients aged 31-43. Sections were thawed on ice and then post-fixed for one hour at 4° C. in 4% paraformaldehyde in PBS. Sections were then washed three times in PBS and then permeabilized in 0.2% Triton X-100 in PBS on a shaker for one hour at room temperature. Sections were then blocked in 10% BSA with 0.2% Triton X-100 in PBS for one hour at room temperature on a shaker and then transferred into a carrier solution of 5% BSA in 0.2% Triton X-100 in PBS containing the primary antibody and were left to incubate overnight at 4° C. For pre-adsorption experiments, purified human C4 protein (Quidel) was pre-incubated with the C4c antibody at double the antibody concentration for 30 minutes at room temperature before being added to the slides for overnight incubation at 4° C. The following day sections were washed three times in PBS and incubated in carrier solution with Alexa-Flour conjugated secondary antibodies (1:500) and Hoechst (1:10,000) for one hour at room temperature on a shaker. The sections were then washed three times in PBS and then incubated in 0.5% Sudan Black dissolved in 70% ethanol to eliminate autoflourescence from lipofuscin vesicles. Sections were then washed 5-7 times in PBS to remove the excess Sudan Black. Coverslips were then added to the slides using 90% glycerol in PBS as the mounting media. Slides were imaged on an Ultraview Vox Spinning Disk Confocal microscope for images of cellular colocalization or Zeiss ELYRA PS1 structured illumination microscope (SIM) for synapse analysis. The following antibodies were used for staining; anti-C4c (Quidel, A211, 1:1000), anti-NeuN (Abcam, AB104225, 1:500), anti-Vglut1 (Millipore, AB5905, 1:1000), anti-Vglut2 (Millipore, AB2251, 1:2000), and anti-PSD95 (Invitrogen, 51-6900, 1:200). IHC was performed in brain tissue slices from 5 individuals affected with schizophrenia and 2 unaffected individuals. These were selected from the same brains as the RNA experiments (SMRI Neuropathology Consortium). Across different donors variable intensity of staining (down to almost no staining) was observed, but qualitatively different patterns were not observed. The level of RNA expression of C4 (in the corresponding RNA sample from the same donor) predicted the level of IHC staining—in tissue from donors with higher C4 RNA expression, the IHC staining was also stronger; in tissue from donors with little-to-no C4 RNA detected, little-to-no IHC staining was also observed.

The images in FIGS. 6A-6D are from tissue from one of the individuals affected with schizophrenia.

### Immunocytochemistry

Primary human cortical neurons were obtained from Sciencell Research Laboratories (catalog no. 1520). The neurons were characterized by Sciencell to be immunopositive for MAP2, neurafilament, and beta-tubulin III; are guaranteed to be negative for HIV-1, HBV, HCV, mycoplasma, bacteria, yeast, and fungi; and are not listed as a commonly misidentified cell line by ICLAC. Human cortical neurons were cultured in vitro on PLL-coated coverslips in neuronal media for up to 48 days. Coverslips were fixed with 4% paraformaldehyde at room temperature for 7 minutes. Non-specific binding sites were blocked with 5% BSA for 1 hour in PBST (0.1% Tween 20) followed by 4° C. overnight incubation with primary antibodies anti-MAP2 (EMD-Millipore, rabbit polyclonal, 1:10,000), anti-200 kD Neurofilament (Abcam, chicken polyclonal, 1:100,000), anti-Synaptotagmin (Synaptic Systems, rabbit polyclonal, 1:500), anti-PSD95 (Abcam, goat polyclonal, 1:500), and/or anti-C4c (Quidel, mouse monoclonal, 1:200). Coverslips were then washed with PBST and incubated for 1 hour at room temperature with secondary antibodies (Abcam, donkey or goat, 1:1000 in 5% BSA-PBST). Coverslips were mounted on slides using Vectashield with DAPI and visualized by fluorescent microscope (Zeiss Confocal).

### Western Blot Analysis

Conditioned media was collected from in-vitro cultured human neurons at days 7 and 30 and frozen at −80° C. until quantification of C4 by western blot. Equal amounts of proteins (20 ug as determined by BCA Protein Assay) were diluted 1:1 with Native Sample Buffer (BioRad 161-0738) and separated on a 4-15% TGX precast polyacrylamide gel. Purified human C4 protein from Quidel (A402) was used as a positive control. Unconditioned neuronal media (Sciencell 1521) provided an appropriate negative control. Electrophoresis was performed using the Mini-PROTEAN Tetra Cell (BioRad). Proteins were then transferred onto polyvinylidene difluoride membranes (Immun-Blot PVDF, BioRad 162-0177) for Western Blot analysis. Membranes were blocked in a 5% milk solution in TBST (0.1% Tween 20) for 1 hour at room temperature and then incubated with anti-C4c (Dako, F016902-2, 1:1000) primary antibody overnight at 4° C. Following washes in TBST, secondary antibody goat-anti-rabbit HRP (Abcam, preadsorbed, 1:10,000) was hybridized for 1 hour at room temperature. Membranes were washed in TBST again and then reactivity was revealed by chemiluminescence reaction performed with ECL detection reagents (BioRad Clarity) and film exposure.

### Mice

The generation of the C4−/− mice that were used to investigate synapse elimination in the retinogeniculate system is described in detail in earlier work⁶⁴. In these mice, the sequence spanning part of exon 23 through exon 29 has been replaced with a PGK-Neo gene. Experiments involved litters created by crossing C4+/− heterozygous parents, so that all comparisons were among littermates of different C4 genotypes. Sample sizes were determined based on power calculations for each data set (to obtain >80% statistical power) and based on recommendations from IACUC to conserve animals. Mice from both sexes were analyzed in these experiments. Experiments were approved by the institutional animal use and care committee in accordance with NIH guidelines for the humane treatment of animals.

### Generation of Human C4 Transgenic Mice

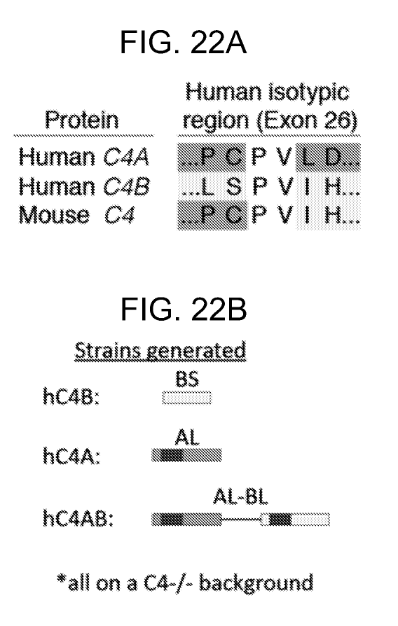

Human C4 transgenic mice were generated using BAC DNA transgenesis. BAC clones containing common human C4 alleles, i.e. C4A allele (MCF258G8), C4B (CH502) allele or C4A and C4B (CH501) were selected and purchased from Childrens Hospital Oakland Research Institute (CHORI) (http://bacpac.chori.org) (Horton et al. Immunogenetics. 2008 January; 60(1):1-18). The human C4 locus encodes two highly conserved isoforms, C4A (acidic) and C4B (basic), whose coding sequences differ by only four amino acids (Belt et al.). The structural differences between the two is conferred by the four amino-acid difference in the isotypic region that drive the efficient binding of C4A and C4B to different chemical targets (FIG. 22B) (Isenman et al., J Immunol 132, 3019-3027 (1984)). C4A preferentially makes amine bonds whereas C4B preferentially binds to carbohydrate. One known target for C4 binding is the synapse. C4 localizes to synapses in the brain and is required for synaptic pruning in the developing visual system, along with other components of the classical complement cascade and microglia (Schafer et al., Neuron 74, 691-705 (2012); Sekar et al., Nature 530, 177-183 (2016)).

In order to understand why increased C4A gene copies, but not C4B, confers schizophrenia risk and because mouse C4 is encoded by only one gene, transgenic mice were generated that express C4A and C4B. BAC DNAs were linearized prior to pronuclear injection into mouse zygotes. Offspring from injections were genotyped using digital droplet PCR (ddPCR) of genomic DNA using primers specific for the C4A or C4B isotypic region to confirm the number of copies of the BAC Tg. Mice were bred with C4−/− C57/B6 mice and backcrossed at least 10 generations (FIG. 22B). Preliminary studies confirm that the human C4A and C4B alleles are expressed in the periphery and CNS as expected and that they function in the murine complement system. The transgenic mice are used to determine how the characterized chemical difference between C4A and C4B affect the developmental process of synapse elimination. In particular, defining the specific role and function of C4A in synapse elimination will help to develop potential therapeutics. Such strategies will be tested in the BAC transgenic mice.

Analysis of Dorsal Lateral Geniculate Nucleus (dLGN)

Visualization and analysis of RGC synaptic inputs in the mouse dLGN was performed as described⁹. Cholera toxin-β subunit (CTB) conjugated to Alexa 488 (green label) and CTB conjugated to Alexa 594 (red label) were intraocularly injected into the left and right eyes, respectively, of P9 mice, which were sacrificed the following day. Images were acquired using a Zeiss Axiocam microscope and quantified blind to experimental conditions and compared to age-matched littermate controls. The degree of left and right eye axon overlap in dLGN was quantified using an R-value analysis as described⁶⁵ and by quantifying the percent overlap as previously described⁶⁶. Pseudocolored images representing the R-value distribution were generated in ImageJ image analysis software.

For measurement of C4 expression in the retinal ganglion cells (RGCs) and LGN, RNA was isolated from tissue with the Qiagen RNeasy Lipid mini kit (cat. No 74804) with optional DNase digestion according to the manufacturer's protocol. RGCs were isolated, lysed, and DNase digested with Ambion Cells to Ct kit⁶⁶. 15 ng of RNA was used as the input for the RT-ddPCR reaction with the primer-probe sets listed in Table 1.

### Measurement of C4 Expression in Mouse Tissues and Cell Populations

Retinal ganglion cells were purified from p5 and p15 C57BL/6 mice through serial immunopanning as previously described⁶⁷. To specifically isolate the lateral geniculate nucleus (LGN) from P5 C57BL/6 mice, LGN was first fluorescently labeled through bi-lateral intraorbital injection of flourophore-conjugated cholera toxin at P4 and then microdissected at P5 during visualization with a fluorescence dissecting microscope. Retinal tissue was harvested from separate P5 C57B16 mice. RNA was isolated from LGN and retinal tissue with the Qiagen RNeasy Lipid mini kit (cat. No 74804) with optional DNase digestion according to the manufacturer's protocol. RGCs were lysed, DNase digested with Ambion Cells to Ct kit, and RNA from the cell-free solution used in subsequent reactions. Mouse C4 expression was calculated as the average of two C4-specific reverse transcription-ddPCR assays, one with the primer-probe set spanning the junction of exons 23 and 24 and the other, the junction of exons 25 and 26, each normalized to the housekeeping mRNA, Eif4h.

### Immunohistochemistry (Mouse Tissue)

Brains were harvested from mice after transcardial perfusion with 4% paraformaldehyde (PFA). Tissue was then immersed in 4% PFA for 2 hours following perfusion, cryoprotected in 30% sucrose, and embedded in a 2:1 mixture of OCT:20% sucrose PBS. Tissue was cryosectioned (12-14 microns), sections were dried, washed three times in PBS, and blocked with 2% BSA+0.2% Triton X in PBS for 1 hr. Primary antibodies were diluted in antibody buffer (+0.05% triton+0.5% BSA) as follows: anti-C3 (Cappel, 1:300), anti-vglut2 (Millipore, 1:2000) and incubated overnight at 4° C. Secondary Alexa-conjugated antibodies (Invitrogen) were added at 1:200 in antibody buffer for 2 hours at room temperature. Slides were mounted in Vectashield (+DAPI) and imaged using the Zeiss Axiocam microscope, Zeiss LSM700. In addition to the analysis of C3 localization, several commercial antibodies for mouse C4 were also tested and it was found that none were sufficiently specific.

### Retinal Cell Counts

Retinal flat mounts were prepared by dissecting out retinas whole from the eyecup and placing four cuts along the major axis, radial to the optic nerve. Each retina was stained with DAPI (Vector Laboratories, Burlingame, Calif.) to reveal cell nuclei. Measurements of RGC density based on Brn3a (goat anti-Brn3a, 1:200, Santa Cruz) immunohistochemistry were carried out blind to genotype from matched locations in the central and peripheral retina for all four retinal quadrants of each retina. Quantification was done on P10 retinas, which is the age at which eye specific segregation analysis was completed. For each retina (1 retina per animal; N=4 mice per treatment condition or genotype), 12 images of peripheral retina and 8 images of central retina were collected. For each field of view collected (20 per retina), Macbiophotonics ImageJ software (NIH) was used to quantify the total number of Brn3a-positive cells using the cell counter plugin. All analyses were performed blind to genotype.

TABLE 1
Primer and probe sequences used
All sequences are provided in the 5′ to 3′ orientation. Assays
identified with an asterisk (*) were based on Wu et al.².

| Assay | Forward Primer | Reverse Primer | Probe |
|---|---|---|---|
| Copy number of human | CCTTTGTGTTGAA | TCCTGTCTAACACT | VIC- |
| C4A* | GGTCCTGAGTT | GGACAGGGGT | CCAGGAGCAGGTA |
| | | | GGAGGCTCGC- |
| | | | MGB |
| Copy number of human | TGCAGGAGACATC | CATGCTCCTATGTA | VIC- |
| C4B* | TAACTGGCTTCT | TCACTGGAGAGA | AGCAGGCTGACGG |
| | | | C-MGB |
| Copy number of human | TTGCTCGTTCTGCT | GTTGAGGCTGGTCC | VIC- |
| C4L* | CATTCCTT | CCAACA | CTCCTCCAGTGGA |
| | | | CATG-MGB |
| Copy number of human | TTGCTCGTTCTGCT | GGCGCAGGCTGCTG | VIC- |
| C4S* | CATTCCTT | TATT | CTCCTCCAGTGGA |
| | | | CATG-MGB |
| Control for copy number | GATTTGGACCTGC | GCGGCTGTCTCCAC | FAM- |
| assays of human DNA | GAGCG | AAGT | CTGACCTGAAGGC |
| (RPP30) | | | TCT-MGB |
| Expression of human C4A | CCTGAGAAACTGC | GTGAGTGCCACAGT | FAM- |
| | AGGAGACAT | CTCATCAT | CAGGACCCCTGTC |
| | | | CAGTGTTAGAC |
| Expression of human C4B | CCTGAGAAACTGC | GTGAGTGCCACAGT | FAM- |
| | AGGAGACAT | CTCATCAT | CTATGTATCACTG |
| | | | GAGAGAGGTCCTG |
| | | | GAAC |
| Expression of mouse C4 | AGCCTGTTTCCAG | GTCCTAAGGCCTCA | FAM- |
| | CTCAAAG | CACCTG | CCCCGGCTGCTGA |
| | | | ACTCCAT |
| Control for expression | CATGTGGAAACTT | CCTTGTTCTATGTC | HEX- |
| assays of human RNA | TGCTTGC | AGCACATCC | TTGTTCCCGTGTTC |
| (ZNF394) | | | CTCACTGTCA |
| Control for expression | GTGCAGCTTGCTT | GTAAATTGCCGAGA | VIC- |
| assays of mouse RNA | GGTAGC | CCTTGC | AGCCTACCCCTTG |
| (Eif4h) | | | GCTCGGG |
| Control for expression | CCCCTGATAGTCA | TGGAGTTTTGAGGG | Hex- |
| assays of mouse RNA | CACAGTCC | TTTTGG | TCCGCTGCTGCTCT |
| (Hs2st1) | | | GGCCTCCT |
| Amplifying human C4S | TCAGCATGTACAG | GAGTGCCACAGTCT | |
| Copies | ACAGGAATACA | CATCATTG | |

TABLE 2
Imputation of C4 structural alleles from SNP data
The correlation (r²) between experimentally derived genotypes
of C4 structural alleles and imputed probabilistic dosages from
leave-one-out trials within the reference panel are shown, together
with a 95% confidence interval for each estimate. Imputation of
C4 structural alleles was tested using SNPs within the extended
MHC locus (chr 6: 25-34 Mb) from the indicated SNP microarrays.
95% confidence intervals around the Pearson r² value are shown
in parentheses. The HapMap-based reference panel included 7,751
SNPs, of which 2,259 to 5,523 were present on the SNP arrays evaluated.
SNP array platform (SNPs in common with MHC reference panel)

| | Illumina | Illumina | Affymetrix |
|---|---|---|---|
| | Omni Express | Immunochip | SNP 6.0 |
| C4 allele | (5,523 SNPs) | (3,703 SNPs) | (2,259 SNPs) |
| BS | 0.85 (0.80-0.90) | 0.86 (0.81-0.91) | 0.92 (0.89-0.95) |
| AL-BS-1 | 0.55 (0.43-0.67) | 0.78 (0.71-0.85) | 0.55 (0.43-0.67) |
| AL-BS-2 | 1.00 (1.00-1.00) | 1.00 (1.00-1.00) | 0.88 (0.84-0.92) |
| AL-BS-3 | 0.84 (0.79-0.89) | 0.74 (0.66-0.82) | 0.67 (0.57-0.77) |
| AL-BS-4 | 0.88 (0.84-0.92) | 0.83 (0.77-0.89) | 0.90 (0.87-0.93) |
| AL-BS-5 | 1.00 (1.00-1.00) | 1.00 (1.00-1.00) | 0.98 (0.97-0.99) |
| AL-BL-1 | 0.71 (0.62-0.8) | 0.71 (0.62-0.8) | 0.57 (0.45-0.69) |
| AL-BL-2 | 0.63 (0.52-0.74) | 0.50 (0.37-0.63) | 0.63 (0.52-0.74) |
| AL-BL-3 | 0.77 (0.7-0.84) | 0.72 (0.63-0.81) | 0.67 (0.57-0.77) |
| AL-AL-1 | 0.54 (0.42-0.66) | 0.58 (0.46-0.70) | 0.65 (0.55-0.75) |
| AL-AL-2 | 0.8 (0.73-0.87) | 0.8 (0.73-0.87) | 0.69 (0.60-0.78) |

TABLE 3
Psychiatric Genomics Consortium cohorts contributing
to association analysis in this study.

| Cohort name | PMID | Site | Genotyping array | Cases | Controls |
|---|---|---|---|---|---|
| scz_aarh_eur | 19571808 | Denmark | Illumina 650K | 876 | 871 |
| scz_aber_eur | 19571811 | Aberdeen, UK | Affymetrix 6.0 | 719 | 697 |
| scz_ajsz_eur | 24253340 | Israel | Illumina 1M | 894 | 1594 |
| scz_asrb_eur | 21034186 | Australia | Illumina 650K | 456 | 287 |
| scz_boco_eur | 19571808 | Bonn/Mannheim, | Illumina 550K | 1773 | 2161 |
| | | Germany | | | |
| scz_buls_eur | | Bulgaria | Affymetrix 6.0 | 195 | 608 |
| scz_cati_eur | 18347602 | US (CATIE) | Affymetrix 500K | 397 | 203 |
| scz_caws_eur | 19571811 | Cardiff, UK | Affymetrix 500K | 396 | 284 |
| scz_cims_eur | | Boston, US (CIDAR) | Illumina | 67 | 65 |
| | | | OmniExpress | | |
| scz_clm2_eur | 22614287 | UK (CLOZUK) | Illumina 1M | 3426 | 4085 |
| scz_clo3_eur | 22614287 | UK (CLOZUK) | Illumina | 2105 | 1975 |
| | | | OmniExpress | | |
| scz_cou3_eur | 21850710 | Cardiff, UK (CogUK) | Illumina | 530 | 678 |
| | | | OmniExpress | | |
| scz_denm_eur | 19571808 | Denmark | Illumina 650K | 471 | 456 |
| scz_dubl_eur | 19571811 | Ireland | Affymetrix 6.0 | 264 | 839 |
| scz_edin_eur | 19571811 | Edinburgh, UK | Affymetrix 6.0 | 367 | 284 |
| scz_egcu_eur | 15133739 | Estonia (EGCUT) | Illumina | 234 | 1152 |
| | | | OmniExpress | | |
| scz_ersw_eur | 19571808 | Sweden (Hubin) | Illumina | 265 | 319 |
| | | | OmniExpress | | |
| scz_fi3m_eur | 19571808 | Finland | Illumina 317K | 186 | 929 |
| scz_fii6_eur | | Finnish | Illumina 550K | 360 | 1082 |
| scz_gras_eur | 20819981 | Germany (GRAS) | Affymetrix Axiom | 1067 | 1169 |
| scz_irwt_eur | 22883433 | Ireland (WTCCC2) | Affymetrix 6.0 | 1291 | 1006 |
| scz_lacw_eur | 22885689 | Six countries, | Illumina 550K | 157 | 245 |
| | | WTCCC controls | | | |
| scz_lie2_eur | 11381111 | NIMH CBDB | Illumina Omni 2.5M | 133 | 269 |
| scz_lie5_eur | 11381111 | NIMH CBDB | Illumina 550K | 497 | 389 |
| scz_mgs2_eur | 19571809 | US, Australia (MGS) | Affymetrix 6.0 | 2638 | 2482 |
| scz_msaf_eur | 20489179 | New York, US & | Affymetrix 6.0 | 325 | 139 |
| | | Israel | | | |
| scz_munc_eur | 19571808 | Munich, Germany | Illumina 317K | 421 | 312 |
| scz_pewb_eur | 23871474 | Seven countries | Illumina 1M | 574 | 1812 |
| | | (PEIC, WTCCC2) | | | |
| scz_pews_eur | 23871474 | Spain (PEIC, | Illumina 1M | 150 | 236 |
| | | WTCCC2) | | | |
| scz_port_eur | 19571811 | Portugal | Affymetrix 6.0 | 346 | 215 |
| scz_s234_eur | 23974872 | Sweden (sw234) | Affymetrix 6.0 | 1980 | 2274 |
| scz_swe1_eur | 23974872 | Sweden (sw1) | Affymetrix 5.0 | 215 | 210 |
| scz_swe5_eur | 23974872 | Sweden (sw5) | Illumina | 1764 | 2581 |
| | | | OmniExpress | | |
| scz_swe6_eur | 23974872 | Sweden (sw6) | Illumina | 975 | 1145 |
| | | | OmniExpress | | |
| scz_top8_eur | 19571808 | Norway (TOP) | Affymetrix 6.0 | 377 | 403 |
| scz_ucla_eur | 19571808 | Netherlands | Illumina 550K | 700 | 607 |
| scz_uclo_eur | 19571811 | London, UK | Affymetrix 6.0 | 509 | 485 |
| scz_umeb_eur | | Umeå, Sweden | Illumina | 341 | 577 |
| | | | OmniExpress | | |
| scz_umes_eur | | Umeå, Sweden | Illumina | 193 | 704 |
| | | | OmniExpress | | |
| scz_zhh1_eur | 17522711 | New York, US | Affymetrix 500K | 190 | 190 |

### REFERENCES

1. Cannon, T. D. et al. Cortex mapping reveals regionally specific patterns of genetic and disease-specific gray-matter deficits in twins discordant for schizophrenia. Proceedings of the National Academy of Sciences of the United States of America 99, 3228-3233, doi:10.1073/pnas.052023499 (2002).
2. Cannon, T. D. et al. Progressive reduction in cortical thickness as psychosis develops: a multisite longitudinal neuroimaging study of youth at elevated clinical risk. Biological psychiatry 77, 147-157, doi:10.1016/j.biopsych.2014.05.023 (2015).
3. Garey, L. J. et al. Reduced dendritic spine density on cerebral cortical pyramidal neurons in schizophrenia. J Neurol Neurosurg Psychiatry 65, 446-453 (1998).
4. Glantz, L. A. & Lewis, D. A. Decreased dendritic spine density on prefrontal cortical pyramidal neurons in schizophrenia. Arch Gen Psychiatry 57, 65-73 (2000).
5. Glausier, J. R. & Lewis, D. A. Dendritic spine pathology in schizophrenia. Neuroscience 251, 90-107, doi: 10.1016/j.neuroscience.2012.04.044 (2013).
6. Schizophrenia Working Group of the Psychiatric Genomics Consortium. Biological insights from 108 schizophrenia-associated genetic loci. Nature 511, 421-427, doi:10.1038/nature 13595 (2014).
7. Shi, J. et al. Common variants on chromosome 6p22.1 are associated with schizophrenia. Nature 460, 753-757, doi:10.1038/nature08192 (2009).
8. Stefansson, H. et al. Common variants conferring risk of schizophrenia. Nature 460, 744747,
doi:10.1038/nature08186 (2009).
9. International Schizophrenia Consortium et al. Common polygenic variation contributes to risk of schizophrenia and bipolar disorder. Nature 460, 748-752,
doi:10.1038/nature08185 (2009).
10. Schizophrenia Psychiatric Genome-Wide Association Study Consortium. Genome-wide association study identifies five new schizophrenia loci. Nature genetics 43, 969-976, doi:10.1038/ng.940 (2011).
11. Howson, J. M., Walker, N. M., Clayton, D. & Todd, J. A. Confirmation of HLA class II independent type 1 diabetes associations in the major histocompatibility complex including HLA-B and HLA-A. Diabetes Obes Metab 11 Suppl 1, 31-45, doi:10.1111/j. 1463-1326.2008.01001.x (2009).
12. Raychaudhuri, S. et al. Five amino acids in three HLA proteins explain most of the association between MHC and seropositive rheumatoid arthritis. Nature genetics 44, 291296, doi:10.1038/ng.1076 (2012).
13. Escudero-Esparza, A., Kalchishkova, N., Kurbasic, E., Jiang, W. G. & Blom, A. M. The novel complement inhibitor human CUB and Sushi multiple domains 1 (CSMD1) protein promotes factor I-mediated degradation of C4b and C3b and inhibits the membrane attack complex assembly. FASEB journal: official publication of the Federation of American Societies for Experimental Biology 27, 5083-5093, doi: 10.1096/fj. 13-230706 (2013).
14. Carroll, M. C., Campbell, R. D., Bentley, D. R. & Porter, R. R. A molecular map of the human major histocompatibility complex class III region linking complement genes C4, C2 and factor B. Nature 307, 237-241 (1984).
15. Carroll, M. C., Belt, T., Palsdottir, A. & Porter, R. R. Structure and organization of the C4 genes. Philos Trans R Soc LondB Biol Sci 306, 379-388 (1984).
16. Dangel, A. W. et al. The dichotomous size variation of human complement C4 genes is mediated by a novel family of endogenous retroviruses, which also establishes species specific
genomic patterns among Old World primates. Immunogenetics 40, 425-436 (1994).
17. Horton, R. et al. Variation analysis and gene annotation of eight MHC haplotypes: the MHC Haplotype Project. Immunogenetics 60, 1-18, doi:10.1007/s00251-007-0262-2 (2008).
18. Banlaki, Z., Doleschall, M., Rajczy, K., Fust, G. & Szilagyi, A. Fine-tuned characterization of RCCX copy number variants and their relationship with extended MHC haplotypes. Genes Immun 13, 530-535, doi:10.1038/gene.2012.29 (2012).
19. Law, S. K., Dodds, A. W. & Porter, R. R. A comparison of the properties of two classes, C4A and C4B, of the human complement component C4. EMBO J3, 1819-1823 (1984).
20. Isenman, D. E. & Young, J. R. The molecular basis for the difference in immune hemolysis activity of the Chido and Rodgers isotypes of human complement component C4. J Immunol 132, 3019-3027 (1984).
21. Illarionova, A. E., Vinogradova, T. V. & Sverdlov, E. D. Only those genes of the KIAA1245 gene subfamily that contain HERV(K) LTRs in their introns are transcriptionally active. Virology 358, 39-47, doi:10.1016/j.viro1.2006.06.027 (2007).
22. Nakamura, A., Okazaki, Y., Sugimoto, J., Oda, T. & Jinno, Y. Human endogenous retroviruses with transcriptional potential in the brain. Journal of human genetics 48, 575-581, doi: 10.1007/s10038-003-0081-8 (2003).
23. Suntsova, M. et al. Human-specific endogenous retroviral insert serves as an enhancer for the schizophrenia-linked gene PRODH. Proceedings of the National Academy of Sciences of the United States of America 110, 19472-19477, doi:10.1073/pnas. 1318172110 (2013).
24. Yang, Y. et al. Diversity in intrinsic strengths of the human complement system: serum C4 protein concentrations correlate with C4 gene size and polygenic variations, hemolytic activities, and body mass index. J Immunol 171, 2734-2745 (2003).
25. Browning, S. R. & Browning, B. L. Rapid and accurate haplotype phasing and missing data inference for whole-genome association studies by use of localized haplotype clustering. Am J Hum Genet 81, 1084-1097, doi:10.1086/521987 (2007).
26. Iossifov, I. et al. The contribution of de novo coding mutations to autism spectrum disorder. Nature 515, 216-221, doi:10.1038/nature13908 (2014).
27. Mayilyan, K. R., Arnold, J. N., Presanis, J. S., Soghoyan, A. F. & Sim, R. B. Increased complement classical and mannan-binding lectin pathway activities in schizophrenia. Neurosci Lett 404, 336-341, doi:10.1016/j.neulet.2006.06.051 (2006).
28. Hakobyan, S., Boyajyan, A. & Sim, R. B. Classical pathway complement activity in schizophrenia. Neurosci Lett 374, 35-37, doi:10.1016/j.neulet.2004.10.024 (2005).
29. Stevens, B. et al. The classical complement cascade mediates CNS synapse elimination. Cell 131, 1164-1178, doi:10.1016/j.cell.2007.10.036 (2007).
30. Schafer, D. P. et al. Microglia sculpt postnatal neural circuits in an activity and complement-dependent manner. Neuron 74, 691-705, doi:10.1016/j.neuron.2012.03.026 (2012).
31. Bialas, A. R. & Stevens, B. TGF-beta signaling regulates neuronal C1q expression and developmental synaptic refinement. Nat Neurosci 16, 1773-1782, doi:10.1038/nn.3560 (2013).
32. Kaiser, T. & Feng, G. Modeling psychiatric disorders for developing effective treatments. Nat Med 21, 979-988, doi:10.1038/nm.3935 (2015).
33. Shatz, C. J. & Kirkwood, P. A. Prenatal development of functional connections in the cat's retinogeniculate pathway. J Neurosci 4, 1378-1397 (1984).
34. Sretavan, D. W. & Shatz, C. J. Prenatal development of retinal ganglion cell axons: segregation into eye-specific layers within the cat's lateral geniculate nucleus. J Neurosci 6, 234-251 (1986).
35. Chen, C. & Regehr, W. G. Developmental remodeling of the retinogeniculate synapse. Neuron 28, 955-966 (2000).
36. Fischer, M. B. et al. Regulation of the B cell response to T-dependent antigens by classical pathway complement. J Immunol 157, 549-556 (1996).
37. Huttenlocher, P. R. & Dabholkar, A. S. Regional differences in synaptogenesis in human cerebral cortex. J Comp Neurol 387, 167-178 (1997).
38. Huttenlocher, P. R. Synaptic density in human frontal cortex—developmental changes and effects of aging. Brain Res 163, 195-205 (1979).
39. Petanjek, Z. et al. Extraordinary neoteny of synaptic spines in the human prefrontal cortex. Proceedings of the National Academy of Sciences of the United States of America 108, 13281-13286, doi:10.1073/pnas.1105108108 (2011).
40. Buckner, R. L. & Krienen, F. M. The evolution of distributed association networks in the human brain. Trends Cogn Sci 17, 648-665, doi:10.1016/j.tics.2013.09.017 (2013).
41. Feinberg, I. Schizophrenia: caused by a fault in programmed synaptic elimination during adolescence? Journal of psychiatric research 17, 319-334 (1982).
42. Kirov, G. et al. De novo CNV analysis implicates specific abnormalities of postsynaptic signalling complexes in the pathogenesis of schizophrenia. Mol Psychiatry 17, 142-153, doi:10.1038/mp.2011.154 (2012).
43. Fromer, M. et al. De novo mutations in schizophrenia implicate synaptic networks. Nature 506, 179-184, doi:10.1038/nature12929 (2014).
44. Purcell, S. M. et al. A polygenic burden of rare disruptive mutations in schizophrenia. Nature 506, 185-190, doi:10.1038/nature12975 (2014).
45. Datwani, A. et al. Classical MHCI molecules regulate retinogeniculate refinement and limit ocular dominance plasticity. Neuron 64, 463-470, doi: 10.1016/j.neuron.2009.10.015 (2009).
46. Lee, H. et al. Synapse elimination and learning rules co-regulated by MHC class I H2-Db. Nature 509, 195-200, doi:10.1038/nature13154 (2014).
47. van den Elsen, J. M. et al. X-ray crystal structure of the C4d fragment of human complement component C4. J Mol. Blol 322, 1103-1115 (2002).
48. Dodds, A. W., Ren, X. D., Willis, A. C. & Law, S. K. The reaction mechanism of the internal thioester in the human complement component C4. Nature 379, 177-179, doi:10.1038/379177a0 (1996).
49. Handsaker, R. E. et al. Large multiallelic copy number variations in humans. Nature genetics 47, 296-303, doi:10.1038/ng.3200 (2015).
50. Torborg, C. L. & Feller, M. B. Unbiased analysis of bulk axonal segregation patterns. J Neurosci Methods 135, 17-26, doi:10.1016/j.jneumeth.2003.11.019 (2004).
51. Fernando, M. M. et al. Assessment of complement C4 gene copy number using the paralog ratio test. Hum Mutat 31, 866-874, doi: 10.1002/humu.21259 (2010).
52. Rudduck, C., Beckman, L., Franzen, G., Jacobsson, L. & Lindstrom, L. Complement factor C4 in schizophrenia. Hum Hered 35, 223-226 (1985).
53. Schroers, R. et al. Investigation of complement C4B deficiency in schizophrenia. Hum Hered 47, 279-282 (1997).
54. Mayilyan, K. R., Dodds, A. W., Boyajyan, A. S., Soghoyan, A. F. & Sim, R. B. Complement C4B protein in schizophrenia. World J Blol Psychiatry 9, 225-230, doi:10.1080/15622970701227803 (2008).
55. Jia, X. et al. Imputing amino acid polymorphisms in human leukocyte antigens. PLoS One 8, e64683, doi:10.1371/journal.pone.0064683 (2013).
56. Nonaka, M., Nakayama, K., Yeul, Y. D. & Takahashi, M. Complete nucleotide and derived amino acid sequences of sex-limited protein (Slp), nonfunctional isotype of the fourth component of mouse complement (C4). J Immunol 136, 2989-2993 (1986).
57. Hindson, B. J. et al. High-throughput droplet digital PCR system for absolute quantitation of DNA copy number. Analytical chemistry 83, 8604-8610, doi:10.1021/ac202028g (2011).
58. Wu, Y. L. et al. Sensitive and specific real-time polymerase chain reaction assays to accurately determine copy number variations (CNVs) of human complement C4A, C4B, C4-long, C4-short, and RCCX modules: elucidation of C4 CNVs in 50 consanguineous subjects with defined HLA genotypes. Journal of immunology (Baltimore, Md.: 1950) 179, 3012-3025 (2007).
59. Fernando, M. M. et al. Assessment of complement C4 gene copy number using the paralog ratio test. Human mutation 31, 866-874, doi:10.1002/humu.21259 (2010).
60. Browning, B. L. & Browning, S. R. A unified approach to genotype imputation and haplotype-phase inference for large data sets of trios and unrelated individuals. American journal of human genetics 84, 210-223, doi:10.1016/j.ajhg.2009.01.005 (2009).
61. Browning, S. R. & Browning, B. L. Rapid and accurate haplotype phasing and missing-data inference for whole-genome association studies by use of localized haplotype clustering. American journal of human genetics 81, 1084-1097, doi:10.1086/521987 (2007).
62. Schizophrenia Working Group of the Psychiatric Genomics Consortium. Biological insights from 108 schizophrenia-associated genetic loci. Nature 511, 421-427, doi:10.1038/nature 13595 (2014).
63. Jia, X. et al. Imputing amino acid polymorphisms in human leukocyte antigens. PloS one 8, e64683, doi:10.1371/journal.pone.0064683 (2013).
64. Fischer, M. B. et al. Regulation of the B cell response to T-dependent antigens by classical pathway complement. Journal of immunology (Baltimore, Md.: 1950) 157, 549-556 (1996).
65. Torborg, C. L. & Feller, M. B. Unbiased analysis of bulk axonal segregation patterns. Journal of neuroscience methods 135, 17-26, doi:10.1016/j.jneumeth.2003.11.019 (2004).
66. Bialas, A. R. & Stevens, B. TGF-beta signaling regulates neuronal C1q expression and developmental synaptic refinement. Nature neuroscience 16, 1773-1782, doi:10.1038/nn.3560 (2013).
67. Barres, B. A., Silverstein, B. E., Corey, D. R. & Chun, L. L. Y. Immunological, morphological, and electrophysiological variation among retinal ganglion cells purified by panning. Neuron 1,791-803 (1988).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of treating schizophrenia in a subject, the method comprising administering to the subject an agent that inhibits the expression or activity of a complement component 4A (C4A) polypeptide or polynucleotide.

2. A method of reducing an interaction between a neuron and a microglia and/or reducing synaptic elimination in a subject, the method comprising contacting a microglia or neuron with an an agent that inhibits the expression or activity of a complement component 4A (C4A) polypeptide or polynucleotide.

3. The method of claim 2, wherein the microglia or neuron is contacted with the agent in vitro or in vivo.

4. The method of claim 3, wherein the microglia or neuron is contacted with the agent in a subject

5. The method of any one of claims 2-5, wherein engulfment of synapses by microglia is reduced.

6. The method of any one of claims 1-5, wherein the agent inhibits the expression or activity of a complement component 4B (C4B) polypeptide or polynucleotide.

7. The method of any one of claims 1-5, wherein the agent does not inhibit the expression or activity of a complement component 4B (C4B) polypeptide or polynucleotide.

8. The method of any one of claims 1-7, wherein the agent is an antibody or an inhibitory nucleic acid.

9. The method of claim 8, wherein the antibody specifically binds an epitope containing the amino acid sequence PCPVLD.

10. The method of claim 8 or 9, wherein the antibody does not bind an epitope containing the amino acid sequence LSPVIH.

11. The method of any one of claims 6-10, wherein the agent is a complement inhibitor.

12. The method of any one of claims 1 and 4-11, wherein the subject is human.

13. A method of treating schizophrenia in a pre-selected subject, the method comprising administering a schizophrenia treatment to the subject, wherein the subject is pre-selected by detecting an increase in a level of a complement component 4A (C4A) polynucleotide or polypeptide, an increase in a combined level of C4A and complement component 4B (C4B) polynucleotide or polypeptide, an increase in copy number of complement component 4A (C4A), and/or an alteration in a sequence of C4A or C4B polynucleotide relative to a reference in a biological sample obtained from the subject.

14. A method of monitoring treatment progress in a subject having schizophrenia and administered with a schizophrenia treatment, the method comprising measuring a level of C4A polypeptide or polynucleotide or a combined level of C4A and C4B polypeptide or polynucleotide relative to a reference level in a biological sample obtained from the subject, wherein a decrease in the level or combined level indicates the subject is responsive to the schizophrenia treatment.

15. A method of determining efficacy of a schizophrenia treatment in a subject, the method comprising measuring a level of C4A polypeptide or polynucleotide or a combined level of C4A and C4B polypeptide or polynucleotide relative to a reference level in a biological sample obtained from the subject, wherein a decrease in the level or combined level indicates the the schizophrenia treatment is efficacious.

16. A method of characterizing a subject having a mental disorder, the method comprising measuring
a level of a complement component 4A (C4A) polynucleotide or polypeptide,
a combined level of C4A and complement component 4B (C4B) polynucleotide or polypeptide,
a copy number of C4A polynucleotide, and/or
a sequence of C4A and/or C4B polynucleotide relative to a reference in a biological sample obtained from the subject,
wherein an increase in the level of C4A polynucleotide or polypeptide, an increase in the combined level of C4A and C4B polynucleotide or polypeptide, an increase in C4A copy number and/or an alteration in a sequence of C4A or C4B polynucleotide indicates the subject has schizophrenia or is at risk of developing schizophrenia.

17. A method of identifying a subject having or at risk of developing schizophrenia, the method comprising measuring
a level of a complement component 4A (C4A) polynucleotide or polypeptide,
a combined level of C4A and complement component 4B (C4B) polynucleotide or polypeptide,
a copy number of C4A polynucleotide, and/or
a sequence of C4A and/or C4B polynucleotide relative to a reference in a biological sample obtained from the subject,
wherein the subject is identified as having or at risk of developing schizophrenia if the level of C4A polynucleotide or polypeptide is increased, the combined level of C4A and C4B polynucleotide or polypeptide is increased, the copy number of C4A polynucleotide is increased, and/or the sequence of C4A or C4B polynucleotide is altered.

18. A method of characterizing risk of schizophrenia in a subject, the method comprising measuring
a level of a complement component 4A (C4A) polynucleotide or polypeptide,
a combined level of C4A and complement component 4B (C4B) polynucleotide or polypeptide,
a copy number of C4A polynucleotide, and/or
a sequence of C4A and/or C4B polynucleotide relative to a reference in a biological sample obtained from the subject,
wherein an increase in the level of C4A polynucleotide or polypeptide, an increase in the combined level of C4A and C4B polynucleotide or polypeptide, an increase in C4A copy number and/or an alteration in a sequence of C4A or C4B polynucleotide indicates the subject has schizophrenia or is at risk of developing schizophrenia.

19. The method of any one of claims 15-18, further comprising recommending the subject for schizophrenia treatment or for further evaluation for schizophrenia if the subject is identified as having or at risk of developing schizophrenia.

20. The method of any one of claims 15-18, further comprising administering a schizophrenia treatment to the subject if the subject is identified as having or at risk of developing schizophrenia.

21. The method of any one of claims 13-18, wherein the alteration in sequence is insertion of a human endogenous retrovirus (HERV) sequence.

22. The method of any one of claims 13-18, wherein an increase in copy number of C4A polynucleotide and insertion of a human endogenous retrovirus (HERV) sequence in a C4A and/or C4B polynucleotide is detected.

23. The method of any one of claims 13-18, wherein an increase in a level of C4A polynucleotide or polypeptide is detected.

24. The method of any one of claims 13-18, wherein an increase in a combined level of C4A and C4B polynucleotide or polypeptide is detected.

25. The method of any one of claims 13-18, wherein the biological sample is plasma, serum, or cerebrospinal fluid (CSF).

26. The method of any one of claims 13-18, wherein the subject is human.

27. The method of any one of claims 13-15 and 19-26, wherein the schizophrenia treatment is an antipsychotic agent or psychosocial therapy.

28. The method of any one of claims 13-15 and 19-26, wherein the schizophrenia treatment comprises inhibiting the expression or activity of a complement component 4A (C4A) polypeptide or polynucleotide.

29. A kit comprising a capture reagent for detecting the sequence of complement component 4A (C4A) polynucleotide or complement component 4B (C4B), and an antipsychotic agent.

30. The kit of claim 29, further comprising a capture reagent for detecting the sequence of a HERV.

31. The kit of claim 29 or 30, wherein the capture reagent is a probe or a primer.

32. The method of any one of claims 13-28, wherein the level, copy number, and/or sequence of complement component 4A (C4A) polynucleotide or complement component 4B (C4B) is measured using the kit of any one of claims 29-31.

33. A method of identifying an agent that inhibits schizophrenia, the method comprising
(a) contacting a cell or organism with a candidate agent, and
(b) measuring a level of complement component 4A (C4A) polynucleotide or polypeptide in the cell or organism contacted with the candidate agent relative to a reference level, wherein a decrease in the level indicates the candidate agent inhibits schizophrenia.

34. An expression vector comprising an polynucleotide encoding complement component 4A (C4A).

35. A host cell or host organism comprising an expression vector comprising an polynucleotide encoding complement component 4A (C4A).

36. The host cell or host organism of claim 35, wherein the cell or organism is mammalian.

37. A transgenic mouse comprising a polynucleotide sequence encoding a human complement component 4A (huC4A) or human complement component 4B (huC4B) polypeptide, wherein the polynucleotide sequence is operatively linked to a promoter sequence.

38. The transgenic mouse of claim 37, wherein the huC4A or huC4B polypeptide is expressed in the central nervous system.

39. The transgenic mouse of claim 37 or 38, wherein the mouse complement component 4 (C4) gene is deleted or inactivated.


SEQUENCE LISTING (47 sequences)

SEQ ID NO: 1 - protein, 6 aa, Homo sapiens
PCPVLD

SEQ ID NO: 2 - protein, 6 aa, Homo sapiens
LSPVIH

SEQ ID NO: 3 - protein, 1744 aa, Homo sapiens [sequence omitted]

SEQ ID NO: 4 - DNA, 27624 bp, Homo sapiens
tgtcttttggggtttgtttttattctctctttgagttttgtttccttatgcgcccagttacttttgaaaatgttctgggcagatttgcctagattaataaatgccctccatgttccaattacttttttttttttgagacagtgtcttaccctgtcaccaagctggagtgcagtggtatgatcttggctcactgcaacctctgcctcctgagttcaagtgattctcctgcctcagcctcccaagtagctggcattacaggcacctgacaccacgcccagctaattttttttttttttttttttttgagacggagtctcgctctgtcacccaggctggagttcagtggcatgatcttggcttactgcaagctctgcctcctgggttcacccattctcccgcctcagcctcccgagtagctgggactacaggtgcccgccactatgcctggctaattgtttttttttttgtatttttagtagagatggggtttcaccgtgttagccaggatggtcttgatctccggacctcgtgatccacccgtctcagcctgccaaagtgctgggattacaggcatgagccaccgcatctggcctatttttgtatttttaatggagaccgggtttcatcatgttggccaggctggtcttgaacttgaacttctgacctcaagtgatccacccttagcgtcccaaagtgctgggattacaggcatgagccaccgtgcccggccccagttatttttatttttattttttgagttagagtctcactctgtcacccaggctggagcgcagtggcatgatctcggctcacagcaactttctgggttcaagcagttctcctgtgtcagcctcctgagtagctgggactacaggcacacatcaccacgcccggctaatttttgtagttttagtagagacggggttttaccatattggtcaggctgatattgaactcctgacctcaggtgatccacccacgtcagcctcccaaagtgccgggattacaggcttgagccatctcgcccggcctacttagatgttatattagtggtaattcctgttatcctgtgagctctttagtgtctaaacaattttttttaagagatggggtctcactgtgttgcccagttgcaatcatatcttactgcagcctcaaactcctgggtcaagtgatcctcttgccttagtctcccaagtagctaggaccataggtgtctgcccccacgcctggctgtttttacattttttgtagagatgtggcgggtgggggggtctcactgtgttgcccagactggtctcgaactcctgtcctcaattgatcctgctacctcagcctcccaaaatgctgaattacaggcatgagccactgtacctggtcttaaacaattttaaaataacatttttatccaggattttagttaattttcaacaggtggattagttcttgctgtattctcgtaaacagaagtcctggtttatttttatttgttttaaacattgaatcccatactcctccccaccttaccctacccagaatttagactgttaatgttttgaagccacagcctgcatcttaatcactattttatcttagtgcctggtcttagaaattatattgactctttgatagaccatatataaggcaggtggatgagaatgtgggtagctagttggaaaaggctgcttggtcatttgcttgattattttctcacacagtttttcctttactaagagaaaatgcccccatattggcaaacaaaatctccctgcctgagagcgcccagagtatagcagagcatcttaccctgatacgcctcttttcactctcttctctgtggagacagaaggagcttcaagagcagggggagatcagaatcgtccagctgggcttcgacttggatgcccatggaattatcttcactgaggactacaggaccagagtatgtgactgtgtgcgtcaggggtgctggggggagggcacaggttgggggagacagggaacttgggaaacagaaataaaaacaaaagaaagaatttccctgcccccacatcccatggagagggcacagggccctggtaaatagtaatatgagggagagagacaggagggaaagagggaggagtgagagggtaaagagggggggagaggagggggaggaggaggaaggaaggagggggaggaggagggggggaggaagagggggaggaggatgaagaggaggaggaagaagaagggtatgagaggtggaaggatctgagcaagaggtaagacaggaagagaaatgctgtcctgggggtggaggttggtagagagtgagggtggggatggaccatgtctctcatctctgcttgtaggtcctcaaggcctgtgatggccgaccgtatgctggggcagtgcagaaatttctagcttcagtacttccagcctgtggggaccttagtttccagcaggaccaaatgacacagacctttggcttcagggactcagaaatcacgtgagacttgtggaaccaaccaaagtcaggcatctggtgcttccctgcctccctccagttccatccagcctgtcctcctgtttttttggtgaacctgccagaaaagctgccaaaaagctgactcttcttgttaataaaatgacccaagtttgtattcctccccacaagagaggaggcctatcttacctgggccttagaaagagccctgaaatagaattcagttcttggtggcttatcaaaagcacacaggggcctggcaggaagtgtaaaagcttgatgttaatcatactgggactaagaggatagagaatggtaggagctgggatacccctaaacattcacattaaaacaaaaaaaacccaaagctaaaaaacaactgggcaggagctaaataaaaatctaattttgagaggctgtatctggctcaggcctcctactttgtaacccatggaatatgtgaaagcatttgaaaaactatagcactgatctcacatgggcagacacactctcagagagatgtggtgggagccatggcgcagtctgcctaggcagtggcaggagcgcagaagactctgattcctctcctcggtcctaagaccgaatgtgtgtcaggacatgtggtcagggaagagaagctatttaactgaaccagtaatagtagcaggaaaagaaaaagtggagggagggcagtccaggtagggggcctggaacaagcaactgcaccaacagaggcagttggtgcgagcacagaaccaccccaggctgggattttgttatccagtctctcttgcatggttgcccgtgtttctggagacttgtgtaaacattaatggatgaggaggagagatggttctcagagcccagccctcatctctgctggcttcccactgccctcaggcatctggtgaatgctggagtcctcaccgtccgagatgctgggagctggtggctagctgtgcctggagctgggagattcatcaagtactttgttaaaggtatcccatctgcagctcaagcctgcagcccctcaccttttggtggctcctcaggcctctaggccttattcacctttcccctttcctgtgccacttctcctctagggcgccaggctgtccttagcatggtccggaaggcaaagtaccgggaactgctcctatcagagctcctgggccggcgggcgcctgtcgtggtgcggcttggcctcacctaccatgtgcacgacctcattggggcccagctagtggactggtgagtctttccctggcctctggcagattatggagcaatgacccaaagtgggatttcctcccagctcatgcttagtttcctagtgaaggccagtggctctcattcttctctggaacccgggagcaccccttcccaagttctaagttctcctcacagcttgagcctaggcgtctggctccagccttgtctttctcctgcacagcatctctaccacttcaggaaccctcctccgcctgccagagacatgaagattctgctcatcattgctcagctcctcagagtgggccgggaggggactagaagagctgcatgatggtggctgagacagggtcaccttgggaaggcttgggagccaggatgagtgtcgggctctcgtgtgtgcaaaaggtcagatgtgactgctgctgtttgcctggtttctgacccagtggtggggtttgagcaatgcttctctgcccttccatggaaagtggaaccagaaatggtgccaaggctgtggctgttccctttcgtgtaaaatggtgctgttattactctgtcttgaaataggaaggtgggatttctggggaggctggtgaaggagggcagggttcttttctctacgtgtcatgttaaaattgccaaataaagtacctctgcctgtgatattttctggatgtcctttatttactgtgacgtgtgtttgggtgccttgtttaggggtagaggtgaagtctgagctttgcctcattcagagaggaaaggggtcaggggttcactctgacgttcaggccattctccctgtggagtggtgagggtgtacctaatctcctaaaccacggaatttctgttagggcctaaaaaagcaaaagcctagtatagttcaatttgtgttggaatgaaagtaagagacaagtgtcttagaagcctgtcattgttttgtgagggcctttaaatatcctgtactcgtgggccatgttgggcccttgtacgcccaggtatacatgagcttgtgtgcacctataccctgatacagatatacctggtagggggaggtgctcaggcactggaatgagaggagttaacggggaaggacagggttatttctgggccaagattcagagtttcccatggacacccaggtgtccggggtgcccccacaactctgggcctgaggccagttgcacttcttggctgtcacgtggtttcccagcttagctgggctgggggaggagcaaggtccagagtcaactctgccccgaggcctagcttggccagaaggtagcagacagacagacggatctaacctctcttggatcctccagccatgaggctgctctgggggctgatctgggcatccagcttcttcaccttatctctgcagaagcccaggtcctggaggcgggatgctgggtgcttggattggggcagggctggcatcgggacccgattcaggagtgagggagagcaggggtggaggtgtcagagcgaagtctgactgctgatcctgtctgttctccccaggttgctcttgttctctccttctgtggttcatctgggggtccccctatcggtgggggtgcagctccaggatgtgccccgaggacaggtagtgaaaggatcagtgttcctgagaaacccatctcgtaataatgtcccctgctccccaaaggtggacttcacccttagctcagaaagagacttcgcactcctcagtctccaggtaaccagaccccatgccctcctgctgcttgtgggggcctcctgccctgttcccatctgtcttgtaagtgtcatcatcttcccactggcctcctcccctcctgtcttcccaccctggcattctccttccacgtttctcccttggtctctgtcctttttggtcagctgtctcttgctctgtgacccgctccctctccctctccctctcctgacaggtgcccttgaaagatgcgaagagctgtggcctccatcaactcctcagaggccctgaggtccagctggtggcccattcgccatggctaaaggactctctgtccagaacgacaaacatccagggtatcaacctgctcttctcctctcgccgggggcacctctttttgcagacggaccagcccatttacaaccctggccagcggggtgagtctcagccccagggcctcaacctttaaccccctccgagccctctcaggatgagtttggtgccccctaagtgagataacctgaaagaaagtgccacacagaaggggtgcttaggaaacatttgtcccctgctccctctgtggagtttgacccaccctccccttgcacatggacccctgctcacctctctcctcctccactcccagttcggtaccgggtctttgctctggatcagaagatgcgcccgagcactgacaccatcacagtcatggtggaggtgagtccccgacctctggccttcctgatcctggccactgatgtgacctcctgcctgtgagcacttctccccttgcagaactctcacggcctccgcgtgcggaagaaggaggtgtacatgccctcgtccatcttccaggatgactttgtgatcccagacatctcagagtgagcgctcccaatgtgggggctgcccccaagctacaccaccccaattcctgttaggctctccacctcccacacagaggcacgtccccagatgccctgaccctcagcctcctgagcctctggttaacccccacagtcctcttcccagggaagcaggctgctggctctccgtgccccactgtacagatgggctgagccccttccttgtccattctcaggccagggacctggaagatctcagcccgattctcagatggcctggaatccaacagcagcacccagtttgaggtgaagaaatatggtgagagctggaaactggagggacaggcagctgctttcctgaaggaaataagggtggaaggagaggtactgggagcagctcagggcagggagatatgggtgccacagccctgagcagaggggagtctttgagctggagtctgacctgcctatcccttcaccctgggtcagtccttcccaactttgaggtgaagatcacccctggaaagccctacatcctgacggtgccaggccatcttgatgaaatgcagttagacatccaggccaggtaatacctccctccccacctctgcccaccagcaccgggtcctgctccctactcagtatgaatgggctcctgcttccctgccctcgggccattattccccccagcccttggcccaccctcttctctctgccacgacaggtacatctatgggaagccagtgcagggggtggcatatgtgcgctttgggctcctagatgaggatggtaagaagactttctttcgggggctggagagtcagaccaaggtaggaaggagaataggggctggggaggggaaggggcaagggaggtgaggtgggagactcagtctcaccctatgtcctgtttctttctatgccccagctggtgaatggacagagccacatttccctctcaaaggcagagttccaggacgccctggagaagctgaatatgggcattactgacctccaggggctgcgcctctacgttgctgcagccatcattgagtctccaggtgggtgactttcccttattgtaaccccagacccttgcctctgacctctgagctaaccctctgtcctccggcaccaacaccaccccacttctcacatctcatctcagactcaaaaccaggaaacacccaggagacctggtttctctccaactctgtctctgtgactcggcccttttccctggctgagtttatttatttctttgctcgttctgctcattccttcactcctccagtggacatgtgttgttcaatgccccgtgctaggcctcagcatgcacagacatgttggggaccagcctcaacgccacccgtagggttcctgaagtccattggtgacacaggaatgagaagagacaggttaagagttcataaagagtgggggccagggggccaattgcaaaatggaggctgcaaaaggctcagagctctggtctccacactattttttgagtacagtcactcagatctaagaagcagatgttcagggagaaacagtgaaagggaggcagtgggtcataggcgtaatctatagcaatagagttttaaatgaatctcctttgtgctcaaacagcatgtctttaaattatcggagagtagctggtggaagtgggcttagctagaagactgcatgtctgtccaatgcttcaaaggagggtctttctccttgaacagagtgtttacagataagacagggggtctcactctgagcatgggaacatgatggcaattaggaggcttttcttctcagaggcctcttgtggctttccacaacttattgtctcatatttttatggacagtttatacaggcaccccacaagtccttttcccaacatgcccccctccctttttttttttttaaccgctattgctattatggcttatttgtggtgtttggtctgttttcagaagtgtcttttgcatctgtagactaaaagtaaacagcataaacagatacacattaaagtaaaatttgtaatagttgatcctttaatggtcttaatctgtttaagaggatttatgtttgaaagtccgtcagtagctccaatgagaatgtcagtctcaggcaggagggttaaatgagcctgagatgctttaaaaacctgtttttttaaaatttggttatatttaatgttaaatttttatttttttcttttagatgatgtctaactttttaaaaatgatgtttagtagtattatacgaatggggagttatgtagaaattggaagtatttcaattacattgtacttctaattgatgttttaagtttattgtacgatcttccatttaaataacagtctgtctaagatcatttgtttgatttgtcaattgttggtctatttgggtctgagaattccacaattttgaggaattttttgttaactatttatatattttgtagtttgaacagaggagtgtaaagcaattccagcagccgcagcagtagctgtgactgcaataaggcccataagactgttataagggtaaaaataaatctctttgttttggtaaacacttttttttaaaacatttttgtgacaatatgaatggaaggagaggctttctaaggtctattgagggaaaccagtatccaaactcctttcttagtttttatcagtaacacagatgtttttacaccgaacgtggaattaatacaggtgaaaaggtgacagttttgacaagtaatagtttgagaattaggtcgaatgtcaatatttttgaccattaacataaaaggagggttgacacaactctgaatgggcactgttttgttggaagaaaactgatacgcaaattgaagtttttaaccttttttttttaaagataatatatttttttctaaacttaaatatgagattgggccattattaactttcataatttggagtgtttagggcctattattggattaattattttgggatgtgggccagctgtactaaaattggtccaaattatgggaaaatgagcacgtttttcagtgtaagtagtgttacctttttgatagtatagtttctgttttagttttgtcttgtatttattattttgatgggtacaattaactgtaaaggtcccctcaggggaccaattaatgacaatttcataggaattattttgtagtaccatagtgtgatcagagatgtaattttttttaattaatatttttaaattatttgaccattgttaaggttgttggcacctcttttttgggggcttaaactgttaattgaattgaactctgtgaatgatccgggctccatccagaaaataaatgataggatactggtctttgattatgacctggaattttaactagtcaatgttgtcggtagccttttaggcaaccgatagttggccttatgtaaagaggggggaactgataacctatggacacatttattaacttttttttttttcctttgggtgagagggcccatgagtatttgtaggcttagggatccaaacgctattattaacataaacttcaactgggggttttaaccatgtgacaggcctaattaaaggcaggaatgggacacatgcccaataggtataattttgggctgttgtagccacaggtttgttaggcgaggaggtcactgtttttattttggctttgtattctaggattagtaaataacagaagacaaacatgagtataattagtaacttttttttttagtaaaagagtgacctgtagtgttacttggcatcttagtttactatatgttattaatgaggaaccccactgggggtatgttaatttattctagctaagcagttatgttattagaagctgagaagggggtgtttgttaaagtaacagggcagaagaaaggcggatttaagatacgagcttaatacagtgtagcaggtataggtagtaggcaaagtgagagaattaaaaatgaataaattatttggcttagacttttgtttttttagtataatgtctgaggcctgtgttgtttgtggaagtcgcattgttgaggctgtagttcctgtagggtcttttttaggctggttcaaatgtttttttattttttaattttttatcctttgatgaggatgtagtctttaggctggtactggaaattttaggagtggcgtctgtgttaagagactttttacaatttttaaagagcaggttagtgttttaagaaaaacttgtgttttattttaatgtttagtttatagaaaactggatgatatctttttaactttagtaaatacgtttacacacggaattttttacaattatcattttaaaacttgtttagatctttaaaacaaaattaaacaaccttttttgtataaattttttataactttttttatgacttttacagacaatttttaacatgtcttaactttttatgttttataatttttttactaaaggtacatttttataactttttaaatttttttacttttttgtatttttttgatttttgtcttagtcttttttttacttttatttttttaaatgtgtaataattagatgagtgttggtaacaatggatgtatgtacatattttagtttttaaaatttagggatgtgtttaacatctgtttgccagaactgactaggttccaattctttacggttaacacctattgaaggagggtatgtgcctgtgagctggtaatctgggcattgtgggataatttgtttagccagcctctgtgtaagttgaaattatttagataagtttctccaattttggtggaataatcgatgtgattgggtggcttggtcaagcagtgatgtcataacctgaaggtctgcttgattattgccgtaagccaatgggccaggcagagagctgtgggctcgaatgtgtgtaataaaagtaggatgtgtaccttggtctagtaattgttgaagttgaagaaaaagaccacacagagtgggctccagagcaaacttaaggctgtaatagtttttaaataaatacacagaataaccttagctctctgaatgttagtaaattcagatcaagtgattggattatgtggtctccaccagactgttgctttttcatgtttaccagacccaccagtaaaaacagctatggctccttccaaaggggcatcacaagtaatttttggaagaacctatgtagttaattttaagaattgaaaagtttttaggataatgattattaatacatccaacaaattttgttaaattaatctgtcatgtaactgagttaataaatgcctgtttaacctgatttttatttattggaactataatttttattgggctcagtgccacaaaatttaataattcatatatgagcctgtccaattagaattgccatctgatttaagtatactgtaagtgcttttatggtattatgtggcaaaaaggaccatttaactaaatcatcattttgaacaataacccccattattgtgtggttagtgtgaagtagggaacacaatgaattataaaggcaagtctgagtcaatcctactgacctgggcttgctgaattttgttttcaattactgataactctttcatggcctcgggtgttagttctctgttactgcgtaagttggtatttcccctcaatattgagaagagattagacatagcataagtaggaattgctaaattgggccaaatccaattaatatcttctaacaatttttgaaaattatttaaggttttgaaagaatctcttctaatttgaaccttttgaggcttaatggctctatcctgtacttgtattttcaaatactgaaaaggagtggttgtttgaattttgtcaggtgctataagtaattcagcatttgtaattgtcttttgcaaagattaataatattgaataagttggtctctactttttgctgcacaaatctggaaactgatctctaacaggctggatagttctgcctacaaaagtttgacaaactgtgggactatttaacataccctggggcaaaactttccaatgatatttggctgcaggttttttgttattaacggcaggaatggtaaaggcaaattttttgaaatctgcctctgctaaaggaattgtaaaaaagcagtcttttaaatctataataacaagcggtcagtctttagggagcacagtgggggatgggagcccaggttgtaaggctcccatcggttgaattacagcgttgacgccatctaccggactttttcttaattacaaatactggggaattccaaggagagaaagtgggtgaaatatatcctttttttagtagtttattttataaagcacccccaacttttccttagggagcggccactgttcaacccagacggggcgccgggtcatccattttaagggaaattgctccttcactgtaataactgtagggtgaacctgaattgccccatctccataatgaactgtgggtcgggcaataatgggcacggtgagccaagtctcgggctccctccccctgcacccactcggctgaggaggaggtggccattctggacatttctctacaggaaccgtgggctgaacaattttttgagtaggtttagggagactggggagattggcataaatcatcttcagactctcctttttgttagtactcggtagaggtggttcagagttctgattatcaaactcctctctctcctcctctgactcagcctcattatctgtctgaaaaggctccagtgctgcatgcaccaatgaccaaagcgaccaaacaggcaaaggaatttcctttccttctctatatgctcttttaaggtcctttccaactccttcttaatgttttaatttcaaagtttcctgttttgggaaccaagggcaaaattgttccatagcatgaaacaaatccataagattttccgtatcaacttttaccccaccatgcatgcttgaagagctgccgtaggaagctcaaatacgtggtgtacttactttcagtttttcccattgtgtccctagctttctctgggcgccccgcttacctgtagaggttaaaacttttatgtccttgggagtcctttgttcgttggtcctctgtttcacatgcttgagcgtttcctcaccagattcttttgggccccacgttgggcgccagaatgttggggaccagcctcaacaccacctgtagggtacctgaagtctggtggtgacaaaggaatgagaagagacaggttaagagttcataaagagtggaggccagggggccaattgcaaaatggaggctgcaaaaggctcagagctctggtctccacactatttattgagtacaataacttagatctaagaagcagatgttcagggcaaaacagtgaaagggtagcagtgcgtcacaggcataatctacagcagaagcgctttaaatgaatctcctttgtgctcaaacagcatatctttaacttatcggagagtagctagtgggagtgggcttaactaggagcctgcacgtctgtccacattccaatgcttcaaaggagggtctttctccttgaatacagtgtttacagataagagagagcaggtctcgctctgagcatggcaattaggaggcttttctcctcagaggcctcttgtggctttccacaacttattgtcccatatttttatggccagtttatacaggcaccccacaagtccttttcccaacacagacaggaatacggcagcctgtgccctgggagctcactgtcttgtgggagggaaccactcaagccactccccacttgtcctcctgtccctctcttcttgggctctgtcccccacctctctctgtcctttgtcttgcaggtggggagatggaggaggcagagctcacatcctggtattttgtgtcatctcccttctccttggatcttagcaagaccaagcgacaccttgtgcctggggcccccttcctgctgcaggtttcttccagaggggaaggatgagtagggaggatgtggtagttaggagggctcagggtctgaccactctcttttgcctgccctcctttacctgcctaggccttggtccgtgagatgtcaggctccccagcttctggcattcctgtcaaagtttctgccacggtgtcttctcctgggtctgttcctgaagtccaggacattcagcaaaacacagacgggagcggccaagtcagcattccaataattatccctcagaccatctcagagctgcagctctcagtaggactcctcggacccctgggagatggtgggggaaggggaggagggtgagctggggtcccaaggatccatggcctgacttggggggaaggtggggtacttggctctgagctactaccctattcgcacctgaccccctctccaggtatctgcaggctccccacatccagcgatagccaggctcactgtggcagccccaccttcaggaggccccgggtttctgtctattgagcggccggattctcgacctcctcgtgttggggacactctgaacctgaacttgcgagccgtgggcagtggggccaccttttctcattactactacatggtgtgcatgagctggggagtcacggagggctggggtgcagggaagagccctctgggtggggctgggggggttcaaggctgaggctgtcccatgaagaggcaaccactcttgtccctcccattcttggcccagatcctatcccgagggcagatcgtgttcatgaatcgagagcccaagaggaccctgacctcggtctcggtgtttgtggaccatcacctggcaccctccttctactttgtggccttctactaccatggagaccacccagtggccaactccctgcgagtggatgtccaggctggggcctgcgagggcaaggtgaccggggtcaggagagatggcacttgtgccgagggggttgaggacagggtgattgccaacagggcatggatttagcttgggggcagtgaggataccgggactgaaggaagctctcccactctgaccgcccccacctgccgcccctgccagctggagctcagcgtggacggtgccaagcagtaccggaacggggagtccgtgaagctccacttagaaaccgactccctagccctggtggcgctgggagccttggacacagctctgtatgctgcaggcagcaagtcccacaagcccctcaacatgggcaaggtttgtccagaccctctccacagctctctcacccctccatggctcatccccctgcttccctgagccttgggcgcagcccctggatcccactgaggctccccacagtctcttccccacttggccctgtggtctccatctcctggctctgtatcctttcctatccccccatgtgctgccctctcacctgtgccgagtgctcagtcctgcccctcagccacacttggctcctagcattcctgcctttcttgcaggtctttgaagctatgaacagctatgacctcggctgtggtcctgggggtggggacagtgcccttcaggtgttccaggcagcgggcctggccttttctgatggagaccagtggaccttatccagaaagagtgagaacagagaaggaaggggagtgggtggcgggaagataaggaaggaggaagggcctgaggggaccagctggaagagtccgggcaggaagggctgggcaggggaaggggaggaggggaggaggccgagtgcctgacggctggactgcagcctttctctctaccaggactaagctgtcccaaggagaagacaacccggaaaaagagaaacgtgaacttccaaaaggcgattaatgagaaatgtgagttgcgggtgcctaggcagtagcttgggctctccacctgggatccgggttgggggtctgcctctctgcccctcggctccttgctgaacccacgtgtggtatttggggccagagatccgaattccgggattacgagtggaaggtgggcagctctctccagcagcctctcttatgttgctggtctcaaggggtcggggcgggggctgaggtgtatgtcctttttgtcctctcatgctcacccccacctggccctgcagtgggtcagtatgcttccccgacagccaagcgctgctgccaggatggggtgacacgtctgcccatgatgcgttcctgcgagcagcgggcagcccgcgtgcagcagccggactgccgggagcccttcctgtcctgctgccaatttgctgagagtctgcgcaagaagagcagggacaagggccaggcgggcctccaacgaggtgaggggctgggtggggctagggcacaggtggcggcgcttggaaaggcagaacggtcccctcctcactcccgtccaccgtggtcccccagccctggagatcctgcaggaggaggacctgattgatgaggatgacattcccgtgcgcagcttcttcccagagaactggctctggagagtggaaacagtggaccgctttcaaatgtgagagtgtgtgccggcccggccttttctctgtgctgtgtctcggggccagccggggtagacgggccttctctgcctttccctacacagattgacactgtggctccccgactctctgaccacgtgggagatccatggcctgagcctgtccaaaaccaaaggtgatgtcaccctgtctgggcctcaggtgaccctgcttccatttccctgtaccccagctccctgttccctttgctcttagtgtaggaagagggtccagtgatctggggaggtctgtgccagcgtgcagctggcgtgggccagagggcagaggcggactgagacagagctgggtcacccccacccctccctcctgtggccctgaagctttgatggcccctctgatctctgcccctgtgcccacgcttcctttccctcaggcctatgtgtggccaccccagtccagctccgggtgttccgcgagttccacctgcacctccgcctgcccatgtctgtccgccgctttgagcagctggagctgcggcctgtcctctataactacctggataaaaacctgactgtgaggccccataggagcctgagcatacaggagttgggggagccagggcccagtgaggggtggggaggctaaccgggccaggactctggccatcctcgttttcctgccctcaggtgagcgtccacgtgtccccagtggaggggctgtgcctggctgggggcggagggctggcccagcaggtgctggtgcctgcgggctctgcccggcctgttgccttctctgtggtgcccacggcagccgccgctgtgtctctgaaggtggtggctcgagggtccttcgaattccctgtgggagatgcggtgtccaaggttctgcagattgaggtgaatggagcacccctgaatataagtccccgggcccccagctttgtcctccaccctcagcactctctctgctggccaggccaggggcccaacacccaaaccaatgccttggtctgttcccatcttctacaattctgatccaactctgtccctggagttgaaactcaaagttctgggggagtctgcgctagcagggcaggctgtagtcctgtgtgacctcacaaccatgttttccctgagacagaaggaaggggccatccatagagaggagctggtctatgaactcaaccccttgggtgagtgaccctctacctccagccattggtttcctaagtgggtacaggtggtgggggatgtggacagcaggacaggctgccaacttcccccatttccccagaccaccgaggccggaccttggaaatacctggcaactctgatcccaatatgatccctgatggggactttaacagctacgtcagggttacaggtgggagtgccctttagtcccttcccagtggccaccttcggattcatgtgggacttgtggatccctgcttggtcccactccccgtgagcctctgacacagagtcctcagacctccaccctctccctcccatgtagcctcagatccattggacactttaggctctgagggggccttgtcaccaggaggcgtggcctccctcttgaggcttcctcgaggctgtggggagcaaaccatgatctacttggctccgacactggctgcttcccgctacctggacaagacagagcagtggagcacactgcctcccgagaccaaggaccacgccgtggatctgatccagaaaggttctgggtgcaagggcaagcaggaggggggccaggaaaggacagttactggaagatggacagcccaggaggctacagagggaaagaaagggggcccctgatgaggatggggagcatggccttgggctcaaacagcagaagggtgagtgtcacctgagcggccacctctcctctccaaggctacatgcggatccagcagtttcggaaggcggatggttcctatgcggcttggttgtcacgggacagcagcacctggtgagcttgggagagtggttccagggttctgagggggtcagggctggggcaggggtgggacagagctggtatgatgggagggtggataaccaggcacctgggggcgtgggcataatgagaagcaagtccttatccccaaccctcctttcctgccctccaggctcacagcctttgtgttgaaggtcctgagtttggcccaggagcaggtaggaggctcgcctgagaaactgcaggagacatctaactggcttctgtcccagcagcaggctgacggctcgttccaggacccctgtccagtgttagacaggagcatgcaggtgcgggcatgctggggctggcccgagaagcgcctgtcggaggactctctttgccccttccccctcctgtttgacatcttttctccccttactaggggggtttggtgggcaatgatgagactgtggcactcacagcctttgtgaccatcgcccttcatcatgggctggccgtcttccaggatgagggtgcagagccattgaagcagagagtggtaagttcagtggcgtttctgccctctgctggcccccagctctctccctttttcctcaggaacccaggggtccaggcccaagaccctcctcccgttttcttccaggaagcctccatctcaaaggcaaactcatttttgggggagaaagcaagtgctgggctcctgggtgcccacgcagctgccatcacggcctatgccctgacactgaccaaggcgcctgtggacctgctcggtgttgcccacaacaacctcatggcaatggcccaggagactggaggtgaggggtgaggcgctcctggcagtgagcctgaggcccaggggaccttaggatccctgagtgtgcccagagggagaggctggatgaagactcagaggaggaatgaagttataagcaggggtgggttgggggagactcaggagagcccagcagggggtggctaagggccaggggaccaggctcttctccctgccttcctgtttactcgtggtctcccttcactttcagataacctgtactggggctcagtcactggttctcagagcaatgccgtgtcgcccaccccggctcctcgcaacccatccgaccccatgccccaggccccagccctgtggattgaaaccacagcctacgccctgctgcacctcctgcttcacgagggcaaagcagagatggcagaccaggcttcggcctggctcacccgtcagggcagcttccaagggggattccgcagtacccaagtaggggccgtccccgggctctggcgggggtgggtagtcctcagaccaagggcttgcttgagtcctggctcaacctccctaggacacggtgattgccctggatgccctgtctgcctactggattgcctcccacaccactgaggagaggggtctcaatgtgactctcagctccacaggccggaatgggttcaagtcccacgcgctgcagctgaacaaccgccagattcgcggcctggaggaggagctgcaggtgaaccactccctggtgaaccactccctcgcctgggtagccaggacacctgggcctcgtggccaggccagaagccgtccccaccctcccacccgtggaatccccgcagcacttcttcctggggtcttcgggggaagactgacttcctggctgtgtgacctggagctctgagcttcagttttctcacttgtagagtaacatacacagagttcaccctacagggtcgttagaaggctgaagtgagataattcatgtgctggtataaactttgtggaaatgtgaggtggggagaggaggtggggctgttttgaggaaggagataagttattggagccgcaaaaacaggtttgcttgtgcccttctaacatcgccttcccttttctgttgctgaagttttccttgggcagcaagatcaatgtgaaggtgggaggaaacagcaaaggaaccctgaaggtgagggccagggaaggggtggggccaggcactggtggaggagagggtgtggagtgagaggcctgtgggcagaggcacatggtccggggaaggaggcagacacctcagggttggtgtcccgtgcttccgtcctgggtgtttttccccctgcttgctttcgcttgctctccccatctctgggtacctgttgtttcctttacccgcctcagtgctggtggctccgaatcccactcctcagcccaggcctcttccctgaaccatgggccccactcgtcccactcccacagcacctcagacgaggcatgtcccaaagcccttcttcattctgtgtctcttgtctggctggtgggagcccctcccagccaggagcccagccactactctagaggccgtgttagtggcccctctcccaagcctgtccttatgtccctagtgactcctcctctgctcccctgctgcctgtggcccttggtgctgcatcctagattctgtgctgagacggccttctccctacctggaacttctctctacctcctgtctcccctgtctgatccactgtccacacggcagtgacactgaccttccaaaagccccagccagatcagccttggggaaaagtcactccccgctgcccacggctcagatggctgggcctctgcccacccctccggccagacagctctccttgtctacacagatccccttgcctttcctgtccttccctgcttcttggcccacaggacaagctctttcttctccttcaagccttggccagaagcctttcctgagcttttcagtccagcctcttcccagcacagtctggagtgttggcctctgggggcaggcccctgcttctttacctctctgtctcgcctgacgcctgtggcgaatgtggtgccactcgtgtgtgtggactgtgcagtgacggggaggaaaaggggctgaaggcctcaaatcctgtagcccagggagatgcccttaggtatggcaccagagaggtctgtggcctcacatgtcccacgtcctctccctgccccttgctgagccaggtccttcgtacctacaatgtcctggacatgaagaacacgacctgccaggacctacagatagaagtgacagtcaaaggccacgtcgagtacacgagtgagtgtgggggttgggaggccttggggccaggcaggggctggcgcagggagccgggtggccatcccagccctcctcacaatgcttccctgtgcagtggaagcaaacgaggactatgaggactatgagtacgatgagcttccagccaaggatgacccagatgcccctctgcagcccgtgacacccctgcagctgtttgagggtcggaggaaccgccgcaggagggaggcgcccaaggtggtggaggagcaggagtccagggtgcactacaccgtgtgcatctggtgggcgccgggagctgccctgggccaggggagggagggcaggacccaggctggggctgggcttctggagcccgcgcaggcagaacctggacgacagctcacacgtctccacaggcggaacggcaaggtggggctgtctggcatggccatcgcggacgtcaccctcctgagtggattccacgccctgcgtgctgacctggagaaggtgtggtcagccacccagggcaaccccctctgtcccaggtactgagccctgtcatgtgcagggcctgtgaccaactccccttttccacagctgacctccctctctgaccgttacgtgagtcactttgagaccgaggggccccacgtcctgctgtattttgactcggtgagtggggagagatgaggcaggaagggactcgatggcaccgggtttactgagtatgcgttaggaggtttctcaggagacagctgtgtcagcggctggtgctcttgagaacttgtgatgtcatcagagagaaggacaagaatgtgagcccgtgagacacagcagagtaaggggcagacctgcaggcggcagggaccgatgccagtcagcagggaccctcagggtttgagagggagtctttcctaatgctggttttattcagcttgaggggctgcctttgtttttttgttgaacttcctatcttttttttaatattaaagcgtattttcctttacaaagtgatggtggccatagatgatagttgtatttgtcttttcacgaccttatttggctaaaatagttatcaaccctcttacggctctcaaaacatttttatttatttatttagtaaagacagggtctcgctctgttgcccaggctggtcttgaactcccggcctcaagcgatcctctggcctaggcctttcaaagtaccggatttacaggccagagccaccatgcccggccttcaaaaaaagttttggaacatttactgtaacctctgggagaaaatgtgagaaaggtgtggtggctgtcattagccagctgtttgtaggtcagggagacccctacccagtgtgtgcagaggggccagcccccatcagctggggaagcctggctgacacatctgggttgaacacaatagaaaacacagagccaacaagattcccggatagggagctgacggtgcagcagcctagctcaggagggacactggcacggcaccgtgtggactgggcccgcgtgggcacgaggaggggtcaggcctgggacctgagtcggggggtcaggcaggatgacagaacctgcagttaggttgtggcaaataaaggaggacccagttgtatccatgacaaagatgaggccgcgaggagggcgagtgggtttgggggcaggcagagtgccttggagaacttacaggtcctgccacaatcctaatgcaaggatggagctgcaagttcagtttgggaatcatcagcctggattggtttggtggaagccagggagtggttgagacccccacaggggagctctgaggaaggaagttccgaaggagggaacgtaagaaatgaccaggtcagaaccaagggtggtccagaagctaacccttagcttagggacagtttcacagagaacacgtccatgatgcaagactctgctgagggcctggagcagtgaagactggggcaaggtcaccctctgggaagtgaagtcaccagagaccttgcggagcagctttgagagttctctgagtaggaaggtaacagaatgtgaaggacactggagagaaggccaataggaagcaaacaaaaacaggccaaggaaacccagtacagggggctgcagggcccagggagtgggtccctcatctctcctccccacgcttggccaggtccccacctcccgggagtgcgtgggctttgaggctgtgcaggaagtgccggtggggctggtgcagccggccagcgcaaccctgtacgactactacaaccccggtgagcactgcaggacaccctgaaattcaggagaactttggcataggtgccctcctatgggacaatggacaccggggtagtgagggggcagagagccctggggctccctgggactgaggaggcagaatggaggggcctgtgccctaactcctctctgttctccagagcgcagatgttctgtgttttacggggcaccaagtaagagcagactcttggccaccttgtgttctgctgaagtctgccagtgtgctgagggtgagactgagggcctggggcggggcagtggaggcgggatggccggggccccccccacactgtctgatgggttccccaacttcagggaagtgccctcgccagcgtcgcgccctggagcggggtctgcaggacgaggatggctacaggatgaagtttgcctgctactacccccgtgtggagtacggtcagtcttcccaccgaggccctggcctgaccctccctcggggaccggccgttttggtctctctgggtgtagcctgctcctcttacaggtcatgcacgcagcctgtttgctctgacaccaacttcctaccctctcagcctcaaagtaactcacctttcccccttctcctcaccccctcttaggcttccaggttaaggttctccgagaagacagcagagctgctttccgcctctttgagaccaagatcacccaagtcctgcacttcagtatgaagcaaaccggagaggcgggcagggctggggggagacagggaggctgaggtgtggccgaggacctgaccatctggaagtgtgaaaatccccttgggctgtcagaagccttgggcttggccataaatagggaggcagtggcacctctccatgggggtggcgaaggtggaatgagaggatctacacagagtccccagcctgggctcaccctgcaccttctcttcccctctgaccacttttgcgcacgtcatccccgcagccaaggatgtcaaggccgctgctaatcagatgcgcaacttcctggttcgagcctcctgccgccttcgcttggaacctgggaaagaatatttgatcatgggtctggatggggccacctatgacctcgagggacagtgagtcatctggtcccctcagtctcttgtcctccccatgcctcgccacctaggccttgcccctcagaagccagatgcctgtgctctccgtttccacctgccatcctcccgagccctgctgactgcccctttgccccctgcagcccccagtacctgctggactcgaatagctggatcgaggagatgccctctgaacgcctgtgccggagcacccgccagcgggcagcctgtgcccagctcaacgacttcctccaggagtatggcactcaggggtgccaggtgtgagggctgccctcccacctccgctgggaggaacctgaacctgggaaccatgaagctggaagcactgctgtgtccgctttcatgaacacagcctgggaccagggcatattaaaggcttttggcagcaaagtgtcagtgttggcagtgaagtgtcagtgtgtgttgctagggctgagagcagtgcccctgcccgatgcagttctgggcaggccaggttgacataaccttagactctctgagccctgatgacccttgggctgttcagctctgctagaacctcccagatgacccgctaggagtctagtgcttcacaggaccaccccgagcagaactgggacccaagagcctgcaccccaaggaccagagtccatgccaagaccacccttcagcttccaaggccctccactgcccggctgtcgccagtcaccacggcctcagacagggcttgtgctcagctgacacctgtgacacagctcttctgcctcatgagctgttgtccagctacacctccccgactctgtcctcgtgctgctggcggttctgaggtctgcagattttagctgagttccgggctgttgaaagcctgctgacgcttggttctgttatcagtggaatgaggtgactttcccggagttgtgcaatcctcaggtccggcagtgtcttcttccagttactggtttcaaacaagccaaaagtctgactttggtgtgtttgtgaatcctctgaggaagccgctgttctcctggggtctccccttcccaccggacctgcctaactttcccccatttagtggcacacctggggtcttcagagatgactccgcgtctgtccaaagaagtttggtgagatcagtttccgtagaggtcatgacagttcagcagcctgccatccagtcattcgacagaaattcgggaatctttcacttcatgccatgccctgtgccaggtgccagagatacagctgctcactccagggctcatcgctggggagacagataagaggacgggcagtccccaccctctgtgaaagatgtgatgtcagggagcagtgtggtcctgtggggcatctaaccaagtcaggggcattgccaggcagggacagggaaggcttcctggagcaggtggcctccaagtggggctctgaagactgagaaggagccaggaaaagagcaggggtagatgagggcatctggggcagaaggagaatatacaaaggcccagaggccgggggcaggacagggtacctttggggacattgcatgtaattgaccacattcggagtttggatttggaagtggtggaagagatggagatggtgagacaagtagtaagcacgtcagccttccaggtgcgctcctttccgatgagcactgtcttatcccacgtaactttgagaagtttgggcctttcccactgtggcagaggtttcctgaggctcttgcatacatggccctatggttgctcatcagatctttctcccagtagctgctcagcatggtggtggcataagcccattttccggagccagggattcagttgcagcaagacctggcccggtctgggaggtcaaccatgaagaaggcagtagctgtcattgcccaaccccagaaatcccaatcctgttttctccctctcagtcctgatcatggattcagcagcagcgaactcgccaatgtagtgggtggcacagccagggtcttgactctggctctgcagtagcacagtctggaaaagctctgaggggagagagacccccactggtccgagggtctggcacagagccagaaatgggggggaaggtatggggctgggtcgcctctgacctctcaggtaccatccaggaggccctggcctctcactgaacccggccactcctctttggcatggcctcttcccaaatccccaaactgcctccttactcacaaaagtggtctctgagtgtcagtccagtgggacccccaccccttatggcttcagttccccaaatagggctggacccttgatcctgatccagctgtggctatccagccccttcctggggactttggactttgagggggggcatgcccagttgtgctgggaatccatactttccctggctggagtagaacctgtggactgtagtcctgagggcagtcatgttc

SEQ ID NO: 5 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 6 - DNA, 27624 bp, Homo sapiens
atggtgctggtcctggaggcaccggctccgttctgcatctcctccccgcagtccctggggaaggggatccgcagcccacctgggagaggagagcaggggccagtccttttccaagccttaggccctggctgcccacccagcccccggccccgggcccgtgcgtccaggtacccgtggtgaaagaggtggacacgggcggcaggaggctctggccccacatggcctggagccgtgcattgtaggaggtggagggaaagaggccaaggagctggtgagatgtgatccctcctgggagcaggatctcctgtgggacagacaagggggggtcaggggagagggaggtggagaccctccgggagggccagaggcagcacctcctggaatcacccagggaggggagttgggtcagtggggccggggcacctggttctgtccaccaggggtgtggaagctgagcaggtagcctgcgggccggactgggggctcagtccaagtgagcagggcggtgcggggggtcacttccttggcctccaagtcccgaggggcctctagccctaggagggaaagcaggaagaggagatggggatgaggcccaacctggctccctctacctcctctccctgtcccacacaccccacagaccctacctgtggtgaaggtgatgctggctggggaagtgaggttggggccccgcaggccacgcactgtggcggtgtagttggtgtggaggacaaggtcatgcagggggtagtccaccgcgctgcctggggtctccgcctgcagaggcggggctgggagtgtagagaggggcatcaaggcctgccccctccatcctcggccagagtccagcctcccccctgcaatccccaccctgaacaagtcccctccagaggcctcaggcctgctcacccccaggggctgtgacctggacgtcataggtgtccacaggattctgggggggcttccagtgcagcacggcgaatccctcggtcaagttcagtgcacgcaactgtgtgggaccgtcaggaactgggggaaggggaggggctcagaagggtccccgcggctctctctactccgtgcctccccagactccactggcctcccgtccgcaatcggagcctccaccacctccctttcaccctcctcgttctctctcaactcccacccatgccgttttcttgactcccacctggagtttctgggtccgggcccggccgtccacctgcacactctgaggctcccctgaaaacgttggggatcgagggttacccagggaaccccagggcggctggagggtgggcagagtgcaggggggagaggaaatgcgaggcgatgagcacatggcaaaggcaccacctccgtccgccagctggtaggagactttgaagctgtccgcccgggatggtgggggcatccagttgaccttggctgaggtctccctgatttcactgaattggaggtcacgggggctctccagaactgcagaggggtcaaggaacaatgacgcaggcaggggcagggaggctcctccctgcgagtccccccctcgcctctgctccagcacaggctcaccaccccttttcctctagtccccaggaatggaagtcgctctgcagattcctccaggcccaccaccaactcgcccacccccaccgctggctgaggcactaggtcccccccgtgaagtacaaagacccccactttggggcagagtgtgtgtgggtccttacctgggctgagggtgcgggcggttccctggatgctgtcggccttgtggggtcctcgcagcccatacagtgtcaggctgtacagagtcccggaacgcaggtcccggagcacggccgagtgccgcgtccccggcaccatcagctcgcgctgcagcagtggacgcggatgcggctccagagtgcttggtgatggaaccccaaagcggagcaggaaggagtcgaaggcccccggtggggcctcccagttgagcctcagtgaactggtggtcacgtcagtcacagacagctgggacaggcggggccttgactcctctgaggtctgaccagcaggagccagccctgcacggagtgggtgggggagaagggattggagacagaagcacaccagcttggtgacccagagcacgtcccttccacccccctccctgcccccgtttctctatctgtaaccagggacttgcagccacaggggggtcctgtggggcagagctaaaggccactcgcatccagcccatccatcctctctccctggtacccgcctcacgctctttccctgcgaccaccccttctgagcccccgtttctcccttctgagtcctaggctagaggccggagacgcctggtggtacctgtggtgccctcagctgagaggggccccaggcgcttcccttcatggaggccatagaggaggaacctgtagcgggtgctgggctccaggcctgagatgaggatcttgctctggtcgccgtccacgagcaaggcctggggctgcccattcgtgtcctcatactggaccacgaaggaatcaaaggggccctgggccacgctccacgagaggcgcatggagtctggggttgtgtcggtcacggtcagcactcctaggcggggctcttcaggaggctcaggggcctctggggctaactctggggctggtgtgtcctcttctggggctgcgtgggagaagcccaggggagaatctgagtgaggggcgccatggggtgctccatttttatcttccaggcttggcccaaggctgaggtgggaagtttataggtccaggcccagtcagacaatgaagtcgctgtggcctcgtgactcctgcgagctcccgcgctgtctgagtcaggtgctcgcttcccccttccacaccccggtgtcctgccgagcccacctcgagatatcacaggctctggccccacccatgccgggatacattcactgagcttgaggagtgtggtgctcccttctgagagaagctgagggtggaactggctggttgaggtgactggcaaatcccaccagccgtgccgtggtcaggcctgtctgaggtgggcatcagcgagctctggaagaggagcctgtaccacaaatgcagccactgctgttggtttctgtgtccccgctcattttgttttccagtgatgttcctcttaagaaaatgctcctgactcatccacggcagggaggtttgccactatctggacaaggccacccttcggggaggcgacagcagccccagcgagtaatgaggagcagcggcagtgacggggcagagtcggggctgggagattagagagcccctcccagggcctttccctcccgcctggcctggctcctgctctggactccttgatggatgttgaagcccacagggctgcagactcctcctccttcctgggcacaggccaggtcaccccactccggcctgcccactcctgcagtcatctttgtcttcagaccaaatgcacaagtactttgttaaaggtatcccatctgcagctcaagcctgcagcccctcaccttttggtggctcctcaggcctctaggccttattcacctttcccctttcctgtgccacttctcctctagggcgccaggctgtccttggcatggtccggaaggcaaagtaccgggagctgctcctatcagagctcctgggccggcgggtgcctgtcgtggtgcggcttggcctcacctaccatgtgcacgacctcattggggcccagctagtggactggtgagtctttccctggcctctggcagattatggagcaatgacccaaagtgggatttcctcccagctcatgcttagtttcctagtgaaggccagtggctctcattcttctctggaacccgggagcaccccttcccaagttctaagttctcctcacagcttgagcctaggcgtctggctccagccttgtctttctcctgcacagcatctctaccacttcaggaaccctcctccgcctgccagagacatgaagattctgctcatcattgctcagctcctcagagtgggccgggaggggactagaagagctgcatgatggtggctgagacagggtcaccttgggaaggcttgggagccaggatgagtgtcgggctctcgtgtgtgcaaaaggtcagatgtgactgctgctgtttgcctggtttctgacccagtggtggggtttgagcaatgcttctctgcccttccatggaaagtggaaccagaaatggtgccaaggctgtggctgttccctttcgtgtaaaatggtgctgttattactctgtcttgaaataggaaggtgggatttctggggaggctggtgaaggagggcagggttcttttctctacgtgtcatgttaaaattgccaaataaagtacctctgcctgtgatattttctggatgtcctttatttactgtgacgtgtgtttgggtgccttgtttaggggtagaggtgaagtctgagctttgcctcattcagagaggaaaggggtcaggggttcactctgacgttcaggccattctccctgtggagtggtgagggtgtacctaatctcctaaaccacggaatttctgttagggcctaaaaaagcaaaagcctagtatagttcaatttgtgttggaatgaaagtaagagacaagtgtcttagaagcctgtcattgttttgtgagggcctttaaatatcctgtactcgtgggccatgttgggcccttgtacgcccaggtatacatgagcttgtgtgcacctataccctgatacagatatacctggtagggggaggtgctcaggcactggaatgagaggagttaacggggaaggacagggttatttctgggccaagattcagagtttcccatggacacccaggtgtccggggtgcccccacaactctgggcctgaggccagttgcacttcttggctgtcacgtggtttcccagcttagctgggctgggggaggagcaaggtccagagtcaactctgccccgaggcctagcttggccagaaggtagcagacagacagacggatctaacctctcttggatcctccagccatgaggctgctctgggggctgatctgggcatccagcttcttcaccttatctctgcagaagcccaggtcctggaggcgggatgctgggtgcttggattggggcagggctggcatcgggacccgattcaggagtgagggagagcaggggtggaggtgtcagagcgaagtctgactgctgatcctgtctgttctccccaggttgctcttgttctctccttctgtggttcatctgggggtccccctatcggtgggggtgcagctccaggatgtgccccgaggacaggtagtgaaaggatcagtgttcctgagaaacccatctcgtaataatgtcccctgctccccaaaggtggacttcacccttagctcagaaagagacttcgcactcctcagtctccaggtaaccagaccccatgccctcctgctgcttgtgggggcctcctgccctgttcccatctgtcttgtaagtgtcatcatcttcccactggcctcctcccctcctgtcttcccaccctggcattctccttccacgtttctcccttggtctctgtcctttttggtcagctgtctcttgctctgtgacccgctccctctccctctccctctcctgacaggtgcccttgaaagatgcgaagagctgtggcctccatcaactcctcagaggccctgaggtccagctggtggcccattcgccatggctaaaggactctctgtccagaacgacaaacatccagggtatcaacctgctcttctcctctcgccgggggcacctctttttgcagacggaccagcccatttacaaccctggccagcggggtgagtctcagccccagggcctcaacctttaaccccctccgagccctctcaggatgagtttggtgccccctaagtgagataacctgaaagaaagtgccacacagaaggggtgcttaggaaacatttgtcccctgctccctctgtggagtttgacccaccctccccttgcacatggacccctgctcacctctctcctcctccactcccagttcggtaccgggtctttgctctggatcagaagatgcgcccgagcactgacaccatcacagtcatggtggaggtgagtccccgacctctggccttcctgatcctggccactgatgtgacctcctgcctgtgagcacttctccccttgcagaactctcacggcctccgcgtgcggaagaaggaggtgtacatgccctcgtccatcttccaggatgactttgtgatcccagacatctcagagtgagcgctcccaatgtgggggctgcccccaagctacaccaccccaattcctgttaggctctccacctcccacacagaggcacgtccccagatgccctgaccctcagcctcctgagcctctggttaacccccacagtcctcttcccagggaagcaggctgctggctctccgtgccccactgtacagatgggctgagccccttccttgtccattctcaggccagggacctggaagatctcagcccgattctcagatggcctggaatccaacagcagcacccagtttgaggtgaagaaatatggtgagagctggaaactggagggacaggcagctgctttcctgaaggaaataagggtggaaggagaggtactgggagcagctcagggcagggagatatgggtgccacagccctgagcagaggggagtctttgagctggagtctgacctgcctatcccttcaccctgggtcagtccttcccaactttgaggtgaagatcacccctggaaagccctacatcctgacggtgccaggccatcttgatgaaatgcagttagacatccaggccaggtaatacctccctccccacctctgcccaccagcaccgggtcctgctccctactcagtatgaatgggctcctgcttccctgccctcgggccattattccccccagcccttggcccaccctcttctctctgccacgacaggtacatctatgggaagccagtgcagggggtggcatatgtgcgctttgggctcctagatgaggatggtaagaagactttctttcgggggctggagagtcagaccaaggtaggaaggagaataggggctggggaggggaaggggcaagggaggtgaggtgggagactcagtctcaccctatgtcctgtttctttctatgccccagctggtgaatggacagagccacatttccctctcaaaggcagagttccaggacgccctggagaagctgaatatgggcattactgacctccaggggctgcgcctctacgttgctgcagccatcattgagtctccaggtgggtgactttcccttattgtaaccccagacccttgcctctgacctctgagctaaccctctgtcctccggcaccaacaccaccccacttctcacatctcatctcagactcaaaaccaggaaacacccaggagacctggtttctctccaactctgtctctgtgactcggcccttttccctggctgagtttatttatttctttgctcgttctgctcattccttcactcctccagtggacatgtgttgttcaatgccccgtgctaggcctcagcatgcacagacatgttggggaccagcctcaacgccacccgtagggttcctgaagtccattggtgacacaggaatgagaagagacaggttaagagttcataaagagtgggggccagggggccaattgcaaaatggaggctgcaaaaggctcagagctctggtctccacactattttttgagtacagtcactcagatctaagaagcagatgttcagggagaaacagtgaaagggaggcagtgggtcataggcgtaatctatagcaatagagttttaaatgaatctcctttgtgctcaaacagcatgtctttaaattatcggagagtagctggtggaagtgggcttagctagaagactgcatgtctgtccaatgcttcaaaggagggtctttctccttgaacagagtgtttacagataagacagggggtctcactctgagcatgggaacatgatggcaattaggaggcttttcttctcagaggcctcttgtggctttccacaacttattgtctcatatttttatggacagtttatacaggcaccccacaagtccttttcccaacatgcccccctccctttttttttttttaaccgctattgctattatggcttatttgtggtgtttggtctgttttcagaagtgtcttttgcatctgtagactaaaagtaaacagcataaacagatacacattaaagtaaaatttgtaatagttgatcctttaatggtcttaatctgtttaagaggatttatgtttgaaagtccgtcagtagctccaatgagaatgtcagtctcaggcaggagggttaaatgagcctgagatgctttaaaaacctgtttttttaaaatttggttatatttaatgttaaatttttatttttttcttttagatgatgtctaactttttaaaaatgatgtttagtagtattatacgaatggggagttatgtagaaattggaagtatttcaattacattgtacttctaattgatgttttaagtttattgtacgatcttccatttaaataacagtctgtctaagatcatttgtttgatttgtcaattgttggtctatttgggtctgagaattccacaattttgaggaattttttgttaactatttatatattttgtagtttgaacagaggagtgtaaagcaattccagcagccgcagcagtagctgtgactgcaataaggcccataagactgttataagggtaaaaataaatctctttgttttggtaaacacttttttttaaaacatttttgtgacaatatgaatggaaggagaggctttctaaggtctattgagggaaaccagtatccaaactcctttcttagtttttatcagtaacacagatgtttttacaccgaacgtggaattaatacaggtgaaaaggtgacagttttgacaagtaatagtttgagaattaggtcgaatgtcaatatttttgaccattaacataaaaggagggttgacacaactctgaatgggcactgttttgttggaagaaaactgatacgcaaattgaagtttttaaccttttttttttaaagataatatatttttttctaaacttaaatatgagattgggccattattaactttcataatttggagtgtttagggcctattattggattaattattttgggatgtgggccagctgtactaaaattggtccaaattatgggaaaatgagcacgtttttcagtgtaagtagtgttacctttttgatagtatagtttctgttttagttttgtcttgtatttattattttgatgggtacaattaactgtaaaggtcccctcaggggaccaattaatgacaatttcataggaattattttgtagtaccatagtgtgatcagagatgtaattttttttaattaatatttttaaattatttgaccattgttaaggttgttggcacctcttttttgggggcttaaactgttaattgaattgaactctgtgaatgatccgggctccatccagaaaataaatgataggatactggtctttgattatgacctggaattttaactagtcaatgttgtcggtagccttttaggcaaccgatagttggccttatgtaaagaggggggaactgataacctatggacacatttattaacttttttttttttcctttgggtgagagggcccatgagtatttgtaggcttagggatccaaacgctattattaacataaacttcaactgggggttttaaccatgtgacaggcctaattaaaggcaggaatgggacacatgcccaataggtataattttgggctgttgtagccacaggtttgttaggcgaggaggtcactgtttttattttggctttgtattctaggattagtaaataacagaagacaaacatgagtataattagtaacttttttttttagtaaaagagtgacctgtagtgttacttggcatcttagtttactatatgttattaatgaggaaccccactgggggtatgttaatttattctagctaagcagttatgttattagaagctgagaagggggtgtttgttaaagtaacagggcagaagaaaggcggatttaagatacgagcttaatacagtgtagcaggtataggtagtaggcaaagtgagagaattaaaaatgaataaattatttggcttagacttttgtttttttagtataatgtctgaggcctgtgttgtttgtggaagtcgcattgttgaggctgtagttcctgtagggtcttttttaggctggttcaaatgtttttttattttttaattttttatcctttgatgaggatgtagtctttaggctggtactggaaattttaggagtggcgtctgtgttaagagactttttacaatttttaaagagcaggttagtgttttaagaaaaacttgtgttttattttaatgtttagtttatagaaaactggatgatatctttttaactttagtaaatacgtttacacacggaattttttacaattatcattttaaaacttgtttagatctttaaaacaaaattaaacaaccttttttgtataaattttttataactttttttatgacttttacagacaatttttaacatgtcttaactttttatgttttataatttttttactaaaggtacatttttataactttttaaatttttttacttttttgtatttttttgatttttgtcttagtcttttttttacttttatttttttaaatgtgtaataattagatgagtgttggtaacaatggatgtatgtacatattttagtttttaaaatttagggatgtgtttaacatctgtttgccagaactgactaggttccaattctttacggttaacacctattgaaggagggtatgtgcctgtgagctggtaatctgggcattgtgggataatttgtttagccagcctctgtgtaagttgaaattatttagataagtttctccaattttggtggaataatcgatgtgattgggtggcttggtcaagcagtgatgtcataacctgaaggtctgcttgattattgccgtaagccaatgggccaggcagagagctgtgggctcgaatgtgtgtaataaaagtaggatgtgtaccttggtctagtaattgttgaagttgaagaaaaagaccacacagagtgggctccagagcaaacttaaggctgtaatagtttttaaataaatacacagaataaccttagctctctgaatgttagtaaattcagatcaagtgattggattatgtggtctccaccagactgttgctttttcatgtttaccagacccaccagtaaaaacagctatggctccttccaaaggggcatcacaagtaatttttggaagaacctatgtagttaattttaagaattgaaaagtttttaggataatgattattaatacatccaacaaattttgttaaattaatctgtcatgtaactgagttaataaatgcctgtttaacctgatttttatttattggaactataatttttattgggctcagtgccacaaaatttaataattcatatatgagcctgtccaattagaattgccatctgatttaagtatactgtaagtgcttttatggtattatgtggcaaaaaggaccatttaactaaatcatcattttgaacaataacccccattattgtgtggttagtgtgaagtagggaacacaatgaattataaaggcaagtctgagtcaatcctactgacctgggcttgctgaattttgttttcaattactgataactctttcatggcctcgggtgttagttctctgttactgcgtaagttggtatttcccctcaatattgagaagagattagacatagcataagtaggaattgctaaattgggccaaatccaattaatatcttctaacaatttttgaaaattatttaaggttttgaaagaatctcttctaatttgaaccttttgaggcttaatggctctatcctgtacttgtattttcaaatactgaaaaggagtggttgtttgaattttgtcaggtgctataagtaattcagcatttgtaattgtcttttgcaaagattaataatattgaataagttggtctctactttttgctgcacaaatctggaaactgatctctaacaggctggatagttctgcctacaaaagtttgacaaactgtgggactatttaacataccctggggcaaaactttccaatgatatttggctgcaggttttttgttattaacggcaggaatggtaaaggcaaattttttgaaatctgcctctgctaaaggaattgtaaaaaagcagtcttttaaatctataataacaagcggtcagtctttagggagcacagtgggggatgggagcccaggttgtaaggctcccatcggttgaattacagcgttgacgccatctaccggactttttcttaattacaaatactggggaattccaaggagagaaagtgggtgaaatatatcctttttttagtagtttattttataaagcacccccaacttttccttagggagcggccactgttcaacccagacggggcgccgggtcatccattttaagggaaattgctccttcactgtaataactgtagggtgaacctgaattgccccatctccataatgaactgtgggtcgggcaataatgggcacggtgagccaagtctcgggctccctccccctgcacccactcggctgaggaggaggtggccattctggacatttctctacaggaaccgtgggctgaacaattttttgagtaggtttagggagactggggagattggcataaatcatcttcagactctcctttttgttagtactcggtagaggtggttcagagttctgattatcaaactcctctctctcctcctctgactcagcctcattatctgtctgaaaaggctccagtgctgcatgcaccaatgaccaaagcgaccaaacaggcaaaggaatttcctttccttctctatatgctcttttaaggtcctttccaactccttcttaatgttttaatttcaaagtttcctgttttgggaaccaagggcaaaattgttccatagcatgaaacaaatccataagattttccgtatcaacttttaccccaccatgcatgcttgaagagctgccgtaggaagctcaaatacgtggtgtacttactttcagtttttcccattgtgtccctagctttctctgggcgccccgcttacctgtagaggttaaaacttttatgtccttgggagtcctttgttcgttggtcctctgtttcacatgcttgagcgtttcctcaccagattcttttgggccccacgttgggcgccagaatgttggggaccagcctcaacaccacctgtagggtacctgaagtctggtggtgacaaaggaatgagaagagacaggttaagagttcataaagagtggaggccagggggccaattgcaaaatggaggctgcaaaaggctcagagctctggtctccacactatttattgagtacaataacttagatctaagaagcagatgttcagggcaaaacagtgaaagggtagcagtgcgtcacaggcataatctacagcagaagcgctttaaatgaatctcctttgtgctcaaacagcatatctttaacttatcggagagtagctagtgggagtgggcttaactaggagcctgcacgtctgtccacattccaatgcttcaaaggagggtctttctccttgaatacagtgtttacagataagagagagcaggtctcgctctgagcatggcaattaggaggcttttctcctcagaggcctcttgtggctttccacaacttattgtcccatatttttatggccagtttatacaggcaccccacaagtccttttcccaacacagacaggaatacggcagcctgtgccctgggagctcactgtcttgtgggagggaaccactcaagccactccccacttgtcctcctgtccctctcttcttgggctctgtcccccacctctctctgtcctttgtcttgcaggtggggagatggaggaggcagagctcacatcctggtattttgtgtcatctcccttctccttggatcttagcaagaccaagcgacaccttgtgcctggggcccccttcctgctgcaggtttcttccagaggggaaggatgagtagggaggatgtggtagttaggagggctcagggtctgaccactctcttttgcctgccctcctttacctgcctaggccttggtccgtgagatgtcaggctccccagcttctggcattcctgtcaaagtttctgccacggtgtcttctcctgggtctgttcctgaagtccaggacattcagcaaaacacagacgggagcggccaagtcagcattccaataattatccctcagaccatctcagagctgcagctctcagtaggactcctcggacccctgggagatggtgggggaaggggaggagggtgagctggggtcccaaggatccatggcctgacttggggggaaggtggggtacttggctctgagctactaccctattcgcacctgaccccctctccaggtatctgcaggctccccacatccagcgatagccaggctcactgtggcagccccaccttcaggaggccccgggtttctgtctattgagcggccggattctcgacctcctcgtgttggggacactctgaacctgaacttgcgagccgtgggcagtggggccaccttttctcattactactacatggtgtgcatgagctggggagtcacggagggctggggtgcagggaagagccctctgggtggggctgggggggttcaaggctgaggctgtcccatgaagaggcaaccactcttgtccctcccattcttggcccagatcctatcccgagggcagatcgtgttcatgaatcgagagcccaagaggaccctgacctcggtctcggtgtttgtggaccatcacctggcaccctccttctactttgtggccttctactaccatggagaccacccagtggccaactccctgcgagtggatgtccaggctggggcctgcgagggcaaggtgaccggggtcaggagagatggcacttgtgccgagggggttgaggacagggtgattgccaacagggcatggatttagcttgggggcagtgaggataccgggactgaaggaagctctcccactctgaccgcccccacctgccgcccctgccagctggagctcagcgtggacggtgccaagcagtaccggaacggggagtccgtgaagctccacttagaaaccgactccctagccctggtggcgctgggagccttggacacagctctgtatgctgcaggcagcaagtcccacaagcccctcaacatgggcaaggtttgtccagaccctctccacagctctctcacccctccatggctcatccccctgcttccctgagccttgggcgcagcccctggatcccactgaggctccccacagtctcttccccacttggccctgtggtctccatctcctggctctgtatcctttcctatccccccatgtgctgccctctcacctgtgccgagtgctcagtcctgcccctcagccacacttggctcctagcattcctgcctttcttgcaggtctttgaagctatgaacagctatgacctcggctgtggtcctgggggtggggacagtgcccttcaggtgttccaggcagcgggcctggccttttctgatggagaccagtggaccttatccagaaagagtgagaacagagaaggaaggggagtgggtggcgggaagataaggaaggaggaagggcctgaggggaccagctggaagagtccgggcaggaagggctgggcaggggaaggggaggaggggaggaggccgagtgcctgacggctggactgcagcctttctctctaccaggactaagctgtcccaaggagaagacaacccggaaaaagagaaacgtgaacttccaaaaggcgattaatgagaaatgtgagttgcgggtgcctaggcagtagcttgggctctccacctgggatccgggttgggggtctgcctctctgcccctcggctccttgctgaacccacgtgtggtatttggggccagagatccgaattccgggattacgagtggaaggtgggcagctctctccagcagcctctcttatgttgctggtctcaaggggtcggggcgggggctgaggtgtatgtcctttttgtcctctcatgctcacccccacctggccctgcagtgggtcagtatgcttccccgacagccaagcgctgctgccaggatggggtgacacgtctgcccatgatgcgttcctgcgagcagcgggcagcccgcgtgcagcagccggactgccgggagcccttcctgtcctgctgccaatttgctgagagtctgcgcaagaagagcagggacaagggccaggcgggcctccaacgaggtgaggggctgggtggggctagggcacaggtggcggcgcttggaaaggcagaacggtcccctcctcactcccgtccaccgtggtcccccagccctggagatcctgcaggaggaggacctgattgatgaggatgacattcccgtgcgcagcttcttcccagagaactggctctggagagtggaaacagtggaccgctttcaaatgtgagagtgtgtgccggcccggccttttctctgtgctgtgtctcggggccagccggggtagacgggccttctctgcctttccctacacagattgacactgtggctccccgactctctgaccacgtgggagatccatggcctgagcctgtccaaaaccaaaggtgatgtcaccctgtctgggcctcaggtgaccctgcttccatttccctgtaccccagctccctgttccctttgctcttagtgtaggaagagggtccagtgatctggggaggtctgtgccagcgtgcagctggcgtgggccagagggcagaggcggactgagacagagctgggtcacccccacccctccctcctgtggccctgaagctttgatggcccctctgatctctgcccctgtgcccacgcttcctttccctcaggcctatgtgtggccaccccagtccagctccgggtgttccgcgagttccacctgcacctccgcctgcccatgtctgtccgccgctttgagcagctggagctgcggcctgtcctctataactacctggataaaaacctgactgtgaggccccatgggagcctgagcatacaggagttgggggagccagggcccagtgaggggtggggaggctaaccgggccaggactctggccatcctcgttttcctgccctcaggtgagcgtccacgtgtccccagtggaggggctgtgcctggctgggggcggagggctggcccagcaggtgctggtgcctgcgggctctgcccggcctgttgccttctctgtggtgcccacggcagccaccgctgtgtctctgaaggtggtggctcgagggtccttcgaattccctgtgggagatgcggtgtccaaggttctgcagattgaggtgaatggagcacccctgaatataagtccccgggcccccagctttgtcctccaccctcagcactctctctgctggccaggccaggggcccaacacccaaaccaatgccttggtctgttcccatcttctacaattctgatccaactctgtccctggagttgaaactcaaagttctgggggagtctgcgctagcagggcaggctgtagtcctgtgtgacctcacaaccatgttttccctgagacagaaggaaggggccatccatagagaggagctggtctatgaactcaaccccttgggtgagtgaccctctacctccagccattggtttcctaagtgggtacaggtggtgggggatgtggacagcaggacaggctgccaacttcccccatttccccagaccaccgaggccggaccttggaaatacctggcaactctgatcccaatatgatccctgatggggactttaacagctacgtcagggttacaggtgggagtgccctttagtcccttcccagtggccaccttcggattcatgtgggacttgtggatccctgcttggtcccactccccgtgagcctctgacacagagtcctcagacctccaccctctccctcccatgtagcctcagatccattggacactttaggctctgagggggccttgtcaccaggaggcgtggcctccctcttgaggcttcctcgaggctgtggggagcaaaccatgatctacttggctccgacactggctgcttcccgctacctggacaagacagagcagtggagcacactgcctcccgagaccaaggaccacgccgtggatctgatccagaaaggttctgggtgcaagggcaagcaggaggggggccaggaaaggacagttactggaagatggacagcccaggaggctacagagggaaagaaagggggcccctgatgaggatggggagcatggccttgggctcaaacagcagaagggtgagtgtcacctgagcggccacctctcctctccaaggctacatgcggatccagcagtttcggaaggcggatggttcctatgcggcttggttgtcacggggcagcagcacctggtgagcttgggagagtggttccagggttctgagggggtcagggctggggcaggggtgggacagagctggtatgatgggagggtggataaccaggcacctgggggcgtgggcataatgagaagcaagtccttatccccaaccctcctttcctgccctccaggctcacagcctttgtgttgaaggtcctgagtttggcccaggagcaggtaggaggctcgcctgagaaactgcaggagacatctaactggcttctgtcccagcagcaggctgacggctcgttccaggacctctctccagtgatacataggagcatgcaggtgcgggcatgctggggctggcccgagaagcgcctgtcggaggactctctttgccccttccccctcctgtttgacatcttttctccccttactaggggggtttggtgggcaatgatgagactgtggcactcacagcctttgtgaccatcgcccttcatcatgggctggccgtcttccaggatgagggtgcagagccattgaagcagagagtggtaagttcagtggcgtttctgccctctgctggcccccagctctctccctttttcctcaggaacccaggggtccaggcccaagaccctcctcccgttttcttccaggaagcctccatctcaaaggcaagctcatttttgggggagaaagcaagtgctgggctcctgggtgcccacgcagctgccatcacggcctatgccctgacactgaccaaggcccctgcggacctgcggggtgttgcccacaacaacctcatggcaatggcccaggagactggaggtgaggggtgaggggctctggcagtgagcctgaggcccaggggaccttaggatccctgagtgtgcccagagggagaggctggatgaagactcagaggaggaatgaagttataagcaggggtgggttgggggagactcaggagagcccagcagggggtggctaagggccaggggaccaggctcttctccctgccttcctgtttactcgtggtctcccttcactttcagataacctgtactggggctcagtcactggttctcagagcaatgccgtgtcgcccaccccggctcctcgcaacccatccgaccccatgccccaggccccagccctgtggattgaaaccacagcctacgccctgctgcacctcctgcttcacgagggcaaagcagagatggcagaccaggctgcggcctggctcacccgtcagggcagcttccaagggggattccgcagtacccaagtaggggccgtccccgggctctggcgggggtgggtagtcctcagaccaagggcttgcttgagtcctggctcaacctccctaggacacggtgattgccctggatgccctgtctgcctactggattgcctcccacaccactgaggagaggggtctcaatgtgactctcagctccacaggccggaatgggttcaagtcccacgcgctgcagctgaacaaccgccagattcgcggcctggaggaggagctgcaggtgaaccactccctggtgaaccactccctcgcctgggtagccaggacacctgggcctcgtggccaggccagaagccgtccccaccctcccacccgtggaatccccgcagcacttcttcctggggtcttcgggggaagactgacttcctggctgcgtgacctggagctctgagcttcagttttctcacttgtagagtaacatacacagagttcaccctacagggtcgttagaaggctgaagtgagataattcatgtgctggtataaactttgtggaaatgtgaggtggggagagggggtggggctgttttgaggaaggagataagttattggagccgcaaaaacaggtttgcttgtgcccttctaacatcgccttcccttttctgttgctgaagttttccttgggcagcaagatcaatgtgaaggtgggaggaaacagcaaaggaaccctgaaggtgagggccagggaaggggtggggccaggcactggtggaggagagggtgtggagtgagaggcctgtgggcagaggcacatggtccggggaaggaggcagacacctcagggttggtgtcccgtgcttccgtcctgggtgtttttccccctgcttgctttcgcttgctctccccatctctgggtacctgttgtttcctttacccgcctcagtgctggtggctccgaatcccactcctcagcccaggcctcttccctgaaccatgggccccactcgtcccactcccacagcacctcagacgaggcatgtcccaaagcccttcttcattctgtgtctcttgtctggctggtgggagcccctcccagccaggagcccagccactactctagaggccgtgttagtggcccctctcccaagcctgtccttatgtccctagtgactcctcctctgctcccctgctgcctgtggcccttggtgctgcatcctagattctgtgctgagacggccttctccctacctggaacttctctctacctcctgtctcccctgtctgatccactgtccacacggcagtgacactgaccttccaaaagccccagccagatcagccttggggaaaagtcactccccgctgcccacggctcagatggctgggcctctgcccacccctccggccagacagctctccttgtctacacagatccccttgcctttcctgtccttccctgcttcttggcccacaggacaagctctttcttctccttcaagccttggccagaagcctttcctgagcttttcagtccagcctcttcccagcacagtctggagtgttggcctctgggggcaggcccctgcttctttacctctctgtctcgcctgacgcctgtggcgaatgtggtgccactcgtgtgtgtggactgtgcagtgacggggaggaaaaggggctgaaggcctcaaatcctgtagcccagggagatgcccttaggtatggcaccagagaggtctgtggcctcacatgtcccacgtcctctccctgccccttgctgagccaggtccttcgtacctacaatgtcctggacatgaagaacacgacctgccaggacctacagatagaagtgacagtcaaaggccacgtcgagtacacgagtgagtgtgggggttgggaggccttggggccaggcaggggctggcgcagggagccgggtggccatcccagccctcctcacaatgcttccctgtgcagtggaagcaaacgaggactatgaggactatgagtacgatgagcttccagccaaggatgacccagatgcccctctgcagcccgtgacacccctgcagctgtttgagggtcggaggaaccgccgcaggagggaggcgcccaaggtggtggaggagcaggagtccagggtgcactacaccgtgtgcatctggtgggcgccgggagctgccctgggccaggggagggagggcaggacccaggctggggctgggcttctggagcccgcgcaggcagaacctggacgacagctcacacgtctccacaggcggaacggcaaggtggggctgtctggcatggccatcgcggacgtcaccctcctgagtggattccacgccctgcgtgctgacctggagaaggtgtggtcagccacccagggcaaccccctctgtcccaggtactgagccctgtcatgtgcagggcctgtgaccaactccccttttccacagctgacctccctctctgaccgttacgtgagtcactttgagaccgaggggccccacgtcctgctgtattttgactcggtgagtggggagagatgaggcaggaagggactcgatggcaccgggtttactgagtatgcgttaggaggtttctcaggagacagctgtgtcagcggctggtgctcttgagaacttgtgatgtcatcagagagaaggacaagaatgtgagcccgtgagacacagcagagtaaggggcagacctgcaggcggcagggaccgatgccagtcagcagggaccctcagggtttgagagggagtctttcctaatgctggttttattcagcttgaggggctgcctttgtttttttgttgaacttcctatcttttttttaatattaaagcgtattttcctttacaaagtgatggtggccatagatgatagttgtatttgtcttttcacgaccttatttggctaaaatagttatcaaccctcttacggctctcaaaacatttttatttatttatttagtaaagacagggtctcgctctgttgcccaggctggtcttgaactcccggcctcaagcgatcctctggcctaggcctttcaaagtaccggatttacaggccagagccaccatgcccggccttcaaaaaaagttttggaacatttactgtaacctctgggagaaaatgtgagaaaggtgtggtggctgtcattagccagctgtttgtaggtcagggagacccctacccagtgtgtgcagaggggccagcccccatcagctggggaagcctggctgacacatctgggttgaacacaatagaaaacacagagccaacaagattcccggatagggagctgacggtgcagcagcctagctcaggagggacactggcacggcaccgtgtggactgggcccgcgtgggcacgaggaggggtcaggcctgggacctgagtcggggggtcaggcaggatgacagaacctgcagttaggttgtggcaaataaaggaggacccagttgtatccatgacaaagatgaggccgcgaggagggcgagtgggtttgggggcaggcagagtgccttggagaacttacaggtcctgccacaatcctaatgcaaggatggagctgcaagttcagtttgggaatcatcagcctggattggtttggtggaagccagggagtggttgagacccccacaggggagctctgaggaaggaagttccgaaggagggaacgtaagaaatgaccaggtcagaaccaagggtggtccagaagctaacccttagcttagggacagtttcacagagaacacgtccatgatgcaagactctgctgagggcctggagcagtgaagactggggcaaggtcaccctctgggaagtgaagtcaccagagaccttgcggagcagctttgagagttctctgagtaggaaggtaacagaatgtgaaggacactggagagaaggccaataggaagcaaacaaaaacaggccaaggaaacccagtacagggggctgcagggcccagggagtgggtccctcatctctcctccccacgcttggccaggtccccacctcccgggagtgcgtgggctttgaggctgtgcaggaagtgccggtggggctggtgcagccggccagcgcaaccctgtacgactactacaaccccggtgagcactgcaggacaccctgaaattcaggagaactttggcataggtgccctcctatgggacaatggacaccggggtagtgagggggcagagagccctggggctccctgggactgaggaggcagaatggaggggcctgtgccctaactcctctctgttctccagagcgcagatgttctgtgttttacggggcaccaagtaagagcagactcttggccaccttgtgttctgctgaagtctgccagtgtgctgagggtgagactgagggcctggggcggggcagtggaggcgggatggccggggccccccccacactgtctgatgggttccccaacttcagggaagtgccctcgccagcgtcgcgccctggagcggggtctgcaggacgaggatggctacaggatgaagtttgcctgctactacccccgtgtggagtacggtcagtcttcccaccgaggccctggcctgaccctccctcggggaccggccgttttggtctctctgggtgtagcctgctcctcttacaggtcatgcacgcagcctgtttgctctgacaccaacttcctaccctctcagcctcaaagtaactcacctttcccccttctcctcaccccctcttaggcttccaggttaaggttctccgagaagacagcagagctgctttccgcctctttgagaccaagatcacccaagtcctgcacttcagtatgaagcaaaccggagaggcgggcagggctggggggagacagggaggctgaggtgtggccgaggacctgaccatctggaagtgtgaaaatccccttgggctgtcagaagccttgggcttggccataaatagggaggcagtggcacctctccatgggggtggcgaaggtggaatgagaggatctacacagagtccccagcctgggctcaccctgcaccttctcttcccctctgaccacttttgcgcacgtcatccccgcagccaaggatgtcaaggccgctgctaatcagatgcgcaacttcctggttcgagcctcctgccgccttcgcttggaacctgggaaagaatatttgatcatgggtctggatggggccacctatgacctcgagggacagtgagtcatctggtcccctcagtctcttgtcctccccatgcctcgccacctaggccttgcccctcagaagccagatgcctgtgctctccgtttccacctgccatcctcccgagccctgctgactgcccctttgccccctgcagcccccagtacctgctggactcgaatagctggatcgaggagatgccctctgaacgcctgtgccggagcacccgccagcgggcagcctgtgcccagctcaacgacttcctccaggagtatggcactcaggggtgccaggtgtgagggctgccctcccacctccgctgggaggaacctgaacctgggaaccatgaagctggaagcactgctgtgtccgctttcatgaacacagcctgggaccagggcatattaaaggcttttggcagcaaagtgtcagtgttggcagtgaagtgtcagtgtgtgttgctagggctgagagcagtgcccctgcccgatgcagttctgggcaggccaggttgacataaccttagactctctgagccctgatgacccttgggctgttcagctctgctagaacctcccagatgacccgctaggagtctagtgcttcacaggaccaccccgagcagaactgggacccaagagcctgcaccccaaggaccagagtccatgccaagaccacccttcagcttccaaggccctccactgcccggctgtcgccagtcaccacggcctcagacagggcttgtgctcagctgacacctgtgacacagctcttctgcctcatgagctgttgtccagctacacctccccgactctgtcctcgtgctgctggcggttctgaggtctgcagattttagctgagttccgggctgttgaaagcctgctgacgcttggttctgttatcagtggaatgaggtgactttcccggagttgtgcaatcctcaggtccggcagtgtcttcttccagttactggtttcaaacaagccaaaagtctgactttggtgtgtttgtgaatcctctgaggaagccgctgttctcctggggtctccccttcccaccggacctgcctaactttcccccatttagtggcacacctggggtcttcagagatgactccgcgtctgtccaaagaagtttggtgagatcagtttccgtagaggtcatgacagttcagcagcctgccatccagtcattcgacagaaattcgggaatctttcacttcatgccatgccctgtgccaggtgccagagatacagctgctcactccagggctcatcgctggggagacagataagaggacgggcagtccccaccctctgtgaaagatgtgatgtcagggagcagtgtggtcctgtggggcatctaaccaagtcaggggcattgccaggcagggacagggaaggcttcctggagcaggtggcctccaagtggggctctgaagactgagaaggagccaggaaaagagcaggggtagatgagggcatctggggcagaaggagaatatacaaaggcccagaggccgggggcaggacagggtacctttggggacattgcatgtaattgaccacattcggagtttggatttggaagtggtggaagagatggagatggtgagacaagtagtaagcacgtcagccttccaggtgcgctcctttccgatgagcactgtcttatcccacgtaactttgagaagtttgggcctttcccactgtggcagaggtttcctgaggctcttgcatacatggccctatggttgctcatcagatctttctcccagtagctgctcagcatggtggtggcataagcccattttccggagccagggattcagttgcagcaagacatggcccggtctgggaggtcaaccatgaagaaggcagtagctgtcattgcccaaccccagaaatcccaatcctgttttctccctctcagtcctgatcatggattcagcagcagcgaactcgccaatgtagtgggtggcacagccagggtcttgactctggctctgcagtagcacagtctggaaaagctctgaggggagagagacccccactggtccgagggtctggcacagagccagaaatgggggggaaggtatggggctgggtcgcctctgacctctcaggtaccatccaggaggccctggcctctcactgaacccggccactcctctttggcatggcctcttcccaaatccccaaactgcctccttacccacaaaagtggtctctgagtgtcagtccagtgggacccccaccccttatggcttcagttccccaaatagggctggacccttgatcctgatccagctgtggctatccagccccttcctggggactttggactttgaggggggcatgcccagttgtgctgggaatccatactttccctggctggagtagaacctgtggactgtagtcctgagggcagtcatgttct

SEQ ID NO: 7 - DNA, 7658 bp, Homo sapiens
tgtggggaaaagcaagagagatcaaattgttactgtgtctgtgtagaaagaagtagacataggagactccattttgttatgtgctaagaaaaattcttctgccttgagattctgttaatctatgaccttacccccaaccccgtgctctctgaaacgtgtgctgtgtcaactcagggttgaatggattaagggcggtgcaggatgtgctttgttaaacagatgcttgaaggcagcatgctccttaagagtcatcaccactccctaatctcaagtacccagggacacaaaaactgcggaaggccgcagggacctctgcctaggaaagccaggtattgtccaaggtttctccccatgtgatagtctgaaatatggcctcgtgggaagggaaagacctgaccgtcccccagcccgacacctgtaaagggtctgtgctgaggaggattagtaaaagaggaaggaatgcctcttgcagttgagacaagaggaaggcatctgtctcctgcctgtccctgggcaatggaatgtctcggtataaaacccgattgtatgctccatctactgagatagggaaaaaccgccttagggctggaggtgggacctgcgggcagcaatactgctttgtaaagcattgagatgtttatgtgtatgcatatccaaaagcacagcacttaatcctttacattgtctatgatgccaagacctttgttcacgtgtttgtctgctgaccctctccccacaattgtcttgtgaccctgacacatccccctctttgagaaacacccacagatgatcaataaatactaagggaactcagaggctggcgggatcctccatatgctgaacgctggttccccgggtccccttatttctttctctatactttgtctctgtgtctttttcttttccaaatctctcgtcccaccttacgagaaacacccacaggtgtgtaggggcaacccacccctacatctggtgcccaacgtggaggcttttctctagggtgaaggtacgctcgagcgtggtcattgaggacaagtcgacgagagatcccgagtacatctacagtcagccttacggtaagcttgcgcgctcggaagaagctagggtgataatggggcaaactaaaagtaaaattaaaagtaaatatgcctcttatctcagctttattaaaattcttttaaaaagagggggagttaaagtatctacaaaaaatctaatcaagctatttcaaataatagaacaattttgcccatggtttccagaacaaggaacttcagatctaaaagattggaaaagaattggtaaggaactaaaacaagcaggtaggaagggtaatatcattccacttacagtatggaatgattgggccattattaaagcagctttagaaccatttcaaacagaagaagatagcatttcagtttctgatgcccctggaagctgtttaatagattgtaatgaaaacacaaggaaaaaatcccagaaagaaaccgaaagtttacattgcgaatatgtagcagagccggtaatggctcagtcaacgcaaaatgttgactataatcaattacaggaggtgatatatcctgaaacgttaaaattagaaggaaaaggtccagaattaatggggccatcagagtctaaaccacgaggcacaagtcctcttccagcaggtcaggtgctcgtaagattacaacctcaaaagcaggttaaagaaaataagacccaaccgcaagtagcctatcaatactggccgctggctgaacttcagtatcggccacccccagaaagtcagtatggatatccaggaatgcccccagcaccacagggcagggcgccataccatcagccgcccactaggagacttaatcctatggcaccacctagtagacagggtagtgaattacatgaaattattgataaatcaagaaaggaaggagatactgaggcatggcaattcccagtaacgttagaaccgatgccacctggagaaggagcccaagagggagagcctcccacagttgaggccagatacaagtctttttcgataaaaatgctaaaagatatgaaagagggagtaaaacagtatggacccaactccccttatatgaggacattattagattccattgcttatggacatagactcattccttatgattgggagattctggcaaaatcgtctctctcaccctctcaatttttacaatttaagacttggtggattgatggggtacaagaacaggtccgaagaaatagggctgccaatcctccagttaacatagatgcagatcaactattaggaataggtcaaaattggagtactattagtcaacaagcattaatgcaaaatgaggccattgagcaagttagagctatctgccttagagcctgggaaaaaatccaagacccaggaagtacctgcccctcatttaatacagtaagacaaggttcaaaagagccctaccctgattttgtggcaaggctccaagatgttgctcaaaagtcaattgccgatgaaaaagccggtaaggtcatagtggagttgatggcatatgaaaacgccaatcctgagtgtcaatcagccattaagccattaaaaggaaaggttcctgcaggatcagatgtaatctcagaatatgtaaaagcctgtgatggaatcggaggagctatgcataaagctatgcttatggctcaagcaataacaggagttgttttaggaggacaagttagaacatttggaggaaaatgttataattgtggtcaaattggtcacttaaaaaagaattgcccagtcttaaacaaacagaatataactattcaagcaactacaacaggtagagagccacctgacttatgtccaagatgtaaaaaaggaaaacattgggctagtcaatgtcgttctaaatttgataaaaatgggcaaccattgtcgggaaacgagcaaaggggccagcctcaggccccacaacaaactggggcattcccaattcagccatttgttcctcagggttttcagggacaacaacccccactgtcccaagtgtttcagggaataagccagttaccacaatacaacaattgtccctcaccacaagcggcagtgcagcagtagatttatgtactatacaagcagtctctctgcttccaggggagcccccacaaaaaatccctacaggggtatatggcccactgcctgaggggactgtaggactaatcttgggaagatcaagtctaaatctaaaaggagttcaaattcatactagtgtggttgattcagactataaaggcgaaattcaattggttattagctcttcaattccttggagtgccagtccaagagacaggattgctcaattattactcctgccatatattaagggtggaaatagtgaaataaaaagaataggagggcttgtaagcactgatccaacaggaaaggctgcatattgggcaagtcaggtctcagagaacagacctgtgtgtaaggccattattcaaggaaaacagtttgaagggttggtagacactggagcagatgtctctattattgctttaaatcagtggccaaaaaactggcctaaacaaaaggctgttacaggacttgtcggcataggcacagcctcagaagtgtatcaaagtatggagattttacattgcttagggccagataatcaagaaagtactgttcagccaatgattacttcaattcctcttaatctgtggggtcgagatttattacaacaatggggtgcggaaatcaccatgcccgctccattatatagccccacgagtcaaaaaatcatgaccaagatgggatatataccaggaaagggactagggaaaaatgaagatggcattaaagttccagttgaggctaaaataaatcaagaaagagaaggaatagggtatcctttttaggggcggtcactgtagagcctcctaaacccataccactaacttggaaaacagaaaaaccggtgtgggtaaatcagtggccgctaccaaaacaaaaactggaggctttacatttattagcaaatgaacagttagaaaagggtcacattgagccttcgttctcaccttggaattctcctgtgtttgtaattcagaagaaatcaggcaaatggcatacgttaactgacttaagggctgtaaacgccgtaattcaacccatggggcctctccaacccgggttgccctctccggccatgatcccaaaagattggcctttaattataattgatctaaaggattgcttttttaccatccctctggcagagcaggattgtgaaaaatttgcctttactataccagccataaataataaagaaccagccaccaggtttcagtggaaagtgttacctcagggaatgcttaatagtccaactatttgtcagacttttgtaggtcgagctcttcaaccagtgagagaaaagttttcagactgttatattattcattatattgatgatattttatgtgctgcagaaacgaaagataaattaattgactgttatacatttctgcaagcagaggttgccaatgctggactggcaatagcatctgataagatccaaacctctactccttttcattatttagggatgcagatagaaaatagaaaaattaagccacaaaaaatagaaataagaaaagacacattaaaaacactaaatgattttcaaaaattactaggagatattaattggattcggccaactctaggcattcctacttatgccatgtcaaatttgttctctatcttaagaggagactcagacttaaatagtcaaagaatattaaccccagaggcaacaaaagaaattaaattagtggaagaaaaaattcagtcagcgcaaataaatagaatagatcccttagccccactccaacttttgatttttgccactgcacattctccaacaggcatcattattcaaaatactgatcttgtggagtggtcattccttcctcacagtacagttaagacttttacattgtacttggatcaaatagctacattaatcggtcagacaagattacgaataacaaaattatgtggaaatgacccagacaaaatagttgtccctttaaccaaggaacaagttagacaagcctttatcaattctggtgcatggcagattggtcttgctaattttgtgggacttattgataatcattacccaaaaacaaagatcttccagttcttaaaattgactacttggattctacctaaaattaccagacgtgaacctttagaaaatgctctaacagtatttactgatggttccagcaatggaaaagcagcttacacagggccgaaagaacgagtaatcaaaactccatatcaatcggctcaaagagcagagttggttgcagtcattacagtgttacaagattttgaccaacctatcaatattatatcagattctgcatatgtagtacaggctacaagggatgttgagacagctctaattaaatatagcatggatgatcagttaaaccagctattcaatttattacaacaaactgtaagaaaaagaaatttcccattttatattactcatattcgagcacacactaatttaccagggcctttgactaaagcaaatgaacaagctgacttactggtatcatctgcactcataaaagcacaagaacttcatgctttgactcatgtaaatgcagcaggattaaaaaacaaatttgatgtcacatggaaacaggcaaaagatattgtacaacattgcacccagtgtcaagtcttacacctgcccactcaagaggcaggagttaatcccagaggtctgtgtcctaatgcattatggcaaatggatgtcacgcatgtaccttcatttggaagattatcatatgttcatgtaacagttgatacttattcttattcacatttcatatgggcaacttgccaaacaggagaaagtacttcccatgttaaaaaacatttattgtcttgttttgctgtaatgggagttccagaaaaaatcaaaactgacaatggaccaggatattgtagtaaagctttccaaaaattcttaagtcagtggaaaatttcacatacaacaggaattccttataattcccaaggacaggccatagttgaaagaactaatagaacactcaaaactcaattagttaaacaaaaagaagggggagacagtaaggagtgtaccactcctcagatgcaacttaatctagcactctatactttaaattttttaaacatttatagaaatcagactactacttctgcagaacaacatcttactggtaaaaagaacagcccacatgaaggaaaactaatttggtggaaagataataaaaataagacatgggaaatagggaaggtgataacgtgagggagaggttttgcttgtgtttcaccaggagaaaatcagcttcctgtttggttacccactagacatttgaagttctacaatgaacccatcggagatgcaaagaaaagggcctccacggagagggtaacaccagtcacatggatggataatcctatagaagtatatgttaatgatagtgtatgggtacctggccccatagatgatcgctgccctgccaaacctgaggaagaagggatgatgataaatatttccattgggtatcgttatcctcctatttgcctagggagagcaccaggatgtttaatgcctgcagtccaaaattggttggtagaagtacctactgtcagtcccatcagtagattcacttatcacatggtaagcgggatgtcactcaggccacgggtaaattatttacaagacttttcttatcaaagatcattaaaatttagacctaaagggaaaccttgccccaaggaaattcccaaagaatcaaaaaatacagaagttttagtttgggaagaatgtgtggccaatagtgcggtgatattataaaacaatgaatttggaactattatagattgggcacctcgaggtcaattctaccacaattgctcaggacaaactcagtcgtgtccaagtgcacaagtgagtccagctgttgatagcgacttaacagaaagtttagacaaacataagcataaaaaattgcagtctttctacccttgggaatggggagaaaaaggaatctctaccccaagaccaaaaatagtaagtcctgtttctggtcctgaacatccagaattatggaggcttactgtggcctcacaccacattagaatttggtctggaaatcaaactttagaaacaagagattgtaagccattttatactgtcgacctaaattccagtctaacagttcctttacaaagttgcgtaaagcccccttatatgctagttgtaggaaatatagttattaaaccagactcccagactataacctgtgaaaattgtagattgcttacttgcattgattcaacttttaattggcaacaccgtattctgctggtgagagcaagagagggcgtgtggatccctgtgtccatggaccgaccgtgggaggcctcaccatccgtccatattttgactgaagtattaaaaggtgttttaaatagatccaaaagattcatttttactttaattgcagtgattatgggattaattgcagtcacagctacggctgctgtagcaggagttacattgcactcttctgttcagtcagta

SEQ ID NO: 8 - protein, 6 aa, Mus sp.
PCPVIH

SEQ ID NO: 9 - DNA, 24 bp, Artificial Sequence
  Description of Artificial Sequence Synthetic primer
cctttgtgttgaaggtcctgagtt

SEQ ID NO: 10 - DNA, 24 bp, Artificial Sequence
  Description of Artificial Sequence Synthetic primer
tcctgtctaacactggacaggggt

SEQ ID NO: 11 - DNA, 23 bp, Artificial Sequence
  Description of Artificial Sequence Synthetic probe
ccaggagcaggtaggaggctcgc

SEQ ID NO: 12 - DNA, 25 bp, Artificial Sequence
  Description of Artificial Sequence Synthetic primer
tgcaggagacatctaactggcttct

SEQ ID NO: 13 - DNA, 26 bp, Artificial Sequence
  Description of Artificial Sequence Synthetic primer
catgctcctatgtatcactggagaga

SEQ ID NO: 14 - DNA, 14 bp, Artificial Sequence
  Description of Artificial Sequence Synthetic probe
agcaggctgacggc

SEQ ID NO: 15 - DNA, 22 bp, Artificial Sequence
  Description of Artificial Sequence Synthetic primer
ttgctcgttctgctcattcctt

SEQ ID NO: 16 - DNA, 20 bp, Artificial Sequence
  Description of Artificial Sequence Synthetic primer
gttgaggctggtccccaaca

SEQ ID NO: 17 - DNA, 17 bp, Artificial Sequence
  Description of Artificial Sequence Synthetic probe
ctcctccagtggacatg

SEQ ID NO: 18 - DNA, 22 bp, Artificial Sequence
  Description of Artificial Sequence Synthetic primer
ttgctcgttctgctcattcctt

SEQ ID NO: 19 - DNA, 18 bp, Artificial Sequence
  Description of Artificial Sequence Synthetic primer
ggcgcaggctgctgtatt

SEQ ID NO: 20 - DNA, 17 bp, Artificial Sequence
  Description of Artificial Sequence Synthetic probe
ctcctccagtggacatg

SEQ ID NO: 21 - DNA, 18 bp, Artificial Sequence
  Description of Artificial Sequence Synthetic primer
gatttggacctgcgagcg

SEQ ID NO: 22 - DNA, 18 bp, Artificial Sequence
  Description of Artificial Sequence Synthetic primer
gcggctgtctccacaagt

SEQ ID NO: 23 - DNA, 16 bp, Artificial Sequence
  Description of Artificial Sequence Synthetic probe
ctgacctgaaggctct

SEQ ID NO: 24 - DNA, 22 bp, Artificial Sequence
  Description of Artificial Sequence Synthetic primer
cctgagaaactgcaggagacat

SEQ ID NO: 25 - DNA, 22 bp, Artificial Sequence
  Description of Artificial Sequence Synthetic primer
gtgagtgccacagtctcatcat

SEQ ID NO: 26 - DNA, 24 bp, Artificial Sequence
  Description of Artificial Sequence Synthetic probe
caggacccctgtccagtgttagac

SEQ ID NO: 27 - DNA, 22 bp, Artificial Sequence
  Description of Artificial Sequence Synthetic primer
cctgagaaactgcaggagacat

SEQ ID NO: 28 - DNA, 22 bp, Artificial Sequence
  Description of Artificial Sequence Synthetic primer
gtgagtgccacagtctcatcat

SEQ ID NO: 29 - DNA, 30 bp, Artificial Sequence
  Description of Artificial Sequence Synthetic probe
ctatgtatcactggagagaggtcctggaac

SEQ ID NO: 30 - DNA, 20 bp, Artificial Sequence
  Description of Artificial Sequence Synthetic primer
agcctgtttccagctcaaag

SEQ ID NO: 31 - DNA, 20 bp, Artificial Sequence
  Description of Artificial Sequence Synthetic primer
gtcctaaggcctcacacctg

SEQ ID NO: 32 - DNA, 20 bp, Artificial Sequence
  Description of Artificial Sequence Synthetic probe
ccccggctgctgaactccat

SEQ ID NO: 33 - DNA, 20 bp, Artificial Sequence
  Description of Artificial Sequence Synthetic primer
catgtggaaactttgcttgc

SEQ ID NO: 34 - DNA, 23 bp, Artificial Sequence
  Description of Artificial Sequence Synthetic primer
ccttgttctatgtcagcacatcc

SEQ ID NO: 35 - DNA, 24 bp, Artificial Sequence
  Description of Artificial Sequence Synthetic probe
ttgttcccgtgttcctcactgtca

SEQ ID NO: 36 - DNA, 19 bp, Artificial Sequence
  Description of Artificial Sequence Synthetic primer
gtgcagcttgcttggtagc

SEQ ID NO: 37 - DNA, 20 bp, Artificial Sequence
  Description of Artificial Sequence Synthetic primer
gtaaattgccgagaccttgc

SEQ ID NO: 38 - DNA, 20 bp, Artificial Sequence
  Description of Artificial Sequence Synthetic probe
agcctaccccttggctcggg

SEQ ID NO: 39 - DNA, 21 bp, Artificial Sequence
  Description of Artificial Sequence Synthetic primer
cccctgatagtcacacagtcc

SEQ ID NO: 40 - DNA, 20 bp, Artificial Sequence
  Description of Artificial Sequence Synthetic primer
tggagttttgagggttttgg

SEQ ID NO: 41 - DNA, 22 bp, Artificial Sequence
  Description of Artificial Sequence Synthetic probe
tccgctgctgctctggcctcct

SEQ ID NO: 42 - DNA, 24 bp, Artificial Sequence
  Description of Artificial Sequence Synthetic primer
tcagcatgtacagacaggaataca

SEQ ID NO: 43 - DNA, 22 bp, Artificial Sequence
  Description of Artificial Sequence Synthetic primer
gagtgccacagtctcatcattg

SEQ ID NO: 44 - DNA, 24 bp, Homo sapiens
  CDS (1)..(24)
accccctgtccagtgttagacagg

SEQ ID NO: 45 - protein, 8 aa, Homo sapiens
TPCPVLDR

SEQ ID NO: 46 - protein, 8 aa, Homo sapiens
TLSPVIHR

SEQ ID NO: 47 - DNA, 24 bp, Homo sapiens
  CDS (1)..(24)
accctctctccagtgatacatagg